(19) United States
(12) Patent Application Publication
(10) Pub. No.: US 2024/0043488 A1
(43) Pub. Date: Feb. 8, 2024

(54) ACTIVATABLE IL-12 POLYPEPTIDES AND METHODS OF USE THEREOF

(71) Applicant: Werewolf Therapeutics, Inc., Cambridge, MA (US)

(72) Inventors: William WINSTON, West Newton, MA (US); Cynthia SEIDEL-DUGAN, Belmont, MA (US); Daniel HICKLIN, Boston, MA (US); Heather BRODKIN, West Newton, MA (US); Jose Andres SALMERON-GARCIA, Acton, MA (US); Philipp STEINER, Cambridge, MA (US)

(21) Appl. No.: 18/054,601

(22) Filed: Nov. 11, 2022

Related U.S. Application Data
(63) Continuation of application No. PCT/US2021/033014, filed on May 18, 2021.
(60) Provisional application No. 63/027,276, filed on May 19, 2020.

(51) Int. Cl. C07K 14/54; C07K 16/24; C12N 15/63; A61P 35/00

(52) U.S. Cl. CPC C07K 14/5434; C07K 16/244; C12N 15/63; A61P 35/00; C07K 2317/622; C07K 2319/31; C07K 2319/30

(57) ABSTRACT

Provided herein are IL-12 polypeptide complexes and/or IL23 polypeptide complexes comprising IL-12 or IL-23, a half-life extension element, an IL-12 or IL-23 blocking element and a protease cleavable linker. Also provided herein are pharmaceutical compositions thereof, as well as nucleic acids, recombinant expression vectors, host cells for making such polypeptide complexes. Also disclosed are methods of using the polypeptide complexes in the treatment of diseases, conditions and disorders.

The present application claims the benefit of U.S. Provisional Application No. 63/027,276 filed on May 19, 2020, which is incorporated herein by reference in its entirety.

### 1. SEQUENCE LISTING

The instant application contains a Sequence Listing which has been submitted electronically in ASCII format and is hereby incorporated by reference in its entirety. Said ASCII copy, created on May 18, 2021, is named 761146_02320_SL.txt and is 1,294,403 bytes in size.

### 2. BACKGROUND

Interleukin-12 (IL-12) is a heterodimeric 70 kDa cytokine composed of two covalently linked glycosylated subunits (p35 and p40) (Lieschke et al., 1997; Jana et al., 2014). It is a potent immune antagonist and has been considered a promising therapeutic agent for oncology. However, IL-12 has shown to have a narrow therapeutic window because they are highly potent and have a short serum half-life. Consequently, therapeutic administration of IL-12 produce undesirable systemic effects and toxicities. This is exacerbated by the need to administer large quantities of cytokines (i.e., IL-12) in order to achieve the desired levels of cytokine at the intended site of cytokine action (e.g., a tumor microenvironment). Unfortunately, due to the biology of cytokine and the inability to effectively target and control their activity, cytokines have not achieved the hoped for clinical advantages in the treatment in tumors.

Inducible IL-12 protein constructs have been described in International Application Nos. PCT/US2019/032320 and PCT/US2019/032322 to overcome the toxicity and short half-life problems that have limited clinical use of IL-12 in oncology. The previously described inducible IL-12 polypeptide constructs comprise a single polypeptide containing IL-12, a blocking element, and a half-life extension element.

The inventors of the present invention surprisingly found that an IL-12 polypeptide complex comprising two or more polypeptides have certain advantages, such as less aggregation and improved expression that result in higher yields.

### 3. SUMMARY

The disclosure relates to inducible IL-12 polypeptide complexes that contain an attenuated IL-12 and that have a long half-life in comparison to naturally occurring IL-12. If desired, the IL-12 can be a mutein. The IL-12 mutein can be aglycosylated or partially aglycosylated. The polypeptide complexes disclosed herein comprise two or more polypeptide chains, and the complex includes IL-12 subunits p35 and p40, a half-life extension element, an IL-12 blocking element and a protease cleavable linker.

The inducible IL-12 polypeptide complex can comprise two different polypeptides. The first polypeptide can comprise an IL-12 subunit, and optionally an IL-12 blocking element. The IL-12 blocking element when present is operably linked to the IL-12 subunit through a first protease cleavable linker. The second polypeptide chain can comprise an IL-12 subunit operably linked to a half-life extension element through a second protease cleavable linker, and optionally a IL-12 blocking element. The IL-12 blocking element when present can be operably linked to the IL-12 subunit through a protease cleavable linker or can be operably linked to the half-life extension element through a linker that is optionally protease cleavable. Only one of the first and second polypeptide contains the IL-12 blocking element. When the IL-12 subunit in the first polypeptide is p35, the IL-12 subunit in the second polypeptide is p40, and when the IL-12 subunit in the first polypeptide is p40, the IL-12 subunit in the second polypeptide is p35. A preferred blocking element of this complex is a single chain antibody that binds IL-12 or an antigen binding fragment thereof. The cleavable linkers in this complex can be the same or different.

The inducible IL-12 polypeptide complex can comprise three different polypeptides. Typically, one polypeptide chain comprises either the p35 or p40 IL-12 subunit, but not both, and a second polypeptide comprises the other IL-12 subunit and the third polypeptide comprises at least a portion (component) of the blocking element. The first polypeptide can comprise an IL-12 subunit, and optionally a half-life extension element. The half-life extension element when present is operably linked to the IL-12 subunit through a protease cleavable linker.

The second polypeptide can comprise a IL-12 subunit, at least an antigen binding portion of an antibody light chain or an antigen binding portion of an antibody heavy chain, and optionally a half-life extension element. When the half-life extension element is present, it is operably linked to the IL-12 subunit through a protease cleavable linker and the antibody heavy chain or light chain is either a) operably linked to the IL-12 subunit through a second protease cleavable linker, or b) operably linked to the half-life extension element through an optionally cleavable linker.

The third polypeptide can comprise can an antigen binding portion of an antibody heavy chain that is complementary to the light chain in the second polypeptide, or an antibody light chain that is complementary to the heavy chain in the second polypeptide and together with said light chain forms an IL-12 binding site. When the IL-12 subunit in the first polypeptide is p35, the IL-12 subunit in the second polypeptide is p40, and when the IL-12 subunit in the first polypeptide is p40, the IL-12 subunit in the second polypeptide is p35. In this complex, the IL-12 blocking element is preferably an antigen binding fragment of an antibody. The antigen binding fragment comprises as separate components, at least an antigen-binding portion of an antibody light chain and at least an antigen-binding portion of a complementary antibody heavy chain. The protease cleavable linkers in this inducible IL-12 polypeptide complex can be the same or different.

The inducible polypeptide complex can comprise two different polypeptides wherein p35 and p40 are located on the same polypeptide chain. A first polypeptide chain can comprise p35, p40, a half-life extension element and at least an antigen binding portion of an antibody light chain. p35 and p40 can be operably linked, and the half-life extension element can be operably linked to p40 through a first protease cleavable linker and the antigen binding portion of an antibody light chain can be operably linked to p35 through a protease cleavable linker. Alternatively, the half-life extension element can be operably linked to p35 through a protease cleavable linker and the antigen binding portion of an antibody light chain is operably linked to p40 through a protease cleavable linker. The second polypeptide comprises at least an antigen binding portion of an antibody heavy chain that is complementary to the light chain in the second polypeptide and together with said light chain forms and IL-12 binding site. The protease cleavable linkers in this complex can be the same or different.

In an alternative format, a first polypeptide chain can comprise p35, p40, a half-life extension element and at least an antigen binding portion of an antibody heavy chain. p35 and p40 can be operably linked, and the half-life extension element can be operably linked to p40 or through a protease cleavable linker and the antigen binding portion of an antibody heavy chain can be operably linked to p35 through a protease cleavable linker. Alternatively, the half-life extension element can be operably linked to p35 through a protease cleavable linker and the antigen binding portion of an antibody heavy chain can be operably linked to p40 through a second protease cleavable linker. A second polypeptide comprises at least an antigen binding portion of an antibody light chain that is complementary to the heavy chain in the second polypeptide and together with said light chain forms and IL-12 binding site. The protease cleavable linkers in this complex can be the same or different.

In one example, the IL-12 polypeptide complex comprises a first polypeptide does not comprise a blocking element and the second polypeptide has the formula: [A]-[L1]-[B]-[L3]-[D] or [D]-[L3]-[B]-[L1]-[A] or [B]-[L1]-[A]-[L2]-[D] or [D]-[L1]-[A]-[L2]-[B], wherein, A is the IL-12 subunit; L1 is the first protease-cleavable linker; L2 is the second protease cleavable linker; L3 is the optionally cleavable linker; B is the half-life extension element; and D is the blocking element.

In another example, the first polypeptide comprises the formula: [A]-[L1]-[D] or [D]-[L1]-[A]; and the second polypeptide has the formula: [A′]-[L2]-[B] or [B]-[L2]-[A′], wherein A is either p35 or p40, wherein when A is p35, A′ is p40 and when A is p40, A′ is p35; A′ is either p35 or p40; L1 is the first protease cleavable linker; L2 is the second protease cleavable linker; B is the half-life extension element; and D is the blocking element.

In embodiments, the IL-12 polypeptide complex comprises a first polypeptide selected from the group consisting of SEQ ID NOs: 95-110, SEQ ID NOs: 119-126, and SEQ ID NOs: 135-143, or an amino acid sequence that has at least 80% identity to SEQ ID NOs: 95-110, SEQ ID NOs: 119-126, and SEQ ID NOs: 135-143. A preferred IL-12 polypeptide complex comprises a first polypeptide comprising SEQ ID NO: 104 or SEQ ID NO: 136. A preferred IL-12 polypeptide complex comprises a first polypeptide chain comprising the amino acid sequence of SEQ ID NO: 104 and a second polypeptide chain comprising the amino acid sequence of SEQ ID NO: 18. Another preferred polypeptide complex comprises a first polypeptide chain comprising the amino acid sequence of SEQ ID NO: 136 and a second polypeptide chain comprising the amino acid sequence of SEQ ID NO: 18.

As described above, IL-12 can be a mutein, if desired. The IL-12 mutein retains IL-12 activity, for example intrinsic IL-12 receptor agonist activity. IL-12 subunits, p35 and/or p40 can be muteins. Preferably, the IL-12 mutein has an altered glycosylation pattern. For example, the IL-12 mutein can be partially aglycosylated or fully aglycosylated.

The p35 and/or the p40 subunits can contain one or more amino acid modifications, e.g., substitutions. For instance, the p35 and/or p40 subunits can comprise about one, about two, about three, about four, about five, about six, about seven or more amino acid substitutions. Although typically, p35 and/or p40 subunits contain about one to about seven amino acid substitutions. The substitutions can be a conservative substitution or a non-conservative substitution, but preferably is a conservative substitution. A typical modification alters the glycosylation pattern of the p35 and/or p40 subunit such that the p35 and/or p40 subunit is partially or fully aglycosylated. Preferably, the amino acid modification includes replacement of an asparagine amino acid. For example, asparagine to glutamine. In particular examples, asparagine at amino acid positions 16, 75, 85, 133, 151, 158, 201, 206, 221, 250, 267, 280, 282, 326, 400, 404, 425, 555, 572, 575, 582, or 602 on IL-12 p35 of SEQ ID NO: 434 can be mutated. In particular examples, asparagine at amino acid positions 103, 114, 163, 219, 227, or 282 of IL-12 p40 of SEQ ID NO: 18 can be mutated.

For example, a partially or fully aglycosylated IL-12 polypeptide can comprise a polypeptide selected from the group consisting of SEQ ID NOs: 104, 434 or 442-445, or an amino acid sequence that has at least 80% identity to SEQ ID NOs: 104, 434 or 442-445.

The disclosure also relates to single chain IL-12 inducible polypeptides. The single chain IL-12 polypeptide preferably comprises the amino acid selected from the group consisting of SEQ ID NOs: 7, 9, 10, 18, 24-94, SEQ ID NOs: 110-118, and SEQ ID NOs: 127-134, or an amino acid sequence that has at least about 80% identity to SEQ ID NOs: 7, 9, 10, 18, 24-94, SEQ ID NOs: 110-118, and SEQ ID NOs: 127-134.

The disclosure also relates to inducible IL-23 polypeptide complexes that contain an attenuated IL-23 and that have a long half-life in comparison to naturally occurring IL-23. If desired, the IL-23 can be a mutein. The IL-23 mutein can be aglycosylated or partially aglycosylated. The polypeptide complexes disclosed herein comprise one or more polypeptide chains, and the complex includes IL-23 subunits p19 and p40, a half-life extension element, an IL-23 blocking element and a protease cleavable linker.

The inducible IL-23 polypeptide complex can comprise two different polypeptides. The first polypeptide can comprise an IL-23 subunit, and optionally an IL-23 blocking element. The IL-23 blocking element when present is operably linked to the IL-23 subunit through a first protease cleavable linker. The second polypeptide chain can comprise an IL-23 subunit operably linked to a half-life extension element through a second protease cleavable linker, and optionally a IL-23 blocking element. The IL-23 blocking element when present can be operably linked to the IL-23 subunit through a protease cleavable linker or can be operably linked to the half-life extension element through a linker that is optionally protease cleavable. Only one of the first and second polypeptide contains the IL-23 blocking element. When the IL-23 subunit in the first polypeptide is p19 the IL-23 subunit in the second polypeptide is p40, and when the IL-23 subunit in the first polypeptide is p40, the IL-23 subunit in the second polypeptide is p40. A preferred blocking element of this complex is a single chain antibody that binds IL-23 or an antigen binding fragment thereof. The cleavable linkers in this complex can be the same or different.

The inducible IL-23 polypeptide complex can comprise three different polypeptides. Typically, one polypeptide chain comprises either the p19 or p40 IL-23 subunit, but not both, and a second polypeptide comprises the other IL-23 subunit and the third polypeptide comprises at least a portion (component) of the blocking element. The first polypeptide can comprise an IL-23 subunit, and optionally a half-life extension element. The half-life extension element when present is operably linked to the IL-23 subunit through a protease cleavable linker.

The second polypeptide can comprise a IL-23 subunit, at least an antigen binding portion of an antibody light chain or an antigen binding portion of an antibody heavy chain, and optionally a half-life extension element. When the half-life extension element is present, it is operably linked to the IL-23 subunit through a protease cleavable linker and the antibody heavy chain or light chain is either a) operably linked to the IL-23 subunit through a second protease cleavable linker, or b) operably linked to the half-life extension element through an optionally cleavable linker.

The third polypeptide can comprise can an antigen binding portion of an antibody heavy chain that is complementary to the light chain in the second polypeptide, or an antibody light chain that is complementary to the heavy chain in the second polypeptide and together with said light chain forms and IL-23 binding site. When the IL-23 subunit in the first polypeptide is p19, the IL-23 subunit in the second polypeptide is p40, and when the IL-23 subunit in the first polypeptide is p40, the IL-23 subunit in the second polypeptide is p19. In this complex, the IL-23 blocking element is preferably an antigen binding fragment of an antibody. The antigen binding fragment comprises as separate components, at least an antigen-binding portion of an antibody light chain and at least an antigen-binding portion of a complementary antibody heavy chain. The protease cleavable linkers in this inducible IL-23 polypeptide complex can be the same or different.

The inducible polypeptide complex can comprise two different polypeptides wherein p19 and p40 are located on the same polypeptide chain. A first polypeptide chain can comprise p19, p40, a half-life extension element and at least an antigen binding portion of an antibody light chain. p19 and p40 can be operably linked, and the half-life extension element can be operably linked to p40 through a first protease cleavable linker and the antigen binding portion of an antibody light chain can be operably linked to p19 through a protease cleavable linker. Alternatively, the half-life extension element can be operably linked to p19 through a protease cleavable linker and the antigen binding portion of an antibody light chain is operably linked to p40 through a protease cleavable linker. The second polypeptide comprises at least an antigen binding portion of an antibody heavy chain that is complementary to the light chain in the second polypeptide and together with said light chain forms and IL-23 binding site. The protease cleavable linkers in this complex can be the same or different.

In an alternative format, a first polypeptide chain can comprise p19, p40, a half-life extension element and at least an antigen binding portion of an antibody heavy chain. P19 and p40 can be operably linked, and the half-life extension element can be operably linked to p40 or a through a protease cleavable linker and the antigen binding portion of an antibody heavy chain can be operably linked to p19 through a protease cleavable linker. Alternatively, the half-life extension element can be operably linked to p19 through a protease cleavable linker and the antigen binding portion of an antibody heavy chain can be operably linked to p40 through a second protease cleavable linker. A second polypeptide comprises at least an antigen binding portion of an antibody light chain that is complementary to the heavy chain in the second polypeptide and together with said light chain forms and IL-23 binding site. The protease cleavable linkers in this complex can be the same or different.

In one example, the IL-23 polypeptide complex comprises a first polypeptide does not comprise a blocking element and the second polypeptide has the formula: [A]-[L1]-[B]-[L3]-[D] or [D]-[L3]-[B]-[L1]-[A] or [B]-[L1]-[A]-[L2]-[D] or [D]-[L1]-[A]-[L2]-[B], wherein, A is the IL-23 subunit; L1 is the first protease-cleavable linker; L2 is the second protease cleavable linker; L3 is the optionally cleavable linker; B is the half-life extension element; and D is the blocking element.

In another example, the first polypeptide comprises the formula: [A]-[L1]-[D] or [D]-[L1]-[A]; and the second polypeptide has the formula: [A′]-[L2]-[B] or [B]-[L2]-[A′], wherein A is either p19 or p40, wherein when A is p19, A′ is p40 and when A is p40, A′ is p19; A′ is either p19 or p40; L1 is the first protease cleavable linker; L2 is the second protease cleavable linker; B is the half-life extension element; and D is the blocking element.

In embodiments, the IL-23 polypeptide complex comprises a first polypeptide selected from the group consisting of SEQ ID NOs: 423-428, or an amino acid sequence that has at least 80% identity to SEQ ID NOs: 423-428. In embodiments, the IL-23 polypeptide complex comprises a second polypeptide selected from the group consisting of SEQ ID NOs: 18 or 433.

As described above, the IL-23 can be a mutein, if desired. The IL-23 mutein retains IL-23 activity, for example intrinsic IL-23 receptor agonist activity. IL-23 subunits, p19 and/or p40 can be muteins. Preferably, the IL-23 mutein has an altered glycosylation pattern. For example, the IL-23 mutein can be partially aglycosylated or fully aglycosylated.

The p19 and/or the p40 subunits can contain one or more amino acid modifications, e.g., substitutions. For instance, the p19 and/or p40 subunits can comprise about one, about two, about three, about four, about five or more amino acid substitutions. Although typically, p19 and/or p40 subunits contain one or two amino acid substitutions. The substitutions can be a conservative substitution or a non-conservative substitution, but preferably is a conservative substitution. A typical modification alters the glycosylation pattern of the p19 and/or p40 subunit such that the p19 and/or p40 subunit is partially or fully aglycosylated. Preferably, the amino acid modification includes replacement of an asparagine amino acid. For example, asparagine to glutamine.

The disclosure also relates to single chain IL-23 inducible polypeptides. The single chain IL-23 polypeptide preferably comprises the amino acid selected from the group consisting of SEQ ID NOs: 422 or 429-432, or an amino acid sequence that has at least about 80% identity to SEQ ID NOs: 422 or 429-432.

The half-life extension element disclosed herein is preferably human serum albumin, an antigen binding polypeptide that binds human serum albumin, or an immunoglobulin Fc or fragment thereof.

The protease cleavable linker comprises a sequence that is capable of being cleaved by a protease selected from kallikrein, thrombin, chymase, carboxypeptidase A, cathepsin, elastase, PR-3, granzyme M, a calpain, a matrix metalloproteinase (MMP), an ADAM, a FAP, a plasminogen activator, a caspase, a tryptase, or a tumor protease. The protease is preferably selected from cathepsin B, cathepsin C, cathepsin D, cathepsin E, cathepsin K, cathepsin L, or cathepsin G. Alternatively, the protease is preferably selected from matrix metalloprotease (MMP) is MMP1, MMP2, MMP3, MMP8, MMP9, MMP10, MMP11, MMP12, MMP13, or MMP14.

In embodiments, the protease cleavable linker comprises at least two sequences that are independently capable of being cleaved by a protease. The protease cleavable linker can comprise a synthetic sequence. In embodiments, each of the protease cleavable linkers are cleaved by two or more different proteases.

The blocking element described herein can be any element that binds to IL-12 or IL-23. The blocking element disclosed herein can bind to p35, p40, or the p35p40 heterodimeric complex. The blocking element disclosed herein can bind to p19, p40, or the 19p40 heterodimeric complex. The blocking element is preferably a single chain variable fragment (scFv) or a Fab.

The disclosure also relates to nucleic acids encoding the IL-12 polypeptide complexes described herein. The disclosure also relates to nucleic acids encoding the IL-23 polypeptide complexes described herein. The nucleic acid composition encoding an IL-12 polypeptide complex or an IL-23 polypeptide complex described herein can comprise a circular vector, DNA, or RNA. Also provided herein is an expression vector comprising the nucleic acid encoding an IL-12 polypeptide complex or an IL-23 polypeptide complex as described herein. In embodiments, provided herein is a host cell comprises the vector. The disclosure also relates to methods of making a pharmaceutical composition, comprising culturing the isolated host cell under suitable conditions for expression of the polypeptide complex.

Also provided herein are pharmaceutical compositions comprising an IL-12 polypeptide complex as disclosed herein. Also provided herein are pharmaceutical compositions comprising an IL-23 polypeptide complex.

The disclosure also relates to methods for treating a tumor, comprising administering to a subject in need thereof an effective amount of the IL-12 polypeptide complex disclosed herein, a nucleic acid encoding the IL-12 polypeptide complex, or a pharmaceutical composition thereof. The disclosure also relates to methods for treating a tumor, comprising administering to a subject in need thereof an effective amount of the IL-23 polypeptide complex disclosed herein, a nucleic acid encoding the IL-23 polypeptide complex, or pharmaceutical compositions thereof. Any suitable tumor can be treated according to the methods disclosed herein, for example, melanoma or breast cancer.

### 4. BRIEF DESCRIPTION OF THE DRAWINGS

The drawings are not necessarily to scale or exhaustive. Instead, the emphasis is generally placed upon illustrating the principles of the inventions described herein. The accompanying drawings, which constitute a part of the specification, illustrate several embodiments consistent with the disclosure and, together with the description, serve to explain the principles of the disclosure. In the drawings:

FIGS. 1A-1J is a schematic illustration depicting various inducible IL-12 complexes that contain two or three polypeptide chains.

FIGS. 2A-2S are a series of graphs showing activity of fusion protein heterodimers in an HEKBlue IL-12 reporter assay. IL-12/STAT4 activation by heterodimeric IL-12 polypeptides in comparison to chimeric IL-12 (mouse p35/human p40)) or recombinant IL-12 (controls). Squares depict IL-12 activity of uncut inducible heterodimers and triangles depict the IL-12 activity of cut heterodimers. Circles depict activity of the control. EC50 values for each are shown in the table.

FIGS. 3A-3F are a series of graphs showing activity of fusion protein heterodimers in an IL-12 luciferase reporter assay. Activation of IL-12 signaling of heterodimeric IL-12 polypeptides in comparison to recombinant human IL-12 (control) is depicted. Closed squares depict activity of the uncut inducible heterodimeric IL-12 polypeptide (intact) and open squares depict the activity of the cut inducible heterodimer (cleaved). Circles depict activity of the control recombinant human IL-12. EC50 values for each are shown in the table.

FIGS. 4A-4G are a series of graphs showing activity of fusion protein heterodimers in an IL-12 T-Blast Assay. Activation of IL-12 signaling by heterodimeric IL-12 polypeptides in comparison to IL-12 (control) is depicted. Squares depict activity of the uncut inducible heterodimeric IL-12 polypeptide (intact) and triangles depict the activity of the cut inducible heterodimeric IL-12 polypeptide. Circles depict activity of the control (IL-12). EC50 values are shown in the table.

FIG. 5 is a series of SDS-PAGE gels comparing WW0663 (SEQ ID NO: 18) (a single polypeptide chain in which the IL-12 subunits are connected using a linker that was designed to be uncleavable) and that were produced in a mammalian host cell line and purified by Protein A chromatography. Reduced and Non-Reduced conditions are compared. The analysis showed unintended cleavage of WW0663 at or near the linker that connected p35 and p40. In contrast, the heterodimer WW0750/WW0636 showed only the intended product when produced in the same mammalian host cell line.

FIG. 6 is a graph showing results of analyzing WW0749/636 in a syngeneic MC38 mouse tumor model. It shows average tumor volume over time in mice treated with 43 μg WW0749/636 (triangle), 170 μg WW0749/636 (upside-down triangle), 340 μg WW0749/636 (diamond), and 510 μg WW0749/636 (square). Vehicle alone is indicated by circle.

FIG. 7A-7E shows a series of spider plots showing activity of inducible IL-12 fusion proteins in an MC38 mouse xenograft model corresponding to the data shown in FIG. 6. Each line in the plots is the tumor volume over time for a single mouse.

FIG. 8 is a graph showing results of analyzing WW0749/636 in a syngeneic MC38 mouse tumor model. It shows average percent body weight over time in mice treated with 43 μg WW0749/636 (triangle), 170 μg WW0749/636 (upside-down triangle), 340 μg WW0749/636 (diamond), and 510 μg WW0749/636 (square). Vehicle alone is indicated by circle.

FIGS. 9A-9E show a series of spider plots showing the impact of inducible IL-12 fusion protein (WW0749/636) on body weight in an MC38 mouse xenograft model corresponding to the data shown in FIG. 8. Each line in the plots is the body weight over time for a single mouse.

FIG. 10 is a graph showing results of analyzing WW0751/636 in a syngeneic MC38 mouse tumor model. It shows average tumor volume over time in mice treated with 43 μg WW0751/636 (triangle), 170 μg WW0751/636 (upside-down triangle), 340 μg WW0751/636 (diamond), and 510 μg WW0751/636 (square). Vehicle alone is indicated by circle. The data show tumor volume decreasing over time in mice treated with WW0751/636 at all concentrations.

FIGS. 11A-11E show a series of spider plots showing activity of fusion protein (WW0751/636) in an MC38 mouse xenograft model corresponding to the data shown in FIG. 10. Each line in the plots is the tumor volume over time for a single mouse.

FIG. 12 is a graph showing results of analyzing WW0751/636 in a syngeneic MC38 mouse tumor model. It shows average percent body weight over time in mice treated with 43 μg WW0751/636 (triangle), 170 μg WW0751/636 (upside-down triangle), 340 μg WW0751/636 (diamond), and 510 μg WW0751/636 (square). Vehicle alone is indicated by circle.

FIGS. 13A-13E show a series of spider plots showing the impact of fusion proteins on body weight in an MC38 mouse xenograft model corresponding to the data shown in FIG. 12. Each line in the plots is the body weight over time for a single mouse.

FIG. 14 is a graph showing results of analyzing WW0753/636/727 in a syngeneic MC38 mouse tumor model. It shows average tumor volume over time in mice treated with 52 μg WW0753/636/727 (triangle), 207 μg WW0753/636/727 (upside-down triangle), 414 μg WW0753/636/727 (diamond), and 621 μg WW0753/636/727 (square). Vehicle alone is indicated by circle. The data show tumor volume decreasing over time in a dose-dependent manner in mice treated with WW0753/636/727 at higher concentrations.

FIG. 15A-15E shows a series of spider plots showing activity of fusion protein (WW0753/636/727) in an MC38 mouse xenograft model corresponding to the data shown in FIG. 14. Each line in the plots is the tumor volume over time for a single mouse.

FIG. 16 is a graph showing results of analyzing WW0753/636/727 in a syngeneic MC38 mouse tumor model. It shows average percent body weight over time in mice treated with 52 μg WW0753/636/727 (triangle), 207 μg WW0753/636/727 (upside-down triangle), 414 μg WW0753/636/727 (diamond), and 621 μg WW0753/636/727 (square). Vehicle alone is indicated by circle.

FIG. 17A-17E show a series of spider plots showing the impact of fusion protein (WW0753/636/727) on body weight in an MC38 mouse xenograft model corresponding to the data shown in FIG. 16. Each line in the plots is the body weight over time for a single mouse.

FIG. 18 is a graph showing results of analyzing WW0755/636/727 in a syngeneic MC38 mouse tumor model. It shows average tumor volume over time in mice treated with 52 μg WW0753/636/727 (triangle), 207 μg WW0755/636/727 (upside-down triangle), 414 μg WW0755/636/727 (diamond), and 621 μg WW0755/636/727 (square). Vehicle alone is indicated by circle.

FIG. 19A-19E shows a series of spider plots showing activity of fusion protein (WW0755/636/727) in an MC38 mouse xenograft model corresponding to the data shown in FIG. 18. Each line in the plots is the tumor volume over time for a single mouse.

FIG. 20 is a graph showing results of analyzing WW0755/636/727 in a syngeneic MC38 mouse tumor model. It shows average percent body weight over time in mice treated with 52 μg WW0755/636/727 (triangle), 207 μg WW0755/636/727 (upside-down triangle), 414 μg WW0755/636/727 (diamond), and 621 μg WW0753/636/727 (square). Vehicle alone is indicated by circle.

FIG. 21A-21E show a series of spider plots showing the impact of fusion protein (WW0755/636/727) on body weight in an MC38 mouse xenograft model corresponding to the data shown in FIG. 20. Each line in the plots is the body weight over time for a single mouse.

FIG. 22 is a graph showing results of analyzing WW0749/636 in a syngeneic MC38 mouse tumor model. It shows average tumor volume over time in mice treated with 3.5 μg WW0749/636 (diamond), 14 μg WW0749/636 (square), and 43 μg WW0749/636 (blue circle). Vehicle alone is indicated by black circle.

FIGS. 23A-23D show a series of spider plots showing activity of fusion protein (WW0749/636) in an MC38 mouse xenograft model corresponding to the data shown in FIG. 22. Each line in the plots is the tumor volume over time for a single mouse.

FIG. 24 is a graph showing results of analyzing WW0749/636 in a syngeneic MC38 mouse tumor model. It shows average percent body weight over time in mice treated with 3.5 μg WW0749/636 (diamond), 14 μg WW0749/636 (square), and 43 μg WW0749/636 (blue circle). Vehicle alone is indicated by black circle.

FIGS. 25A-25D show a series of spider plots showing the impact of fusion protein (WW0749/636) on body weight in an MC38 mouse xenograft model corresponding to the data shown in FIG. 24. Each line in the plots is the body weight over time for a single mouse.

FIG. 26 is a graph showing results of analyzing WW0753/636/727 in a syngeneic MC38 mouse tumor model. It shows average tumor volume over time in mice treated with 4.3 μg WW0753/636/727 (diamond), 17 μg WW0753/636/727 (square), and 52 μg WW0753/636/727 (blue circle). Vehicle alone is indicated by black circle.

FIGS. 27A-27D show a series of spider plots showing activity of fusion protein (WW0753/636/727) in an MC38 mouse xenograft model corresponding to the data shown in FIG. 26. Each line in the plots is the tumor volume over time for a single mouse.

FIG. 28 is a graph showing results of analyzing WW0753/636/727 in a syngeneic MC38 mouse tumor model. It shows average percent body weight over time in mice treated with 4.3 μg WW0753/636/727 (diamond), 17 μg WW0753/636/727 (square), and 52 μg WW0753/636/727 (blue circle). Vehicle alone is indicated by black circle.

FIG. 29A-29D shows a series of spider plots showing the impact of fusion protein (WW0753/636/727) on body weight in an MC38 mouse xenograft model corresponding to the data shown in FIG. 28. Each line in the plots is the body weight over time for a single mouse.

FIG. 30 is a graph showing results of analyzing WW0757/636 in a syngeneic MC38 mouse tumor model. It shows average tumor volume over time in mice treated with 14 μg WW0757/636 (diamond), 43 μg WW0757/636 (square), 86 μg WW0757/636 (circle), 170 μg WW0757/636 (up triangle), 510 μg WW0757/636 (down triangle), 765 μg WW0757/636 (star), and 1,020 μg WW0757/636 (asterix). Vehicle alone is indicated by circle.

FIGS. 31A-31H show a series of spider plots showing activity of fusion protein (WW0757/636) in an MC38 mouse xenograft model corresponding to the data shown in FIG. 30. Each line in the plots is the tumor volume over time for a single mouse. WW0757/636 at 1,020 μg had two dosing holidays on Day 7 and Day 11 due to poor tolerability.

FIG. 32 is a graph showing results of analyzing WW0757/636 in a syngeneic MC38 mouse tumor model. It shows average percent body weight over time in mice treated with 14 μg WW0757/636 (diamond), 43 μg WW0757/636 (square), 86 μg WW0757/63 6 (circle), 170 μg WW0757/636 (up triangle), 510 μg WW0757/636 (down triangle), 765 μg WW0757/636 (star), and 1,020 μg WW0757/636 (asterix). Vehicle alone is indicated by black circle.

FIGS. 33A-33H show a series of spider plots showing the impact of fusion protein (WW0757/636) on body weight in an MC38 mouse xenograft model corresponding to the data shown in FIG. 31. Each line in the plots is the body weight over time for a single mouse.

FIG. 34 is a graph showing results of analyzing WW0804/636 in a syngeneic MC38 mouse tumor model. It shows average tumor volume over time in mice treated with 42 μg WW0804/636 (diamond), 168 μg WW0804/636 (square), 505 μg WW0804/636 (circle), 757 μg WW0804/636 (up triangle), and 1,010 μg WW0804/636 (down triangle). Vehicle alone is indicated by circle.

FIGS. 35A-35F show a series of spider plots showing activity of fusion protein (WW0804/636) in an MC38 mouse xenograft model corresponding to the data shown in FIG. 33. Each line in the plots is the tumor volume over time for a single mouse. WW0804/636 at 767 μg and 1,020 μg had a dosing holidays on Day 11 due to poor tolerability.

FIG. 36 is a graph showing results of analyzing WW0804/636 in a syngeneic MC38 mouse tumor model. It shows average percent body weight over time in mice treated with 42 μg WW0804/636 (diamond), 168 μg WW0804/636 (square), 505 μg WW0804/636 (circle), 757 μg WW0804/636 (up triangle), and 1,010 μg WW0804/636 (down triangle). Vehicle alone is indicated by black circle.

FIG. 37A-37F shows a series of spider plots showing the impact of fusion protein (WW0804/636) on body weight in an MC38 mouse xenograft model corresponding to the data shown in FIG. 35. Each line in the plots is the body weight over time for a single mouse. WW0804/636 at 757 μg and 1,010 μg had a dosing holiday on Days 11, respectively.

FIG. 38 is an image of SDS-PAGE gel of aglycosylated IL-12 polypeptide constructs. The gel shows WW0924 (SEQ ID NO: 442)/WW0925 (SEQ ID NO: 443) in the first column. The gel shows WW0935 (SEQ ID NO: 444)/WW0936 (SEQ ID NO: 445) in the second column. The gel shows WW0924 (SEQ ID NO: 442)/WW0636 (SEQ ID NO: 18) in the third column. The gel shows WW0758 (SEQ ID NO: 104)/WW0925 (SEQ ID NO: SEQ ID NO: 443) in the fourth column.

FIGS. 39A-39D show a series of graphs from a SEC analysis of aglycosylated IL-12 polypeptide constructs derived from CHO cells. FIG. 39A depicts fully aglycosylated WW0924 (SEQ ID NO: 442)/WW0925 (SEQ ID NO: 443). FIG. 39B depicts partially aglycosylated WW0935 (SEQ ID NO: 444)/WW0936 (SEQ ID NO: 445). FIG. 39C depicts fully aglycosylated WW0924 (SEQ ID NO: 442)/WW0636 (SEQ ID NO: 18). FIG. 39D depicts fully WW0758 (SEQ ID NO: 104)/WW0925 (SEQ ID NO: SEQ ID NO: 443).

FIGS. 40A and 40B are a series of graphs showing activity of fusion proteins in an HEKBlue IL23 reporter assay. FIG. 40A depicts IL-23/STAT3 activation in a comparison of WW50009 (a half-life extended mouse IL23 fusion protein (squares)) to mouse IL23 (control (circles)) in the absence of albumin. FIG. 40B depicts IL-23/STAT3 activation in a comparison of WW50009 (a half-life extended mouse IL23 fusion protein (squares)) to mouse IL23 (control (circles)) in the presence of albumin. EC50 values for each are shown in the tables. Analysis was performed based on quantification of Secreted Alkaline Phosphatase (SEAP) activity using the reagent QUANTI-Blue® (InvivoGen). Results confirm that half-life extended mouse IL23 fusion protein is active, independent of the presence of albumin.

FIG. 41 is a graph showing results of analyzing WW0757/636 in a syngeneic CT26 mouse tumor model. It shows average tumor volume over time in mice treated with 50 μg WW0757/636 (diamond) and 100 μg WW0757/636 (square). Vehicle alone is indicated by circle. The data show tumor volume increased inhibited over time in a dose-dependent manner in mice treated with WW0757/636 at the higher concentrations.

FIGS. 42A-42C shows a series of spider plots showing activity of fusion proteins in a CT26 mouse xenograft model corresponding to the data shown in FIG. 41. Each line in the plots is the tumor volume over time for a single mouse.

FIG. 43 is a graph showing results of analyzing WW0757/636 in a syngeneic B16F10 mouse tumor model. It shows average tumor volume over time in mice treated with 50 μg WW0757/636 (diamond) and 100 μg WW0757/636 (square). Vehicle alone is indicated by circle. The data show tumor volume increased inhibited over time in a dose-dependent manner in mice treated with WW0757/636 at the higher concentrations.

FIGS. 44A-44C shows a series of spider plots showing activity of fusion proteins in a B16F10 mouse xenograft model corresponding to the data shown in FIG. 43. Each line in the plots is the tumor volume over time for a single mouse.

FIG. 45 is a graph showing results of analyzing WW0757/636 in a syngeneic EMT6 mouse tumor model. It shows average tumor volume over time in mice treated with 50 μg WW0757/636 (diamond) and 100 μg WW0757/WW0636 (square). Vehicle alone is indicated by circle. The data show tumor volume increased inhibited over time in a dose-dependent manner in mice treated with WW0757/WW0636 at the higher concentrations.

FIGS. 46A-46C shows a series of spider plots showing activity of fusion proteins in a EMT6 mouse xenograft model corresponding to the data shown in FIG. 45. Each line in the plots is the tumor volume over time for a single mouse.

FIGS. 47A-47I are a series of graphs depicting the immune profiling and nanaostring analysis of MC38 mouse tumor extracts treated with WW0757/WW0636. FIGS. 47A-47C show that IFNg production by total CD8+ T Cells, Tetramer+CD8+ T cells, and NK cells was increased. FIGS. 47D and 47E show that CD25 and Tbet expression by Tetramer+CD8+ T cells were activated. FIGS. 47F-47I show CD25, Tbet, IFNg, and TNF production by CD4+ NonTregs. P values represent an unpaired students T test. *=p<0.05; **=p<0.01; ***=p<0.001; ****=p<0.0001.

FIGS. 48A-48H are a series of graphs that show IL-12 polypeptide complex WW0757/WW0636 drives a transcriptional shift towards immune activation. FIG. 48A shows a heatmap analysis of statistically significant changes in transcript expression between vehicle and WW0757/WW000636 treated animals. FIGS. 48B-48E shows pathway scoring analysis of the differences in interferon signaling (FIG. 48B), and immune cell functions (FIGS. 48C-48E) between vehicle and WW0757/0636 treated tumors. FIGS. 48F-48H shows the pathway scoring analysis of the differences in dendritic cell function between vehicle and WW0757/0636 treated tumors.

FIGS. 49A-49B is a graph showing results of analyzing WW5009 in a syngeneic MC38 mouse tumor model. FIG. 49A shows average tumor volume over time in mice treated with 1 μg WW5009 (closed circles), 10 μg WW5009 (squares) and 100 μg WW5009 (stars). Vehicle alone is indicated by open circles. The data show tumor volume decreasing over time in the 2 top dose groups of 10 and 100 μg. FIG. 49B shows the impact of WW5009 dosing on the average body weight of the animals.

FIGS. 50A-50D are a series of spider plots showing activity of WW5009 in an MC38 mouse xenograft model corresponding to the data shown in FIGS. 49A-49B. Each line in the plots is the tumor volume over time for a single mouse.

### 5. DETAILED DESCRIPTION

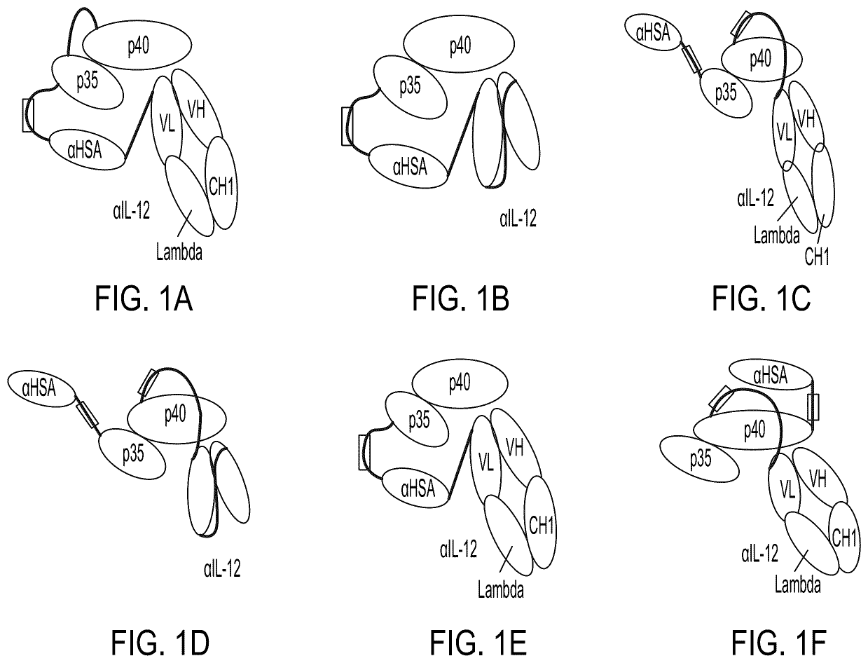

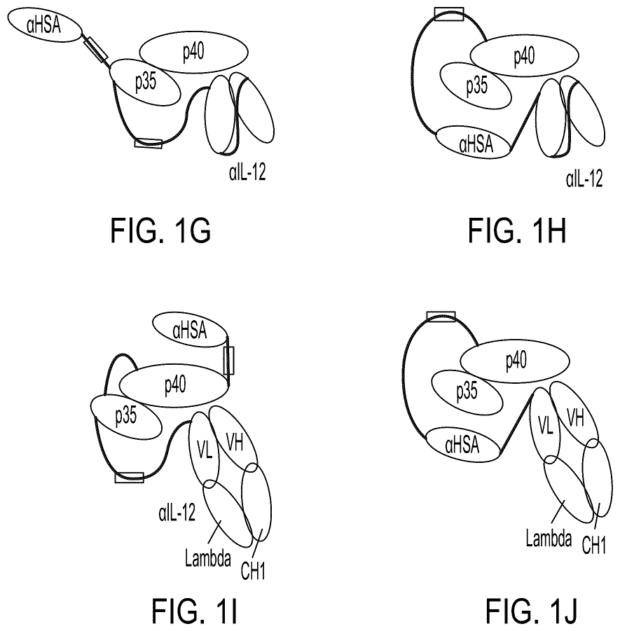

The disclosure relates to inducible IL-12 polypeptide complexes that contain an attenuated IL-12 and that have a long half-life in comparison to naturally occurring IL-12. The IL-12 polypeptide complexes disclosed herein comprise two or more polypeptide chains, and the complex includes IL-12 subunits p35 and p40, a half-life extension element, an IL-12 blocking element and a protease cleavable linker. The activity of IL-12 (e.g., receptor binding activity and/or receptor agonist activity) in the complex is attenuated by the action of the blocking element, which is tethered to the complex by a protease cleavable linker. Upon cleavage of the protease cleavable linker(s), the blocking element and the half-life extension element are separated from IL-12 and can diffuse away from the IL-12, producing active IL-12. That active IL-12 typically has biological activity and half-life that is substantially similar to naturally occurring IL-12. FIGS. 1A-1J depict non-limiting examples of IL-12 polypeptide complexes, as disclosed herein. This disclosure further relates to pharmaceutical compositions that contain the inducible IL-12 polypeptide complexes, as well as nucleic acids that encode the polypeptides, and recombinant expression vectors and host cells for making such polypeptides and complexes. Also provided herein are methods of using the disclosed IL-12 polypeptide complexes in the treatment of diseases, conditions, and disorders.

The IL-12 polypeptide complex disclosed herein overcomes toxicity and short half-life problems that have severely limited the clinical use of IL-12, particularly in the field of oncology. The IL-12 polypeptide complex comprises IL-12 polypeptides that have receptor agonist activity. But in the context of the IL-12 polypeptide complex, the IL-12 receptor agonist activity is attenuated, and the circulating half-life is extended.

The IL-12 polypeptide complexes disclosed herein contain at least two polypeptide chains and can contain three or more polypeptide chains if desired.

The disclosure also relates to inducible IL-23 polypeptide complexes that contain an attenuated IL-23 and that have a long half-life in comparison to naturally occurring IL-23.

The IL-23 polypeptide complexes disclosed herein comprise one or more polypeptide chains, and the complex includes IL-23 subunits p19 and p40, a half-life extension element, an IL-23 blocking element and a protease cleavable linker. The activity of IL-23 (e.g., receptor binding activity and/or receptor agonist activity) in the complex is attenuated by the action of the blocking element, which is tethered to the complex by a protease cleavable linker. Upon cleavage of the protease cleavable linker(s), the blocking element and the half-life extension element are separated from IL-23 and can diffuse away from the IL-23, producing active IL-23. That active IL-23 typically has biological activity and half-life that is substantially similar to naturally occurring IL-23. This disclosure further relates to pharmaceutical compositions that contain the inducible IL-23 polypeptide complexes, as well as nucleic acids that encode the polypeptides, and recombinant expression vectors and host cells for making such polypeptides and complexes. Also provided herein are methods of using the disclosed IL-23 polypeptide complexes in the treatment of diseases, conditions, and disorders.

The IL-23 polypeptide complex disclosed herein overcomes toxicity and short half-life problems that have severely limited the clinical use of IL-23, particularly in the field of oncology. The IL-23 polypeptide complex comprises IL-23 polypeptides that have receptor agonist activity, but in the context of the IL-23 polypeptide complex, the IL-23 receptor agonist activity is attenuated, and the circulating half-life is extended.

The IL-23 polypeptide complexes disclosed herein contain at least one polypeptide chain, and can contain two or more polypeptide chains, if desired.

Certain illustrative and preferred embodiments are described in detail herein. The embodiments within the specification should not be construed to limit the scope of the disclosure.

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

As used herein, the terms “activatable,” “activate,” “induce,” and “inducible” refers to a polypeptide complex that has an attenuated activity form (e.g., attenuated receptor binding and/or agonist activity) and an activated form. The polypeptide complex is activated by protease cleavage of the linker that causes the blocking element and half-life extension element to dissociate from the polypeptide complex. The induced/activated polypeptide complex can bind with increased affinity/avidity to the IL-12 receptor. The induced/activated polypeptide complex can bind with increased affinity/avidity to the IL-23 receptor.

The terms “antibody” and “immunoglobulin” are used interchangeably herein. An antibody or immunoglobulin, as used herein, is intended to refer to immunoglobulin molecules comprised of two heavy (H) chains. Typically, antibodies in mammals (e.g., humans, rodents, and monkey's) comprise four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CHI, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. Antibodies can include, for example, monoclonal antibodies, recombinantly produced antibodies, monospecific antibodies, multi specific antibodies (including bispecific antibodies), human antibodies, humanized antibodies, chimeric antibodies, immunoglobulins, synthetic antibodies, or tetrameric antibodies comprising two heavy chain and two light chain molecules. One of skill in the art would recognize that other forms of antibodies exist (e.g. camelid and shark antibodies).

The term “attenuated” as used herein is an IL-12 receptor agonist or an IL-23 receptor agonist that has decreased receptor agonist activity as compared to the IL-12 receptor's or IL-23 receptor's naturally occurring agonist. An attenuated IL-12 agonist or an attenuated IL-23 agonist can have at least about 10×, at least about 50×, at least about 100×, at least about 250×, at least about 500×, at least about 1000× or less agonist activity as compared to the receptor's naturally occurring agonist. When a IL-12 polypeptide complex that contains IL-12 as described herein is described as “attenuated” or having “attenuated activity”, it is meant that the IL-12 polypeptide complex is an attenuated IL-12 receptor agonist. When a IL-23 polypeptide complex that contains IL-23 as described herein is described as “attenuated” or having “attenuated activity”, it is meant that the IL-23 polypeptide complex is an attenuated IL-23 receptor agonist.

The term “cancer” refers to the physiological condition in mammals in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer), osteosarcoma, melanoma, colon cancer, colorectal cancer, endometrial (e.g., serous) or uterine cancer, salivary gland carcinoma, kidney cancer, liver cancer, prostate cancer, vulval cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu.

A “conservative” amino acid substitution, as used herein, generally refers to substitution of one amino acid residue with another amino acid residue from within a recognized group which can change the structure of the peptide but biological activity of the peptide is substantially retained. Conservative substitutions of amino acids are known to those skilled in the art. Conservative substitutions of amino acids can include, but not limited to, substitutions made amongst amino acids within the following groups: (a) M, I, L, V; (b) F, Y, W; (c) K, R, H; (d) A, G; (e) S, T; (f) Q, N; and (g) E, D. For instance, a person of ordinary skill in the art reasonably expect that an isolated replacement of a leucine with an isoleucine or valine, an aspartate with a glutamate, a threonine with a serine, or a similar replacement of an amino acid with a structurally related amino acid will not have a major effect on the biological activity of the resulting molecule.

As used herein, the term “half-life extension element” in the context of the polypeptide complex disclosed herein, refers to a chemical element, preferable a polypeptide that increases the serum half-life and improve pK, for example, by altering its size (e.g., to be above the kidney filtration cutoff), shape, hydrodynamic radius, charge, or parameters of absorption, biodistribution, metabolism, and elimination.

As used herein, the term “operably linked” in the context of a polypeptide complex refers to the orientation of the components of a polypeptide complex that permits the components to function in their intended manner. For example, a polypeptide comprising an IL-12 subunit and an IL-12 blocking element are operably linked by a protease cleavable linker in a polypeptide complex when the IL-12 blocking element is capable of inhibiting the IL-12 receptor-activating activity of the IL-12 polypeptide, but upon cleavage of the protease cleavable linker the inhibition of the IL-12 receptor-activating activity of the IL-12 polypeptide by the IL-12 blocking element is decreased or eliminated, for example because the IL-12 blocking element can diffuse away from the IL-12.

As used herein, the terms “peptide”, “polypeptide”, or “protein” are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

The term “subject” herein to refers to any animal, such as any mammal, including but not limited to, humans, non-human primates, rodents, and the like. In some embodiments, the mammal is a mouse. In some embodiments, the mammal is a human.

As used herein, the term “therapeutically effective amount” refers to an amount of a compound described herein (i.e., a IL-12 polypeptide complex) that is sufficient to achieve a desired pharmacological or physiological effect under the conditions of administration. For example, a “therapeutically effective amount” can be an amount that is sufficient to reduce the signs or symptoms of a disease or condition (e.g., a tumor). Those skilled in the art will appreciate that the therapeutic effects need not be complete or curative, as long as some benefit is provided to the subject. A therapeutically effective amount of a pharmaceutical composition can vary according to factors such as the disease state, age, sex, and weight of the individual, and the ability of the pharmaceutical composition to elicit a desired response in the individual. An ordinarily skilled clinician can determine appropriate amounts to administer to achieve the desired therapeutic benefit based on these and other considerations.

A. IL-12 Polypeptide Complex

The disclosure relates to inducible IL-12 polypeptide complexes that contain at least two polypeptide chains, and can contain three polypeptide chains or more polypeptide chains, if desired. The two or more polypeptide chains disclosed herein are different, i.e., the complexes can be heterodimers, heterotrimers, and the like. The inducible IL-12 polypeptide complex comprises a p35 IL-12 subunit, a p40 IL-12 subunit, a half-life extension element, an IL-12 blocking element, and a protease cleavable linker. The p35 subunit and the p40 subunit associate to form the IL-12 heterodimer, which has intrinsic IL-12 receptor agonist activity. In the context of the IL-12 polypeptide complex, the IL-12 receptor agonist activity is attenuated and the circulating half-life is extended. The IL-12 receptor agonist activity is attenuated through the blocking element. The half-life extension element can also contribute to attenuation, for example through steric effects. The blocking element is capable of blocking the activity of all or some of the receptor agonist activity of IL-12 by sterically blocking and/or noncovalently binding to IL-12 (e.g., to p35, p40, or the p35p40 complex). Upon cleavage of the protease cleavable linker a form of IL-12 is released from the IL-12 polypeptide complex that is active (e.g., more active than the IL-12 polypeptide complex). Typically, the released IL-12 is at least 10× more active than the IL-12 polypeptide complex. Preferably, the released IL-12 is at least 20×, at least 30×, at least 50×, at least 100×, at least 200×, at least 300×, at least 500×, at least 1000×, at least about 10,000× or more active than the IL-12 polypeptide complex.

The form of IL-12 that is released upon cleavage of the IL-12 polypeptide complex typically has a short half-life, which is often substantially similar to the half-life of naturally occurring IL-12. Even though the half-life of the IL-12 polypeptide complex is extended, toxicity is reduced or eliminated because the circulating IL-12 polypeptide complex is attenuated and active IL-12 is targeted to the desired site (e.g., tumor microenvironment).

It will be appreciated by those skilled in the art, that the number of polypeptide chains, and the location of the p35 and p40 subunits, the half-life extension element, the protease cleavable linker(s), and the blocking element (and components of such elements, such as a VH or VL domain) on the polypeptide chains can vary and is often a matter of design preference. All such variations are encompassed by this disclosure.

In embodiments, the IL-12 polypeptide complex comprises two different polypeptide chains. Typically, the first polypeptide chain comprises p35 and the second polypeptide chain comprises p40. The p35 and p40 subunits associate to form a biologically active heterodimer. The p35p40 heterodimer complex can be covalently linked, for example through a disulfide bond.

In embodiments, either the first of the second polypeptide can comprise an IL-12 blocking element (e.g., an scFV that binds IL-12) that is operably linked to the IL-12 subunit through a protease cleavable linker. The other polypeptide chain can further comprise a half-life extension element that is operably linked to the IL-12 subunit through a protease cleavable linker. Preferably, the complex includes one functional blocking element and one functional half-life extension element. For example, when the first polypeptide chain comprises an IL-12 blocking element, the second polypeptide chain does not comprise an IL-12 blocking element. In other embodiments, one polypeptide chain includes either p35 or p40, and further includes a half-life extension element and a blocking element, each of which is operably linked to the p35 or p40 through a protease cleavable linker (e.g., one or more protease cleavable linker), and the other polypeptide include the complementary IL-12 subunit (e.g., either p40 or p35). The IL-12 blocking element on the second polypeptide can be operably linked to the IL-12 subunit through a protease cleavable linker. Alternatively, the IL-12 blocking element can be operably linked to the half-life extension element through an optional protease cleavable linker. The protease cleavable linkers on the first and second polypeptide chains can be the same or can be different. Preferably, the protease cleavable linkers on the first and second polypeptide chains are the same. The blocking element in this IL-12 polypeptide complex can be a single chain antibody. Any single chain antibody that has binding specificity for IL-12 can be a blocking element. Preferably, the blocking element is a scFv.

While the complexes disclosed herein preferably contain one half-life extension element and one blocking element, such elements can contain two or more components that are present on the same polypeptide chain or on different polypeptide chains. Illustrative of this, and as disclosed and exemplified herein, components of the blocking element can present on separate polypeptide chains. For example, a first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the peptide complex to form an antigen-binding site, such as a Fab that binds IL-12 and attenuates IL-12 activity.

In embodiments, the p35 and p40 subunit can be located on the same polypeptide chain, and linked through and optionally protease cleavable linker. In such embodiments of two or multichain complexes, at least one of the half-life extension element, the blocking element, or a component of the half-life extension or blocking element is on a separate polypeptide. For example, a first polypeptide can include p35 and p40, linked through an optionally cleavable polypeptide chain, and other elements of the IL-12 polypeptide complex are located on a second polypeptide chain. In another example, the first polypeptide chain comprises the p35 subunit, the p40 subunit, the half-life extension element, and a portion of an antibody light chain. The second polypeptide contains a portion of an antibody heavy chain that is complementary to the antibody light chain. The portion of the antibody light chain together with the complementary heavy chain associate in the complex to form a binding site for IL-12. In another example, the first polypeptide comprises the p35 subunit, the p40 subunit, the half-life extension element, and a portion of an antibody heavy chain. In this example the second polypeptide contains a portion of an antibody light chain that is complementary to the antibody heavy chain. The portion of the antibody heavy chain together with the complementary light chain associate in the complex to form a binding site for IL-12. In these complexes, the p35 subunit and p40 subunit can be operably linked through an optional protease cleavable linker. Preferably, the p35 subunit and the p40 subunit are operably linked by a non-cleavable linker.

In the complexes disclosed herein, the half-life extension element is preferably operably linked to either the p35 subunit or the p40 subunit through a protease cleavable linker. For example, the complex can include a first polypeptide in which p35 or p40 is operably linked to a half-life extension element through a protease cleavable linker. In another example, the complex can include a first polypeptide in which p35 or p40 is operably linked to a half-life extension element through a protease cleavable linker, and the half-life extension element is further operably linked to a blocking element (or component of a blocking element) through an optionally protease cleavable linker. In such exemplary embodiments, the complex comprises at least one additional polypeptide that includes the IL-12 subunit (p40 or p35) that is not present on the first polypeptide. Additional arrangements of the elements of the complex are envisioned and encompassed by this disclosure. For example, the blocking element can be operably linked to either the p35 subunit or the p40 subunit through a protease cleavable linker. One of the half-life extension element or the blocking element can be operably linked to the p35 subunit, and the other of the half-life or extension element or the blocking element can be operably linked to the p40 subunit. When the half-life extension element is operably linked to the p35 subunit, the blocking element can be operably linked to the p40 subunit. When the half-life extension element is operably linked the p40 subunit, the blocking element can be operably linked to the p35 subunit. The blocking element in this complex is preferably a Fab.

The inducible IL-12 polypeptide complex can comprise three polypeptide chains. Typically, one polypeptide chain comprises either the p35 or p40 IL-12 subunit, but not both, and a second polypeptide comprises the other IL-12 subunit and the third polypeptide comprises at least a portion (component) of the blocking element. When the IL-12 subunit on the first polypeptide is p35, the IL-12 subunit on the second polypeptide is p40. When the IL-12 subunit on the first polypeptide is p40, the IL-12 subunit on the second polypeptide is p35. When the polypeptides are expressed and folded, the p35 and p40 subunits can associate to form a biologically active heterodimer. The p35p40 heterodimer complex can be covalently linked, for example through a disulfide bond.

In some embodiments, the first polypeptide can additionally comprise a half-life extension element that when present is operably linked to the IL-12 subunit through a protease cleavable linker. The second polypeptide further comprises a portion of the blocking element, and the third polypeptide can comprise the remainder of the blocking element. In such a complex, the IL-12 blocking element can be antigen binding fragment of an antibody that is formed by the interaction of polypeptide two and polypeptide three, e.g. a Fab fragment. In embodiments, the second polypeptide can comprise at least an antigen binding portion of an antibody light chain. Alternatively, the second polypeptide can comprise at least an antigen binding portion of an antibody heavy chain. The antigen binding portion of an antibody light chain or the antigen binding portion of the heavy chain can be operably linked to the IL-12 subunit through a protease cleavable linker. In some embodiments, the second polypeptide can contain a half-life extension element. When the second polypeptide contains the half-life extension element, the first polypeptide does not contain the half-life extension element. The half-life extension element can be operably linked to the IL-12 subunit through a protease cleavable linker. Alternatively or in addition, the half-life extension element can be operably linked to a portion of the blocking element (e.g., an antigen binding portion of an antibody light chain or the antigen binding portion of the heavy chain) through an optional protease cleavable linker. When the half-life extension element is present and operably linked to the IL-12 subunit, the antibody heavy chain or light chain can be operably linked to the IL-12 subunit through a protease cleavable linker, Alternatively, when the half-life extension element is present and operably linked to the IL-12 subunit, the antibody heavy chain or light chain can be operably linked to the IL-12 subunit through an optionally cleavable linker. The protease cleavable linkers on the first, second, and/or polypeptide chains can be the same or can be different.

In some embodiments, the IL-12 polypeptide complex comprises a first polypeptide chain comprising the amino acid selected from SEQ ID NOs: 95-110, SEQ ID NOs: 119-126, and SEQ ID NOs: 135-143. Certain preferred IL-12 polypeptide complexes comprise the amino acid sequence of SEQ ID NO: 104 or SEQ ID NO: 136. In some embodiments, the IL-12 polypeptide complex comprises a first polypeptide sequence comprising the amino acid sequence selected from SEQ ID NOs: 119-126, and SEQ ID NOs: 135-143 and a second polypeptide comprising the amino acid sequence of SEQ ID NO: 18. A preferred IL-12 polypeptide complex comprise a first polypeptide chain comprising the amino acid sequence of SEQ ID NO: 104 and a second polypeptide chain comprising the amino acid sequence of SEQ ID NO: 18. Another preferred IL-12 polypeptide comprises a first polypeptide chain comprising the amino acid sequence of SEQ ID NO: 136 and a second polypeptide chain comprising the amino acid sequence of SEQ ID NO: 18.

In some embodiments, the first polypeptide chain of the IL-12 polypeptide complex comprises an amino acid sequence that is at least about 70%, at least about 75%, at least about 80%, at least about 85%, at least about 90%, at least about 95%, at least about 96%, at least about 98%, or at least 99% identical to amino acid sequences selected from SEQ ID NOs: 95-110, SEQ ID NOs: 119-126, and SEQ ID NOs: 135-143. In some embodiments, the second polypeptide chain of the IL-12 polypeptide complex comprises an amino acid sequence that is at least about 70%, at least about 75%, at least about 80%, at least about 85%, at least about 90%, at least about 95%, at least about 96%, at least about 98%, or at least 99% identical to amino acid sequence of SEQ ID NO: 18.

As described above, the IL-12 can be a mutein, if desired. The IL-12 mutein retains IL-12 activity, for example intrinsic IL-12 receptor agonist activity. IL-12 subunits, p35 and/or p40 can be muteins. Preferably, the IL-12 mutein has an altered glycosylation pattern. For example, the IL-12 mutein can be partially aglycosylated or fully aglycosylated. For example, a partially or fully aglycosylated IL-12 polypeptide can comprise a polypeptide selected from the group consisting of SEQ ID NOs: 104, 434 or 442-445, or an amino acid sequence that has at least 80% identity to SEQ ID NOs: 104, 434 or 442-445.

The p35 and/or the p40 subunits can contain one or more amino acid modifications, e.g., substitutions. For instance, the p35 and/or p40 subunits can comprise about one, about two, about three, about four, about five or more amino acid substitutions. Although typically, p35 and/or p40 subunits contain one or two amino acid substitutions. The substitutions can be a conservative substitution or a non-conservative substitution, but preferably is a conservative substitution. A typical modification alters the glycosylation pattern of the p35 and/or p40 subunit such that the p35 and/or p40 subunit is partially or fully aglycosylated. Preferably, the amino acid modification includes replacement of an asparagine amino acid. For example, asparagine to glutamine. In particular examples, asparagine at amino acid positions 16, 75, 85, 133, 151, 158, 201, 206, 221, 250, 267, 280, 282, 326, 400, 404, 425, 555, 572, 575, 582, or 602 on IL-12 p35 of SEQ ID NO: 434 can be mutated. In particular examples, asparagine at amino acid positions 103, 114, 163, 219, 227, or 282 of IL-12 p40 of SEQ ID NO: 18 can be mutated.

The invention also relates to certain single chain IL-12 inducible polypeptides. The single chain IL-12 polypeptides disclosed herein comprise IL-12, a blocking element, a half-life extension element, and a protease cleavable linker. IL-12 has receptor agonist activity for its cognate IL-12 receptor. IL-12 receptor activating activity is attenuated when the blocking element binds to IL-12. Upon cleavage of the protease cleavable linkers, active IL-12 polypeptide is released. Single chain inducible IL-12 polypeptides have been disclosed in International Application No.: PCT/US2019/032320 and International Application No.: PCT/US2019/032322.

The single chain IL-12 inducible polypeptides disclosed herein comprise the amino acid sequence selected SEQ ID NOs: 7, 9, 10, 18, 24-94, SEQ ID NOs: 110-118, and SEQ ID NOs: 127-134. In some embodiments, the single chain IL-12 inducible polypeptide comprises a sequence that is at least 70%, at least 75%, at least 80%, at least, 85%, at least about 90%, at least about 95%, at least about 96%, at least about 97%, at least about 98%, or at least 99% identical to SEQ ID NOs: 7, 9, 10, 18, 24-94, SEQ ID NOs: 110-118, and SEQ ID NOs: 127-134.

B. IL-23 Polypeptide Complex

The disclosure relates to inducible IL-23 polypeptide complexes that contain at least two polypeptide chains, and can contain three polypeptide chains or more polypeptide chains, if desired. The two or more polypeptide chains disclosed herein are different, i.e., the complexes can be heterodimers, heterotrimers, and the like. The inducible IL-23 polypeptide complex comprises a p19 IL-23 subunit, a p40 IL-23 subunit, a half-life extension element, an IL-23 blocking element, and a protease cleavable linker. The p19 subunit and the p40 subunit associate to form the IL-23 heterodimer, which has intrinsic IL-23 receptor agonist activity. As will be well-understood by persons of skill in the art, IL-23 and IL-12 share the same p40 subunit. In the context of the IL-23 polypeptide complex, the IL-23 receptor agonist activity is attenuated and the circulating half-life is extended. The IL-23 receptor agonist activity is attenuated through the blocking element. The half-life extension element can also contribute to attenuation, for example through steric effects. The blocking element is capable of blocking the activity of all or some of the receptor agonist activity of IL-23 by sterically blocking and/or noncovalently binding to IL-23 (e.g., to p19, p40, or the p19p40 complex). Upon cleavage of the protease cleavable linker a form of IL-23 is released from the IL-23 polypeptide complex that is active (e.g., more active than the IL-23 polypeptide complex). Typically, the released IL-23 is at least 10× more active than the IL-23 polypeptide complex. Preferably, the released IL-23 is at least 20×, at least 30×, at least 50×, at least 100×, at least 200×, at least 300×, at least 500×, at least 1000×, at least about 10,000× or more active than the IL-23 polypeptide complex.

The form of IL-23 that is released upon cleavage of the IL-23 polypeptide complex typically has a short half-life, which is often substantially similar to the half-life of naturally occurring IL-23. Even though the half-life of the IL-23 polypeptide complex is extended, toxicity is reduced or eliminated because the circulating IL-23 polypeptide complex is attenuated and active IL-23 is targeted to the desired site (e.g., tumor microenvironment).

It will be appreciated by those skilled in the art, that the number of polypeptide chains, and the location of the p19 and p40 subunits, the half-life extension element, the protease cleavable linker(s), and the blocking element (and components of such elements, such as a VH or VL domain) on the polypeptide chains can vary and is often a matter of design preference. All such variations are encompassed by this disclosure.

In embodiments, the IL-23 polypeptide complex comprises two different polypeptide chains. Typically, the first polypeptide chain comprises p19 and the second polypeptide chain comprises p40. The p19 and p40 subunits associate to form a biologically active heterodimer. The p19p40 heterodimer complex can be covalently linked, for example through a disulfide bond.

In embodiments, either the first of the second polypeptide can comprise an IL-23 blocking element (e.g., an scFV that binds IL-23) that is operably linked to the IL-23 subunit through a protease cleavable linker. The other polypeptide chain can further comprise a half-life extension element that is operably linked to the IL-23 subunit through a protease cleavable linker. Preferably, the complex includes one functional blocking element and one functional half-life extension element. For example, when the first polypeptide chain comprises an IL-23 blocking element, the second polypeptide chain does not comprise an IL-23 blocking element. In other embodiments, one polypeptide chain includes either p19 or p40, and further includes a half-life extension element and a blocking element, each of which is operably linked to the p19 or p40 through a protease cleavable linker (e.g., one or more protease cleavable linker), and the other polypeptide include the complementary IL-23 subunit (e.g., either p40 or p19). The IL-23 blocking element on the second polypeptide can be operably linked to the IL-23 subunit through a protease cleavable linker. Alternatively, the IL-23 blocking element can be operably linked to the half-life extension element through an optional protease cleavable linker. The protease cleavable linkers on the first and second polypeptide chains can be the same or can be different. Preferably, the protease cleavable linkers on the first and second polypeptide chains are the same. The blocking element in this IL-23 polypeptide complex can be a single chain antibody. Any single chain antibody that has binding specificity for IL-23 can be a blocking element. Preferably, the blocking element is a scFv.

While the complexes disclosed herein preferably contain one half-life extension element and one blocking element, such elements can contain two or more components that are present on the same polypeptide chain or on different polypeptide chains. Illustrative of this, and as disclosed and exemplified herein, components of the blocking element can present on separate polypeptide chains. For example, a first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the peptide complex to form an antigen-binding site, such as a Fab that binds IL-23 and attenuates IL-23 activity.

In embodiments, the p19 and p40 subunit can be located on the same polypeptide chain, and linked through and optionally protease cleavable linker. In such embodiments of two or multichain complexes, at least one of the half-life extension element, the blocking element, or a component of the half-life extension or blocking element is on a separate polypeptide. For example, a first polypeptide can include p19 and p40, linked through an optionally cleavable polypeptide chain, and other elements of the IL-23 polypeptide complex are located on a second polypeptide chain. In another example, the first polypeptide chain comprises the p19 subunit, the p40 subunit, the half-life extension element, and a portion of an antibody light chain. The second polypeptide contains a portion of an antibody heavy chain that is complementary to the antibody light chain. The portion of the antibody light chain together with the complementary heavy chain associate in the complex to form a binding site for IL-23. In another example, the first polypeptide comprises the p19 subunit, the p40 subunit, the half-life extension element, and a portion of an antibody heavy chain. In this example the second polypeptide contains a portion of an antibody light chain that is complementary to the antibody heavy chain. The portion of the antibody heavy chain together with the complementary light chain associate in the complex to form a binding site for IL-23. In these complexes, the p19 subunit and p40 subunit can be operably linked through an optional protease cleavable linker. Preferably, the p19 subunit and the p40 subunit are operably linked by a non-cleavable linker.

In the complexes disclosed herein, the half-life extension element is preferably operably linked to either the p19 subunit or the p40 subunit through a protease cleavable linker. For example, the complex can include a first polypeptide in which p19 or p40 is operably linked to a half-life extension element through a protease cleavable linker. In another example, the complex can include a first polypeptide in which p19 or p40 is operably linked to a half-life extension element through a protease cleavable linker, and the half-life extension element is further operably linked to a blocking element (or component of a blocking element) through an optionally protease cleavable linker. In such exemplary embodiments, the complex comprises at least one additional polypeptide that includes the IL-23 subunit (p40 or p19) that is not present on the first polypeptide. Additional arrangements of the elements of the complex are envisioned and encompassed by this disclosure. For example, the blocking element can be operably linked to either the p19 subunit or the p40 subunit through a protease cleavable linker. One of the half-life extension element or the blocking element can be operably linked to the p19 subunit, and the other of the half-life or extension element or the blocking element can be operably linked to the p40 subunit. When the half-life extension element is operably linked to the p19 subunit, the blocking element can be operably linked to the p40 subunit. When the half-life extension element is operably linked the p40 subunit, the blocking element can be operably linked to the p19 subunit. The blocking element in this complex is preferably a Fab.

The inducible IL-23 polypeptide complex can comprise three polypeptide chains. Typically, one polypeptide chain comprises either the p19 or p40 IL-23 subunit, but not both, and a second polypeptide comprises the other IL-23 subunit and the third polypeptide comprises at least a portion (component) of the blocking element. When the IL-23 subunit on the first polypeptide is p19, the IL-23 subunit on the second polypeptide is p40. When the IL-23 subunit on the first polypeptide is p40, the IL-23 subunit on the second polypeptide is p19. When the polypeptides are expressed and folded, the p19 and p40 subunits can associate to form a biologically active heterodimer. The p19p40 heterodimer complex can be covalently linked, for example through a disulfide bond.

In some embodiments, the first polypeptide can additionally comprise a half-life extension element that when present is operably linked to the IL-23 subunit through a protease cleavable linker. The second polypeptide further comprises a portion of the blocking element, and the third polypeptide can comprise the remainder of the blocking element. In such a complex, the IL-23 blocking element can be antigen binding fragment of an antibody that is formed by the interaction of polypeptide two and polypeptide three, e.g. a Fab fragment. In embodiments, the second polypeptide can comprise at least an antigen binding portion of an antibody light chain. Alternatively, the second polypeptide can comprise at least an antigen binding portion of an antibody heavy chain. The antigen binding portion of an antibody light chain or the antigen binding portion of the heavy chain can be operably linked to the IL-23 subunit through a protease cleavable linker. In some embodiments, the second polypeptide can contain a half-life extension element. When the second polypeptide contains the half-life extension element, the first polypeptide does not contain the half-life extension element. The half-life extension element can be operably linked to the IL-23 subunit through a protease cleavable linker. Alternatively or in addition, the half-life extension element can be operably linked to a portion of the blocking element (e.g., an antigen binding portion of an antibody light chain or the antigen binding portion of the heavy chain) through an optional protease cleavable linker. When the half-life extension element is present and operably linked to the IL-23 subunit, the antibody heavy chain or light chain can be operably linked to the IL-23 subunit through a protease cleavable linker, Alternatively, when the half-life extension element is present and operably linked to the IL-23 subunit, the antibody heavy chain or light chain can be operably linked to the IL-23 subunit through an optionally cleavable linker. The protease cleavable linkers on the first, second, and/or polypeptide chains can be the same or can be different.

In embodiments, the IL-23 polypeptide complex comprises a first polypeptide selected from the group consisting of SEQ ID NOs: 423-428, or an amino acid sequence that has at least 80% identity to SEQ ID NOs: 423-428. In embodiments, the IL-23 polypeptide complex comprises a second polypeptide selected from the group consisting of SEQ ID NOs: 18 or 433.

In some embodiments, the first polypeptide chain of the IL-23 polypeptide complex comprises an amino acid sequence that is at least about 70%, at least about 75%, at least about 80%, at least about 85%, at least about 90%, at least about 95%, at least about 96%, at least about 98%, or at least 99% identical to amino acid sequences selected from SEQ ID NOs: 423-428. In some embodiments, the second polypeptide chain of the IL-23 polypeptide complex comprises an amino acid sequence that is at least about 70%, at least about 75%, at least about 80%, at least about 85%, at least about 90%, at least about 95%, at least about 96%, at least about 98%, or at least 99% identical to amino acid sequence of SEQ ID NOs: 18 or 433.

As described above, the IL-23 can be a mutein, if desired. The IL-23 mutein retains IL-23 activity, for example intrinsic IL-23 receptor agonist activity. IL-23 subunits, p19 and/or p40 can be muteins. Preferably, the IL-23 mutein has an altered glycosylation pattern. For example, the IL-23 mutein can be partially aglycosylated or fully aglycosylated.

The p19 and/or the p40 subunits can contain one or more amino acid modifications, e.g., substitutions. For instance, the p19 and/or p40 subunits can comprise about one, about two, about three, about four, about five or more amino acid substitutions. Although typically, p19 and/or p40 subunits contain one or two amino acid substitutions. The substitutions can be a conservative substitution or a non-conservative substitution, but preferably is a conservative substitution. A typical modification alters the glycosylation pattern of the p19 and/or p40 subunit such that the p19 and/or p40 subunit is partially or fully aglycosylated. Preferably, the amino acid modification includes replacement of an asparagine amino acid. For example, asparagine to glutamine. For example, asparagine to glutamine. In particular examples, asparagine at amino acid positions 47 or 66 on IL-12 p19 of SEQ ID NO: 424 can be mutated. In particular examples, asparagine at amino acid positions 103, 114, 163, 219, 227, or 282 of IL-12 p40 of SEQ ID NO: 18 can be mutated.

The invention also relates to certain single chain IL-23 inducible polypeptides. The single chain IL-23 polypeptides disclosed herein comprise IL-23, a blocking element, a half-life extension element, and a protease cleavable linker. IL-23 has receptor agonist activity for its cognate IL-23 receptor. IL-23 receptor activating activity is attenuated when the blocking element binds to IL-23. Upon cleavage of the protease cleavable linkers, active IL-23 polypeptide is released.

The single chain IL-23 inducible polypeptides disclosed herein comprise the amino acid sequence selected of SEQ ID NOs: 422 or 429-432. In some embodiments, the single chain IL-23 inducible polypeptide comprises a sequence that is at least 70%, at least 75%, at least 80%, at least, 85%, at least about 90%, at least about 95%, at least about 96%, at least about 97%, at least about 98%, or at least 99% identical to SEQ ID NOs: 422 or 429-432.

C. Half-Life Extension Element

Contemplated herein are domains which extend the half-life of the IL-12 polypeptide complex. Also contemplated herein are domains which extend the half-life of the IL-23 polypeptide. Increasing the in vivo half-life of therapeutic molecules with naturally short half-lives allows for a more acceptable and manageable dosing regimen without sacrificing effectiveness.

The half-life extension element, increases the in vivo half-life and provides altered pharmacodynamics and pharmacokinetics of the IL-12 polypeptide complex or the IL-23 polypeptide complex. Without being bound by theory, the half-life extension element alters pharmacodynamics properties including alteration of tissue distribution, penetration, and diffusion of the IL-12 polypeptide complex or the IL-23 polypeptide complex. In some embodiments, the half-life extension element can improve tissue targeting, tissue penetration, diffusion within the tissue, and enhanced efficacy as compared with a protein without a half-life extension element. Without being bound by theory, an exemplary way to improve the pharmacokinetics of a polypeptide is by expression of an element in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins, or fragments retain FcRn binding and are routinely linked to other polypeptides to extend their serum half-life. HSA may also be directly bound to the pharmaceutical compositions or bound via a short linker. Fragments of HSA may also be used. HSA and fragments thereof can function as both a blocking element and a half-life extension element. Human IgGs and Fc fragments can also carry out a similar function.

The serum half-life extension element can also be antigen-binding polypeptide that binds to a protein with a long serum half-life such as serum albumin, transferrin and the like. Examples of such polypeptides include antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

The half-life extension element as provided herein is preferably a human serum albumin (HSA) binding domain, and antigen binding polypeptide that binds human serum albumin or an immunoglobulin Fc or fragment thereof.

The half-life extension element of a IL-12 polypeptide complex or a IL-23 polypeptide complex extends the half-life of IL-12 polypeptide complex or the IL-23 polypeptide complex by at least about two days, about three days, about four days, about five days, about six days, about seven days, about eight days, about nine days, about 10 days or more. In some embodiments, the half-life extension element extends the half-life of a IL-12 polypeptide complex or a IL-23 polypeptide complex to at least 2-3 days, 3-4 days, 4-5 days, 5-6 days, 6-7 days, 7-8 days or more.

D. Blocking Element

The blocking element can be any element that binds to IL-12 or IL-23 and inhibits the ability of the IL-12 polypeptide complex or the IL-23 polypeptide complex to bind and activate its receptor. The blocking element can inhibit the ability of the IL-12 or IL-23 to bind and/or activate its receptor e.g., by sterically blocking and/or by noncovalently binding to the IL-12 polypeptide complex. The blocking element disclosed herein can bind to p19, p35, p40, the p35p40 heterodimeric complex, or the p19p40 heterodimeric complex.

Examples of suitable blocking elements include the full length or an IL-12-binding fragment or mutein of the cognate receptor of IL-12. Other examples of suitable blocking elements include the full length or an IL-23-binding fragment or mutein of the cognate receptor of IL-23. Antibodies and antigen-binding fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like that bind IL-12 or IL-23 can also be used. Other suitable antigen-binding domain that bind IL-12 or IL-23 can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of suitable blocking polypeptides include polypeptides that sterically inhibit or block binding of IL-12 or IL-23 to its cognate receptor. Advantageously, such moieties can also function as half-life extending elements. For example, a peptide that is modified by conjugation to a water-soluble polymer, such as PEG, can sterically inhibit or prevent binding of the cytokine to its receptor. Polypeptides, or fragments thereof, that have long serum half-lives can also be used, such as serum albumin (human serum albumin), immunoglobulin Fc, transferrin and the like, as well as fragments and muteins of such polypeptides.

Preferred IL-12 blocking elements are single chain variable fragments (scFv) or Fab fragments. Preferred IL-23 blocking elements are single chain variable fragments (scFv) or Fab fragments. The scFv blocking elements comprise the amino acid sequence as set forth in SEQ ID NOs: 145-188. Alternatively, the Fab blocking element comprises the amino acid sequence as set forth in SEQ ID NOs: 189-194. The IL-12 antibody fragments encompassed by SEQ ID NOs: 145-194 have been optimized to enhance the developability of the IL-12 polypeptide complex disclosed herein.

Preferred antibody light chain blocking elements comprise SEQ ID NOs: 192-193. These preferred components can be located on one polypeptide chain and the complementary antigen binding portion of the heavy chain can be located on a second polypeptide chain. Preferred heavy chain blocking elements comprise SEQ ID NOs: 189-191 and 194. These preferred components can be located on one polypeptide chain and the complementary light chain is located on a second polypeptide chain. The antibody light chain and the antibody heavy chain together form a binding site for IL-12.

In some embodiments, the IL-12 blocking element comprises an amino acid sequence that is at least about 70%, at least about 75%, at least about 80%, at least about 85%, at least about 90%, at least about 95%, at least about 96%, at least about 97%, at least about 98%, at least about 99% identical to SEQ ID NOs: 145-194, e.g., over the full length of SEQ ID Nos:145-194. Typically, the amino acid sequence of the CDRs in not altered, and amino acid substitutions are present in the framework regions.

The disclosure also relates to functional variants of IL-12 blocking elements comprising SEQ ID NOs: 145-194. The functional variants of IL-12 blocking elements comprising SEQ ID NOs: 145-194 generally differ from SEQ ID NOs: 145-194 by one or a few amino acids (including substitutions, deletions, insertions, or any combination thereof), and substantially retain their ability to bind to the IL-12 polypeptide (e.g., the p35 subunit, the p40 subunit, or the p35p40 complex) and inhibit binding of IL-12 to its cognate receptor.

The functional variant can contain at least one or more amino acid substitutions, deletions, or insertions relative to the IL-12 blocking element comprising SEQ ID NOs: 145-194. The functional variant can comprise 1, 2, 3, 4, 5, 6, 7, 8, 9, or 10 amino acid alterations compared to the IL-12 blocking element comprising SEQ ID NOs: 145-194. In some preferred embodiments, the functional variant differs from the IL-12 blocking element comprising SEQ ID NOs: 145-194 by less than 10, less, than 8, less than 5, less than 4, less than 3, less than 2, or one amino acid alterations, e.g., amino acid substitutions or deletions. In other embodiments, the functional variant may comprise 1, 2, 3, 4, 5, 6, 7, 8, 9, or 10 amino acid substitutions compared to SEQ ID NOs: 145-194. The amino acid substitution can be a conservative substitution or a non-conservative substitution, but preferably is a conservative substitution.

In other embodiments, the functional variants of the IL-12 blocking element may comprise 1, 2, 3, 4, or 5 or more non-conservative amino acid substitutions compared the IL-12 blocking elements comprising SEQ ID NOs: 145-194. Non-conservative amino acid substitutions could be recognized by one of skill in the art. The functional variant of the separation moiety preferably contains no more than 1, 2, 3, 4, or 5 amino acid deletions.

Also disclosed herein is an inducible IL-12 polypeptide that contains a blocking element having specificity for IL-12 and contains a half-life extension element. Also disclosed herein is an inducible IL-12 polypeptide that contains a blocking element having specificity for IL-23 and contains a half-life extension element. The blocking element is an antibody or antigen binding fragment that has binding specificity for IL-12, specifically the IL-12 subunit beta precursor (p40) as defined by SEQ ID NO: 421, disclosed herein. The antibody or antigen binding fragment comprises an antigen binding domain that binds to the residues shown in Table 1 of SEQ ID NO: 421. This disclosure relates to an antibody or antigen-binding fragment that binds the IL-12 epitope defined by the amino acid residues shown in Table 1, and to an inducible IL-12 polypeptide complex that contains such an antibody or antigen-binding fragment, and to the use of such an antibody or antigen-binding fragment for the preparation of an inducible IL-12 polypeptide complex, or a medicament containing such an inducible IL-12 polypeptide complex.

TABLE 1
Epitope binding residues in the IL-12 subunit beta precursor

| | # with | # without |
|---|---|---|
| | signal | signal |
| | sequence | sequence |
| ASP | 36 | 14 |
| TRP | 37 | 15 |
| TYR | 38 | 16 |
| PRO | 39 | 17 |
| ASP | 40 | 18 |
| LYS | 106 | 84 |
| LYS | 107 | 85 |
| GLU | 108 | 86 |
| ASP | 109 | 87 |
| GLY | 110 | 88 |
| ILE | 111 | 89 |
| THR | 114 | 92 |
| ASP | 115 | 93 |
| LYS | 124 | 102 |
| ASN | 125 | 103 |
| LYS | 126 | 104 |
| LYS | 219 | 197 |

E. Protease Cleavable Linker

As disclosed herein, the IL-12 polypeptide complex or the IL-23 polypeptide complex comprises one or more linker sequences. A linker sequence serves to provide flexibility between the polypeptides, such that, for example, the blocking element is capable of inhibiting the activity of IL-12 or IL-23. The linker can be located between the IL-12 subunit or the IL-23 subunit, the half-life extension element, and/or the blocking element. As described herein the IL-12 polypeptide complex comprises a protease cleavable linker. As described herein the IL-23 polypeptide complex comprises a protease cleavable linker. The protease cleavable linker can comprise one or more cleavage sites for one or more desired protease. Preferably, the desired protease is enriched or selectively expressed at the desired target site of IL-12 or IL-23 activity (e.g., the tumor microenvironment). Thus, the IL-12 polypeptide complex or the IL-23 polypeptide complex is preferentially or selectively cleaved at the target site of desired IL-12 activity or IL-23 activity.

Suitable linkers are typically less than about 100 amino acids. Such linkers can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domain. In a preferred embodiment, the linker is cleavable by a cleaving agent, e.g., an enzyme. Preferably, the separation moiety comprises a protease cleavage site. In some cases, the separation moiety comprises one or more cleavage sites. The separation moiety can comprise a single protease cleavage site. The separation moiety can also comprise 2 or more protease cleavage sites. For example, 2 cleavage sites, 3 cleavage sites, 4, cleavage sites, 5 cleavage sites, or more. In cases the separation moiety comprises 2 or more protease cleavage sites, the cleavage sites can be cleaved by the same protease or different proteases. A separation moiety comprising two or more cleavage sites is referred to as a “tandem linker.” The two or more cleavage sites can be arranged in any desired orientation, including, but not limited tom one cleavage site adjacent to another cleavage site, one cleavage site overlapping another cleavage site, or one cleavage site following by another cleavage site with intervening amino acids between the two cleavage sites.

Of particular interest in the present invention are disease specific protease-cleavable linkers. Also preferred are protease-cleavable linkers that are preferentially cleaved at a desired location in the body, such as the tumor microenvironment, relative to the peripheral circulation. For example, the rate at which the protease-cleavable linker is cleaved in the tumor microenvironment can be at least about 10 times, at least about 100 times, at least about 1000 times or at least about 10,000 times faster in the desired location in the body, e.g., the tumor microenvironment, in comparison to in the peripheral circulation (e.g., in plasma).

Proteases known to be associated with diseased cells or tissues include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin G, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAPα), dipeptidyl peptidase, meprins, granzymes and dipeptidyl peptidase IV (DPPIV/CD26). Proteases capable of cleaving linker amino acid sequences (which can be encoded by the chimeric nucleic acid sequences provided herein) can, for example, be selected from the group consisting of a prostate specific antigen (PSA), a matrix metalloproteinase (MMP), an A Disintigrin and a Metalloproteinase (ADAM), a plasminogen activator, a cathepsin, a caspase, a tumor cell surface protease, and an elastase. The MMP can, for example, be matrix metalloproteinase 2 (MMP2), matrix metalloproteinase 9 (MMP9), matrix metalloproteinase 14 (MMP14). In addition, or alternatively, the linker can be cleaved by a cathepsin, such as, Cathepsin B, Cathepsin C, Cathepsin D, Cathepsin E, Cathepsin G, Cathepsin K and/or Cathepsin L. Preferably, the linker can be cleaved by MMP14 or Cathepsin L.

Proteases useful for cleavage of linkers and for use in the IL-12 polypeptide complex disclosed herein are presented in Table 2, and exemplary proteases and their cleavage site are presented in Table 3.

TABLE 2
Proteases relevant to inflammation and cancer

| | | |
|---|---|---|
| Protease | Specificity | Other aspects |

Secreted by killer T cells:

| | | |
|---|---|---|
| Granzyme B (grB) | Cleaves after Asp | Type of serine protease; strongly |
| | residues (asp-ase) | implicated in inducing perforin-dependent |
| | | target cell apoptosis |
| Granzyme A (grA) | trypsin-like, cleaves after | Type of serine protease; |
| | basic residues | |
| Granzyme H (grH) | Unknown substrate | Type of serine protease; |
| | specificity | Other granzymes are also secreted by |
| | | killer T cells, but not all are present in |
| | | humans |
| Caspase-8 | Cleaves after Asp | Type of cysteine protease; plays essential |
| | residues | role in TCR-induced cellular expansion- |
| | | exact molecular role unclear |
| Mucosa-associated | Cleaves after arginine | Type of cysteine protease; likely acts both |
| lymphoid tissue | residues | as a scaffold and proteolytically active |
| (MALT1) | | enzyme in the CBM-dependent signaling |
| | | pathway |
| Tryptase | Targets: angiotensin I, | Type of mast cell-specific serine protease; |
| | | trypsin-like; resistant to inhibition by |
| | fibrinogen, prourokinase, | macromolecular protease inhibitors |
| | TGFβ; preferentially | expressed in mammals due to their |
| | cleaves proteins after | tetrameric structure, with all sites facing |
| | lysine or arginine | narrow central pore; also associated with |
| | residues | inflammation |

Associated with inflammation:

| | | |
|---|---|---|
| Thrombin | Targets: FGF-2, | Type of serine protease; modulates |
| | HB-EGF, Osteo-pontin, | activity of vascular growth factors, |
| | PDGF, VEGF | chemokines and extracellular proteins; |
| | | strengthens VEGF-induced proliferation; |
| | | induces cell migration; angiogenic factor; |
| | | regulates hemostasis |
| Chymase | Exhibit chymotrypsin- | Type of mast cell-specific serine protease |
| | like specificity, cleaving | |
| | proteins after aromatic | |
| | amino acid residues | |
| Carboxypeptidase A | Cleaves amino acid | Type of zinc-dependent metalloproteinase |
| (MC-CPA) | residues from C-terminal | |
| | end of peptides and | |
| | proteins | |
| Kallikreins | Targets: high molecular | Type of serine protease; modulate |
| | weight | relaxation response; contribute to |
| | kininogen, pro-urokinase | inflammatory response; fibrin degradation |
| Elastase | Targets: E-cadherin, GM- | Type of neutrophil serine protease; |
| | CSF, IL-1, IL-2, IL-6, | degrades ECM components; regulates |
| | IL8, p38^{MAPK}, TNFα, VE- | inflammatory response; activates pro- |
| | cadherin | apoptotic signaling |
| Cathepsin G | Targets: EGF, ENA-78, | Type of serine protease; degrades ECM |
| | IL-8, MCP-1, MMP-2, | components; chemo-attractant of |
| | MT1-MMP, | leukocytes; regulates inflammatory |
| | PAI-1, RANTES, TGFβ, | response; promotes apoptosis |
| | TNFα | |
| PR-3 | Targets: ENA-78, IL-8, | Type of serine protease; promotes |
| | IL-18, JNK, p38^{MAPK}, | inflammatory response; activates pro- |
| | TNFα | apoptotic signaling |
| Granzyme M (grM) | Cleaves after Met and | Type of serine protease; only expressed in |
| | other long, unbranched | NK cells |
| | hydrophobic residues | |
| Calpains | Cleave between Arg and | Family of cysteine proteases; calcium- |
| | Gly | dependent; activation is involved in the |
| | | process of numerous inflammation- |
| | | associated diseases |

TABLE 3
Exemplary Proteases and Protease Recognition Sequences

| Protease | Cleavage Domain Sequence | SEQ ID NO: |
|---|---|---|
| MMP7 | KRALGLPG | 375 |
| MMP7 | (DE)8RPLALWRS(DR)8 | 376 |
| MMP9 | PR(S/T)(L/I)(S/T) | 377 |
| MMP9 | LEATA | 378 |
| MMP11 | GGAANLVRGG | 379 |
| MMP14 | SGRIGFLRTA | 380 |
| MMP | PLGLAG | 381 |
| MMP | PLGLAX | 382 |
| MMP | PLGC(me)AG | 383 |
| MMP | ESPAYYTA | 384 |
| MMP | RLQLKL | 385 |
| MMP | RLQLKAC | 386 |
| MMP2, MMP9, MMP14 | EP(Cit)G(Hof)YL | 387 |
| Urokinase plasminogen activator (uPA) | SGRSA | 388 |
| Urokinase plasminogen activator (uPA) | DAFK | 389 |
| Urokinase plasminogen activator (uPA) | GGGRR | 390 |
| Lysosomal Enzyme | GFLG | 391 |
| Lysosomal Enzyme | ALAL | 392 |
| Lysosomal Enzyme | FK | 393 |
| Cathepsin B | NLL | 394 |
| Cathepsin D | PIC(Et)FF | 395 |
| Cathepsin K | GGPRGLPG | 396 |
| Prostate Specific Antigen | HSSKLQ | 397 |
| Prostate Specific Antigen | HSSKLQL | 398 |
| Prostate Specific Antigen | HSSKLQEDA | 399 |
| Herpes Simplex Virus Protease | LVLASSSFGY | 400 |
| HIV Protease | GVSQNYPIVG | 401 |
| CMV Protease | GVVQASCRLA | 402 |
| Thrombin | F(Pip)RS | 403 |
| Thrombin | DPRSFL | 404 |
| Thrombin | PPRSFL | 405 |
| Caspase-3 | DEVD | 406 |
| Caspase-3 | DEVDP | 407 |
| Caspase-3 | KGSGDVEG | 408 |
| Interleukin 1ß converting enzyme | GWEHDG | 409 |
| Enterokinase | EDDDDKA | 410 |
| FAP | KQEQNPGST | 411 |
| Kallikrein 2 | GKAFRR | 412 |
| Plasmin | DAFK | 413 |
| Plasmin | DVLK | 414 |
| Plasmin | DAFK | 415 |
| TOP | ALLLALL | 416 |
| | GPLGVRG | 417 |
| | IPVSLRSG | 418 |
| | VPLSLYSG | 419 |
| | SGESPAYYTA | 420 |

Exemplary protease cleavable linkers include, but are not limited to kallikrein cleavable linkers, thrombin cleavable linkers, chymase cleavable linkers, carboxypeptidase A cleavable linkers, cathepsin cleavable linkers, elastase cleavable linkers, FAP cleavable linkers, ADAM cleavable linkers, PR-3 cleavable linkers, granzyme M cleavable linkers, a calpain cleavable linkers, a matrix metalloproteinase (MMP) cleavable linkers, a plasminogen activator cleavable linkers, a caspase cleavable linkers, a tryptase cleavable linkers, or a tumor cell surface protease. Specifically, MMP9 cleavable linkers, ADAM cleavable linkers, CTSL1 cleavable linkers, FAPα cleavable linkers, and cathepsin cleavable linkers. Some preferred protease-cleavable linkers are cleaved by a MMP and/or a cathepsin.

The separation moieties disclosed herein are typically less than 100 amino acids. Such separation moieties can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domains.

In some embodiments, the separation moiety comprises the sequence GPAGLYAQ (SEQ ID NO: 195); GPAGMKGL (SEQ ID NO: 196); PGGPAGIG (SEQ ID NO: 197); ALFKSSFP (SEQ ID NO: 198); ALFFSSPP (SEQ ID NO: 199); LAQRLRSS (SEQ ID NO: 200); LAQKLKSS (SEQ ID NO; 201); GALFKSSFPSGGGPAGLYAQGGSGKGGSGK (SEQ ID NO: 202); RGSGGGPAGLYAQGSGGGPAGLYAQGGSGK (SEQ ID NO: 203); KGGGPAGLYAQGPAGLYAQGPAGLYAQGSR (SEQ ID NO: 204); RGGPAGLYAQGGPAGLYAQGGGPAGLYAQK (SEQ ID NO: 205); KGGALFKSSFPGGPAGIGPLAQKLKSSGGS (SEQ ID NO: 206); SGGPGGPAGIGALFKSSFPLAQKLKSSGGG (SEQ ID NO: 207); RGPLAQKLKSSALFKSSFPGGPAGIGGGGK (SEQ ID NO: 208); GGGALFKSSFPLAQKLKSSPGGPAGIGGGR (SEQ ID NO: 209); RGPGGPAGIGPLAQKLKSSALFKSSFPGGG (SEQ ID NO: 210); RGGPLAQKLKSSPGGPAGIGALFKSSFPGK (SEQ ID NO: 211); RSGGPAGLYAQALFKSSFPLAQKLKSSGGG (SEQ ID NO: 212); GGPLAQKLKSSALFKSSFPGPAGLYAQGGR (SEQ ID NO: 213); GGALFKSSFPGPAGLYAQPLAQKLKSSGGK (SEQ ID NO: 214); RGGALFKSSFPLAQKLKSSGPAGLYAQGGK (SEQ ID NO: 215); RGGGPAGLYAQPLAQKLKSSALFKSSFPGG (SEQ ID NO: 216); SGPLAQKLKSSGPAGLYAQALFKSSFPGSK (SEQ ID NO: 217); KGGPGGPAGIGPLAQRLRSSALFKSSFPGR (SEQ ID NO: 218); KSGPGGPAGIGALFFSSPPLAQKLKSSGGR (SEQ ID NO: 219); or SGGFPRSGGSFNPRTFGSKRKRRGSRGGGG (SEQ ID NO: 220)

Certain preferred separation moieties comprises the sequence GPAGLYAQ (SEQ ID NO: 195) or ALFKSSFP (SEQ ID NO: 198). The separation moieties disclosed herein can comprise one or more cleavage motif or functional variants that are the same or different. The separation moieties can comprise 1, 2, 3, 4, 5, or more cleavage motifs or functional variants. Separation moieties comprising 30 amino acids can contain 2 cleavage motifs or functional variants, 3 cleavage motifs or functional variants or more. A “functional variant” of a separation moiety retains the ability to be cleaved with high efficiency at a target site (e.g., a tumor microenvironment that expresses high levels of the protease) and are not cleaved or cleaved with low efficiency in the periphery (e.g., serum). For example, the functional variants retain at least about 50%, about 55%, about 60%, about 70%, about 80%, about 85%, about 95% or more of the cleavage efficiency of a separation moiety comprising any one of SEQ ID NOs: 195-220 or 447-448.

The separation moieties comprising more than one cleavage motif can be selected from SEQ ID NOs: 195-201 or 447-448 and combinations thereof. Preferred separation moieties comprising more than one cleavage motif comprise the amino acids selected from SEQ ID NO: 202-220.

The separation moiety can comprise both ALFKSSFP (SEQ ID NO: 198) and GPAGLYAQ (SEQ ID NO: 195). The separation moiety can comprise two cleavage motifs that each have the sequence GPAGLYAQ (SEQ ID NO: 195). Alternatively or additionally, the separation moiety can comprise two cleavage motifs that each have the sequence ALFKSSFP (SEQ ID NO: 198). The separation moiety can comprise a third cleavage motif that is the same or different.

In some embodiments, the separation moiety comprises an amino acid sequence that is at least about 90%, at least about 95%, at least about 96%, at least about 97%, at least about 98%, or at least 99% identical to SEQ ID NOs: 195 to SEQ ID NO: 220 or 447-448 over the full length of SEQ ID NO: 195-220 or SEQ ID NOS 447-448.

The disclosure also relates to functional variants of separation moieties comprising SEQ ID NOs: 195-220 or 447-448. The functional variants of separation moieties comprising SEQ ID NOs: 195-220 or 447-448 generally differ from SEQ ID NOs: 195-220 or 447-448 by one or a few amino acids (including substitutions, deletions, insertions, or any combination thereof), and substantially retain their ability to be cleaved by a protease.

The functional variants can contain at least one or more amino acid substitutions, deletions, or insertions relative to the separation moieties comprising SEQ ID NOs: 195-220 or 447-448. The functional variant can comprise 1, 2, 3, 4, 5, 6, 7, 8, 9, or 10 amino acid alterations comparted to the separation moieties comprising SEQ ID NOs: 195-220 or 447-448. In some preferred embodiments, the functional variant differs from the separation moiety comprising SEQ ID NOs: 195-220 by less than 10, less, than 8, less than 5, less than 4, less than 3, less than 2, or one amino acid alterations, e.g., amino acid substitutions or deletions. In other embodiments, the functional variant may comprise 1, 2, 3, 4, 5, 6, 7, 8, 9, or 10 amino acid substitutions compared to SEQ ID NOs: 195-220 or 447-448. The amino acid substitution can be a conservative substitution or a non-conservative substitution, but preferably is a conservative substitution.

In other embodiments, the functional variants of the separation moieties may comprise 1, 2, 3, 4, or 5 or more non-conservative amino acid substitutions compared the separation moieties comprising SEQ ID NOs: 195-220 or 447-448. Non-conservative amino acid substitutions could be recognized by one of skill in the art. The functional variant of the separation moiety preferably contains no more than 1, 2, 3, 4, or 5 amino acid deletions.

The amino acid sequences disclosed in the separation moieties can be described by the relative linear position in the separation moiety with respect to the sissile bond. As will be well-understood by persons skilled in the art, separation moieties comprising 8 amino acid protease substrates (e.g., SEQ ID Nos: 195-201 or 447-448) contain amino acid at positions P4, P3, P2, P1, P1′, P2′, P3′, P4′, wherein the sissile bond is between P1 and P1′. For example, amino acid positions for the separation moiety comprising the sequence GPAGLYAQ (SEQ ID NO: 195) can be described as follows:

| G | P | A | G | L | Y | A | Q |
|---|---|---|---|---|---|---|---|
| P4 | P3 | P2 | P1 | P1′ | P2′ | P3′ | P4′ |

Amino acids positions for the separation moiety comprising the sequence ALFKSSFP (SEQ ID NO: 198) can be described as follows:

| A | L | F | K | S | S | F | P |
|---|---|---|---|---|---|---|---|
| P4 | P3 | P2 | P1 | P1′ | P2′ | P3′ | P4′ |

Preferably, the amino acids surrounding the cleavage site (e.g., positions P1 and P1′ for SEQ ID NOs: 195-201 or 447-448) are not substituted.

In embodiments, the separation moiety comprises the sequence GPAGLYAQ (SEQ ID NO: 195) or ALFKSSFP (SEQ ID NO: 198) or a functional variant of SEQ ID NO: 195 or a function variant of SEQ ID NO: 198. As described herein, a functional variant of PAGLYAQ (SEQ ID NO: 447) or ALFKSSFP (SEQ ID NO: 198) can comprise one or more amino acid substitutions, and substantially retain their ability to be cleaved by a protease. Specifically, the functional variants of GPAGLYAQ (SEQ ID NO: 195) is cleaved by MMP14, and the functional variant of ALFKSSFP (SEQ ID NO: 198) is cleaved by Capthepsin L (CTSL1). The functional variants also retain their ability to be cleaved with high efficiency at a target site (e.g., a tumor microenvironment that expresses high levels of the protease). For example, the functional variants of GPAGLYAQ (SEQ ID NO: 195) or ALFKSSFP (SEQ ID NO: 198) retain at least about 50%, about 55%, about 60%, about 70%, about 80%, about 85%, about 95% or more of the cleavage efficiency of a separation moiety comprising amino acid sequence GPAGLYAQ (SEQ ID NO: 195) or ALFKSSFP (SEQ ID NO: 198), respectively.

Preferably, the functional variant of GPAGLYAQ (SEQ ID NO: 195) or ALFKSSFP (SEQ ID NO: 198) comprise no more than 1, 2, 3, 4, or 5 conservative amino acid substitutions compared to GPAGLYAQ (SEQ ID NO: 195) or ALFKSSFP (SEQ ID NO: 198). Preferably, the amino acids at position P1 and P1′ are not substituted. The amino acids at positions P1 and P1′ in SEQ ID NO: 195 are G and L, and the amino acids at positions P1 and P1′ in SEQ ID NO: 198 are K and S.

The functional variant of GPAGLYAQ (SEQ ID NO: 195) can preferably comprise one or more of the following: a) an arginine amino acid substitution at position P4, b) a leucine, valine, asparagine, or proline amino acid substitution at position P3, c) a asparagine amino acid substitution at position P2, d) a histidine, asparagine, or glycine amino acid substitution at position P1, e) a asparagine, isoleucine, or leucine amino acid substitution at position P1′, f) a tyrosine or arginine amino acid substitution at position P2′, g) a glycine, arginine, or alanine amino acid substitution at position P3′, h) or a serine, glutamine, or lysine amino acid substitution at position P4′. The following amino acid substitutions are disfavored in functional variants of GPAGLYAQ (SEQ ID NO: 195): a) arginine or isoleucine at position P3, b) alanine at position P2, c) valine at position P1, d) arginine, glycine, asparagine, or threonine at position P1′, e) aspartic acid or glutamic acid at position P2′, f) isoleucine at position P3′, g) valine at position P4′. In some embodiments, the functional variant of GPAGLYAQ (SEQ ID NO: 195) does not comprise an amino acid substitution at position P1 and/or P1′.

The amino acid substitution of the functional variant of GPAGLYAQ (SEQ ID NO: 195) preferably comprises an amino acid substitution at position P4 and/or P4′. For example, the functional variant of GPAGLYAQ (SEQ ID NO: 195) can comprise a leucine at position P4, or serine, glutamine, lysine, or phenylalanine at position P4. Alternatively or additionally, the functional variant of GPAGLYAQ (SEQ ID NO: 195) can comprise a glycine, phenylalanine, or a proline at position P4′.

In some embodiments, the amino acid substitutions at position P2 or P2′ of GPAGLYAQ (SEQ ID NO: 195) are not preferred.

In some embodiments, the functional variant of GPAGLYAQ (SEQ ID NO: 195) comprises the amino acid sequence selected from SEQ ID NOs: 221-295. Specific functional variants of GPAGLYAQ (SEQ ID NO: 195) include GPLGLYAQ (SEQ ID NO: 259), and GPAGLKGA (SEQ ID NO: 249).

The functional variants of LFKSSFP (SEQ ID NO: 448) preferably comprises hydrophobic amino acid substitutions. The functional variant of LFKSSFP (SEQ ID NO: 448) can preferably comprise one or more of the following: (a) lysine, histidine, serine, glutamine, leucine, proline, or phenylalanine at position P4; (b) lysine, histidine, glycine, proline, asparagine, phenylalanine at position P3; (c) arginine, leucine, alanine, glutamine, or histatine at position P2; (d) phenylalanine, histidine, threonine, alanine, or glutamine at position P1; (e) histidine, leucine, lysine, alanine, isoleucine, arginine, phenylalanine, asparagine, glutamic acid, or glycine at position P1′, (f) phenylalanine, leucine, isoleucine, lysine, alanine, glutamine, or proline at position P2′; (g) phenylalanine, leucine, glycine, serine, valine, histidine, alanine, or asparagine at position P3′; and phenylalanine, histidine, glycine, alanine, serine, valine, glutamine, lysine, or leucine.

The inclusion of aspartic acid and/or glutamic acid in functional variants of SEQ ID NO: 448 are generally disfavored and avoided. The following amino acid substitutions are also disfavored in functional variants of LFKSSFP (SEQ ID NO: 448): (a) alanine, serine, or glutamic acid at position P3; (b) proline, threonine, glycine, or aspartic acid at position P2; (c) proline at position P1; (d) proline at position P1′; (e) glycine at position P2′; (f) lysine or glutamic acid at position P3′; (g) aspartic acid at position P4′.

The amino acid substitution of the functional variant of LFKSSFP (SEQ ID NO: 448) preferably comprises an amino acid substitution at position P4 and/or P1. In some embodiments, an amino acid substitution of the functional variant of LFKSSFP (SEQ ID NO: 448) at position P4′ is not preferred.

In some embodiments, the functional variant of LFKSSFP (SEQ ID NO: 448) comprises the amino acid sequence selected from SEQ ID NOs: 296-374. Specific functional variants of LFKSSFP (SEQ ID NO: 448) include ALFFSSPP (SEQ ID NO: 199), ALFKSFPP (SEQ ID NO: 346), ALFKSLPP (SEQ ID NO: 347); ALFKHSPP (SEQ ID NO: 335); ALFKSIPP (SEQ ID NO: 348); ALFKSSLP (SEQ ID NO: 356); or SPFRSSRQ (SEQ ID NO: 297).

The separation moieties disclosed herein can form a stable complex under physiological conditions with the amino acid sequences (e.g. domains) that they link, while being capable of being cleaved by a protease. For example, the separation moiety is stable (e.g., not cleaved or cleaved with low efficiency) in the circulation and cleaved with higher efficiency at a target site (i.e. a tumor microenvironment). Accordingly, fusion polypeptides that include the linkers disclosed herein can, if desired, have a prolonged circulation half-life and/or lower biological activity in the circulation in comparison to the components of the fusion polypeptide as separate molecular entities. Yet, when in the desired location (e.g., tumor microenvironment) the linkers can be efficiently cleaved to release the components that are joined together by the linker and restoring or nearly restoring the half-life and biological activity of the components as separate molecular entities.

The separation moiety desirably remains stable in the circulation for at least 2 hours, at least 5, hours, at least 10 hours, at least 15 hours, at least 20 hours, at least 24 hours, at least 30 hours, at least 35 hours, at least 40 hours, at least 45 hours, at least 50 hours, at least 60 hours, at least 65 hours, at least 70 hours, at least 80 hours, at least 90 hours, or longer.

In some embodiments, the separation moiety is cleaved by less than 90%, 80%, 70%, 60%, 50%, 40%, 30%, 20%, 20%, 5%, or 1% in the circulation as compared to the target location. The separation moiety is also stable in the absence of an enzyme capable of cleaving the linker. However, upon expose to a suitable enzyme (i.e., a protease), the separation moiety is cleaved resulting in separation of the linked domain.

F. Pharmaceutical Compositions

Also provided herein, are pharmaceutical compositions comprising a IL-12 polypeptide complex or an IL-23 polypeptide complex described herein, a vector comprising the polynucleotide encoding the IL-12 polypeptide complex or the IL-23 polypeptide complex or a host cell transformed by this vector and at least one pharmaceutically acceptable carrier.

Provided herein are pharmaceutical formulations or compositions containing the IL-12 polypeptide complexes or the IL-23 polypeptide complexes as described herein and a pharmaceutically acceptable carrier. Compositions comprising the IL-12 polypeptide complexes or the IL-23 polypeptide complexes as described herein are suitable for administration in vitro or in vivo. The term “pharmaceutically acceptable carrier” includes, but is not limited to, any carrier that does not interfere with the effectiveness of the biological activity of the ingredients and that is not toxic to the subject to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Suitable carriers and their formulations are described in Remington: The Science and Practice of Pharmacy, 21st Edition, David B. Troy, ed., Lippicott Williams & Wilkins (2005). Typically, an appropriate amount of a pharmaceutically-acceptable salt is used in the formulation to render the formulation isotonic, although the formulate can be hypertonic or hypotonic if desired. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the IL-12 or IL-23 polypeptide complexes or nucleic acid sequences encoding the IL-12 or IL-23 polypeptide complexes to humans or other subjects.

In some embodiments of the pharmaceutical compositions, the IL-12 polypeptide complex or the IL-23 polypeptide complex described herein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the IL-12 polypeptide complex or the IL-23 polypeptide complex is attached to liposomes. In some instances, the IL-12 polypeptide complex or the IL-23 polypeptide complex are conjugated to the surface of liposomes. In some instances, the IL-12 polypeptide complex or the IL-23 polypeptide complex are encapsulated within the shell of a liposome. In some instances, the liposome is a cationic liposome.

The IL-12 polypeptide complex or the IL-23 polypeptide complexes described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

Optionally, the IL-12 polypeptide complex or nucleic acid sequences encoding the IL-12 polypeptide complex are administered by a vector. Optionally, the IL-23 polypeptide complex or nucleic acid sequences encoding the IL-23 polypeptide complex are administered by a vector. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein. Such compositions and methods can be used to transfect or transduce cells in vitro or in vivo, for example, to produce cell lines that express and preferably secrete the encoded chimeric polypeptide or to therapeutically deliver nucleic acids to a subject. The components of the IL-12 polypeptide or the IL-23 polypeptide disclosed herein are typically operably linked in frame to encode a fusion protein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general and methods of making them are described by Coffin et al., Retroviruses, Cold Spring Harbor Laboratory Press (1997). The construction of replication-defective adenoviruses has been described (Berkner et al., J. Virol. 61:1213-20 (1987); Massie et al., Mol. Cell. Biol. 6:2872-83 (1986); Haj-Ahmad et al., J. Virol. 57:267-74 (1986); Davidson et al., J. Virol. 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided IL-12 polypeptide complexes and/or nucleic acid molecules can be delivered via virus like particles. The provided IL-23 polypeptide complexes and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The IL-12 polypeptide complexes or the IL-23 polypeptide complexes disclosed herein can be delivered by subviral dense bodies (DBs). DBs transport proteins into target cells by membrane fusion. Methods for making and using DBs are described in, for example, Pepperl-Klindworth et al., Gene Therapy 10:278-84 (2003). The provided polypeptides can be delivered by tegument aggregates. Methods for making and using tegument aggregates are described in International Publication No. WO 2006/110728.

Non-viral based delivery methods, can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Pal Alto, Calif.), Stratagene (La Jolla, Calif), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Such vectors can also be used to make the IL-12 polypeptide complexes or the IL-23 polypeptide complexes by expression in a suitable host cell, such as CHO cells.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g., β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

G. Therapeutic Applications

Also provided herein, are methods and uses for the treatment of a disease, disorder or condition associated with a target antigen comprising administering to a subject in need thereof a IL-12 polypeptide complex or a IL-23 polypeptide complex as described herein. Diseases, disorders, or conditions include, but are not limited to, cancer, inflammatory disease, an immunological disorder, autoimmune disease, infectious disease (i.e., bacterial, viral, or parasitic disease). Preferably, the disease, disorder, or condition is cancer.

Any suitable cancer may be treated with the IL-12 polypeptide complexes or the IL-23 polypeptide complexes provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. In embodiments, the cancer is melanoma or breast cancer.

In some embodiments, provided herein is a method of enhancing an immune response in a subject in need thereof by administering an effective amount of an IL-12 polypeptide complex or an IL-23 polypeptide complex provided herein to the subject. The enhanced immune response may prevent, delay, or treat the onset of cancer, a tumor, or a viral disease. Without being bound by theory, the IL-12 polypeptide complex or the IL-23 polypeptide complex enhances the immune response by activating the innate and adaptive immunities. In some embodiments, the methods described herein increase the activity of Natural Killer Cells and T lymphocytes. In some embodiments, the IL-12 polypeptide complex or the IL-23 polypeptide complex provided herein, can induce IFNγ release from Natural Killer cells as well as CD4+ and CD8+ T cells.

The method can further involve the administration of one or more additional agents to treat cancer, such as chemotherapeutic agents (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, Procarabizine), immuno-oncology agents (e.g., anti-PD-L1, anti-CTLA4, anti-PD-1, anti-CD47, anti-GD2), cellular therapies (e.g., CAR-T, T-cell therapy), oncolytic viruses and the like. Non-limiting examples of anti-cancer agents that can be used include acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1 interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinzolidine sulfate; vinzolidine sulfate; vorozole; zeniplatin; zinostatin; zorubicin hydrochloride.

In some embodiments of the methods described herein, the IL-12 polypeptide complex or the IL-23 polypeptide complex is administered in combination with an agent for the treatment of the particular disease, disorder, or condition. Agents include, but are not limited to, therapies involving antibodies, small molecules (e.g., chemotherapeutics), hormones (steroidal, peptide, and the like), radiotherapies (γ-rays, C-rays, and/or the directed delivery of radioisotopes, microwaves, UV radiation and the like), gene therapies (e.g., antisense, retroviral therapy and the like) and other immunotherapies. In some embodiments, the IL-12 polypeptide complex or the IL-23 polypeptide complex is administered in combination with anti-diarrheal agents, anti-emetic agents, analgesics and/or non-steroidal anti-inflammatory agents.

### 6. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### 7. EXAMPLES

The present invention is further described by the following examples, which are not intended to be limiting in any way.

### Example 1: HEK-Blue Assay

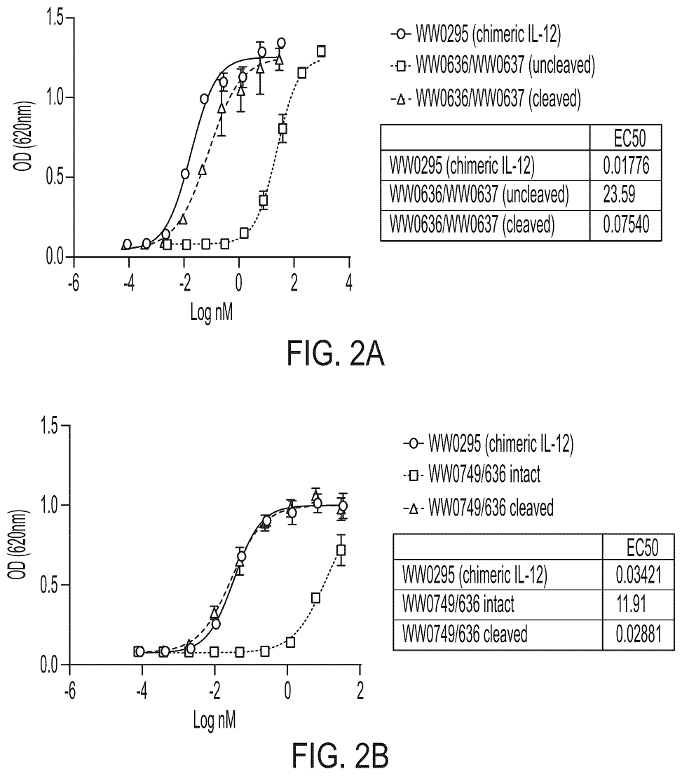

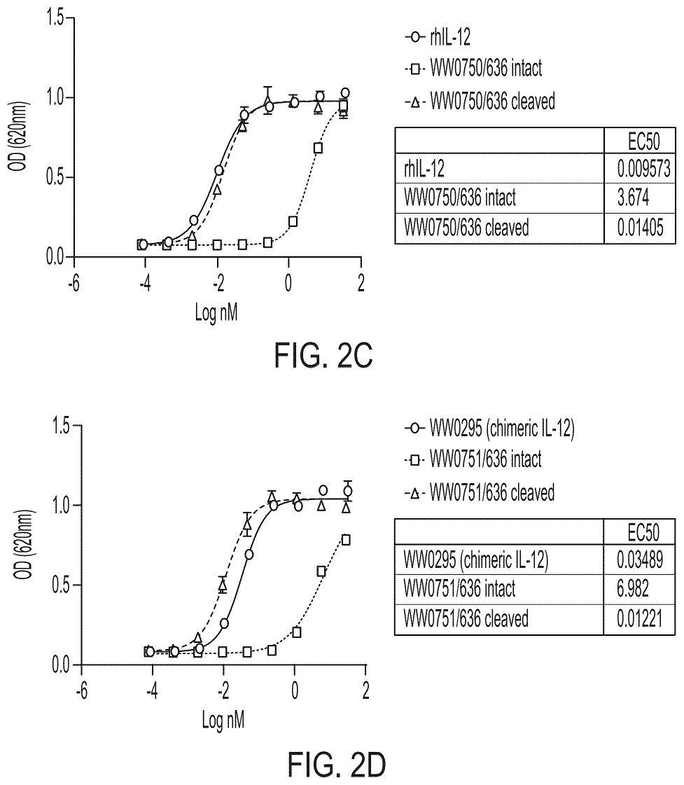

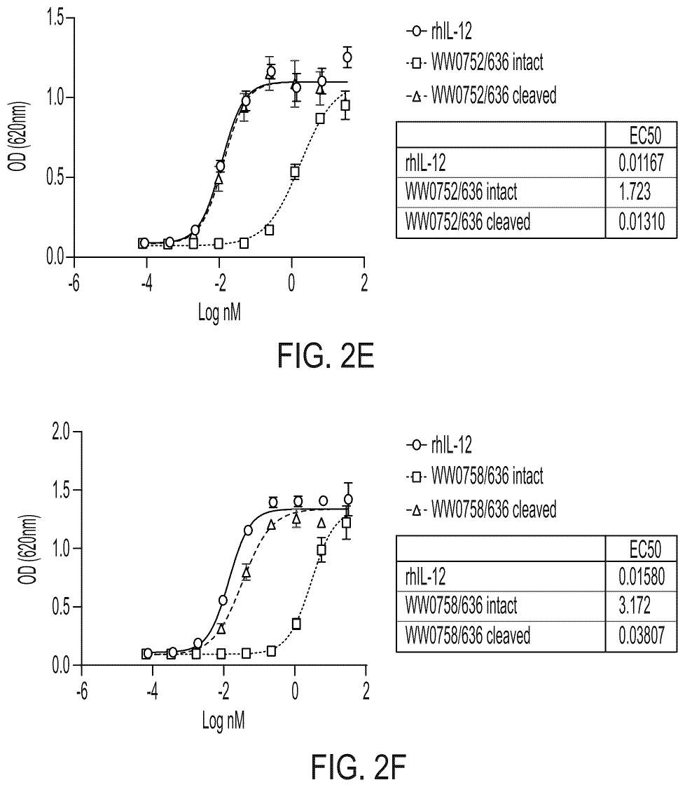

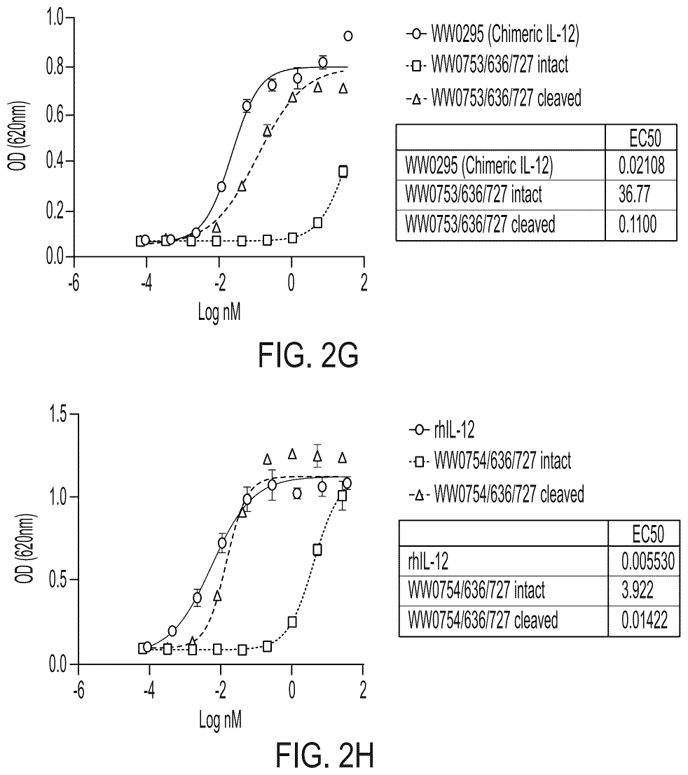

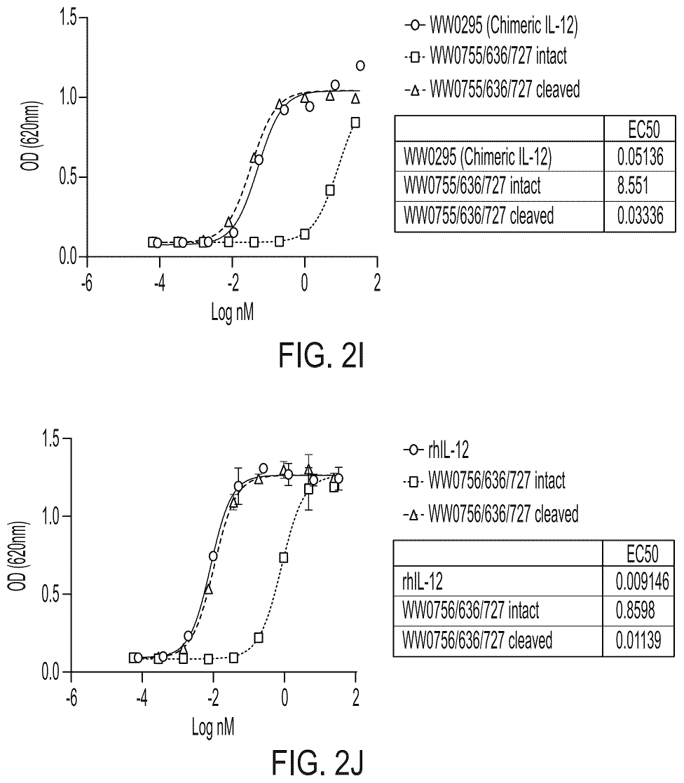

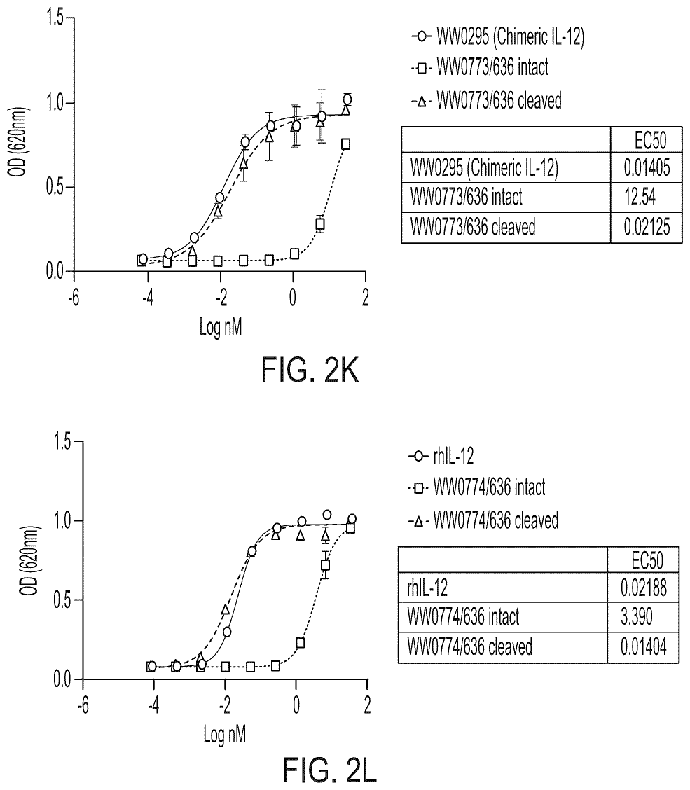

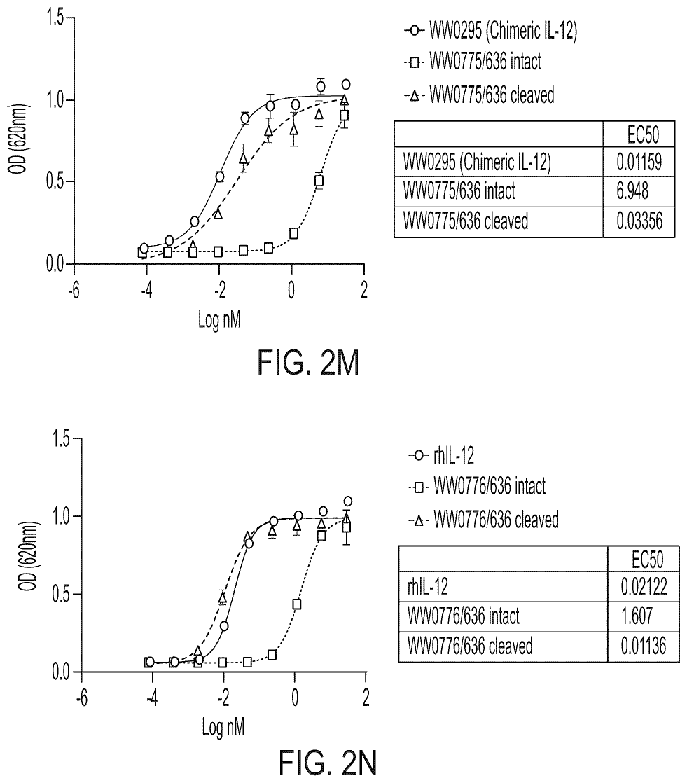

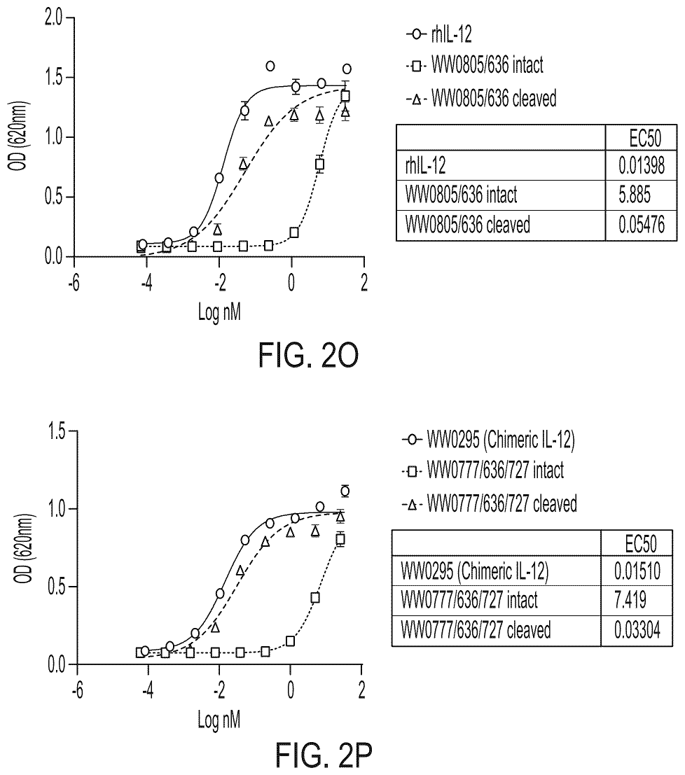

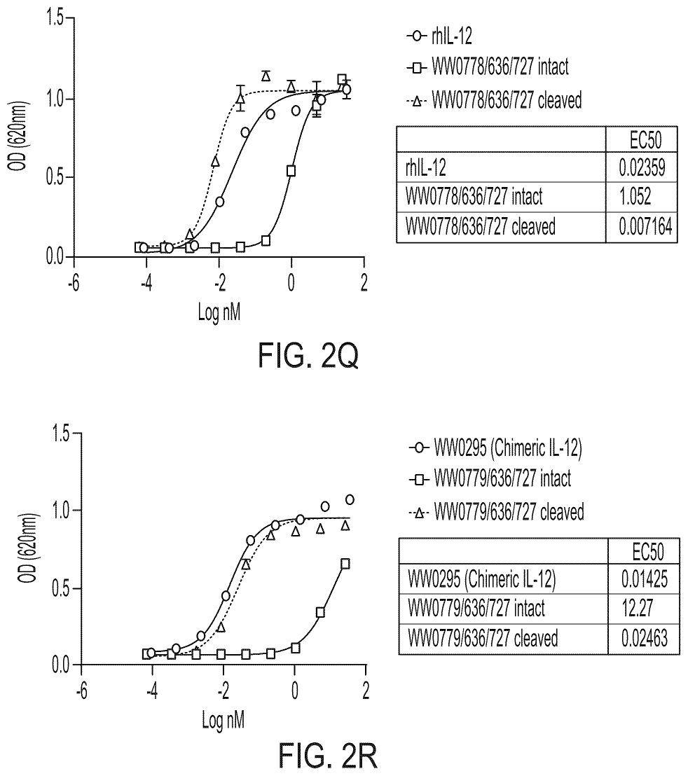

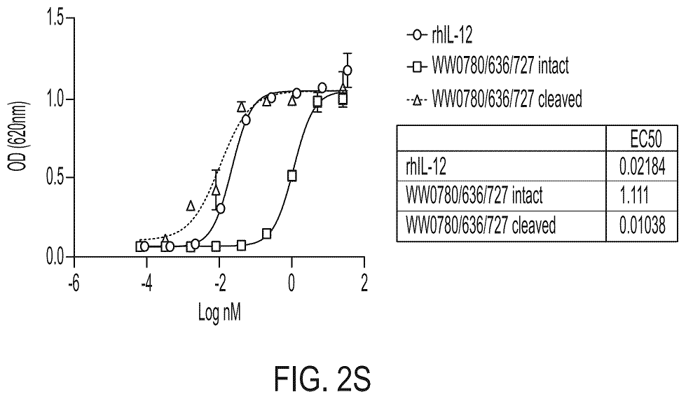

HEK-Blue IL-12 cells (InvivoGen) were plated in suspension at a density of 50,000 cells/well in culture media with or without 15 or 40 mg/ml human serum albumin (HSA) and stimulated with a dilution series of recombinant hIL-12, chimeric IL-12 (mouse p35/human p40), activatable chimeric IL-12, or activatable hIL-12 for 20-24 hours at 37° C. and 5% CO2. Activity of uncleaved and cleaved activatable hIL-12 was tested. Cleaved inducible hIL-12 was generated by incubation with active MMP9 or CTSL-1. IL-12 activity was assessed by quantification of Secreted Alkaline Phosphatase (SEAP) activity using the reagent QUANTI-Blue (InvivoGen), a colorimetric based assay. Results confirm that IL-12 fusion proteins are active and inducible. Results are shown in FIGS. 2A-2S.

### Example 2: IL-12 Luciferase Reporter Assay

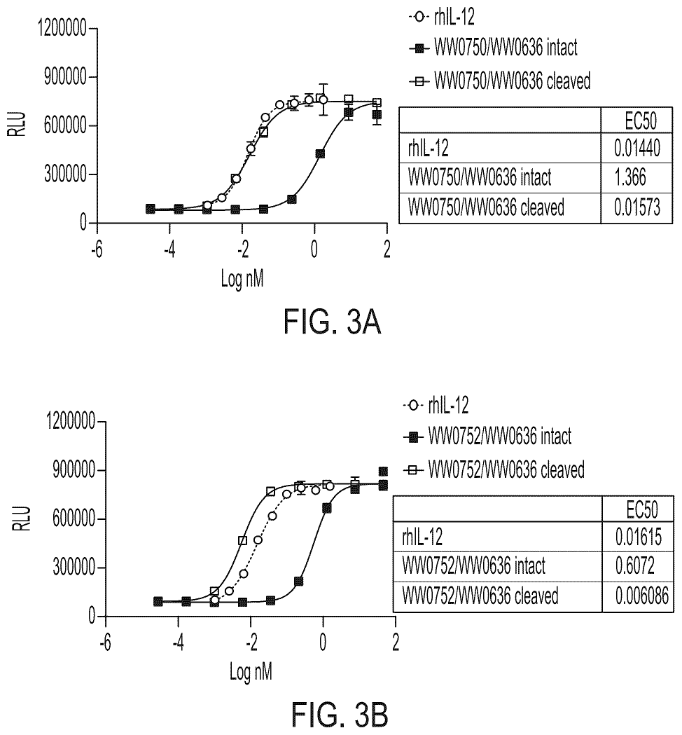

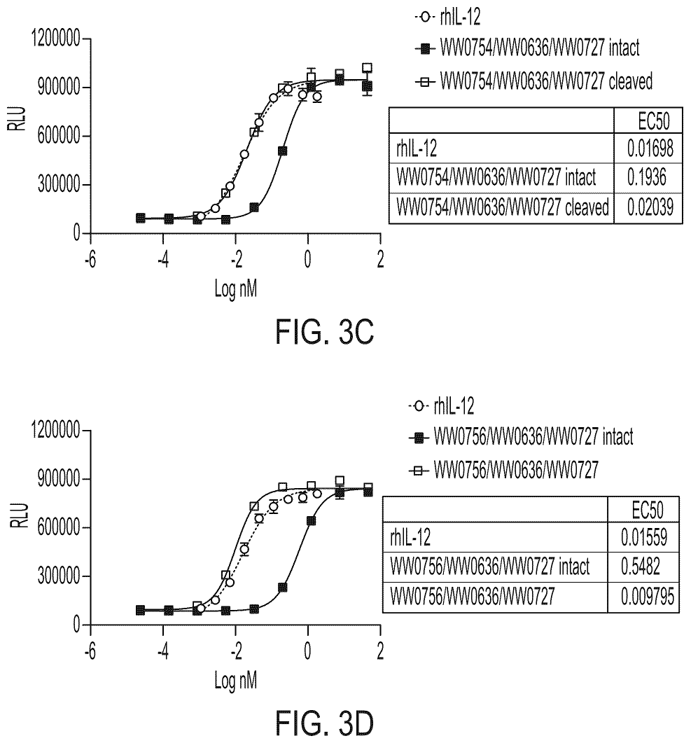

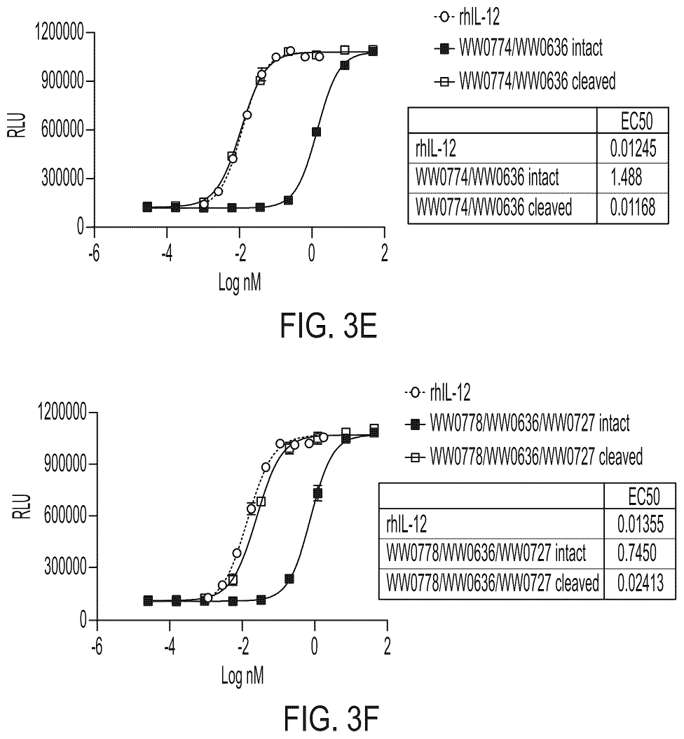

IL-12 luciferase reporter cells (Promega), purchased from the manufacturer in a “Thaw and Use” format, were plated according to the manufacturer's directions and stimulated with a dilution series of recombinant hIL-12 or activatable hIL-12 for 6 hours at 37° C. and 5% CO₂. Activity of uncleaved and cleaved activatable IL-12 was tested. Cleaved inducible IL-12 was generated by incubation with active MMP9 or CTSL-1. IL-12 activity was assessed by quantification of luciferase activity using Bio-Glo™ Reagent (Promega), which allows for the measurement of luciferase activity by luminescence readout. Results confirm that IL-12 protein fusion proteins are active and inducible. Results are shown in FIGS. 3A-3F.

### Example 3: Human T-Blast Assay

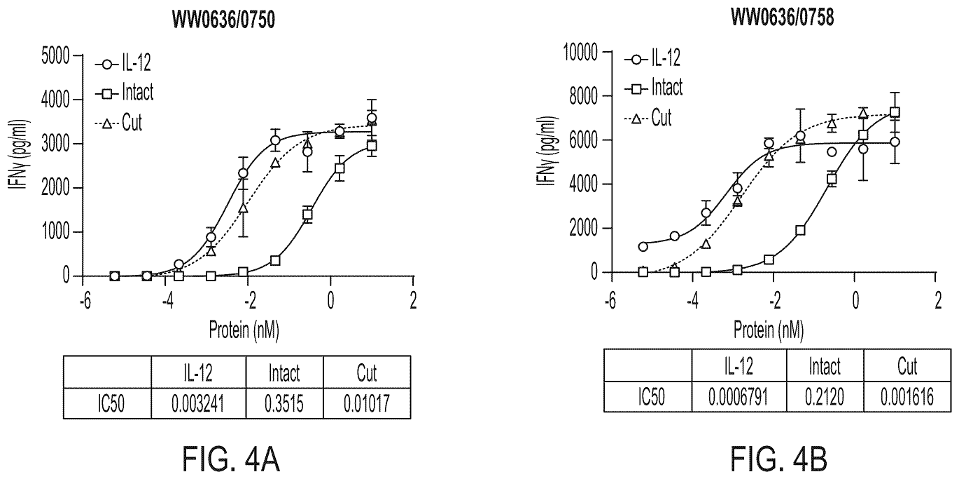

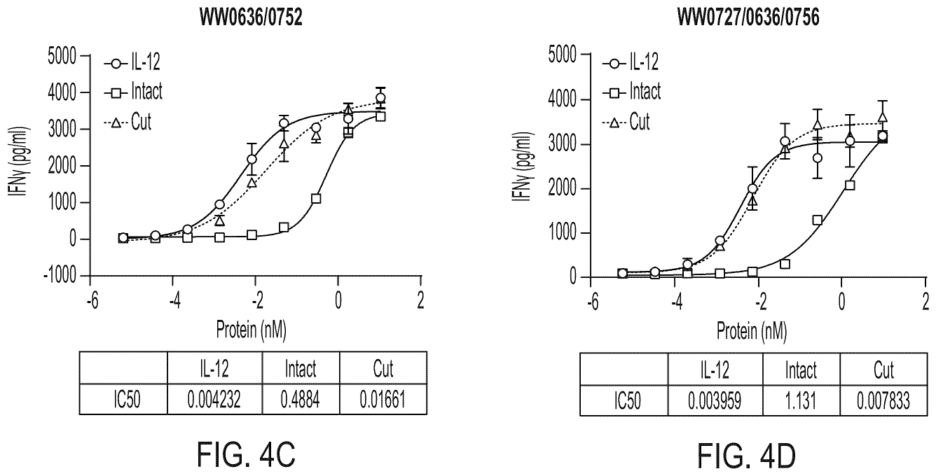

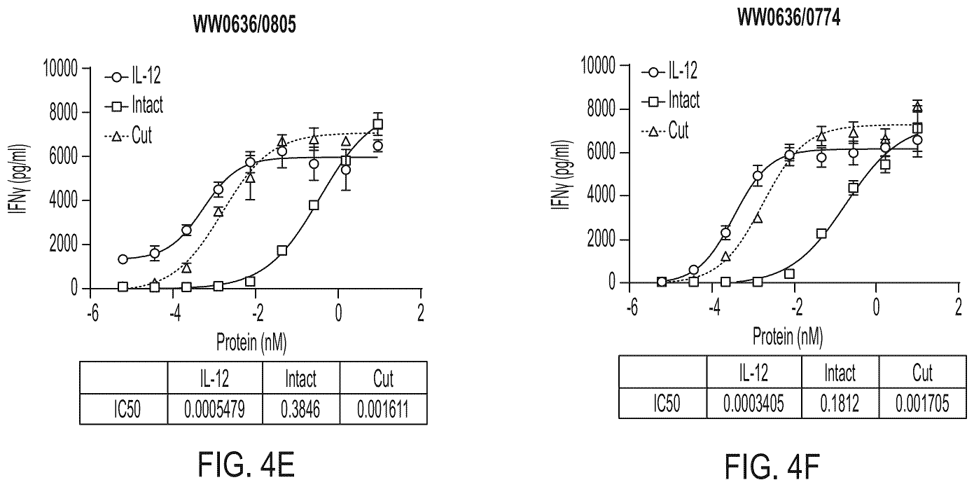

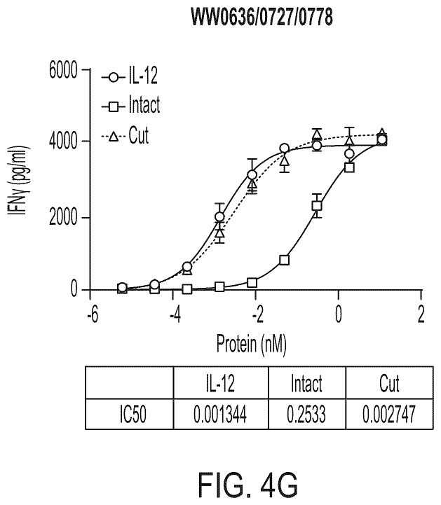

T-Blasts were induced from human PBMCs through PHA stimulation for 72 hours. T-blasts were then washed and frozen prior use. For the assay, T-Blasts were thaw and plated in suspension at 100,000 cells/well in culture media containing human albumin and stimulated with a dilution series of recombinant hIL-12 or chimeric activatable IL-12 (mouse p35/human p40) or activatable human IL-12 for 72 hours at 37° C. and 5% CO2. Activity of uncleaved and cleaved IL-12 fusion proteins was tested. Cleaved inducible hIL-12 was generated by incubation with active MMP9 or CTSL-1 enzyme. IL-12 activity was assessed by quantification of IFNγ production in supernatants using a hIFNγ Alpha-LISA kit. Results confirm that IL-12 fusion proteins are active and inducible. Results are shown in FIGS. 4A-4G.

### Example 4: Protease Cleavage of Fusion Protein by MMP9 Protease

One of skill in the art would be familiar with methods of setting up protein cleavage assay. 100 μg of protein in 1×PBS pH 7.4 were cleaved with 1 μg active MMP9 (Sigma catalog #SAE0078-50 or Enzo catalog BML-SE360) and incubated at room temperature for up to 16 hours. Digested protein was subsequently used in functional assays or stored at −80° C. prior to testing. Extent of cleavage was monitored by SDS PAGE using methods well known in the art. Full cleavage of the fusion proteins by MMP9 was seen.

### Example 5: Expression Comparison in Mammalian Host Cell Line

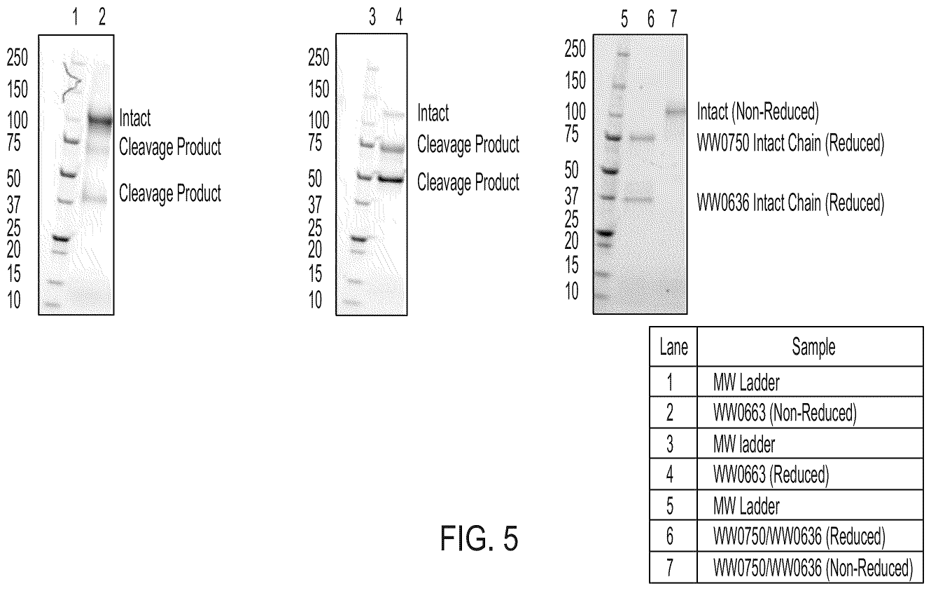

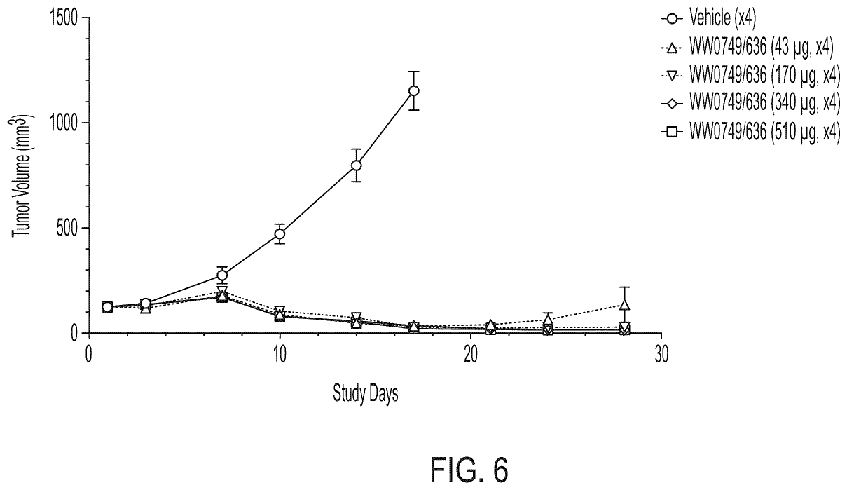

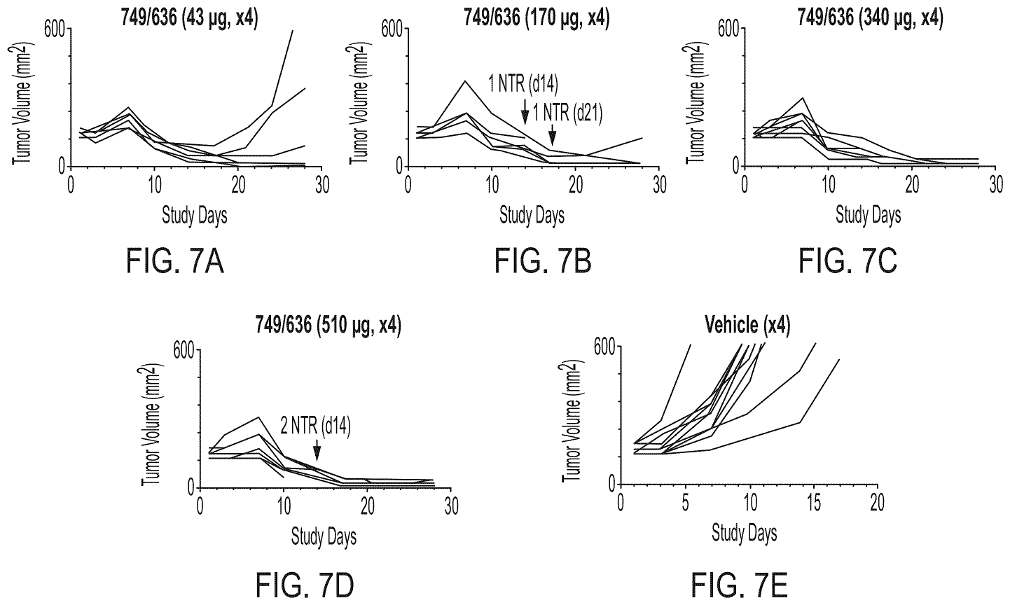

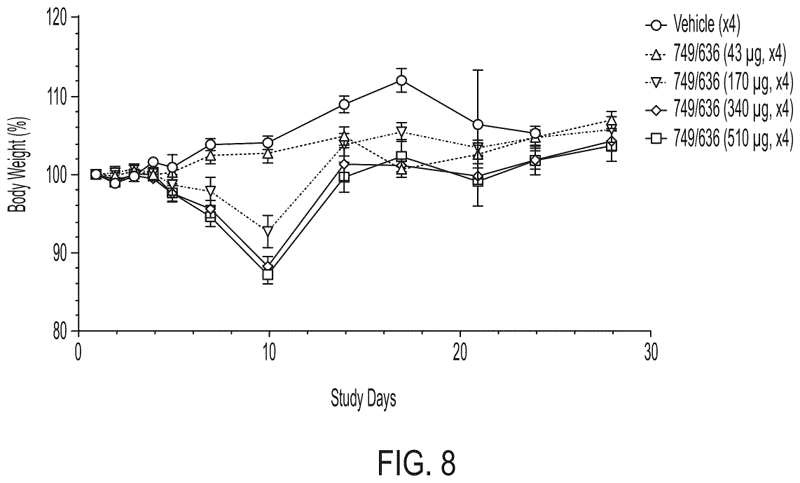

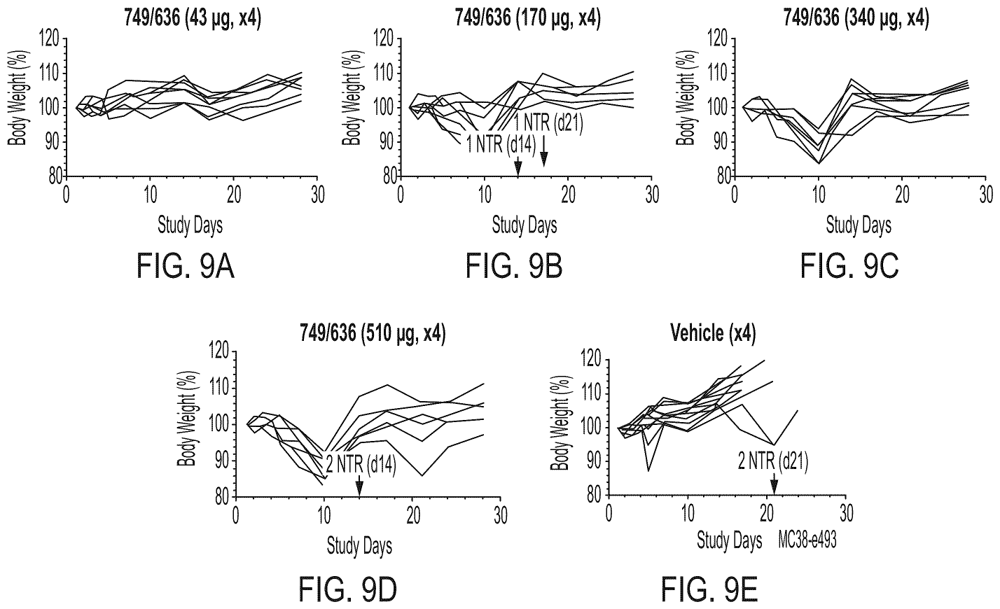

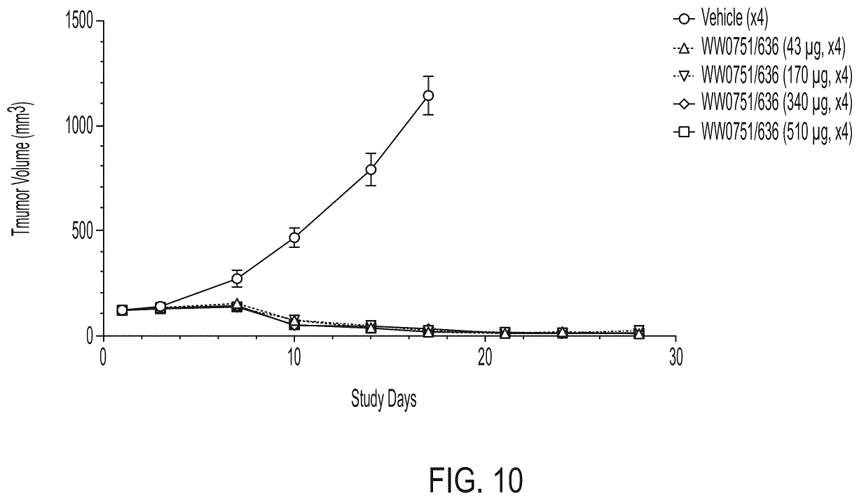

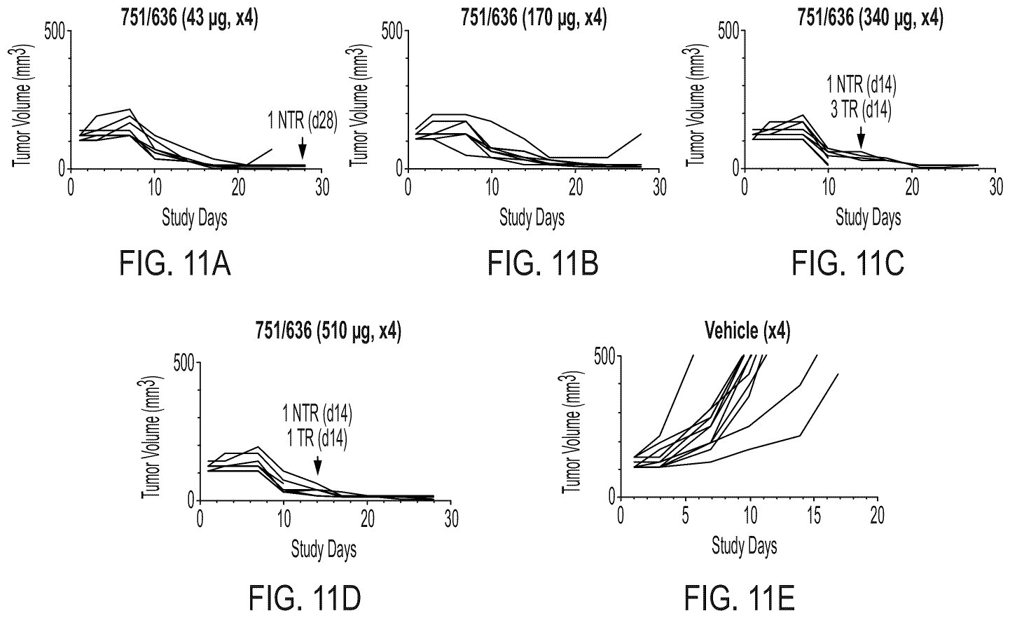

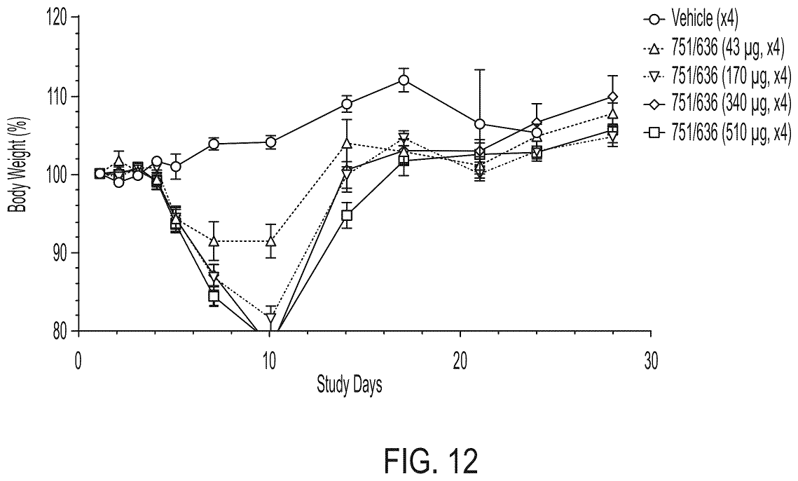

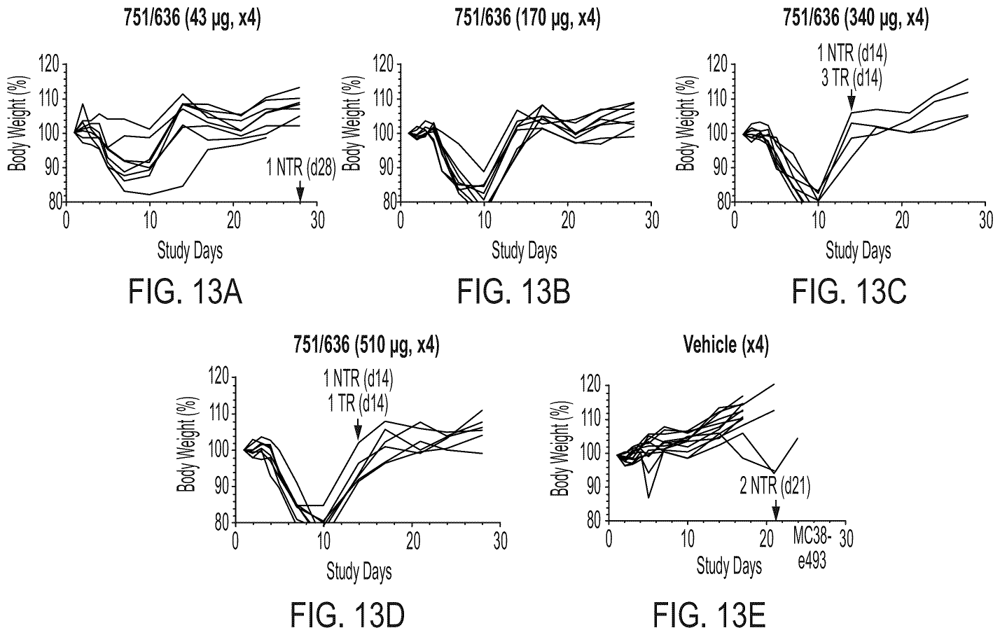

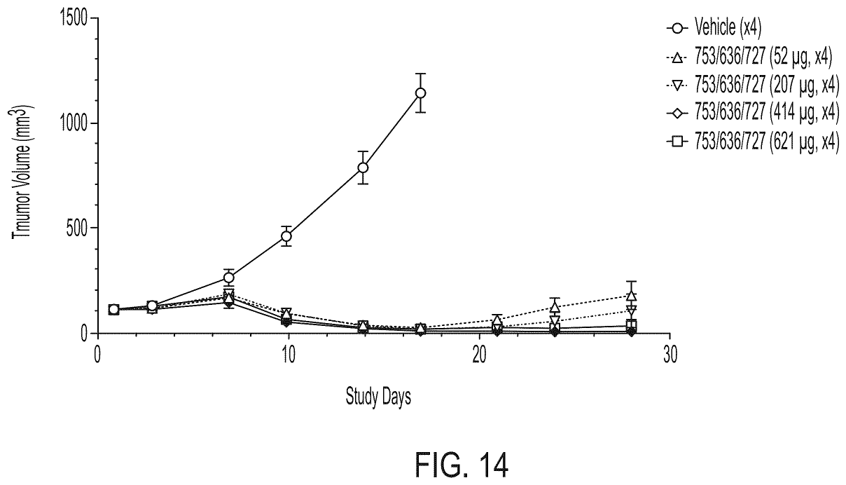

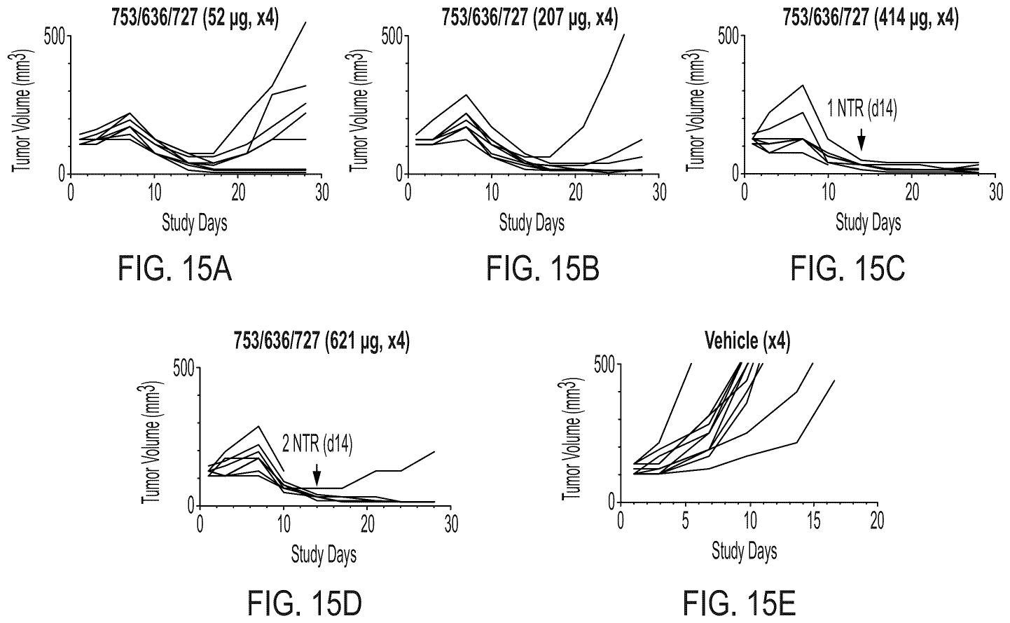

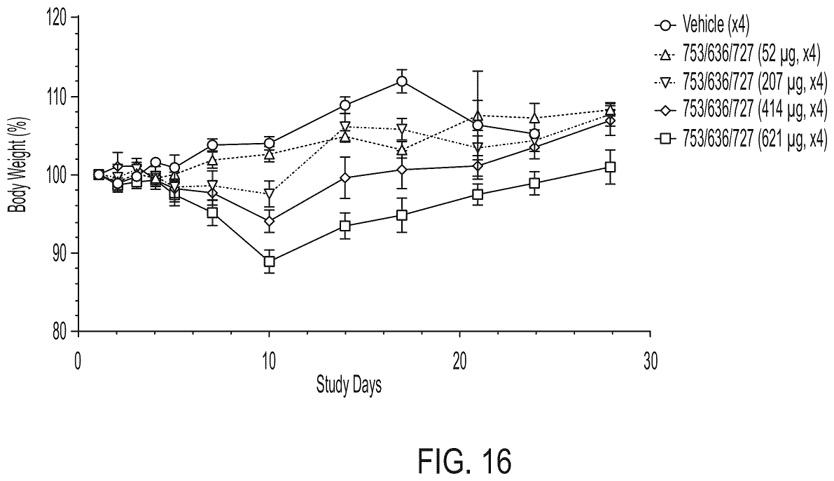

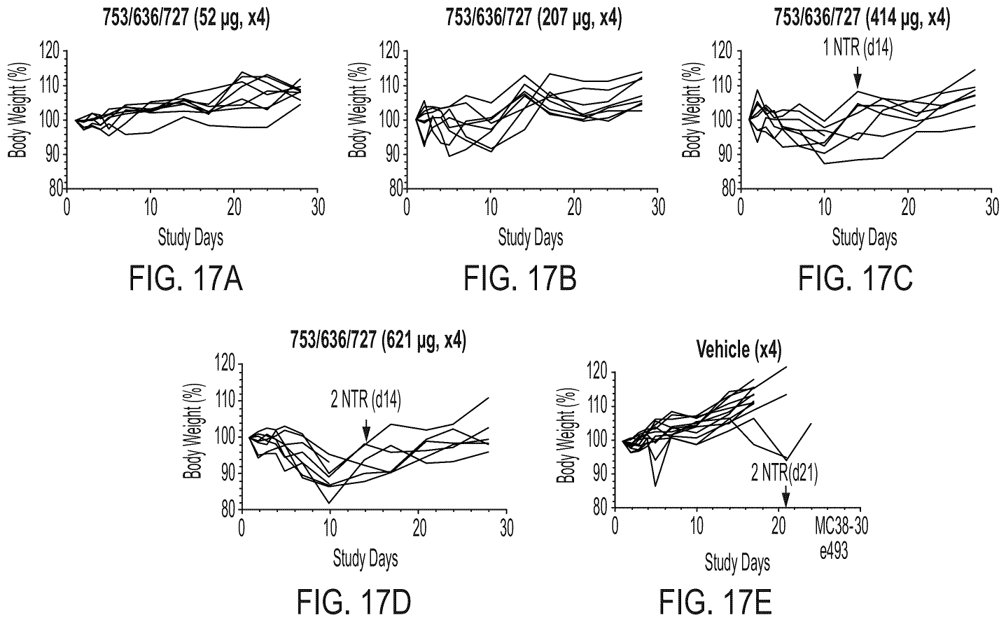

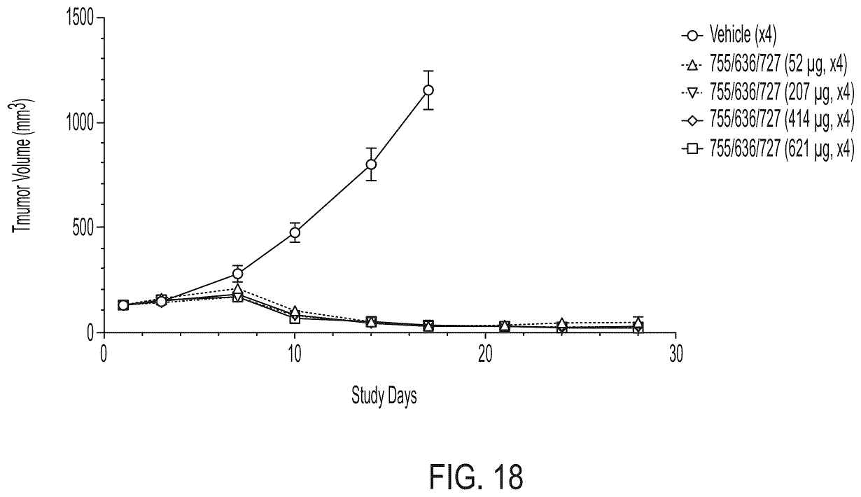

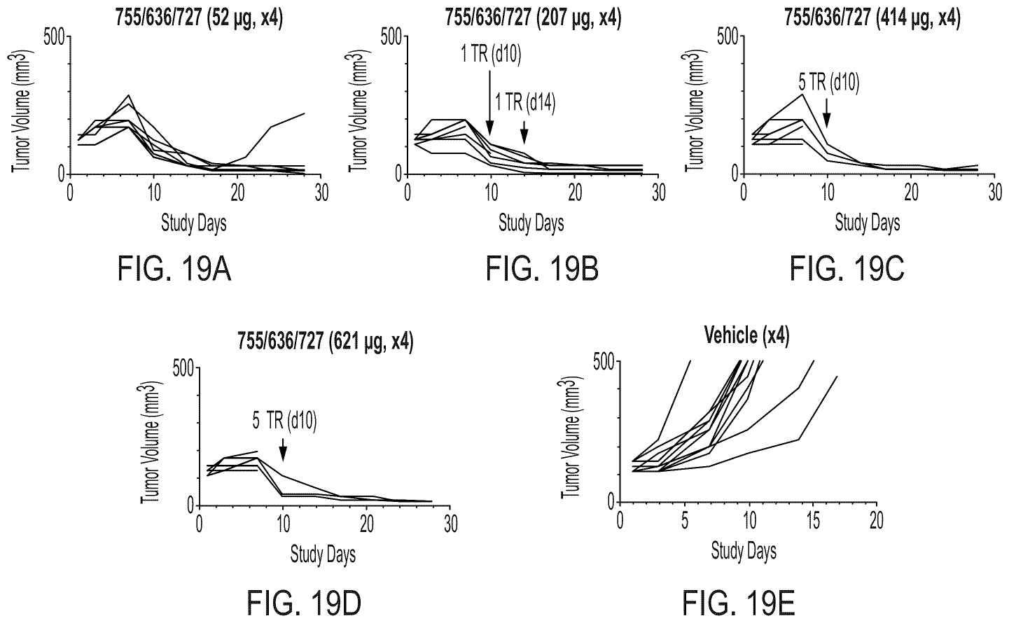

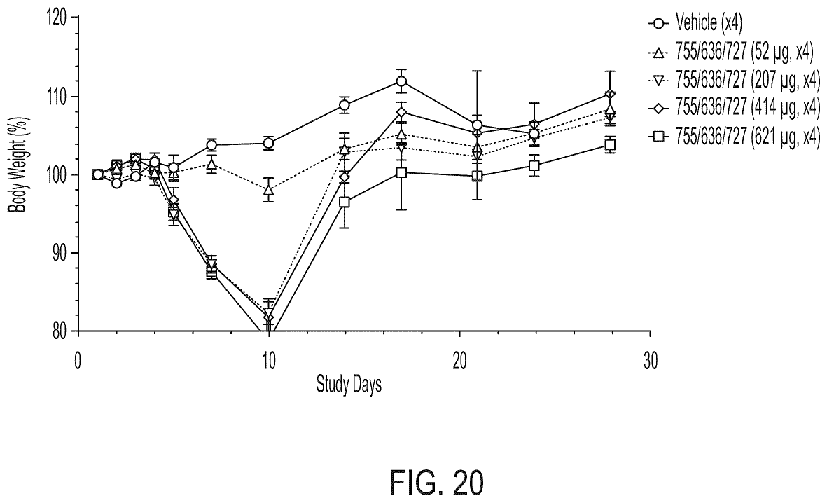

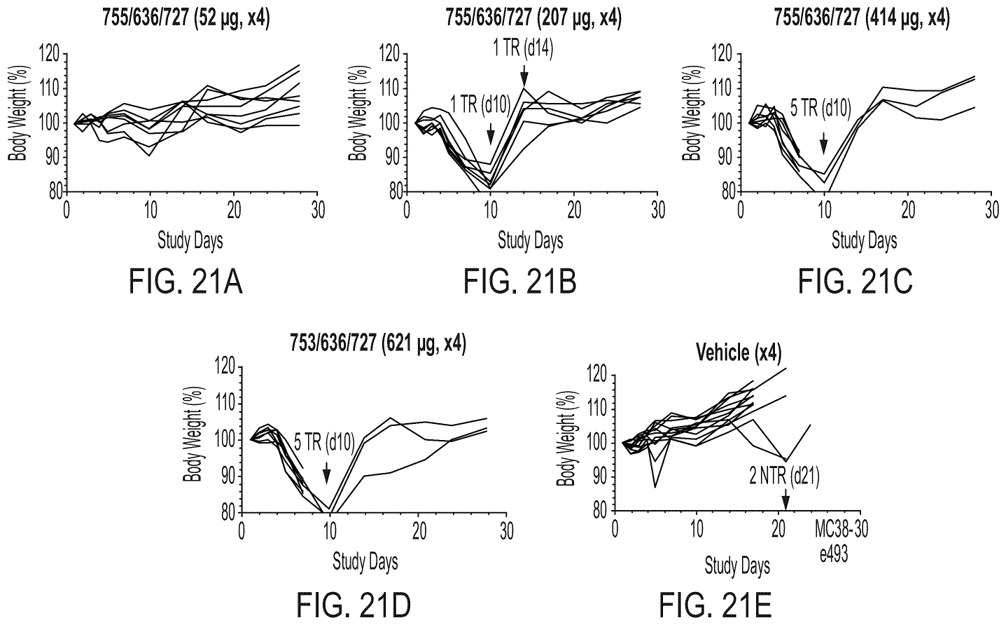

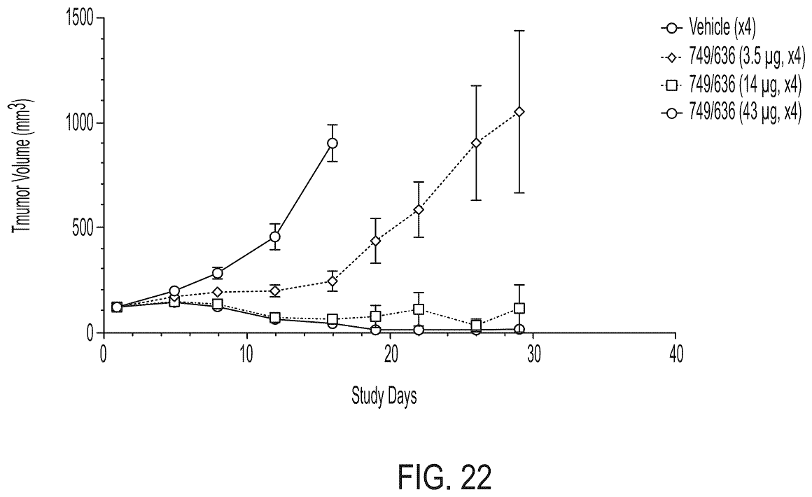

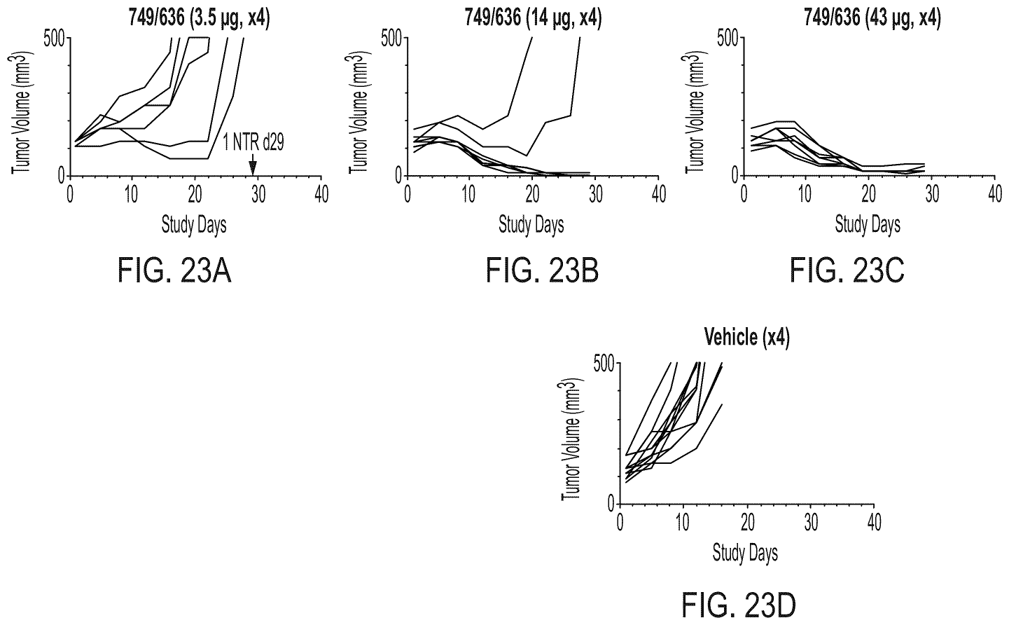

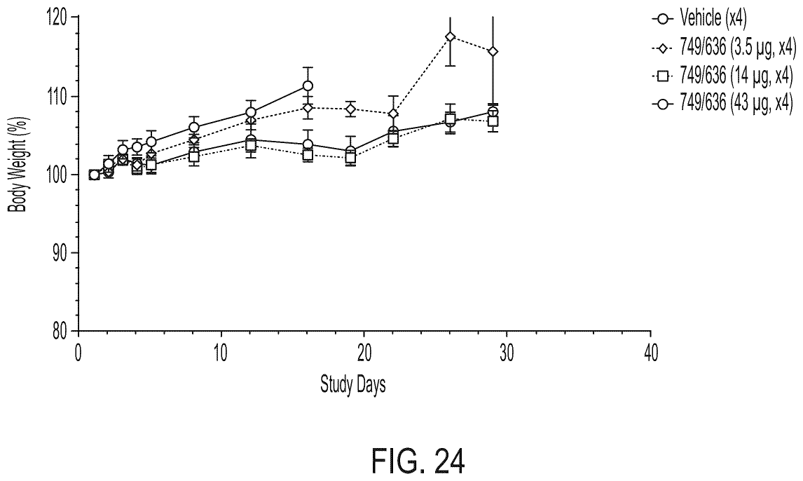

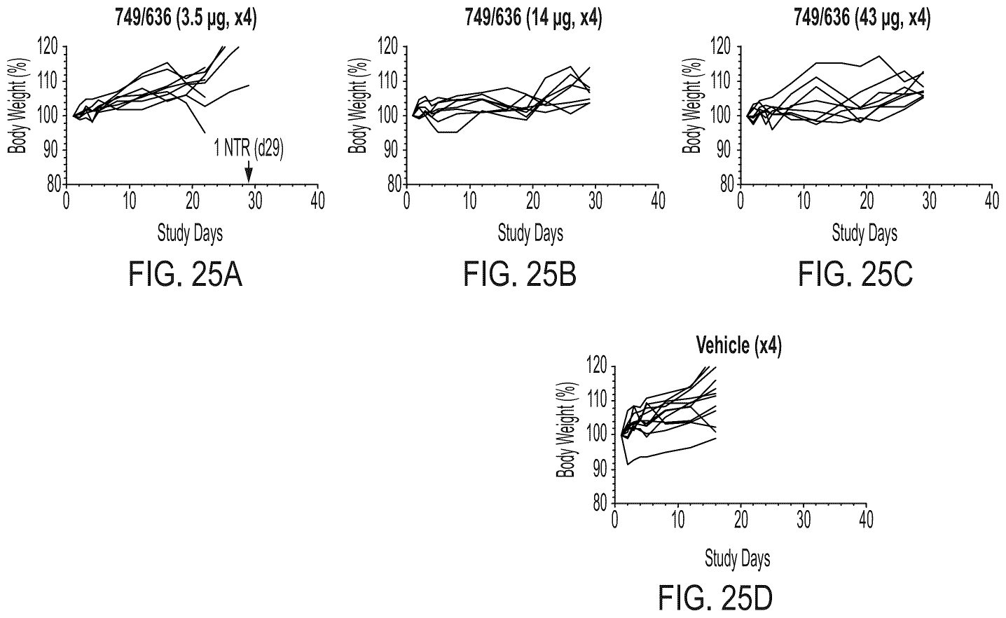

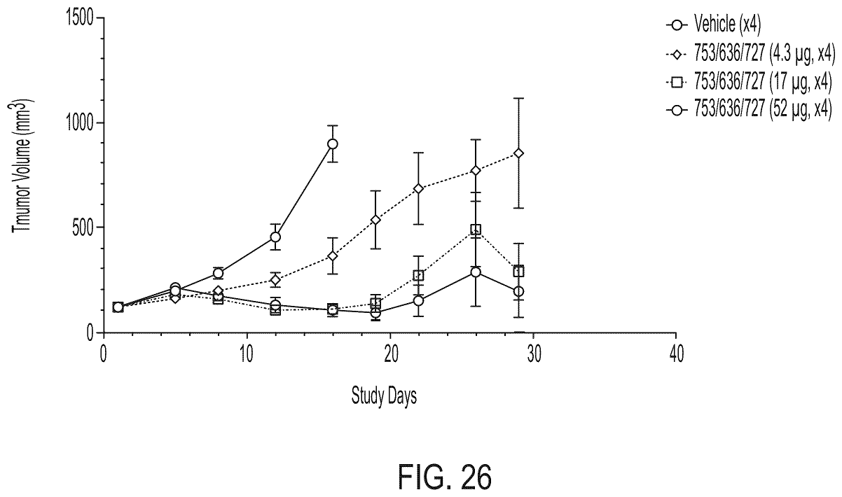

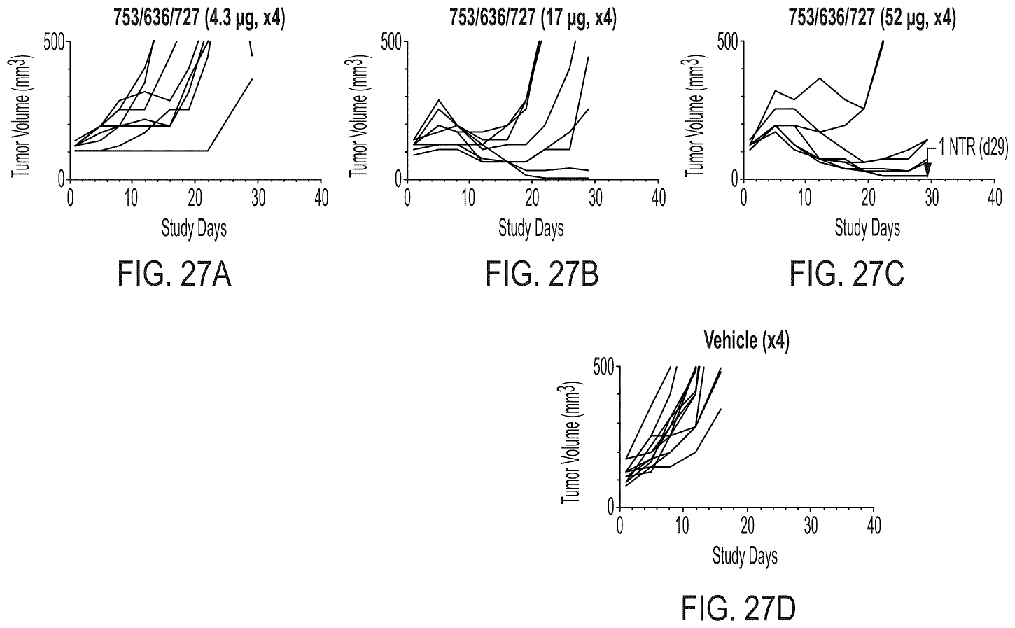

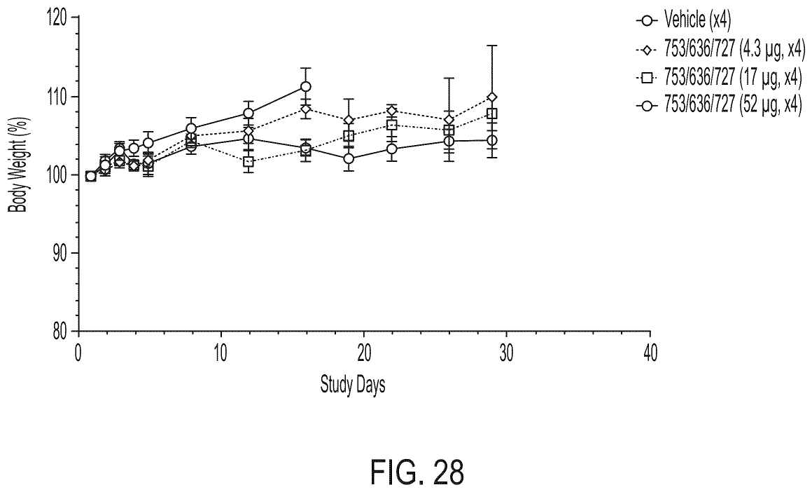

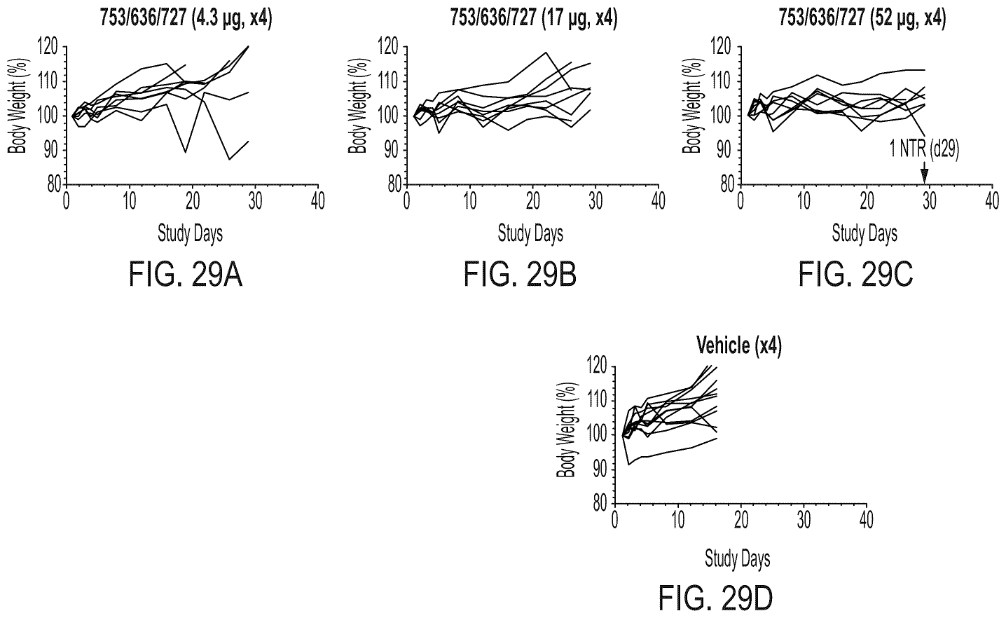

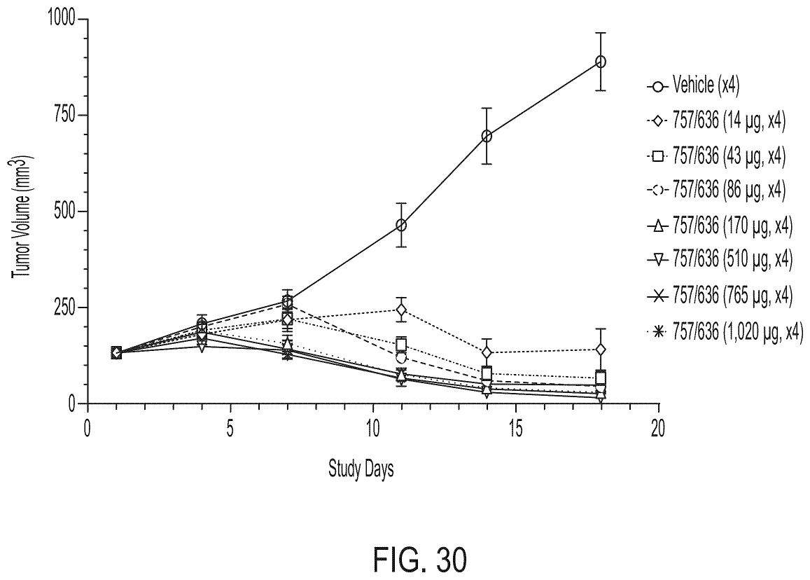

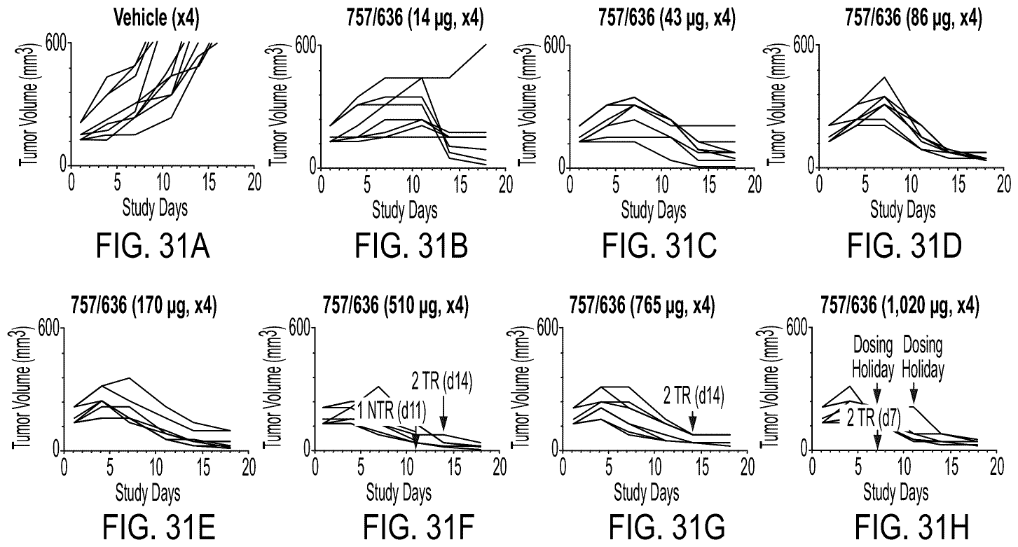

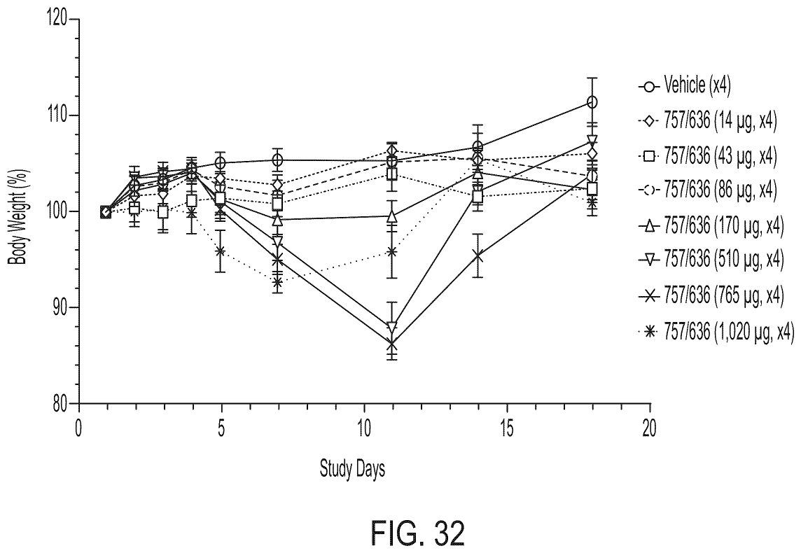

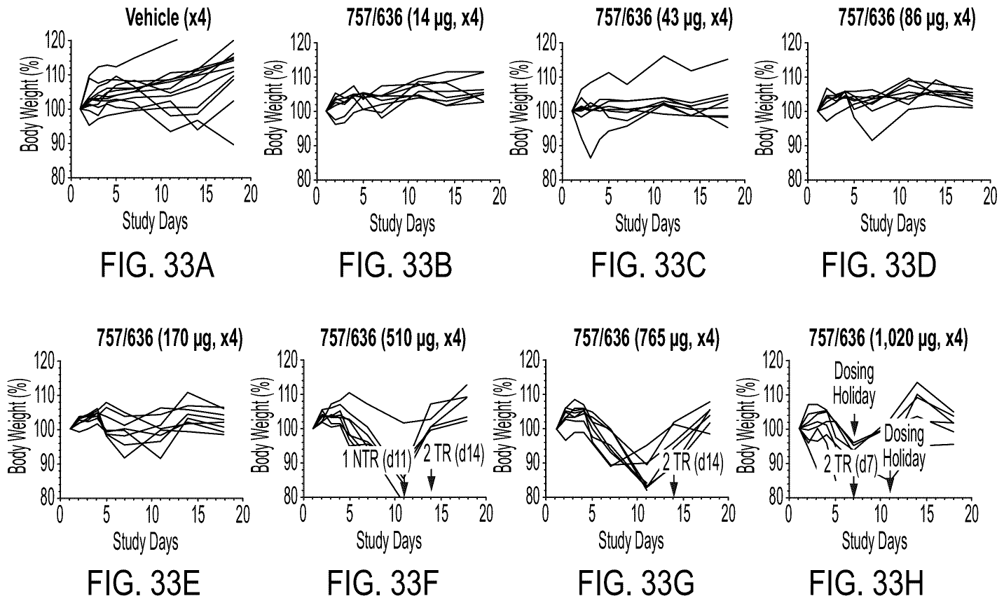

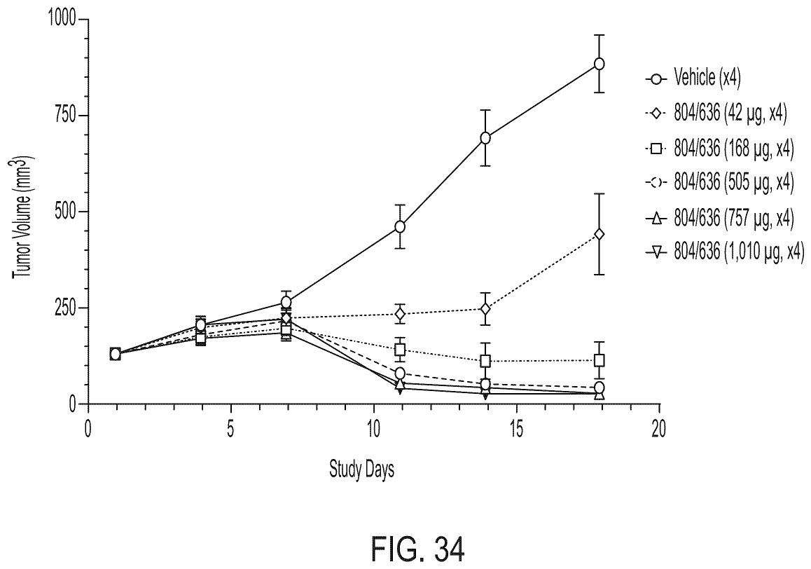

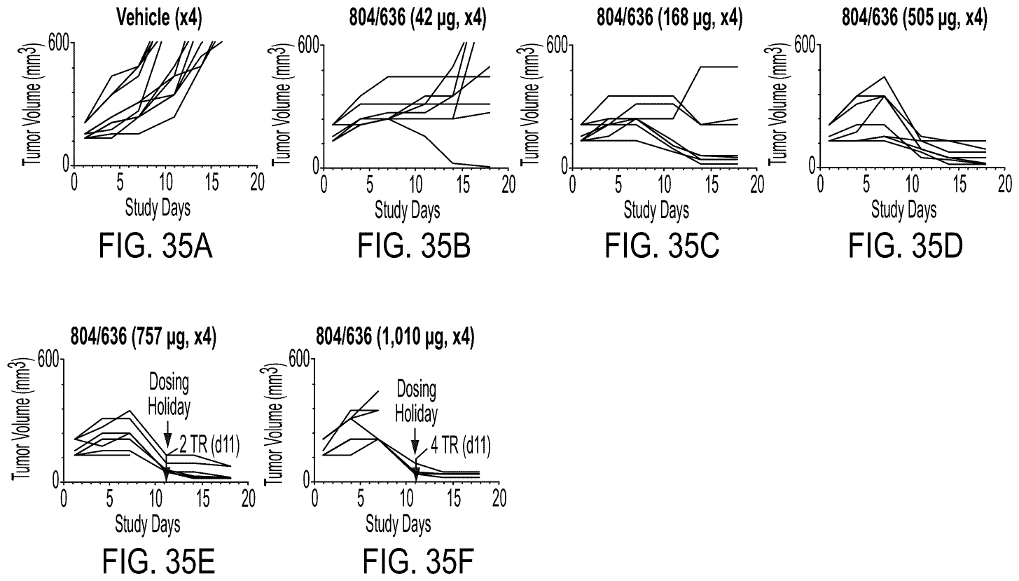

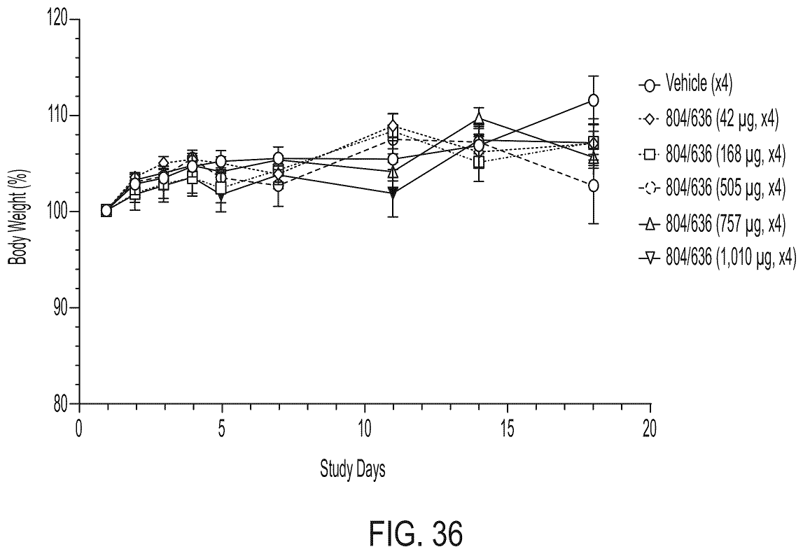

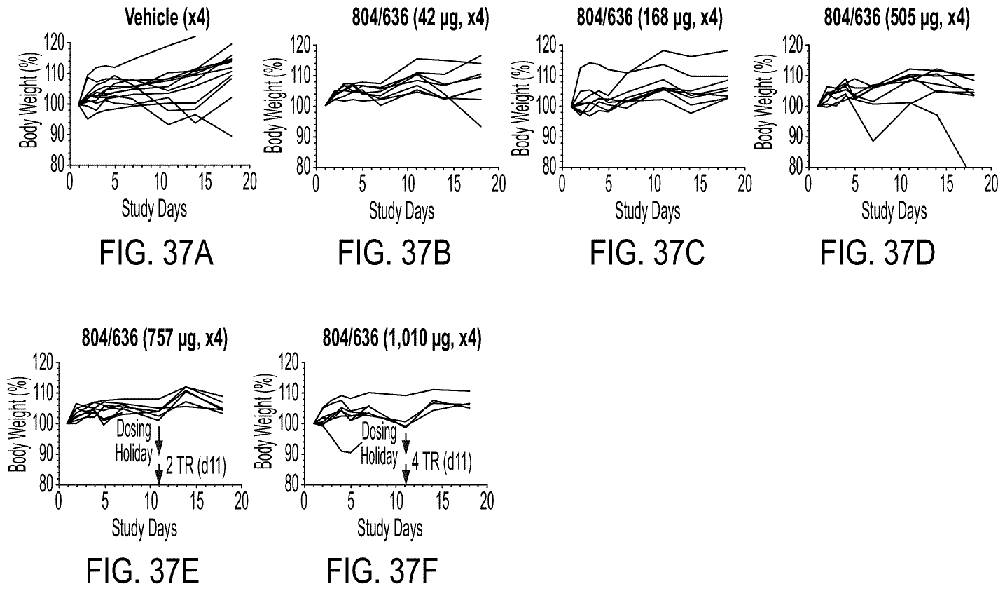

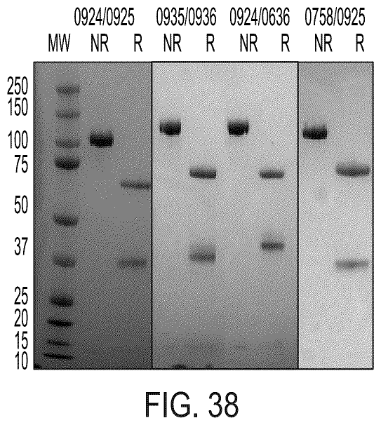

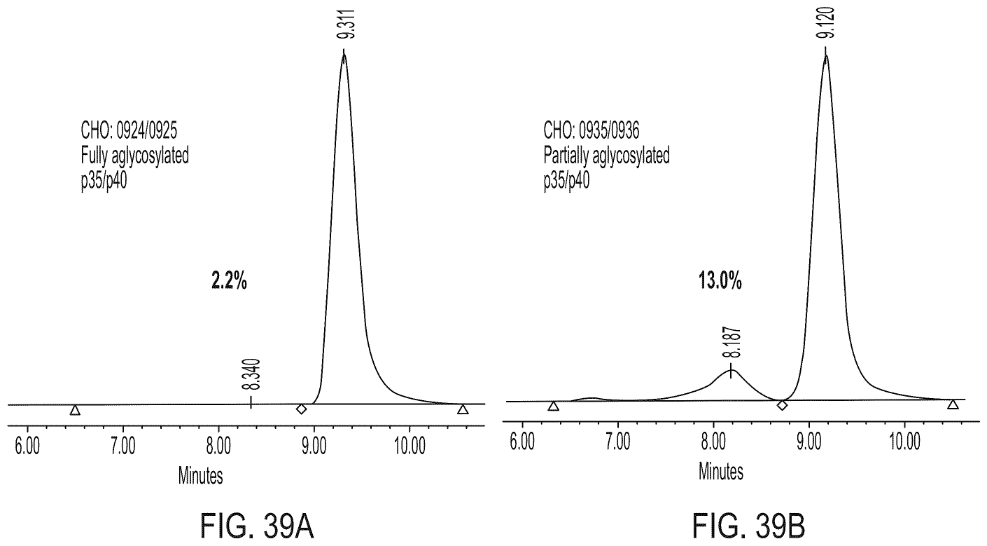

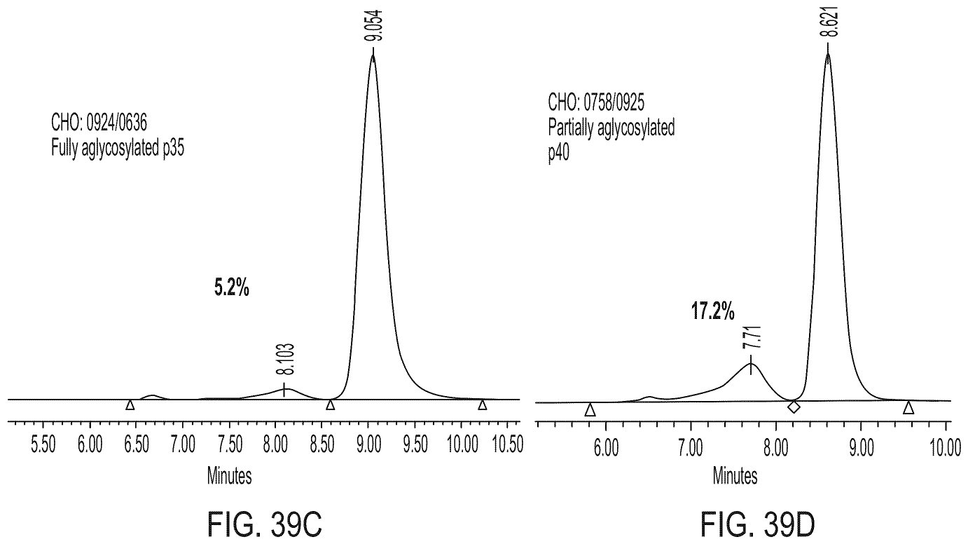

An expression plasmid for WW0663, an IL-12 fusion protein where human p40 and p35 subunits are connect by a non-cleavable linker, was transiently transfected in a mammalian expression host cell line and purified from cell supernatant by Protein A chromatography. Similarly, the expression plasmids for WW0750 and WW0636 were transiently co-transfected in the same parental mammalian host cell line as above to express an IL-12 fusion protein were human p40 and p35 subunits were not connected by a linker sequence but were assembled by a native disulfide bond. WW0750/WW0636 was purified from cell supernatant by Protein A chromatography. Both WW0663 and WW0750/WW0636 were run on non-reducing and reducing SDS-PAGE gels to compare proper assembly and any unintended cleavage products (FIG. 5). WW0663 has two unintended molecular weight fragments (cleavage products). Furthermore, in reduced conditions the intact band for WW0663 is diminished suggesting that there is an unintended cleavage at or near the linker between p40 and p35 subunits, generating two equally sized products (lowest molecular weight shown in lane 4) where p40 and p35 have been decoupled by the reduction of the p40/p35 disulfide band. Reducing and non-reducing conditions for WW0750/WW0636 (lanes 6 and 7, respectively) show the expected sizes.

### Example 6: MC38 Experiments (Study MC38-e493)

The MC38 cell line, a rapidly growing colon adenocarcinoma cell line, was used. Using this tumor model, the ability of fusion proteins to affect tumor growth and body weight was examined.

TABLE 4
Agents and treatment regime

| Group | N | Agent | Dose | Route | Schedule |
|---|---|---|---|---|---|
| 1 | 8 | Vehicle | — | ip | biwk × 2 |
| 2 | 8 | WW0749/636 | 43 μg/animal | ip | biwk × 2 |
| 3 | 8 | WW0749/636 | 170 μg/animal | ip | biwk × 2 |
| 4 | 8 | WW0749/636 | 340 μg/animal | ip | biwk × 2 |
| 5 | 8 | WW0749/636 | 510 μg/animal | ip | biwk × 2 |
| 6 | 8 | WW0751/636 | 43 μg/animal | ip | biwk × 2 |
| 7 | 8 | WW0751/636 | 170 μg/animal | ip | biwk × 2 |
| 8 | 8 | WW0751/636 | 340 μg/animal | ip | biwk × 2 |
| 9 | 8 | WW0751/636 | 510 μg/animal | ip | biwk × 2 |
| 10 | 8 | WW0753/636/727 | 52 μg/animal | ip | biwk × 2 |
| 11 | 8 | WW0753/636/727 | 207 μg/animal | ip | biwk × 2 |
| 12 | 8 | WW0753/636/727 | 414 μg/animal | ip | biwk × 2 |
| 13 | 8 | WW0753/636/727 | 621 μg/animal | ip | biwk × 2 |
| 14 | 8 | WW0755/636/727 | 52 μg/animal | ip | biwk × 2 |
| 15 | 8 | WW0755/636/727 | 207 μg/animal | ip | biwk × 2 |
| 16 | 8 | WW0755/636/727 | 414 μg/animal | ip | biwk × 2 |
| 17 | 8 | WW0755/636/727 | 621 μg/animal | ip | biwk × 2 |

Mice were anaesthetized with isoflurane for implant of cells to reduce the ulcerations. 326 CR female C57BL/6 mice were set up with 5×10⁵ MC38 tumor cells in 0% Matrigel sc in flank. Cell injection volume was 0.1 mL/mouse. Mouse age at start date was 8 to 12 weeks. Pair matches were performed when tumors reach an average size of 100-150 mm3 and begin treatment. This is Day 1 of study start. Body weights were taken at initiation and then biweekly to the end. Caliper measurements were taken biweekly to the end. Any adverse reactions were reported immediately. Any individual animal with a single observation of >than 25% body weight loss or three consecutive measurements of >20% body weight loss was euthanized. Any group with a mean body weight loss of >20% or >10% mortality stopped dosing; the group was not euthanized, and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 40 days, whichever comes first. When the endpoint was reached, the animals were euthanized.

### Example 7: MC38 Experiments (Study MC38-e495)

The MC38 cell line, a rapidly growing colon adenocarcinoma cell line, was used. Using this tumor model, the ability of fusion proteins to affect tumor growth and body weight was examined.

TABLE 5
Agents and treatment regime

| Group | N | Agent | Dose | Route | Schedule |
|---|---|---|---|---|---|
| 1 | 8 | Vehicle | — | ip | biwk × 2 |
| 2 | 8 | WW0662 | 3.5 μg/animal | ip | biwk × 2 |
| 3 | 8 | WW0662 | 14 μg/animal | ip | biwk × 2 |
| 4 | 8 | WW0662 | 43 μg/animal | ip | biwk × 2 |
| 5 | 8 | WW0749/636 | 3.5 μg/animal | ip | biwk × 2 |
| 6 | 8 | WW0749/636 | 14 μg/animal | ip | biwk × 2 |
| 7 | 8 | WW0749/636 | 43 μg/animal | ip | biwk × 2 |
| 8 | 8 | WW0753/636/727 | 4.3 μg/animal | ip | biwk × 2 |
| 9 | 8 | WW0753/636/727 | 17 μg/animal | ip | biwk × 2 |
| 10 | 8 | WW0753/636/727 | 52 μg/animal | ip | biwk × 2 |
| 11 | 8 | WW0773/636 | 14 μg/animal | ip | biwk × 2 |
| 12 | 8 | WW0773/636 | 42 μg/animal | ip | biwk × 2 |
| 13 | 8 | WW0773/636 | 168 μg/animal | ip | biwk × 2 |
| 14 | 8 | WW0773/636 | 505 μg/animal | ip | biwk × 2 |
| 15 | 8 | WW0777/636/727 | 17 μg/animal | ip | biwk × 2 |
| 16 | 8 | WW0777/636/727 | 51 μg/animal | ip | biwk × 2 |
| 17 | 8 | WW0777/636/727 | 204 μg/animal | ip | biwk × 2 |
| 18 | 8 | WW0777/636/727 | 613 μg/animal | ip | biwk × 2 |

Mice were anaesthetized with isoflurane for implant of cells to reduce the ulcerations. 326 CR female C57BL/6 mice were set up with 5×10⁵ MC38 tumor cells in 0% Matrigel sc in flank. Cell injection volume was 0.1 mL/mouse. Mouse age at start date was 8 to 12 weeks. Pair matches were performed when tumors reach an average size of 100-150 mm³ and begin treatment. This is Day 1 of study start. Body weights were taken at initiation and then biweekly to the end. Caliper measurements were taken biweekly to the end. Any adverse reactions were reported immediately. Any individual animal with a single observation of >than 25% body weight loss or three consecutive measurements of >20% body weight loss was euthanized. Any group with a mean body weight loss of >20% or >10% mortality stopped dosing; the group was not euthanized, and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 40 days, whichever comes first. When the endpoint was reached, the animals were euthanized

### Example 8: MC38 Experiments (Study MC38-e503)

The MC38 cell line, a rapidly growing colon adenocarcinoma cell line, was used. Using this tumor model, the ability of fusion proteins to affect tumor growth and body weight was examined.

TABLE 6
Agents and treatment regime

| Group | N | Agent | Dose | Route | Schedule |
|---|---|---|---|---|---|
| 1 | 12 | Vehicle | — | ip | biwk × 2 |
| 2 | 8 | WW0757/636 | 14 μg/animal | ip | biwk × 2 |
| 3 | 8 | WW0757/636 | 43 μg/animal | ip | biwk × 2 |
| 4 | 8 | WW0757/636 | 86 μg/animal | ip | biwk × 2 |
| 5 | 8 | WW0757/636 | 170 μg/animal | ip | biwk × 2 |
| 6 | 8 | WW0757/636 | 510 μg/animal | ip | biwk × 2 |
| 7 | 8 | WW0757/636 | 765 μg/animal | ip | biwk × 2 |
| 8 | 8 | WW0757/636 | 1,020 μg/animal | ip | biwk × 2 |
| 9 | 8 | WW0804/636 | 42 μg/animal | ip | biwk × 2 |
| 10 | 8 | WW0804/636 | 168 μg/animal | ip | biwk × 2 |
| 11 | 8 | WW0804/636 | 505 μg/animal | ip | biwk × 2 |
| 12 | 8 | WW0804/636 | 757 μg/animal | ip | biwk × 2 |
| 13 | 8 | WW0804/636 | 1,010 μg/animal | ip | biwk × 2 |

Mice were anaesthetized with isoflurane for implant of cells to reduce the ulcerations. 326 CR female C57BL/6 mice were set up with 5×10⁵ MC38 tumor cells in 0% Matrigel sc in flank. Cell injection volume was 0.1 mL/mouse. Mouse age at start date was 8 to 12 weeks. Pair matches were performed when tumors reach an average size of 100-150 mm³ and begin treatment. This is Day 1 of study start. Body weights were taken at initiation and then biweekly to the end. Caliper measurements were taken biweekly to the end. Any adverse reactions were reported immediately. Any individual animal with a single observation of >than 25% body weight loss or three consecutive measurements of >20% body weight loss was euthanized. Any group with a mean body weight loss of >20% or >10% mortality stopped dosing; the group was not euthanized, and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 40 days, whichever comes first. When the endpoint was reached, the animals were euthanized.

### Example 9: Octet Binding Kinetics Assay

KD measurements were performed with scFvs using multi-concentration kinetics. The binding affinities for human IL-12 were measured using an Octet QKe instrument (ForteBio). A strategy of capturing 6×His tagged (SEQ ID NO: 446) scFvs on sensors followed by association/dissociation of IL-12 was used. The BLI analysis was performed at 30° C. using 1× kinetics buffer (ForteBio) as assay buffer. Ni-NTA (NTA) biosensors (ForteBio) were first presoaked in assay buffer for greater than 5 minutes. Test scFv (5 μg/mL) was captured on the sensor for 300 seconds. Sensors were then dipped in assay buffer for 120 seconds to establish a baseline before measuring binding to IL-12. Sensors were then dipped into varying concentrations of IL-12 (50 to 0.78 nM, 2-fold dilutions in assay buffer) and a blank buffer well for reference subtraction for 300 seconds to measure association. Dissociation of IL-12 was then measured by dipping sensors into assay buffer for 300 seconds. Agitation at all steps was 1000 rpm. Kinetic parameters were generated with Octet Data Analysis Software Version 8.2 using reference subtraction (scFv “binding” to buffer), dissociation based inter-step correction, 1 to 1 binding model, and global fit (Rmax unlinked by sensor). KD values are shown in Table 7.

TABLE 7
Summarizes scFv IL-12 blocker kinetics

| scFv | kon (1/Ms) | koff (1/s) | KD (M) |
|---|---|---|---|
| WW0478 | 3.70E+05 | 6.00E−04 | 1.60E−09 |
| WW0479 | 3.20E+05 | 2.50E−04 | 7.70E−10 |
| WW0480 | NB | NB | NB |
| WW0481 | 3.50E+05 | 8.30E−05 | 2.30E−10 |
| WW0482 | 3.30E+05 | 1.00E−04 | 3.10E−10 |
| WW0483 | 2.80E+05 | 2.50E−04 | 9.00E−10 |
| WW0484 | 3.30E+05 | 1.40E−04 | 4.40E−10 |
| WW0485 | 2.90E+05 | 7.70E−05 | 2.70E−10 |
| WW0486 | 3.20E+05 | 4.50E−05 | 1.40E−10 |
| WW0487 | 3.20E+05 | 7.80E−05 | 2.40E−10 |
| WW0488 | 3.20E+05 | 8.00E−05 | 2.50E−10 |
| WW0489 | 3.40E+05 | 2.90E−04 | 8.50E−10 |
| WW0490 | 2.50E+05 | 1.20E−04 | 4.90E−10 |
| WW0491 | 3.20E+05 | 1.10E−04 | 3.60E−10 |
| WW0492 | 6.70E+05 | 2.50E−04 | 3.70E−09 |
| WW0493 | 6.90E+05 | 2.70E−03 | 3.90E−09 |
| WW0494 | 3.20E+05 | 2.50E−04 | 7.80E−10 |
| WW0495 | 3.00E+05 | 1.50E−04 | 4.90E−10 |
| WW0496 | 5.50E+05 | 5.00E−05 | 9.00E−11 |
| WW0497 | NB | NB | NB |
| WW0498 | 3.10E+05 | 1.00E−04 | 3.30E−10 |
| WW0499 | 2.60E+05 | 7.20E−04 | 2.80E−09 |
| WW0500 | 2.90E+05 | 1.70E−04 | 5.80E−10 |
| WW0501 | 3.50E+05 | 4.20E−05 | 1.20E−10 |
| WW0502 | 3.60E+05 | 7.70E−05 | 2.20E−10 |
| WW0503 | 3.50E+05 | 7.30E−05 | 2.10E−10 |
| WW0504 | 3.40E+05 | 1.90E−04 | 5.60E−10 |
| WW0505 | 3.00E+05 | 7.20E−05 | 2.40E−10 |
| WW0506 | 4.30E+05 | 7.60E−05 | 1.80E−10 |
| WW0507 | 3.00E+05 | 1.10E−04 | 3.80E−10 |
| WW0508 | 4.60E+05 | 5.00E−06 | 1.10E−11 |
| WW0509 | 3.00E+05 | 1.40E−04 | 4.80E−10 |
| WW0510 | 3.90E+05 | 2.30E−04 | 5.80E−10 |
| WW0511 | 4.50E+05 | 9.60E−04 | 2.10E−09 |
| WW0512 | 4.80E+05 | 4.90E−05 | 1.00E−10 |
| WW0653 | 3.00E+05 | 5.27E−05 | 1.76E−10 |
| WW0654 | 3.07E+05 | 2.13E−04 | 6.94E−10 |
| WW0655 | 2.87E+05 | 1.17E−04 | 4.09E−10 |
| WW0656 | 2.79E+05 | 3.90E−04 | 1.40E−09 |
| WW0657 | 2.90E+05 | 4.15E−04 | 1.43E−09 |
| WW0658 | 2.40E+05 | 2.50E−04 | 1.04E−09 |
| WW0659 | 3.46E+05 | 1.42E−04 | 4.12E−10 |
| WW0660 | 2.99E+05 | 3.10E−04 | 1.04E−09 |
| WW0661 | 3.00E+05 | 2.50E−04 | 8.33E−10 |

### Example 10: HEKBlue IL-23 Reporter Assay

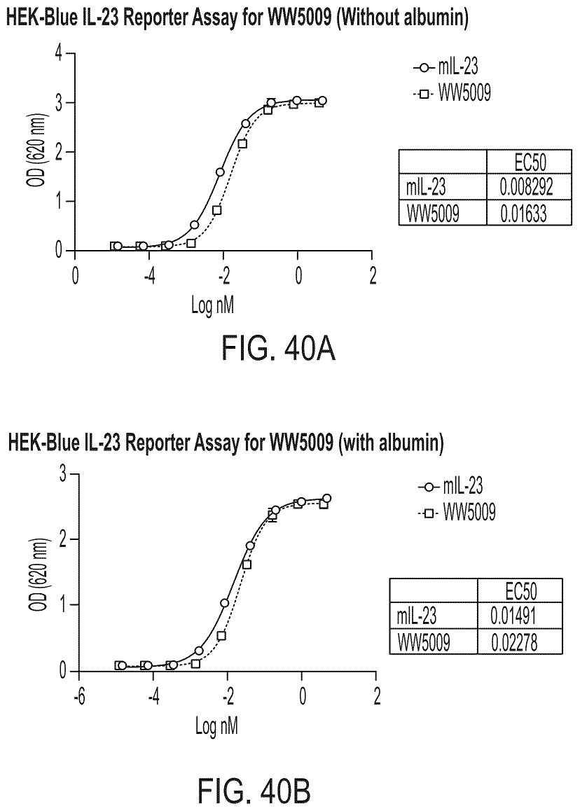

HEK-Blue IL23 cells (InvivoGen) were plated in suspension at a density of 50,000 cells/well in culture media with or without 15 mg/ml human serum albumin (HSA) and stimulated with a dilution series of recombinant mouse IL-23 or half-life extended mouse IL23 (anti-HSA-L-mIL23) for 20-24 hours at 37° C. and 5% CO2. IL-23 activity was assessed by quantification of Secreted Alkaline Phosphatase (SEAP) activity using the reagent QUANTI-Blue (InvivoGen), a colorimetric based assay. Results are shown in FIGS. 40A and 40B.

### Example 11: MC38 Efficacy Study Using Half-Life Extended IL-23 Protein WW5009

The MC38 cell line, a rapidly growing colon adenocarcinoma cell line, were used. Using this tumor model, the ability of fusion proteins to affect tumor growth was examined.

TABLE 8
Agents and treatment regime

| Group | N | Agent | Dose | Route | Schedule |
|---|---|---|---|---|---|
| 1 | 8 | Vehicle | — | ip | biwk × 3 |
| 2 | 8 | WW5009 | 1 μg/animal | ip | biwk × 3 |
| 3 | 8 | WW5009 | 10 μg/animal | ip | biwk × 3 |
| 4 | 8 | WW5009 | 100 μg/animal | ip | biwk × 3 |

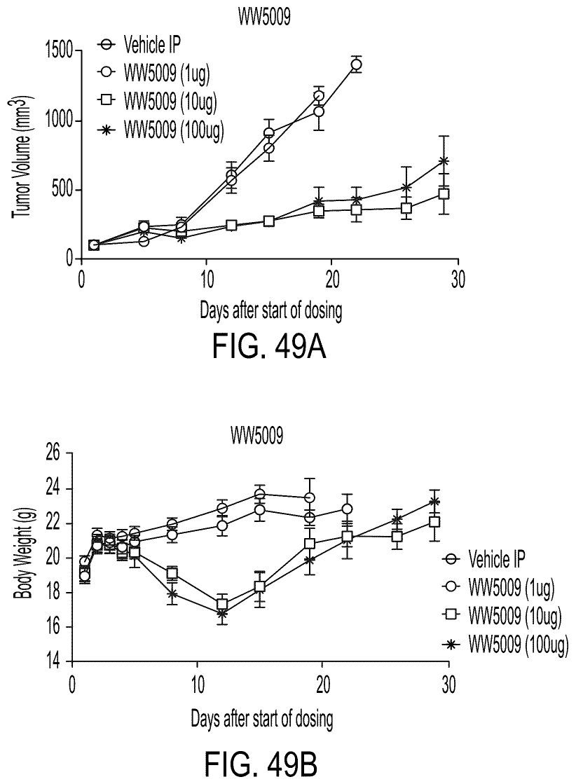

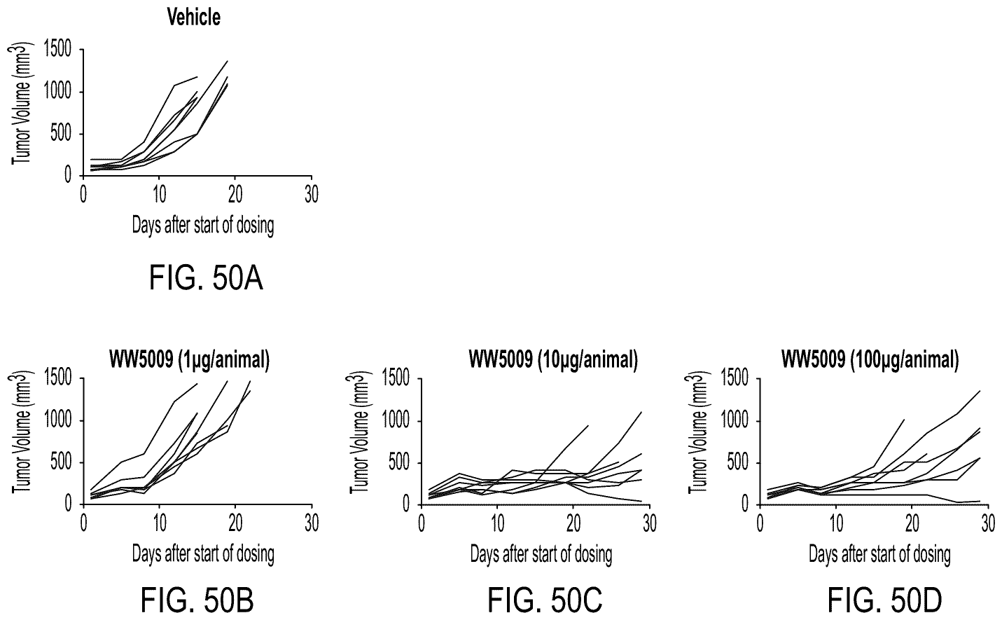

Mice were anaesthetized with isoflurane for implant of cells to reduce the ulcerations. Charles River female C57BL/6 mice were set up with 5×10⁵ MC38 tumor cells in 0% Matrigel sc in flank. Cell Injection Volume will be 0.1 mL/mouse. Mouse age at start date will be 8 to 12 weeks. Pair matches were performed when tumors reach an average size of 100-150 mm³ and begin treatment. Body weights were taken at initiation and then biweekly to the end. Caliper measurements were taken biweekly to the end. Any adverse reactions were reported immediately. Any individual animal with a single observation of >than 30% body weight loss or three consecutive measurements of >25% body weight loss were euthanized. Any group with a mean body weight loss of >20% or >10% mortality stopped dosing; the group was not euthanized, and recovery was allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint is reached, the animals were euthanized. Results are shown in FIGS. 49A, 49B, and 50A-50D.

### Example 12: CT26 Experiments (Study CT26-e676)

The CT26 cell line, a rapidly growing colon adenocarcinoma cell line, was used. Using this tumor model, the ability of fusion proteins to affect tumor growth was examined.

TABLE 9
Agents and Treatment

| Group | N | Agent | Dose | Route | Schedule |
|---|---|---|---|---|---|
| 1 | 10 | Vehicle | — | ip | biwk × 2 |
| 2 | 10 | WW0757/636 | 50 μg/animal | ip | biwk × 2 |
| 3 | 10 | WW0757/636 | 100 μg/animal | ip | biwk × 2 |

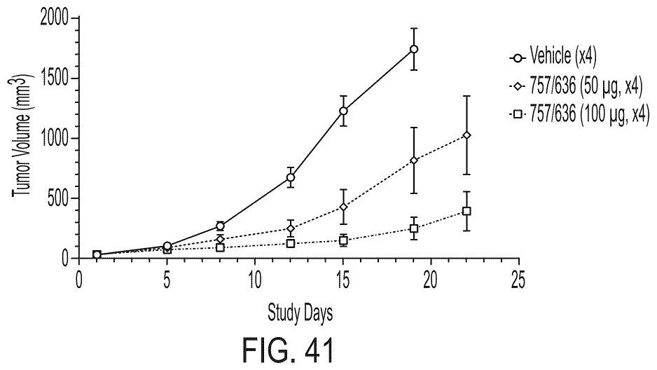

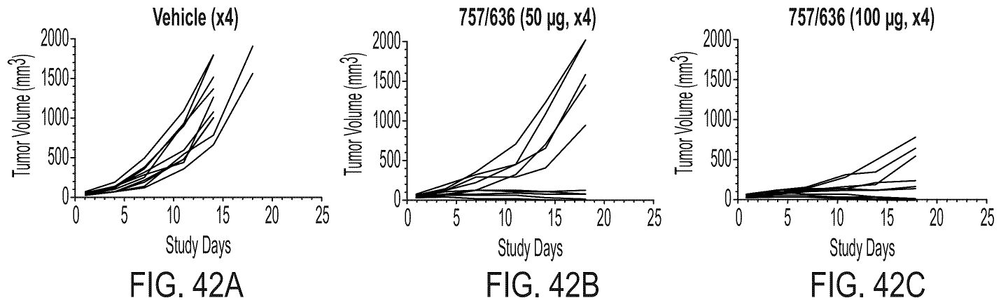

30 CR female BALB/c mice were set up with 3×10⁵ CT26 tumor cells in 0% Matrigel sc in flank. Cell injection volume was 0.1 mL/mouse. Mouse age at start date was 8 to 12 weeks. Pair matches were performed when tumors reach an average size of 30-60 mm³ and begin treatment. This is Day 1 of study start. Caliper measurements were taken biweekly to the end. Any adverse reactions were reported immediately. Any individual animal with a single observation of >than 25% body weight loss or three consecutive measurements of >20% body weight loss was euthanized. Any group with a mean body weight loss of >20% or >10% mortality stopped dosing; the group was not euthanized, and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 2000 mm³ or 22 days, whichever comes first. When the endpoint was reached, the animals were euthanized. Results are shown in FIGS. 41 and 42A-42C.

### Example 13: B16F10 Experiments (Study B16F10-ITAA-0215)

The B16F10 cell line, a rapidly growing melanoma cell line, was used. Using this tumor model, the ability of fusion proteins to affect tumor growth was examined.

TABLE 10
Agents and Treatment

| Group | N | Agent | Dose | Route | Schedule |
|---|---|---|---|---|---|
| 1 | 10 | Vehicle | — | ip | biwk × 2 |
| 2 | 10 | WW0757/636 | 50 μg/animal | ip | biwk × 2 |
| 3 | 10 | WW0757/636 | 100 μg/animal | ip | biwk × 2 |

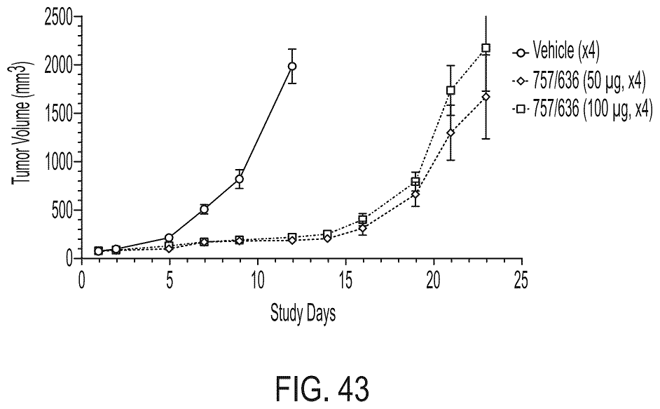

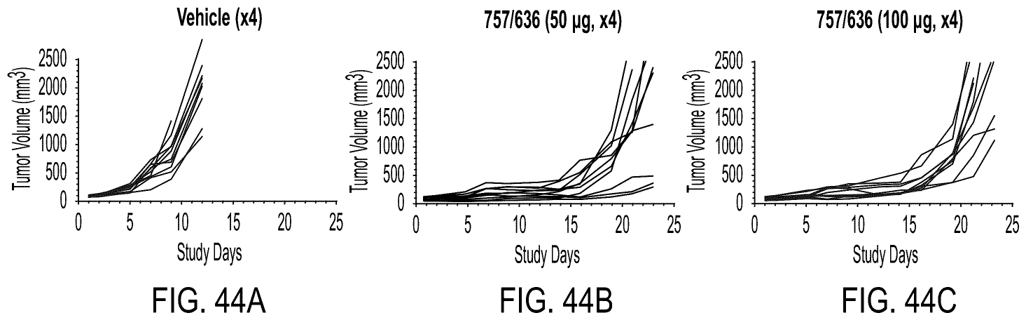

30 CR female C57Bl/6 mice were set up with 1×10⁵ B16F10 tumor cells in 50% Matrigel sc in flank. Cell injection volume was 0.1 mL/mouse. Mouse age at start date was 8 to 12 weeks. Pair matches were performed when tumors reach an average size of 100 mm³ and begin treatment. This is Day 1 of study start. Caliper measurements were taken biweekly to the end. Any adverse reactions were reported immediately. Any individual animal with a single observation of >than 25% body weight loss or three consecutive measurements of >20% body weight loss was euthanized. Any group with a mean body weight loss of >20% or >10% mortality stopped dosing; the group was not euthanized, and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 2000 mm³ or 22 days, whichever comes first. When the endpoint was reached, the animals were euthanized. Results are shown in FIGS. 43 and 44A-44C.

### Example 14: EMT6 Experiments (Study EMT6-ITAA-0216)

The EMT6 cell line, a rapidly growing breast adenocarcinoma cell line, was used. Using this tumor model, the ability of fusion proteins to affect tumor growth was examined.

TABLE 11
Agents and Treatment

| Group | N | Agent | Dose | Route | Schedule |
|---|---|---|---|---|---|
| 1 | 10 | Vehicle | — | ip | biwk × 2 |
| 2 | 10 | WW0757/636 | 50 μg/animal | ip | biwk × 2 |
| 3 | 10 | WW0757/636 | 100 μg/animal | ip | biwk × 2 |

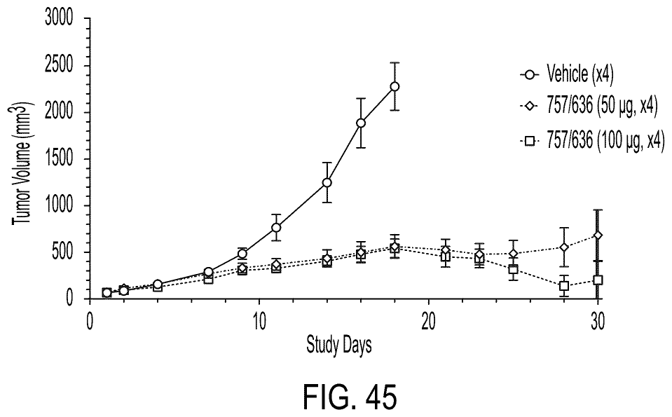

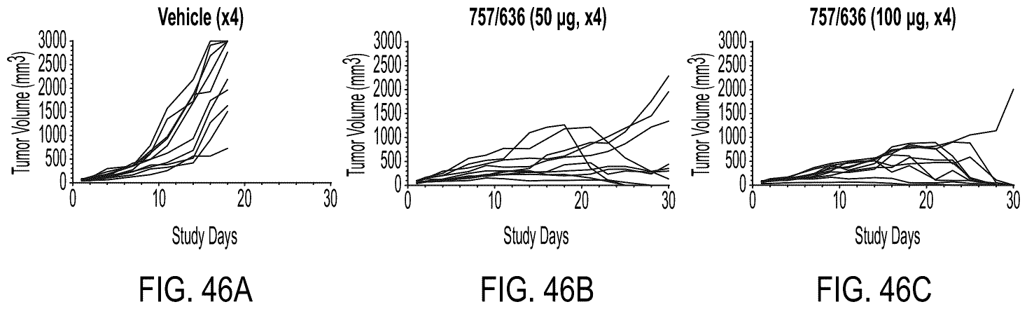

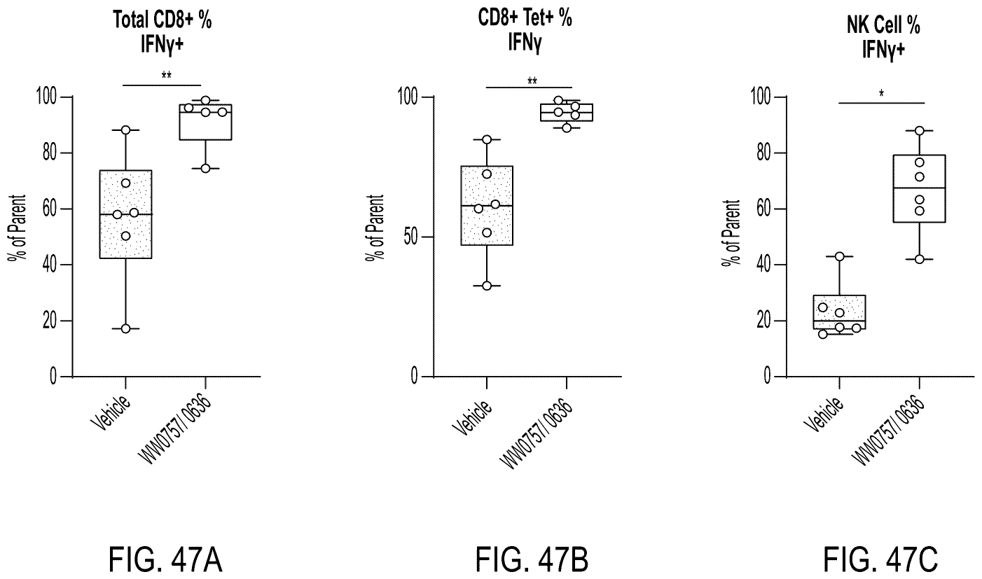

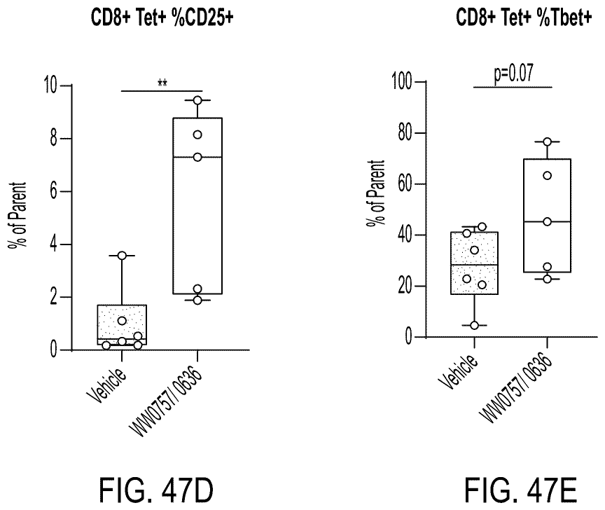

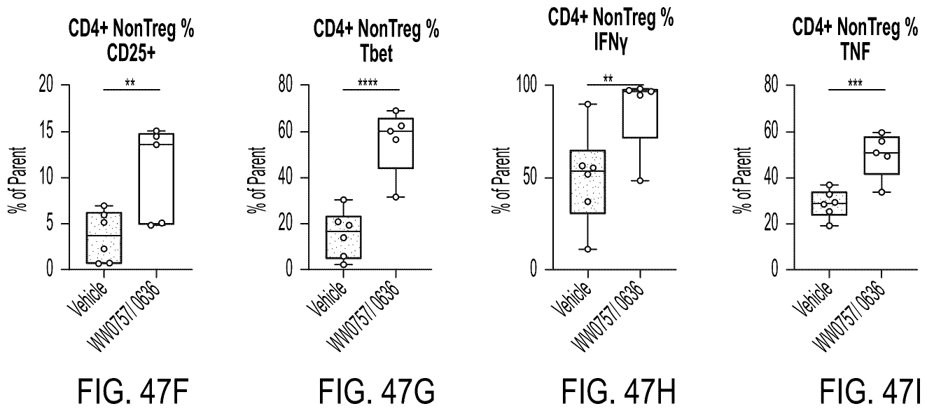

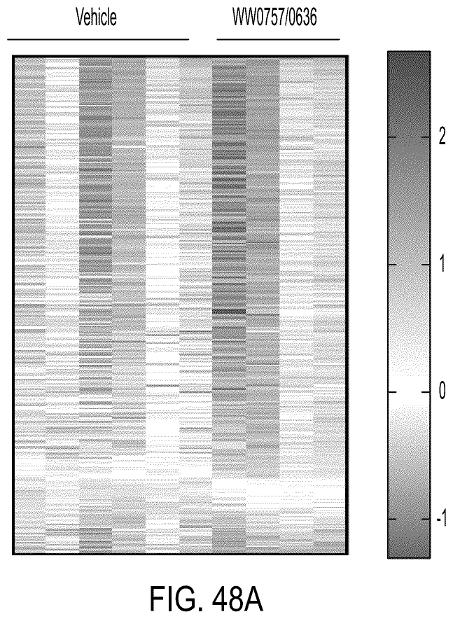

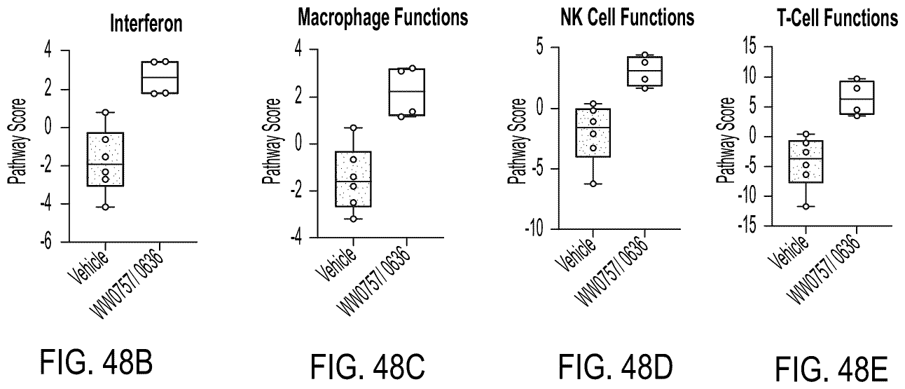

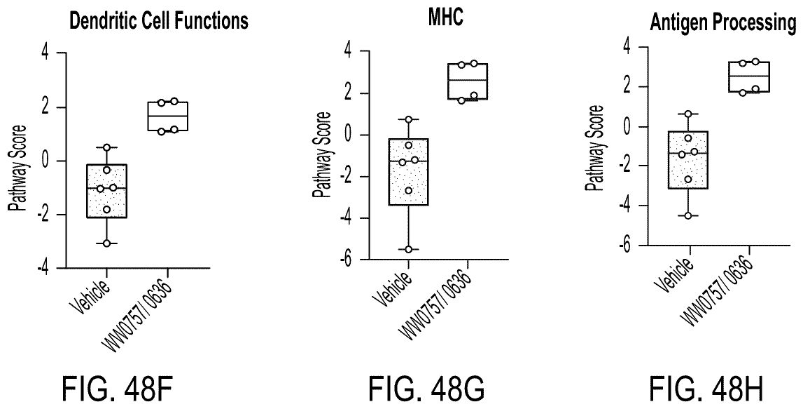

30 CR female BALB/c mice were set up with 1×10⁵ EMT6 tumor cells in 50% Matrigel sc in flank. Cell injection volume was 0.1 mL/mouse. Mouse age at start date was 8 to 12 weeks. Pair matches were performed when tumors reach an average size of 100 mm³ and begin treatment. This is Day 1 of study start. Caliper measurements were taken biweekly to the end. Any adverse reactions were reported immediately. Any individual animal with a single observation of >than 25% body weight loss or three consecutive measurements of >20% body weight loss was euthanized. Any group with a mean body weight loss of >20% or >10% mortality stopped dosing; the group was not euthanized, and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 2000 mm³ or 22 days, whichever comes first. When the endpoint was reached, the animals were euthanized. Results are shown in FIGS. 45 and 46A-46C.

### Example 15: Nanostring Analysis of Total Tumor RNA

Murine tumors from treated animals were harvested and dissociated into single cell suspensions. Briefly, tumors were minced into pieces <5 mm³ before being enzymatically digested. Samples were incubated with 3 mg/mL Collagenase IV for 35 minutes at 37° C. while shaking, before being mechanically dissociated through a 70 μM nylon mesh filter. Samples were then washed and counted, and 3-5e5 total live cells from each sample were spun down, and frozen in RLT+buffer for later RNA extraction. RNA isolation and nanostring processing was run by LakePharma. RNA was isolated using an RNEasy Micro Kit according to the manufacturer's protocol, and 100 ng of total RNA was run using the Murine PanCancer Immune Profiling Codeset on an nCounter system. Data analysis was performed by Werewolf Therapeutics using nSolver software with the Advanced Analysis module installed. All statistical analysis is derived from the nSolver software (see, nCounter Advanced Analysis 2.0 Plugin for nSolver Software, User Manual, NanoString Technologies, 2018). Heatmaps and other graphs were generated using Prism software.

### Example 16: Murine Tumor Processing and Flow Cytometric Analysis

MC38 tumors were implanted into C57BL/6 mice and allowed to grow to an average size of 150 mm³ before mice were randomized into treatment groups (Day 0). Mice were treated with either vehicle or attenuated IL-12 on Day 1 and Day 4 by intraperitoneal injection, and tumors were harvested 24 hours following the second dose (Day 5). Tumors from were harvested and minced into pieces <5 mm³ before being enzymatically digested in phenol free RPMI. Samples were incubated with 3 mg/mL Collagenase IV for 35 minutes at 37° C. while shaking, before being mechanically dissociated through a 70 μM nylon mesh filter. Samples were then washed, counted, and plated for flow cytometry analysis. A maximum of 5×10⁶ cells were plated per well in a 96 well round bottom plate. For intracellular cytokine staining, samples were stimulated for 4 hours with Phorbol 12-myristate 13-acetate (PMA), Ionomycin, and Brefeldin A before being stained. For cell staining, FC receptors were first blocked before extracellular markers were stained. Following extracellular staining, cells were washed, fixed, and permeabilized before intracellular markers were stained. Samples were run on a Cytek Aurora system running SpectroFlo® software, and data was analyzed using FlowJo™ Software. All graphs and statistical analysis were performed using GraphPad Prism software.

### 8. CONSTRUCT PERMUTATIONS

The elements of the polypeptide constructs provided in Table 8 contain the abbreviations as follows: “L,” “X,” “LX,” and “XL” each refer to a linker. “X” refers to a cleavable linker. “L” refers a linker that is optionally cleavable. When L is the only linker in a polypeptide, L is cleavable. “LX” or “XL” each refer to a cleavable linker with an extended non-cleavable sequence adjacent to it. Linker 1 refers to a linker that comprises a MMP9 substrate motif sequence, Linker 2 refers to a linker that comprises a MMP14 substrate motif sequence. Linker 3 refers to a linker that comprises a CTSL-1 substrate motif sequence.

TABLE 12
Exemplary IL-12 polypeptide complex constructs

| Construct # | Construct Description |
|---|---|
| WW0025 | human_p40-murine_p35_Fusion_protein-6xHis |
| WW0026 | human p40-human_p35_Fusion_protein-6xHis |
| WW0101 | Blocker-LX-human_p40-L-mouse_p35-X-anti-HSA_(Blocker = Vl-Vh_X = Linker1) |
| WW0104 | anti-HSA-L-Blocker-LX-human_p40-L-mouse_p35_(Blocker = Vl-Vh_X = Linker1) |
| WW0105 | anti-HSA-X-human_p40-L-mouse_p35-XL-Blocker_(Blocker = Vl/Vh; X = Linker1) |
| WW0106 | human_p40-L-mouse_p35-XL-Blocker-L-anti-HSA_(Blocker = Vl/Vh; X = Linker1) |
| WW0162 | human_p40-L-mouse_p35-LL-Blocker-L-anti-HSA (non- |
| | cleavable_control_Blocker = Vl-Vh) |
| WW0171 | human_p40-L-mouse_p35-XL-Blocker_(Blocker = Vl-Vh_X = Linker1)) |
| WW0295 | Human_p40-L-mouse_p35 |
| WW0309 | anti-HSA-L-human_p40-L-mouse_p35-LL-Blocker_(non-cleavable; Blocker = Vl/Vh) |
| WW0314 | human_p40-L-mouse_p35-XL-Blocker-X-anti-HSA_(X = Linker1; Blocker = Vl/Vh) |
| WW0328 | mAlb-X-human_p40-L-mouse_p35-XL-Blocker_(X = Linker1; Blocker = Vl/Vh) |
| WW0329 | human_p40-L-mouse_p35-XL-Blocker-X-mAlb_(X = Linker1; Blocker = Vl/Vh) |
| WW0330 | mIgG1_Fc-X-human_p40-L-mouse_p35-XL-Blocker_(X = Linker1; Blocker = Vl/Vh) |
| WW0331 | human_p40-L-mouse_p35-XL-Blocker-X-mIgG1_Fc_(X = Linker1; Blocker = Vl/Vh) |
| WW0402 | anti-HSA-X-human_p40-L-mouse_p35-XL- |
| | Blocker(cleavable)_(X = Linker1; Blocker = Vl-X-Vh) |
| WW0461 | anti-HSA-X-human_p40-L-mouse_p35-XL-Blocker_(Blocker = 3CYT5; X = Linker1) |
| WW0636 | Human_IL12B_(p40) |
| WW0637 | anti-HSA-X-mouse_p35-XL-Blocker_(Blocker = Vl/Vh; X = Linker1) |
| WW0638 | anti-HSA-X-human_p40_C199S-L-mouse_p35_C92S-XL- |
| | Blocker_(Blocker = Vl/Vh; X = Linker1) |
| WW0639 | anti-HSA-X-human_p40-L(4xG4S)-mouse_p35-XL- |
| | Blocker_(Blocker = Vl/Vh; X = Linker1) |
| WW0640 | anti-HSA-X-human_p40_mouse_p35-XL-Blocker_(Blocker = Vl/Vh_VH44- |
| | VL100_disulfide; X = Linker1) |
| WW0641 | anti-HSA-X-human_p40_mouse_p35-XL-Blocker_(Blocker = Vl/Vh_VH105- |
| | VL43_disulfide; X = Linker1) |
| WW0649 | anti-HSA-X-human_p40-L-mouse_p35-XL-Blocker_(Blocker = Vl/Vh_X = Linker2) |
| WW0650 | anti-HSA-X-human_p40-L-Human_p35-XL-Blocker_(Blocker = Vl/Vh_X = Linker2) |
| WW0651 | anti-HSA-X-human_p40-L-mouse_p35-XL-Blocker_(Blocker = Vl/Vh_X = Linker3) |
| WW0652 | anti-HSA-X-human_p40-L-Human_p35-XL-Blocker_(Blocker = Vl/Vh_X = Linker3) |
| WW0662 | anti-HSA-X-human_p40-L-mouse_p35-XL- |
| | Blocker_(Blocker-Opt1_Hv_D53E_D61E_Vl/Vh_X = Linker2) |
| WW0663 | anti-HSA-X-human_p40-L-human_p35-XL- |
| | Blocker_(Blocker-Opt1_Hv_D53E_D61E_Vl/Vh_X = Linker2) |
| WW0664 | anti-HSA-X-human_p40-L-mouse_p35-XL-Blocker_(Blocker = Opt2_Lv_N31E- |
| | Hv_D53E_D61E_Vl/Vh_X = Linker2) |
| WW0665 | anti-HSA-X-human_p40-L-human_p35-XL-Blocker_(Blocker = Opt2_Lv_N31E- |
| | Hv_D53E_D61E_Vl/Vh_X = Linker2) |
| WW0666 | anti-HSA-X-human_p40-L-mouse_p35-XL-Blocker_(Blocker = Opt3_LV_S30D- |
| | Hv_D53E_D61E_Vl/Vh_X = Linker2) |
| WW0667 | anti-HSA-X-human_p40-L-human_p35-XL-Blocker_(Blocker = Opt3_LV_S30D- |
| | Hv_D53E_D61E_Vl/Vh_X = Linker2) |
| WW0668 | anti-HSA-X-human_p40-L-mouse_p35-XL- |
| | Blocker_(Blocker = Opt4_LV_S30D_N31E_Vl/Vh_X = Linker2) |
| WW0669 | anti-HSA-X-human_p40-L-human_p35-XL- |
| | Blocker_(Blocker = Opt4_LV_S30D_N31E_Vl/Vh_X = Linker2) |
| WW0670 | anti-HSA-X-human_p40-L-mouse_p35-XL- |
| | Blocker_(Blocker = Opt5_Lv_S30D_N31E-Hv_D53E_D61E_Vl/Vh_X = Linker2) |
| WW0671 | anti-HSA-X-human_p40-L-human_p35-XL- |
| | Blocker_(Blocker = Opt5_Lv_S30D_N31E-Hv_D53E_D61E_Vl/Vh_X = Linker2) |
| WW0672 | anti-HSA-X-human_p40-L-mouse_p35-XL- |
| | Blocker_(Blocker = Opt6_Lv_R27E_T32D(LCharge_16(combo2))_Vl/Vh_X = Linker2) |
| WW0673 | anti-HSA-X-human_p40-L-human_p35-XL- |
| | Blocker_(Blocker = Opt6_Lv_R27E_T32D(LCharge_16(combo2))_Vl/Vh_X = Linker2) |
| WW0674 | anti-HSA-X-human_p40-L-mouse_p35-XL-Blocker_(Blocker = Opt7_Lv_S30E- |
| | Hv_D53E_D61E_Vl/Vh_X = Linker2) |
| WW0675 | anti-HSA-X-human_p40-L-human_p35-XL-Blocker_(Blocker = Opt7_Lv_S30E- |
| | Hv_D53E_D61E_Vl/Vh_X = Linker2) |
| WW0676 | anti-HSA-X-human_p40-L-mouse_p35-XL-Blocker_(Blocker = Opt8_Lv_S30E |
| | N31E_Vl/Vh_X = Linker2) |
| WW0677 | anti-HSA-X-human_p40-L-human_p35-XL-Blocker_(Blocker = Opt8_Lv_S30E |
| | N31E_Vl/Vh_X = Linker2) |
| WW0678 | anti-HSA-X-human_p40-L-mouse_p35-XL-Blocker_(Blocker = Opt9_Lv_N31E- |
| | Hv_D53E_Vl/Vh_X = Linker2) |
| WW0679 | anti-HSA-X-human_p40-L-human_p35-XL-Blocker_(Blocker = Opt9_Lv_N31E- |
| | Hv_D53E_Vl/Vh_X = Linker2) |
| WW0680 | anti-HSA-X-human_p40-L-mouse_p35-XL- |
| | Blocker_(Blocker = Opt1_Hv_D53E_D61E_Vl/Vh_X = Linker3 |
| WW0681 | anti-HSA-X-human_p40-L-human_p35-XL- |
| | Blocker_(Blocker = Opt1_Hv_D53E_D61E_Vl/Vh_X = Linker3) |
| WW0682 | anti-HSA-X-human_p40-L-mouse_p35-XL-Blocker_(Blocker = Opt2_Lv_N31E- |
| | Hv_D53E_D61E_Vl/Vh_X = Linker3 |
| WW0683 | anti-HSA-X-human_p40-L-human_p35-XL-Blocker_(Blocker = Opt2_Lv_N31E- |
| | Hv_D53E_D61E_Vl/Vh_X = Linker3) |
| WW0684 | anti-HSA-X-human_p40-L-mouse_p35-XL-Blocker_(Blocker = Opt3_LV_S30D- |
| | Hv_D53E_D61E_Vl/Vh_X = Linker3 |
| WW0685 | anti-HSA-X-human_p40-L-human_p35-XL-Blocker_(Blocker = Opt3_LV_S30D- |
| | Hv_D53E_D61E_Vl/Vh_X = Linker3) |
| WW0686 | anti-HSA-X-human_p40-L-mouse_p35-XL- |
| | Blocker_(Blocker = Opt4_LV_S30D_N31E_Vl/Vh_X = Linker3 |
| WW0687 | anti-HSA-X-human_p40-L-human_p35-XL- |
| | Blocker_(Blocker = Opt4_LV_S30D_N31E_Vl/Vh_X = Linker3) |
| WW0688 | anti-HSA-X-human_p40-L-mouse_p35-XL- |
| | Blocker_(Blocker = Opt5_Lv_S30D_N31E-Hv_D53E_D61E_Vl/Vh_X = Linker3 |
| WW0689 | anti-HSA-X-human_p40-L-human_p35-XL- |
| | Blocker_(Blocker = Opt5_Lv_S30D_N31E-Hv_D53E_D61E_Vl/Vh_X = Linker3) |
| WW0690 | anti-HSA-X-human_p40-L-mouse_p35-XL- |
| | Blocker_(Blocker = Opt6_Lv_R27E_T32D(LCharge_16(combo2))_Vl/Vh_X = Linker3 |
| WW0691 | anti-HSA-X-human_p40-L-human_p35-XL- |
| | Blocker_(Blocker = Opt6_Lv_R27E_T32D(LCharge_16(combo2))_Vl/Vh_X = Linker3) |
| WW0692 | anti-HSA-X-human_p40-L-mouse_p35-XL-Blocker_(Blocker = Opt7_Lv_S30E- |
| | Hv_D53E_D61E_Vl/Vh_X = Linker3 |
| WW0693 | anti-HSA-X-human_p40-L-human_p35-XL-Blocker_(Blocker = Opt7_Lv_S30E- |
| | Hv_D53E_D61E_Vl/Vh_X = Linker3) |
| WW0694 | anti-HSA-X-human_p40-L-mouse_p35-XL- |
| | Blocker_(Blocker = Opt8_Lv_S30E_N31E_Vl/Vh_X = Linker3 |
| WW0695 | anti-HSA-X-human_p40-L-human_p35-XL- |
| | Blocker_(Blocker = Opt8_Lv_S30E_N31E_Vl/Vh_X = Linker3) |
| WW0696 | anti-HSA-X-human_p40-L-mouse_p35-XL-Blocker_(Blocker = Opt9_Lv_N31E- |
| | Hv_D53E_Vl/Vh_X = Linker3 |
| WW0697 | anti-HSA-X-human_p40-L-human_p35-XL-Blocker_(Blocker = Opt9_Lv_N31E- |
| | Hv_D53E_Vl/Vh_X = Linker3) |
| WW0698 | anti-HSA-X-human_p40-L-mouse_p35-XL- |
| | Fab_Lambda_Blocker_(Blocker = Lambda_Fab_IGLC2-01_X = Linker2) |
| WW0699 | anti-HSA-X-human_p40-L-human_p35-XL- |
| | Fab_Lambda_Blocker_(Blocker = Lambda_Fab_IGLC2-01_X = Linker2) |
| WW0700 | anti-HSA-X-human_p40-L-mouse_p35-XL- |
| | Fab_Lambda_Blocker_(Blocker = Lambda_Fab_N31E_IGLC2-01_X = Linker2) |
| WW0701 | anti-HSA-X-human_p40-L-human_p35-XL- |
| | Fab_Lambda_Blocker_(Blocker = Lambda_Fab_N31E_IGLC2-01_X = Linker2) |
| WW0702 | anti-HSA-X-human_p40-L-mouse_p35-XL- |
| | Fab_Lambda_Blocker_(Blocker = Lambda_Fab_S30D_IGLC2-01_X = Linker2) |
| WW0703 | anti-HSA-X-human_p40-L-human_p35-XL- |
| | Fab_Lambda_Blocker_(Blocker = Lambda_Fab_S30D_IGLC2-01_X = Linker2) |
| WW0704 | anti-HSA-X-human_p40-L-mouse_p35-XL- |
| | Fab_Lambda_Blocker_(Blocker = Lambda_Fab_S30D_N31E_IGLC2- |
| | 01_X = Linker2) |
| WW0705 | anti-HSA-X-human_p40-L-human p35-XL- |
| | Fab_Lambda_Blocker_(Blocker = Lambda_Fab_S30D_N31E_IGLC2- |
| | 01_X = Linker2) |
| WW0706 | anti-HSA-X-human_p40-L-mouse_p35-XL- |
| | Fab_Lambda_Blocker_(Blocker = Lambda_Fab_R27E_T32D_IGLC2- |
| | 01_X = Linker2) |
| WW0707 | anti-HSA-X-human_p40-L-human_p35-XL- |
| | Fab_Lambda_Blocker_(Blocker = Lambda_Fab_R27E_T32D_IGLC2- |
| | 01_X = Linker2) |
| WW0708 | anti-HSA-X-human_p40-L-mouse_p35-XL- |
| | Fab_Lambda_Blocker_(Blocker = Lambda_Fab_S30E_IGLC2-01_X = Linker2) |
| WW0709 | anti-HSA-X-human_p40-L-human_p35-XL- |
| | Fab_Lambda_Blocker_(Blocker = Lambda_Fab_S30E_IGLC2-01_X = Linker2) |
| WW0710 | anti-HSA-X-human_p40-L-mouse_p35-XL- |
| | Fab_Lambda_Blocker_(Blocker = Lambda_Fab_S30E_N31E_IGLC2- |
| | 01_X = Linker2) |
| WW0711 | anti-HSA-X-human_p40-L-human_p35-XL- |
| | Fab_Lambda_Blocker_(Blocker = Lambda_Fab_S30E_N31E_IGLC2- |
| | 01_X = Linker2) |
| WW0712 | anti-HSA-X-human_p40-L-mouse_p35-XL- |
| | Fab_Lambda_Blocker_(Blocker = Lambda_Fab_IGLC2-01_X = Linker3) |
| WW0713 | anti-HSA-X-human_p40-L-human_p35-XL- |
| | Fab_Lambda_Blocker_(Blocker = Lambda_Fab_IGLC2-01 X = Linker3) |
| WW0714 | anti-HSA-X-human_p40-L-mouse_p35-XL- |
| | Fab_Lambda_Blocker_(Blocker = Lambda_Fab_N31E_IGLC2-01_X = Linker3) |
| WW0715 | anti-HSA-X-human_p40-L-human_p35-XL- |
| | Fab_Lambda_Blocker_(Blocker = Lambda_Fab_N31E_IGLC2-01_X = Linker3) |
| WW0716 | anti-HSA-X-human_p40-L-mouse_p35-XL- |
| | Fab_Lambda_Blocker_(Blocker = Lambda_Fab_S30D_IGLC2-01_X = Linker3) |
| WW0717 | anti-HSA-X-human_p40-L-human_p35-XL- |
| | Fab_Lambda_Blocker_(Blocker = Lambda_Fab_S30D_IGLC2-01_X = Linker3) |
| WW0718 | anti-HSA-X-human_p40-L-mouse_p35-XL- |
| | Fab_Lambda_Blocker_(Blocker = Lambda_Fab_S30D_N31E_IGLC2- |
| | 01_X = Linker3) |
| WW0719 | anti-HSA-X-human_p40-L-human_p35-XL- |
| | Fab_Lambda_Blocker_(Blocker = Lambda_Fab_S30D_N31E_IGLC2- |
| | 01_X = Linker3) |
| WW0720 | anti-HSA-X-human_p40-L-mouse_p35-XL- |
| | Fab_Lambda_Blocker_(Blocker = Lambda_Fab_R27E_T32D_IGLC2- |
| | 01_X = Linker3) |
| WW0721 | anti-HSA-X-human_p40-L-human_p35-XL- |
| | Fab_Lambda_Blocker_(Blocker = Lambda_Fab_R27E_T32D_IGLC2- |
| | 01_X = Linker3) |
| WW0722 | anti-HSA-X-human_p40-L-mouse_p35-XL- |
| | Fab_Lambda_Blocker_(Blocker = Lambda_Fab_S30E_IGLC2-01_X = Linker3) |
| WW0723 | anti-HSA-X-human_p40-L-human_p35-XL- |
| | Fab_Lambda_Blocker_(Blocker = Lambda_Fab_S30E_IGLC2-01_X = Linker3) |
| WW0724 | anti-HSA-X-human_p40-L-mouse_p35-XL- |
| | Fab_Lambda_Blocker_(Blocker = Lambda_Fab_S30E_N31E_IGLC2- |
| | 01_X = Linker3) |
| WW0725 | anti-HSA-X-human_p40-L-human_p35-XL- |
| | Fab_Lambda_Blocker_(Blocker = Lambda_Fab_S30E_N31E_IGLC2- |
| | 01_X = Linker3) |
| WW0726 | Fab_Heavy_Blocker_(Blocker = IL-12_Heavy_Fab_IgG1_Fab) |
| WW0727 | Fab_Heavy_Blocker_(Blocker = IL-12_Heavy_Fab_D53E_D61E_IgG1_Fab) |
| WW0728 | Fab_Heavy_Blocker_(Blocker = IL-12_Heavy_Fab_D53E_IgG1_Fab) |
| WW0749 | anti-HSA-X-mouse_p35-XL- |
| | Blocker_(Blocker = Opt1_Hv_D53E_D61E_Vl/Vh_X = Linker2) |
| WW0750 | anti-HSA-X-Human_p35-XL- |
| | Blocker_(Blocker = Opt1_Hv_D53E_D61E_Vl/Vh_X = Linker2) |
| WW0751 | anti-HSA-X-mouse_p35-XL-Blocker_(Blocker = Opt5_Lv_S30D_N31E- |
| | Hv_D53E_D61E_Vl/Vh_X = Linker2) |
| WW0752 | anti-HSA-X-Human_p35-XL-Blocker_(Blocker = Opt5_Lv_S30D_N31E- |
| | Hv_D53E_D61E_Vl/Vh_X = Linker2) |
| WW0753 | anti-HSA-X-mouse_p35-XL- |
| | Fab_Lambda_Blocker_(Blocker = Lambda_Fab_IGLC2-01_X = Linker2) |
| WW0754 | anti-HSA-X-Human_p35-XL- |
| | Fab_Lambda_Blocker_(Blocker = Lambda_Fab_IGLC2-01_X = Linker2) |
| WW0755 | anti-HSA-X-mouse_p35-XL- |
| | Fab_Lambda_Blocker_(Blocker = Lambda_Fab_S30D_N31E_IGLC2- |
| | 01_X = Linker2) |
| WW0756 | anti-HSA-X-Human_p35-XL- |
| | Fab_Lambda_Blocker_(Blocker = Lambda_Fab_S30D_N31E_IGLC2- |
| | 01_X = Linker2) |
| WW0757 | anti-HSA-X-mouse_p35-XL- |
| | Blocker_(Blocker = Opt1_Hv_D53E_D61E_Vl/Vh_X = Linker3) |
| WW0758 | anti-HSA-X-Human_p35-XL- |
| | Blocker_(Blocker = Opt1_Hv_D53E_D61E_Vl/Vh_X = Linker3) |
| WW0759 | anti-HSA-X-mouse_p35-XL-Blocker_(Blocker = Opt5_Lv_S30D_N31E- |
| | Hv_D53E_D61E_Vl/Vh_X = Linker3) |
| WW0760 | anti-HSA-X-Human_p35-XL-Blocker_(Blocker = Opt5_Lv_S30D_N31E- |
| | Hv_D53E_D61E_Vl/Vh_X = Linker3) |
| WW0761 | anti-HSA-X-mouse_p35-XL- |
| | Fab_Lambda_Blocker_(Blocker = Lambda_Fab_IGLC2-01_X = Linker3) |
| WW0762 | anti-HSA-X-Human_p35-XL- |
| | Fab_Lambda_Blocker_(Blocker = Lambda_Fab_IGLC2-01_X = Linker3) |
| WW0763 | anti-HSA-X-mouse_p35-XL- |
| | Fab_Lambda_Blocker_(Blocker = Lambda_Fab_S30D_N31E_IGLC2- |
| | 01_X = Linker3) |
| WW0764 | anti-HSA-X-Human p35-XL- |
| | Fab_Lambda_Blocker_(Blocker = Lambda_Fab_S30D_N31E_IGLC2- |
| | 01_X = Linker3) |
| WW0765 | human_p40-L-mouse_p35-X-anti-HSA-L- |
| | Blocker_(Blocker = Opt1_Hv_D53E_D61E_Vl/Vh_X = Linker2) |
| WW0766 | human_p40-L-human_p35-X-anti-HSA-L- |
| | Blocker_(Blocker = Opt1_Hv_D53E_D61E_Vl/Vh_X = Linker2) |
| WW0767 | human_p40-L-mouse_p35-X-anti-HSA-L- |
| | Blocker_(Blocker = Opt5_Lv_S30D_N31E-Hv_D53E_D61E_Vl/Vh_X = Linker2) |
| WW0768 | human_p40-L-human_p35-X-anti-HSA-L- |
| | Blocker_(Blocker = Opt5_Lv_S30D_N31E-Hv_D53E_D61E_Vl/Vh_X = Linker2) |
| WW0769 | human_p40-L-mouse_p35-X-anti-HSA-L- |
| | Fab_Lambda_Blocker_(Blocker = Lambda_Fab_IGLC2-01_X = Linker2) |
| WW0770 | human_p40-L-human p35-X-anti-HSA-L- |
| | Fab_Lambda_Blocker_(Blocker = Lambda_Fab_IGLC2-01_X = Linker2) |
| WW0771 | human_p40-L-mouse_p35-X-anti-HSA-L- |
| | Fab_Lambda_Blocker_(Blocker = Lambda_Fab_S30D_N31E_IGLC2- |
| | 01_X = Linker2) |
| WW0772 | human_p40-L-human_p35-X-anti-HSA-L- |
| | Fab_Lambda_Blocker_(Blocker = Lambda_Fab_S30D_N31E_IGLC2- |
| | 01_X = Linker2) |
| WW0773 | mouse_p35-X-anti-HSA-L- |
| | Blocker_(Blocker = Opt1_Hv_D53E_D61E_Vl/Vh_X = Linker2) |
| WW0774 | human_p35-X-anti-HSA-L- |
| | Blocker_(Blocker = Opt1_Hv_D53E_D61E_Vl/Vh_X = Linker2) |
| WW0775 | mouse_p35-X-anti-HSA-L-Blocker_(Blocker = Opt5_Lv_S30D_N31E- |
| | Hv_D53E_D61E_Vl/Vh_X = Linker2) |
| WW0776 | human_p35-X-anti-HSA-L-Blocker_(Blocker = Opt5_Lv_S30D_N31E- |
| | Hv_D53E_D61E_Vl/Vh_X = Linker2) |
| WW0777 | mouse_p35-X-anti-HSA-L-Fab_Lambda_Blocker_(Blocker = Lambda_Fab_IGLC2- |
| | 01_X = Linker2) |
| WW0778 | human_p35-X-anti-HSA-L-Fab_Lambda_Blocker_(Blocker = Lambda_Fab_IGLC2- |
| | 01_X = Linker2) |
| WW0779 | mouse_p35-X-anti-HSA-L- |
| | Fab_Lambda_Blocker_(Blocker = Lambda_Fab_S30D_N31E_IGLC2- |
| | 01_X = Linker2) |
| WW0780 | human_p35-X-anti-HSA-L- |
| | Fab_Lambda_Blocker_(Blocker = Lambda_Fab_S30D_N31E_IGLC2- |
| | 01_X = Linker2) |
| WW0796 | human_p40-L-mouse_p35-X-anti-HSA-L- |
| | Blocker_(Blocker = Opt1_Hv_D53E_D61E_Vl-Vh_X = Linker3) |
| WW0797 | human_p40-L-human_p35-X-anti-HSA-L- |
| | Blocker_(Blocker = Opt1_Hv_D53E_D61E_Vl-Vh_X = Linker3) |
| WW0798 | human_p40-L-mouse_p35-X-anti-HSA-L- |
| | Blocker_(Blocker = Opt5_Lv_S30D_N31E-Hv_D53E_D61E_Vl-Vh_X = Linker3) |
| WW0799 | human_p40-L-human_p35-X-anti-HSA-L- |
| | Blocker_(Blocker = Opt5_Lv_S30D_N31E-Hv_D53E_D61E_Vl-Vh_X = Linker3) |
| WW0800 | human_p40-L-mouse_p35-X-anti-HSA-L- |
| | Fab_Lambda_Blocker_(Blocker = Lambda_Fab_IGLC2-01_X = Linker3) |
| WW0801 | human_p40-L-human_p35-X-anti-HSA-L- |
| | Fab_Lambda_Blocker_(Blocker = Lambda_Fab_IGLC2-01_X = Linker3) |
| WW0802 | human_p40-L-mouse_p35-X-anti-HSA-L- |
| | Fab_Lambda_Blocker_(Blocker = Lambda_Fab_S30D_N31E_IGLC2- |
| | 01_X = Linker3) |
| WW0803 | human_p40-L-human_p35-X-anti-HSA-L- |
| | Fab_Lambda_Blocker_(Blocker = Lambda_Fab_S30D_N31E_IGLC2- |
| | 01_X = Linker3) |
| WW0804 | mouse_p35-X-anti-HSA-L-Blocker_(Blocker = Opt1_Hv_D53E_D61E_Vl- |
| | Vh_X = Linker3) |
| WW0805 | human_p35-X-anti-HSA-L-Blocker_(Blocker = Opt1_Hv_D53E_D61E_Vl- |
| | Vh_X = Linker3) |
| WW0806 | mouse_p35-X-anti-HSA-L-Blocker_(Blocker = Opt5_Lv_S30D_N31E- |
| | Hv_D53E_D61E_Vl-Vh_X = Linker3) |
| WW0807 | human_p35-X-anti-HSA-L-Blocker_(Blocker = Opt5_Lv_S30D_N31E- |
| | Hv_D53E_D61E_Vl-Vh_X = Linker3) |
| WW0808 | mouse_p35-X-anti-HSA-L-Fab_Lambda_Blocker_(Blocker = Lambda_Fab_IGLC2- |
| | 01_X = Linker3) |
| WW0809 | human_p35-X-anti-HSA-L-Fab_Lambda_Blocker_(Blocker = Lambda_Fab_IGLC2- |
| | 01_X = Linker3) |
| WW0810 | mouse_p35-X-anti-HSA-L- |
| | Fab_Lambda_Blocker_(Blocker = Lambda_Fab_S30D_N31E_IGLC2- |
| | 01_X = Linker3) |
| WW0811 | human_p35-X-anti-HSA-L- |
| | Fab_Lambda_Blocker_(Blocker = Lambda_Fab_S30D_N31E_IGLC2- |
| | 01_X = Linker3) |
| WW0814 | Human_IL12A (p35)_His |
| WW50009 | HSA-L-Mouse_IL23 |
| WW50055 | IL23A_mouse_p19 |
| WW50056 | IL23A_human_p19 |
| WW50057 | HSA-L-IL23A_mouse_p19 |
| WW50058 | HSA-L-IL23A_human_p19 |
| WW50059 | HSA-X-Mouse_p19-XL-Blocker_(Blocker = Opt1_Hv_D53E_D61E_Vl- |
| | Vh_3xG4S_X = Linker3) |
| WW50060 | HSA-X-Human_p19-XL-Blocker_(Blocker = Opt1_Hv_D53E_D61E_Vl- |
| | Vh_3xG4S_X = Linker3) |
| WW50087 | HSA-L-Chimeric_IL23 |
| WW50088 | HSA-L-Human_IL23 |
| WW50089 | HSA-X-Chimeric_IL-23-XL-Blocker_(Blocker = Opt1_Hv_D53E_D61E_Vl- |
| | Vh_3xG4S_X = Linker3) |
| WW50090 | HSA-X-Human_IL-23-XL-Blocker_(Blocker = Opt1_Hv_D53E_D61E_Vl- |
| | Vh_3xG4S_X = Linker3) |
| WW00924 | HSA-X-Human_p35-XL-Blocker_(Blocker = Opt1_Hv_D53E_D61E_Vl-Vh_X = |
| | Linker3)_Deglycosylated |
| WW00925 | Human_IL12B_Deglycosylated |
| WW00935 | Human_IL12B_(WW0636)_partially_Deglycosylated |
| WW00936 | HSA-X-Human_p35-XL-Blocker_(Blocker = Opt1_Hv_D53E_D61E_Vl- |
| | Vh_X = Linker3)_Partially_deglycosylated |

9. SEQUENCE DISCLOSURE

| SEQ ID | Construct | | |
|---|---|---|---|
| NO: | Code | Description | Sequence |
| 1 | WW0025 | human_p40 | iwelkkdvyvveldwypdapgemvvltcdtpeedgitwtldqssevl |
| | | murine_p35_Fusion_ | gsgktltiqvkefgdagqytchkggevlshsllllhkkedgiwstdilkd |
| | | protein-6xHis | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssaHHHHHH** |
| 2 | WW0026 | human_p40- | iwelkkdvyvveldwypdapgemvvltcdtpeedgitwtldqssevl |
| | | human_p35_Fusion_ | gsgktltiqvkefgdagqytchkggevlshsllllhkkedgiwstdilkd |
| | | protein-6xHis | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnasHHHHHH** |
| 3 | WW0101 | Monomeric IL-12 | QSVLTQPPSVSGAPGQRVTISCSGSRSNIGSNTV |
| | | (chimeric) | KWYQQLPGTAPKLLIYYNDQRPSGVPDRFSGS |
| | | polypeptide, anti- | KSGTSASLAITGLQAEDEADYYCQSYDRYTHP |
| | | HSA sdAb, scFv | ALLFGTGTKVTVLggggsggggsggggsQVQLVESG |
| | | Blocker, 2 | GGVVQPGRSLRLSCAASGFTFSSYGMHWVRQ |
| | | cleavage sites | APGKGLEWVAFIRYDGSNKYYADSVKGRFTIS |
| | | | RDNSKNTLYLQMNSLRAEDTAVYYCKTHGSH |
| | | | DNWGQGTMVTVSSggggsggggsggggsggggsggggs |
| | | | ggggsSGGPGPAGMKGLPGSiwelkkdvyvveldwypd |
| | | | apgemvvltcdtpeedgitwtldqssevlgsgktltiqvkefgdagqyt |
| | | | chkggevlshsllllhkkedgiwstdilkdqkepknktflrceaknysgr |
| | | | ftcwwlttistdltfsvkssrgssdpqgvtcgaatlsaervrgdnkeyeys |
| | | | vecqedsacpaaeeslpievmvdavhklkyenytssffirdiikpdpp |
| | | | knlqlkplknsrqvevsweypdtwstphsyfsltfcvqvqgkskrekk |
| | | | drvftdktsatvicrknasisvraqdryyssswsewasvpcsggggsg |
| | | | gggsggggsrvipvsgparclsqsrnllkttddmvktareklkhyscta |
| | | | edidheditrdqtstlktclplelhknesclatretssttrgsclppqktslm |
| | | | mtlclgsiyedlkmyqtefqainaalqnhnhqqiildkgmlvaidelm |
| | | | qslnhngetlrqkppvgeadpyrvkmklcillhafstrvvtinrvmgyl |
| | | | ssaSGGPGPAGMKGLPGSEVQLVESGGGLVQPG |
| | | | NSLRLSCAASGFTFSKFGMSWVRQAPGKGLE |
| | | | WVSSISGSGRDTLYAESVKGRFTISRDNAKTTL |
| | | | YLQMNSLRPEDTAVYYCTIGGSLSVSSQGTLV |
| | | | TVSSHHHHHHEPEA** |
| 4 | WW0104 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSggggsggggsggggsQSV |
| | | Blocker, 1 | LTQPPSVSGAPGQRVTISCSGSRSNIGSNTVKW |
| | | cleavage site | YQQLPGTAPKLLIYYNDQRPSGVPDRFSGSKSG |
| | | | TSASLAITGLQAEDEADYYCQSYDRYTHPALL |
| | | | FGTGTKVTVLggggsggggsggggsQVQLVESGGGV |
| | | | VQPGRSLRLSCAASGFTFSSYGMHWVRQAPGK |
| | | | GLEWVAFIRYDGSNKYYADSVKGRFTISRDNS |
| | | | KNTLYLQMNSLRAEDTAVYYCKTHGSHDNW |
| | | | GQGTMVTVSSggggsggggsggggsggggsggggsggggs |
| | | | SGGPGPAGMKGLPGSiwelkkdvyvveldwypdapgem |
| | | | vvltcdtpeedgitwtldqssevlgsgktltiqvkefgdagqytchkgge |
| | | | vlshsllllhkkedgiwstdilkdqkepknktflrceaknysgrftcwwl |
| | | | ttistdltfsvkssrgssdpqgvtcgaatlsaervrgdnkeyeysvecqed |
| | | | sacpaaeeslpievmvdavhklkyenytssffirdiikpdppknlqlkp |
| | | | Iknsrqvevsweypdtwstphsyfsltfcvqvqgkskrekkdrvftdk |
| | | | tsatvicrknasisvraqdryyssswsewasvpcsggggsggggsggg |
| | | | gsrvipvsgparclsqsrnllkttddmvktareklkhysctaedidhedit |
| | | | rdqtstlktclplelhknesclatretssttrgsclppqktslmmtlclgsiy |
| | | | edlkmyqtefqainaalqnhnhqqiildkgmlvaidelmqslnhnget |
| | | | lrqkppvgeadpyrvkmklcillhafstrvvtinrvmgylssaHHH |
| | | | HHHEPEA** |
| 5 | WW0105 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSSGGPGPAGMKGLPG |
| | | Blocker, 2 | Siwelkkdvyvveldwypdapgemvvltcdtpeedgitwtldqsse |
| | | cleavage sites | vlgsgktltiqvkefgdagqytchkggevlshsllllhkkedgiwstdil |
| | | | kdqkepknktflrceaknysgrftcwwlttistdltfsvkssrgssdpqg |
| | | | vtcgaatlsaervrgdnkeyeysvecqedsacpaaeeslpievmvdav |
| | | | hklkyenytssffirdiikpdppknlqlkplknsrqvevsweypdtwst |
| | | | phsyfsltfcvqvqgkskrekkdrvftdktsatvicrknasisvraqdry |
| | | | yssswsewasvpcsggggsggggsggggsrvipvsgparclsqsrnll |
| | | | kttddmvktareklkhysctaedidheditrdqtstlktclplelhknesc |
| | | | latretssttrgsclppqktslmmtlclgsiyedlkmyqtefqainaalqn |
| | | | hnhqqiildkgmlvaidelmqslnhngetlrqkppvgeadpyrvkm |
| | | | klcillhafstrvvtinrvmgylssaSGGPGPAGMKGLPGSg |
| | | | gggsggggggggggggsggggggggsQSVLTQPPSVSG |
| | | | APGQRVTISCSGSRSNIGSNTVKWYQQLPGTAP |
| | | | KLLIYYNDQRPSGVPDRFSGSKSGTSASLAITG |
| | | | LQAEDEADYYCQSYDRYTHPALLFGTGTKVT |
| | | | VLggggsggggsggggsQVQLVESGGGVVQPGRSLR |
| | | | LSCAASGFTFSSYGMHWVRQAPGKGLEWVAFI |
| | | | RYDGSNKYYADSVKGRFTISRDNSKNTLYLQM |
| | | | NSLRAEDTAVYYCKTHGSHDNWGQGTMVTV |
| | | | SSHHHHHHEPEA** |
| 6 | WW0106 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, scFv | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssaSGGPGPAGMKGLPGSggggsg |
| | | | gggsggggsggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSSg |
| | | | gggsggggsggggsEVQLVESGGGLVQPGNSLRLSC |
| | | | AASGFTFSKFGMSWVRQAPGKGLEWVSSISGS |
| | | | GRDTLYAESVKGRFTISRDNAKTTLYLQMNSL |
| | | | RPEDTAVYYCTIGGSLSVSSQGTLVTVSSHHHH |
| | | | HHEPEA** |
| 7 | WW0162 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, scFv | gaatlsaervrgdnkeyeysvecqedsacpaaeeslpievmvdavhkl |
| | | Blocker, no | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggsggggggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssaggggsggggsggggsggggggggsgg |
| | | | ggsggggsggggsggggsQSVLTQPPSVSGAPGQRVTI |
| | | | SCSGSRSNIGSNTVKWYQQLPGTAPKLLIYYND |
| | | | QRPSGVPDRFSGSKSGTSASLAITGLQAEDEAD |
| | | | YYCQSYDRYTHPALLFGTGTKVTVLggggggggs |
| | | | ggggsQVQLVESGGGVVQPGRSLRLSCAASGFTF |
| | | | SSYGMHWVRQAPGKGLEWVAFIRYDGSNKYY |
| | | | ADSVKGRFTISRDNSKNTLYLQMNSLRAEDTA |
| | | | VYYCKTHGSHDNWGQGTMVTVSSggggsggggsg |
| | | | gggsEVQLVESGGGLVQPGNSLRLSCAASGFTFS |
| | | | KFGMSWVRQAPGKGLEWVSSISGSGRDTLYA |
| | | | ESVKGRFTISRDNAKTTLYLQMNSLRPEDTAV |
| | | | YYCTIGGSLSVSSQGTLVTVSSHHHHHHEPEA** |
| 8 | WW0171 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, scFv | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssaSGGPGPAGMKGLPGSggggsg |
| | | | gggsggggggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSS |
| | | | HHHHHHEPEA** |
| 9 | WW0295 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssahhhhhh** |
| 10 | WW0309 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, scFv | KGRFTISRDNAKTTLYLQMNSLRPEDTAVYYC |
| | | Blocker, no | TIGGSLSVSSQGTLVTVSSggggsggggsggggsiwelk |
| | | cleavage site | kdvyvveldwypdapgemvvltcdtpeedgitwtldqssevlgsgkt |
| | | | ltiqvkefgdagqytchkggevlshsllllhkkedgiwstdilkdqkep |
| | | | knktflrceaknysgrftcwwlttistdltfsvkssrgssdpqgvtcgaatl |
| | | | saervrgdnkeyeysvecqedsacpaaeeslpievmvdavhklkyen |
| | | | ytssffirdiikpdppknlqlkplknsrqvevsweypdtwstphsyfslt |
| | | | fcvqvqgkskrekkdrvftdktsatvicrknasisvraqdryyssswse |
| | | | wasvpcsggggggggsggggsrvipvsgparclsqsrnllkttddm |
| | | | vktareklkhysctaedidheditrdqtstlktclplelhknesclatretss |
| | | | ttrgsclppqktslmmtlclgsiyedlkmyqtefqainaalqnhnhqqii |
| | | | ldkgmlvaidelmqslnhngetlrqkppvgeadpyrvkmklcillhaf |
| | | | strvvtinrvmgylssaggggsggggsggggsggggsggggsgggg |
| | | | sggggsggggsggggsQSVLTQPPSVSGAPGQRVTISC |
| | | | SGSRSNIGSNTVKWYQQLPGTAPKLLIYYNDQ |
| | | | RPSGVPDRFSGSKSGTSASLAITGLQAEDEADY |
| | | | YCQSYDRYTHPALLFGTGTKVTVLggggsggggsg |
| | | | gggsQVQLVESGGGVVQPGRSLRLSCAASGFTF |
| | | | SSYGMHWVRQAPGKGLEWVAFIRYDGSNKYY |
| | | | ADSVKGRFTISRDNSKNTLYLQMNSLRAEDTA |
| | | | VYYCKTHGSHDNWGQGTMVTVSSHHHHHH** |
| 11 | WW0314 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, scFv | gaatlsaervrgdnkeyeysvecqedsacpaaeeslpievmvdavhkl |
| | | Blocker, 2 | kyenytssffirdiikpdppknlqlkplknsrqvevsweypdtwstphs |
| | | cleavage sites | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssaSGGPGPAGMKGLPGSggggsg |
| | | | gggsggggggggggggsggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSSS |
| | | | GGPGPAGMKGLPGSEVQLVESGGGLVQPGNSL |
| | | | RLSCAASGFTFSKFGMSWVRQAPGKGLEWVSS |
| | | | ISGSGRDTLYAESVKGRFTISRDNAKTTLYLQM |
| | | | NSLRPEDTAVYYCTIGGSLSVSSQGTLVTVSSH |
| | | | HHHHH** |
| 12 | WW0328 | Monomeric IL-12 | EAHKSEIAHRYNDLGEQHFKGLVLIAFSQYLQ |
| | | (chimeric) | KCSYDEHAKLVQEVTDFAKTCVADESAANCD |
| | | polypeptide, | KSLHTLFGDKLCAIPNLRENYGELADCCTKQEP |
| | | Albumin, scFv | ERNECFLQHKDDNPSLPPFERPEAEAMCTSFKE |
| | | Blocker, 2 | NPTTFMGHYLHEVARRHPYFYAPELLYYAEQY |
| | | cleavage sites | NEILTQCCAEADKESCLTPKLDGVKEKALVSS |
| | | | VRQRMKCSSMQKFGERAFKAWAVARLSQTFP |
| | | | NADFAEITKLATDLTKVNKECCHGDLLECADD |
| | | | RAELAKYMCENQATISSKLQTCCDKPLLKKAH |
| | | | CLSEVEHDTMPADLPAIAADFVEDQEVCKNYA |
| | | | EAKDVFLGTFLYEYSRRHPDYSVSLLLRLAKK |
| | | | YEATLEKCCAEANPPACYGTVLAEFQPLVEEP |
| | | | KNLVKTNCDLYEKLGEYGFQNAILVRYTQKAP |
| | | | QVSTPTLVEAARNLGRVGTKCCTLPEDQRLPC |
| | | | VEDYLSAILNRVCLLHEKTPVSEHVTKCCSGSL |
| | | | VERRPCFSALTVDETYVPKEFKAETFTFHSDIC |
| | | | TLPEKEKQIKKQTALAELVKHKPKATAEQLKT |
| | | | VMDDFAQFLDTCCKAADKDTCFSTEGPNLVTR |
| | | | CKDALASGGPGPAGMKGLPGSiwelkkdvyvveldw |
| | | | ypdapgemvvltcdtpeedgitwtldqssevlgsgktltiqvkefgdag |
| | | | qytchkggevlshsllllhkkedgiwstdilkdqkepknktflrceakn |
| | | | ysgrftcwwlttistdltfsvkssrgssdpqgvtcgaatlsaervrgdnke |
| | | | yeysvecqedsacpaaeeslpievmvdavhklkyenytssffirdiikp |
| | | | dppknlqlkplknsrqvevsweypdtwstphsyfsltfcvqvqgkskr |
| | | | ekkdrvftdktsatvicrknasisvraqdryyssswsewasvpcsggg |
| | | | gsggggsggggsrvipvsgparclsqsrnllkttddmvktareklkhys |
| | | | ctaedidheditrdqtstlktclplelhknesclatretssttrgsclppqkts |
| | | | lmmtlclgsiyedlkmyqtefqainaalqnhnhqqiildkgmlvaide |
| | | | lmqslnhngetlrqkppvgeadpyrvkmklcillhafstrvvtinrvm |
| | | | gylssaSGGPGPAGMKGLPGSggggsggggsggggsggg |
| | | | gsggggsggggsQSVLTQPPSVSGAPGQRVTISCSGS |
| | | | RSNIGSNTVKWYQQLPGTAPKLLIYYNDQRPS |
| | | | GVPDRFSGSKSGTSASLAITGLQAEDEADYYC |
| | | | QSYDRYTHPALLFGTGTKVTVLggggsggggsgggg |
| | | | sQVQLVESGGGVVQPGRSLRLSCAASGFTFSSY |
| | | | GMHWVRQAPGKGLEWVAFIRYDGSNKYYAD |
| | | | SVKGRFTISRDNSKNTLYLQMNSLRAEDTAVY |
| | | | YCKTHGSHDNWGQGTMVTVSSHHHHHH** |
| 13 | WW0329 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, | qkepknktflrceaknysgrftcwwlttistdltfsvkssrgssdpqgvtc |
| | | Albumin, scFv | gaatlsaervrgdnkeyeysvecqedsacpaaeeslpievmvdavhkl |
| | | Blocker, 2 | kyenytssffirdiikpdppknlqlkplknsrqvevsweypdtwstphs |
| | | cleavage sites | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssaSGGPGPAGMKGLPGSggggsg |
| | | | gggsggggsggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSSS |
| | | | GGPGPAGMKGLPGSEAHKSEIAHRYNDLGEQH |
| | | | FKGLVLIAFSQYLQKCSYDEHAKLVQEVTDFA |
| | | | KTCVADESAANCDKSLHTLFGDKLCAIPNLRE |
| | | | NYGELADCCTKQEPERNECFLQHKDDNPSLPP |
| | | | FERPEAEAMCTSFKENPTTFMGHYLHEVARRH |
| | | | PYFYAPELLYYAEQYNEILTQCCAEADKESCLT |
| | | | PKLDGVKEKALVSSVRQRMKCSSMQKFGERA |
| | | | FKAWAVARLSQTFPNADFAEITKLATDLTKVN |
| | | | KECCHGDLLECADDRAELAKYMCENQATISSK |
| | | | LQTCCDKPLLKKAHCLSEVEHDTMPADLPAIA |
| | | | ADFVEDQEVCKNYAEAKDVFLGTFLYEYSRR |
| | | | HPDYSVSLLLRLAKKYEATLEKCCAEANPPAC |
| | | | YGTVLAEFQPLVEEPKNLVKTNCDLYEKLGEY |
| | | | GFQNAILVRYTQKAPQVSTPTLVEAARNLGRV |
| | | | GTKCCTLPEDQRLPCVEDYLSAILNRVCLLHEK |
| | | | TPVSEHVTKCCSGSLVERRPCFSALTVDETYVP |
| | | | KEFKAETFTFHSDICTLPEKEKQIKKQTALAEL |
| | | | VKHKPKATAEQLKTVMDDFAQFLDTCCKAAD |
| | | | KDTCFSTEGPNLVTRCKDALAHHHHHH** |
| 14 | WW0330 | Monomeric IL-12 | vprdcgckpcictvpevssvfifppkpkdvltitltpkvtcvvvdiskdd |
| | | (chimeric) | pevqfswfvddvevhtaqtqpreeqfnstfrsvselpimhqdwlngk |
| | | polypeptide, Fc, | efkcrvnsaafpapiektisktkgrpkapqvytipppkeqmakdkvsl |
| | | scFv Blocker, 2 | tcmitdffpeditvewqwngqpaenykntqpimdtdgsyfvyskln |
| | | cleavage sites | vqksnweagntftcsvlheglhnhhtekslshspgkSGGPGPAG |
| | | | MKGLPGSiwelkkdvyvveldwypdapgemvvltcdtpeedg |
| | | | itwtldqssevlgsgktltiqvkefgdagqytchkggevlshsllllhkke |
| | | | dgiwstdilkdqkepknktflrceaknysgrftcwwlttistdltfsvkss |
| | | | rgssdpqgvtcgaatlsaervrgdnkeyeysvecqedsacpaaeeslpi |
| | | | evmvdavhklkyenytssffirdiikpdppknlqlkplknsrqvevsw |
| | | | eypdtwstphsyfsltfcvqvqgkskrekkdrvftdktsatvicrknasi |
| | | | svraqdryyssswsewasvpcsggggsggggsggggsrvipvsgpa |
| | | | rclsqsrnllkttddmvktareklkhysctaedidheditrdqtstlktclpl |
| | | | elhknesclatretssttrgsclppqktslmmtlclgsiyedlkmyqtefq |
| | | | ainaalqnhnhqqiildkgmlvaidelmqslnhngetlrqkppvgead |
| | | | pyrvkmklcillhafstrvvtinrvmgylssaSGGPGPAGMKG |
| | | | LPGSggggsggggsggggggggggggsggggsQSVLTQP |
| | | | PSVSGAPGQRVTISCSGSRSNIGSNTVKWYQQL |
| | | | PGTAPKLLIYYNDQRPSGVPDRFSGSKSGTSAS |
| | | | LAITGLQAEDEADYYCQSYDRYTHPALLFGTG |
| | | | TKVTVLggggsggggggggsQVQLVESGGGVVQPG |
| | | | RSLRLSCAASGFTFSSYGMHWVRQAPGKGLE |
| | | | WVAFIRYDGSNKYYADSVKGRFTISRDNSKNT |
| | | | LYLQMNSLRAEDTAVYYCKTHGSHDNWGQG |
| | | | TMVTVSSHHHHHH** |
| 15 | WW0331 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, Fc, | qkepknktflrceaknysgrftcwwlttistdltfsvkssrgssdpqgvtc |
| | | scFv Blocker, 2 | gaatlsaervrgdnkeyeysvecqedsacpaaeeslpievmvdavhkl |
| | | cleavage sites | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssaSGGPGPAGMKGLPGSggggsg |
| | | | gggsggggsggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSSS |
| | | | GGPGPAGMKGLPGSvprdcgckpcictvpevssvfifppkp |
| | | | kdvltitltpkvtcvvvdiskddpevqfswfvddvevhtaqtqpreeqf |
| | | | nstfrsvselpimhqdwlngkefkcrvnsaafpapiektisktkgrpka |
| | | | pqvytipppkeqmakdkvsltcmitdffpeditvewqwngqpaeny |
| | | | kntqpimdtdgsyfvysklnvqksnweagntftcsvlheglhnhhtek |
| | | | slshspgkHHHHHH** |
| 16 | WW0402 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSSGGPGPAGMKGLPG |
| | | Blocker, 3 | Siwelkkdvyvveldwypdapgemvvltcdtpeedgitwtldqsse |
| | | cleavage sites | vlgsgktltiqvkefgdagqytchkggevlshsllllhkkedgiwstdil |
| | | | kdqkepknktflrceaknysgrftcwwlttistdltfsvkssrgssdpqg |
| | | | vtcgaatlsaervrgdnkeyeysvecqedsacpaaeeslpievmvdav |
| | | | hklkyenytssffirdiikpdppknlqlkplknsrqvevsweypdtwst |
| | | | phsyfsltfcvqvqgkskrekkdrvftdktsatvicrknasisvraqdry |
| | | | yssswsewasvpcsggggsggggsggggsrvipvsgparclsqsrnll |
| | | | kttddmvktareklkhysctaedidheditrdqtstlktclplelhknesc |
| | | | latretssttrgsclppqktslmmtlclgsiyedlkmyqtefqainaalqn |
| | | | hnhqqiildkgmlvaidelmqslnhngetlrqkppvgeadpyrvkm |
| | | | klcillhafstrvvtinrvmgylssaSGGPGPAGMKGLPGSg |
| | | | gggsggggggggsggggggggggggsQSVLTQPPSVSG |
| | | | APGQRVTISCSGSRSNIGSNTVKWYQQLPGTAP |
| | | | KLLIYYNDQRPSGVPDRFSGSKSGTSASLAITG |
| | | | LQAEDEADYYCQSYDRYTHPALLFGTGTKVT |
| | | | VLSGGPGPAGMKGLPGSQVQLVESGGGVVQP |
| | | | GRSLRLSCAASGFTFSSYGMHWVRQAPGKGLE |
| | | | WVAFIRYDGSNKYYADSVKGRFTISRDNSKNT |
| | | | LYLQMNSLRAEDTAVYYCKTHGSHDNWGQG |
| | | | TMVTVSSHHHHHH** |
| 17 | WW0461 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, sdAb | TIGGSLSVSSQGTLVTVSSSGGPGPAGMKGLPG |
| | | Blocker, 2 | Siwelkkdvyvveldwypdapgemvvltcdtpeedgitwtldqsse |
| | | cleavage sites | vlgsgktltiqvkefgdagqytchkggevlshsllllhkkedgiwstdil |
| | | | kdqkepknktflrceaknysgrftcwwlttistdltfsvkssrgssdpqg |
| | | | vtcgaatlsaervrgdnkeyeysvecqedsacpaaeeslpievmvdav |
| | | | hklkyenytssffirdiikpdppknlqlkplknsrqvevsweypdtwst |
| | | | phsyfsltfcvqvqgkskrekkdrvftdktsatvicrknasisvraqdry |
| | | | yssswsewasvpcsggggsggggsggggsrvipvsgparclsqsrnll |
| | | | kttddmvktareklkhysctaedidheditrdqtstlktclplelhknesc |
| | | | latretssttrgsclppqktslmmtlclgsiyedlkmyqtefqainaalqn |
| | | | hnhqqiildkgmlvaidelmqslnhngetlrqkppvgeadpyrvkm |
| | | | klcillhafstrvvtinrvmgylssaSGGPGPAGMKGLPGSg |
| | | | gggsggggggggggggsggggggggsQVQLQESGGGL |
| | | | VQAGGSLRLSCAASGRTFSSVYDMGWFRQAP |
| | | | GKDREFVARITESARNTRYADSVRGRFTISRDN |
| | | | AKNTVYLQMNNLELEDAAVYYCAADPQTVV |
| | | | VGTPDYWGQGTQVTVSSHHHHHH** |
| 18 | WW0636 | | iwelkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcs** |
| 19 | WW0637 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSSGGPGPAGMKGLPG |
| | | Blocker, 2 | Srvipvsgparclsqsrnllkttddmvktareklkhysctaedidheditr |
| | | cleavage sites | dqtstlktclplelhknesclatretssttrgsclppqktslmmtlclgsiye |
| | | | dlkmyqtefqainaalqnhnhqqiildkgmlvaidelmqslnhngetl |
| | | | rqkppvgeadpyrvkmklcillhafstrvvtinrvmgylssaSGGP |
| | | | GPAGMKGLPGSggggsggggsggggsggggggggsgggg |
| | | | SQSVLTQPPSVSGAPGQRVTISCSGSRSNIGSNT |
| | | | VKWYQQLPGTAPKLLIYYNDQRPSGVPDRFSG |
| | | | SKSGTSASLAITGLQAEDEADYYCQSYDRYTH |
| | | | PALLFGTGTKVTVLggggsggggsggggsQVQLVES |
| | | | GGGVVQPGRSLRLSCAASGFTFSSYGMHWVR |
| | | | QAPGKGLEWVAFIRYDGSNKYYADSVKGRFTI |
| | | | SRDNSKNTLYLQMNSLRAEDTAVYYCKTHGS |
| | | | HDNWGQGTMVTVSS** |
| 20 | WW0638 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSSGGPGPAGMKGLPG |
| | | Blocker, 2 | Siwelkkdvyvveldwypdapgemvvltcdtpeedgitwtldqsse |
| | | cleavage sites | vlgsgktltiqvkefgdagqytchkggevlshsllllhkkedgiwstdil |
| | | | kdqkepknktflrceaknysgrftcwwlttistdltfsvkssrgssdpqg |
| | | | vtcgaatlsaervrgdnkeyeysvecqedsaSpaaeeslpievmvda |
| | | | vhklkyenytssffirdiikpdppknlqlkplknsrqvevsweypdtw |
| | | | stphsyfsltfcvqvqgkskrekkdrvftdktsatvicrknasisvraqdr |
| | | | yyssswsewasvpcsggggsggggsggggsrvipvsgparclsqsrn |
| | | | llkttddmvktareklkhysctaedidheditrdqtstlktclplelhknes |
| | | | Slatretssttrgsclppqktslmmtlclgsiyedlkmyqtefqainaalq |
| | | | nhnhqqiildkgmlvaidelmqslnhngetlrqkppvgeadpyrvk |
| | | | mklcillhafstrvvtinrvmgylssaSGGPGPAGMKGLPGS |
| | | | ggggggggsggggsggggsggggggggsQSVLTQPPSVS |
| | | | GAPGQRVTISCSGSRSNIGSNTVKWYQQLPGT |
| | | | APKLLIYYNDQRPSGVPDRFSGSKSGTSASLAIT |
| | | | GLQAEDEADYYCQSYDRYTHPALLFGTGTKV |
| | | | TVLggggsggggsggggsQVQLVESGGGVVQPGRSL |
| | | | RLSCAASGFTFSSYGMHWVRQAPGKGLEWVA |
| | | | FIRYDGSNKYYADSVKGRFTISRDNSKNTLYLQ |
| | | | MNSLRAEDTAVYYCKTHGSHDNWGQGTMVT |
| | | | VSS** |
| 21 | WW0639 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSSGGPGPAGMKGLPG |
| | | Blocker, 2 | Siwelkkdvyvveldwypdapgemvvltcdtpeedgitwtldqsse |
| | | cleavage sites | vlgsgktltiqvkefgdagqytchkggevlshsllllhkkedgiwstdil |
| | | | kdqkepknktflrceaknysgrftcwwlttistdltfsvkssrgssdpqg |
| | | | vtcgaatlsaervrgdnkeyeysvecqedsacpaaeeslpievmvdav |
| | | | hklkyenytssffirdiikpdppknlqlkplknsrqvevsweypdtwst |
| | | | phsyfsltfcvqvqgkskrekkdrvftdktsatvicrknasisvraqdry |
| | | | yssswsewasvpcsggggsggggsggggggggsrvipvsgparcl |
| | | | sqsrnllkttddmvktareklkhysctaedidheditrdqtstlktclplel |
| | | | hknesclatretssttrgsclppqktslmmtlclgsiyedlkmyqtefqai |
| | | | naalqnhnhqgiildkgmlvaidelmqslnhngetlrgkppvgeadp |
| | | | yrvkmklcillhafstrvvtinrvmgylssaSGGPGPAGMKGL |
| | | | PGSggggsggggsggggsggggsggggsggggsQSVLTQPP |
| | | | SVSGAPGQRVTISCSGSRSNIGSNTVKWYQQLP |
| | | | GTAPKLLIYYNDQRPSGVPDRFSGSKSGTSASL |
| | | | AITGLQAEDEADYYCQSYDRYTHPALLFGTGT |
| | | | KVTVLggggsggggsggggsQVQLVESGGGVVQPG |
| | | | RSLRLSCAASGFTFSSYGMHWVRQAPGKGLE |
| | | | WVAFIRYDGSNKYYADSVKGRFTISRDNSKNT |
| | | | LYLQMNSLRAEDTAVYYCKTHGSHDNWGQG |
| | | | TMVTVSS** |
| 22 | WW0640 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSSGGPGPAGMKGLPG |
| | | Blocker, 2 | Siwelkkdvyvveldwypdapgemvvltcdtpeedgitwtldqsse |
| | | cleavage sites | vlgsgktltiqvkefgdagqytchkggevlshsllllhkkedgiwstdil |
| | | | kdqkepknktflrceaknysgrftcwwlttistdltfsvkssrgssdpqg |
| | | | vtcgaatlsaervrgdnkeyeysvecqedsacpaaeeslpievmvdav |
| | | | hklkyenytssffirdiikpdppknlqlkplknsrqvevsweypdtwst |
| | | | phsyfsltfcvqvqgkskrekkdrvftdktsatvicrknasisvraqdry |
| | | | yssswsewasvpcsggggsggggsggggsrvipvsgparclsqsrnll |
| | | | kttddmvktareklkhysctaedidheditrdqtstlktclplelhknesc |
| | | | latretssttrgsclppqktslmmtlclgsiyedlkmyqtefqainaalqn |
| | | | hnhqqiildkgmlvaidelmqslnhngetlrqkppvgeadpyrvkm |
| | | | klcillhafstrvvtinrvmgylssaSGGPGPAGMKGLPGSg |
| | | | gggsggggggggsggggggggsggggsQSVLTQPPSVSG |
| | | | APGQRVTISCSGSRSNIGSNTVKWYQQLPGTAP |
| | | | KLLIYYNDQRPSGVPDRFSGSKSGTSASLAITG |
| | | | LQAEDEADYYCQSYDRYTHPALLFGcGTKVTV |
| | | | LggggsggggsggggsQVQLVESGGGVVQPGRSLRL |
| | | | SCAASGFTFSSYGMHWVRQAPGKcLEWVAFIR |
| | | | YDGSNKYYADSVKGRFTISRDNSKNTLYLQM |
| | | | NSLRAEDTAVYYCKTHGSHDNWGQGTMVTV |
| | | | SS** |
| 23 | WW0641 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSSGGPGPAGMKGLPG |
| | | Blocker, 2 | Siwelkkdvyvveldwypdapgemvvltcdtpeedgitwtldqsse |
| | | cleavage sites | vlgsgktltiqvkefgdagqytchkggevlshsllllhkkedgiwstdil |
| | | | kdqkepknktflrceaknysgrftcwwlttistdltfsvkssrgssdpqg |
| | | | vtcgaatlsaervrgdnkeyeysvecqedsacpaaeeslpievmvdav |
| | | | hklkyenytssffirdiikpdppknlqlkplknsrqvevsweypdtwst |
| | | | phsyfsltfcvqvqgkskrekkdrvftdktsatvicrknasisvraqdry |
| | | | yssswsewasvpcsggggsggggsggggsrvipvsgparclsqsrnll |
| | | | kttddmvktareklkhysctaedidheditrdqtstlktclplelhknesc |
| | | | latretssttrgsclppqktslmmtlclgsiyedlkmyqtefqainaalqn |
| | | | hnhqqiildkgmlvaidelmqslnhngetlrqkppvgeadpyrvkm |
| | | | klcillhafstrvvtinrvmgylssaSGGPGPAGMKGLPGSg |
| | | | gggsggggggggggggsggggggggsQSVLTQPPSVSG |
| | | | APGQRVTISCSGSRSNIGSNTVKWYQQLPGTCP |
| | | | KLLIYYNDQRPSGVPDRFSGSKSGTSASLAITG |
| | | | LQAEDEADYYCQSYDRYTHPALLFGTGTKVT |
| | | | VLggggsggggsggggsQVQLVESGGGVVQPGRSLR |
| | | | LSCAASGFTFSSYGMHWVRQAPGKGLEWVAFI |
| | | | RYDGSNKYYADSVKGRFTISRDNSKNTLYLQM |
| | | | NSLRAEDTAVYYCKTHGSHDNWGcGTMVTVS |
| | | | S** |
| 24 | WW0649 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGSNTVKWYQQLPGTAPKLLIY |
| | | | YNDQRPSGVPDRFSGSKSGTSASLAITGLQAED |
| | | | EADYYCQSYDRYTHPALLFGTGTKVTVLggggs |
| | | | ggggsggggsQVQLVESGGGVVQPGRSLRLSCAAS |
| | | | GFTFSSYGMHWVRQAPGKGLEWVAFIRYDGS |
| | | | NKYYADSVKGRFTISRDNSKNTLYLQMNSLRA |
| | | | EDTAVYYCKTHGSHDNWGQGTMVTVSS** |
| 25 | WW0650 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggggggggggsggggsQSVLTQPPSVSGAP |
| | | | GQRVTISCSGSRSNIGSNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSS** |
| 26 | WW0651 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggggggsggggggggsQSVLTQPPSVSGAPGQRV |
| | | | TISCSGSRSNIGSNTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYDGS |
| | | | NKYYADSVKGRFTISRDNSKNTLYLQMNSLRA |
| | | | EDTAVYYCKTHGSHDNWGQGTMVTVSS** |
| 27 | WW0652 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSS** |
| 28 | WW0662 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdaggytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGSNTVKWYQQLPGTAPKLLIY |
| | | | YNDQRPSGVPDRFSGSKSGTSASLAITGLQAED |
| | | | EADYYCQSYDRYTHPALLFGTGTKVTVLggggs |
| | | | ggggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 29 | WW0663 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggsggggsQSVLTQPPSVSGAP |
| | | | GQRVTISCSGSRSNIGSNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 30 | WW0664 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefgainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGSeTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 31 | WW0665 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggggggsggggsggggsggggsQSVLTQPPSVSGAP |
| | | | GQRVTISCSGSRSNIGSeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 32 | WW0666 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggggggsggggsggggsQSVLTQPPSVSGAPGQR |
| | | | VTISCSGSRSNIGdNTVKWYQQLPGTAPKLLIY |
| | | | YNDQRPSGVPDRFSGSKSGTSASLAITGLQAED |
| | | | EADYYCQSYDRYTHPALLFGTGTKVTVLggggs |
| | | | ggggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 33 | WW0667 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggggggsQSVLTQPPSVSGAP |
| | | | GQRVTISCSGSRSNIGdNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 34 | WW0668 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGdeTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYDGS |
| | | | NKYYADSVKGRFTISRDNSKNTLYLQMNSLRA |
| | | | EDTAVYYCKTHGSHDNWGQGTMVTVSS** |
| 35 | WW0669 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggsggggsQSVLTQPPSVSGAP |
| | | | GQRVTISCSGSRSNIGdeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSS** |
| 36 | WW0670 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGdeTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 37 | WW0671 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggsggggsQSVLTQPPSVSGAP |
| | | | GQRVTISCSGSRSNIGdeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 38 | WW0672 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSeSNIGSNdVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYDGS |
| | | | NKYYADSVKGRFTISRDNSKNTLYLQMNSLRA |
| | | | EDTAVYYCKTHGSHDNWGQGTMVTVSS** |
| 39 | WW0673 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggggggsggggsQSVLTQPPSVSGAP |
| | | | GQRVTISCSGSeSNIGSNdVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSS** |
| 40 | WW0674 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGeNTVKWYQQLPGTAPKLLIY |
| | | | YNDQRPSGVPDRFSGSKSGTSASLAITGLQAED |
| | | | EADYYCQSYDRYTHPALLFGTGTKVTVLggggs |
| | | | ggggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 41 | WW0675 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggsggggsQSVLTQPPSVSGAP |
| | | | GQRVTISCSGSRSNIGeNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 42 | WW0676 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGeeTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYDGS |
| | | | NKYYADSVKGRFTISRDNSKNTLYLQMNSLRA |
| | | | EDTAVYYCKTHGSHDNWGQGTMVTVSS** |
| 43 | WW0677 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggsggggsQSVLTQPPSVSGAP |
| | | | GQRVTISCSGSRSNIGeeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRY |
| | | | DGSNKYYADSVKGRFTISRDNSKNTLYLQMNS |
| | | | LRAEDTAVYYCKTHGSHDNWGQGTMVTVSS** |
| 44 | WW0678 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdaggytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGSeTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYeGSN |
| | | | KYYADSVKGRFTISRDNSKNTLYLQMNSLRAE |
| | | | DTAVYYCKTHGSHDNWGQGTMVTVSS** |
| 45 | WW0679 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggggggsQSVLTQPPSVSGAP |
| | | | GQRVTISCSGSRSNIGSeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRYe |
| | | | GSNKYYADSVKGRFTISRDNSKNTLYLQMNSL |
| | | | RAEDTAVYYCKTHGSHDNWGQGTMVTVSS** |
| 46 | WW0680 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggggggsggggggggsQSVLTQPPSVSGAPGQRV |
| | | | TISCSGSRSNIGSNTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 47 | WW0681 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 48 | WW0682 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggsggggsggggsggggsQSVLTQPPSVSGAPGQRV |
| | | | TISCSGSRSNIGSeTVKWYQQLPGTAPKLLIYYN |
| | | | DQRPSGVPDRFSGSKSGTSASLAITGLQAEDEA |
| | | | DYYCQSYDRYTHPALLFGTGTKVTVLggggggg |
| | | | gsggggsQVQLVESGGGVVQPGRSLRLSCAASGF |
| | | | TFSSYGMHWVRQAPGKGLEWVAFIRYeGSNK |
| | | | YYAeSVKGRFTISRDNSKNTLYLQMNSLRAED |
| | | | TAVYYCKTHGSHDNWGQGTMVTVSS** |
| 49 | WW0683 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | Imdpkrqifldqnmlavidelmqalnfnsetvpqkssleepdfyktki |
| | | | klcillhafriravtidrvmsylnassggpALFKSSFPpgsggggsg |
| | | | gggsggggsggggsggggggggsQSVLTQPPSVSGAPG |
| | | | QRVTISCSGSRSNIGSeTVKWYQQLPGTAPKLLI |
| | | | YYNDQRPSGVPDRFSGSKSGTSASLAITGLQAE |
| | | | DEADYYCQSYDRYTHPALLFGTGTKVTVLgggg |
| | | | sggggsggggsQVQLVESGGGVVQPGRSLRLSCAA |
| | | | SGFTFSSYGMHWVRQAPGKGLEWVAFIRYeGS |
| | | | NKYYAeSVKGRFTISRDNSKNTLYLQMNSLRA |
| | | | EDTAVYYCKTHGSHDNWGQGTMVTVSS** |
| 50 | WW0684 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtogaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggggggsggggggggsQSVLTQPPSVSGAPGQRV |
| | | | TISCSGSRSNIGdNTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 51 | WW0685 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | Imdpkrqifldqnmlavidelmqalnfnsetvpqkssleepdfyktki |
| | | | klcillhafriravtidrvmsylnassggpALFKSSFPpgsggggsg |
| | | | gggsggggsggggsggggsggggsQSVLTQPPSVSGAPG |
| | | | QRVTISCSGSRSNIGdNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 52 | WW0686 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggggggs |
| | | | ggggggggsggggggggsQSVLTQPPSVSGAPGQRV |
| | | | TISCSGSRSNIGdeTVKWYQQLPGTAPKLLIYYN |
| | | | DQRPSGVPDRFSGSKSGTSASLAITGLQAEDEA |
| | | | DYYCQSYDRYTHPALLFGTGTKVTVLggggsggg |
| | | | gsggggsQVQLVESGGGVVQPGRSLRLSCAASGF |
| | | | TFSSYGMHWVRQAPGKGLEWVAFIRYDGSNK |
| | | | YYADSVKGRFTISRDNSKNTLYLQMNSLRAED |
| | | | TAVYYCKTHGSHDNWGQGTMVTVSS** |
| 53 | WW0687 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggsggggsggggggggsQSVLTQPPSVSGAPG |
| | | | QRVTISCSGSRSNIGdeTVKWYQQLPGTAPKLLI |
| | | | YYNDQRPSGVPDRFSGSKSGTSASLAITGLQAE |
| | | | DEADYYCQSYDRYTHPALLFGTGTKVTVLgggg |
| | | | sggggsggggsQVQLVESGGGVVQPGRSLRLSCAA |
| | | | SGFTFSSYGMHWVRQAPGKGLEWVAFIRYDG |
| | | | SNKYYADSVKGRFTISRDNSKNTLYLQMNSLR |
| | | | AEDTAVYYCKTHGSHDNWGQGTMVTVSS** |
| 54 | WW0688 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggsggggsggggsggggsQSVLTQPPSVSGAPGQRV |
| | | | TISCSGSRSNIGdeTVKWYQQLPGTAPKLLIYYN |
| | | | DQRPSGVPDRFSGSKSGTSASLAITGLQAEDEA |
| | | | DYYCQSYDRYTHPALLFGTGTKVTVLggggggg |
| | | | gsggggsQVQLVESGGGVVQPGRSLRLSCAASGF |
| | | | TFSSYGMHWVRQAPGKGLEWVAFIRYeGSNK |
| | | | YYAeSVKGRFTISRDNSKNTLYLQMNSLRAED |
| | | | TAVYYCKTHGSHDNWGQGTMVTVSS** |
| 55 | WW0689 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggsggggsggggsggggsQSVLTQPPSVSGAPG |
| | | | QRVTISCSGSRSNIGdeTVKWYQQLPGTAPKLLI |
| | | | YYNDQRPSGVPDRFSGSKSGTSASLAITGLQAE |
| | | | DEADYYCQSYDRYTHPALLFGTGTKVTVLgggg |
| | | | sggggsggggsQVQLVESGGGVVQPGRSLRLSCAA |
| | | | SGFTFSSYGMHWVRQAPGKGLEWVAFIRYeGS |
| | | | NKYYAeSVKGRFTISRDNSKNTLYLQMNSLRA |
| | | | EDTAVYYCKTHGSHDNWGQGTMVTVSS** |
| 56 | WW0690 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggggggsggggggggsQSVLTQPPSVSGAPGQRV |
| | | | TISCSGSeSNIGSNdVKWYQQLPGTAPKLLIYYN |
| | | | DQRPSGVPDRFSGSKSGTSASLAITGLQAEDEA |
| | | | DYYCQSYDRYTHPALLFGTGTKVTVLggggsggg |
| | | | gsggggsQVQLVESGGGVVQPGRSLRLSCAASGF |
| | | | TFSSYGMHWVRQAPGKGLEWVAFIRYDGSNK |
| | | | YYADSVKGRFTISRDNSKNTLYLQMNSLRAED |
| | | | TAVYYCKTHGSHDNWGQGTMVTVSS** |
| 57 | WW0691 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggggggsggggggggsQSVLTQPPSVSGAPG |
| | | | QRVTISCSGSeSNIGSNdVKWYQQLPGTAPKLLI |
| | | | YYNDQRPSGVPDRFSGSKSGTSASLAITGLQAE |
| | | | DEADYYCQSYDRYTHPALLFGTGTKVTVLgggg |
| | | | sggggsggggsQVQLVESGGGVVQPGRSLRLSCAA |
| | | | SGFTFSSYGMHWVRQAPGKGLEWVAFIRYDG |
| | | | SNKYYADSVKGRFTISRDNSKNTLYLQMNSLR |
| | | | AEDTAVYYCKTHGSHDNWGQGTMVTVSS** |
| 58 | WW0692 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggggggsggggggggsQSVLTQPPSVSGAPGQRV |
| | | | TISCSGSRSNIGeNTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 59 | WW0693 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggsggggsggggggggsQSVLTQPPSVSGAPG |
| | | | QRVTISCSGSRSNIGeNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 60 | WW0694 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggggggsggggsggggsQSVLTQPPSVSGAPGQRV |
| | | | TISCSGSRSNIGeeTVKWYQQLPGTAPKLLIYYN |
| | | | DQRPSGVPDRFSGSKSGTSASLAITGLQAEDEA |
| | | | DYYCQSYDRYTHPALLFGTGTKVTVLggggsggg |
| | | | gsggggsQVQLVESGGGVVQPGRSLRLSCAASGF |
| | | | TFSSYGMHWVRQAPGKGLEWVAFIRYDGSNK |
| | | | YYADSVKGRFTISRDNSKNTLYLQMNSLRAED |
| | | | TAVYYCKTHGSHDNWGQGTMVTVSS** |
| 61 | WW0695 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | Imdpkrqifldqnmlavidelmqalnfnsetvpqkssleepdfyktki |
| | | | klcillhafriravtidrvmsylnassggpALFKSSFPpgsggggsg |
| | | | gggsggggsggggggggsggggsQSVLTQPPSVSGAPG |
| | | | QRVTISCSGSRSNIGeeTVKWYQQLPGTAPKLLI |
| | | | YYNDQRPSGVPDRFSGSKSGTSASLAITGLQAE |
| | | | DEADYYCQSYDRYTHPALLFGTGTKVTVLgggg |
| | | | sggggsggggsQVQLVESGGGVVQPGRSLRLSCAA |
| | | | SGFTFSSYGMHWVRQAPGKGLEWVAFIRYDG |
| | | | SNKYYADSVKGRFTISRDNSKNTLYLQMNSLR |
| | | | AEDTAVYYCKTHGSHDNWGQGTMVTVSS** |
| 62 | WW0696 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggggggsggggsggggsQSVLTQPPSVSGAPGQRV |
| | | | TISCSGSRSNIGSeTVKWYQQLPGTAPKLLIYYN |
| | | | DQRPSGVPDRFSGSKSGTSASLAITGLQAEDEA |
| | | | DYYCQSYDRYTHPALLFGTGTKVTVLggggsggg |
| | | | gsggggsQVQLVESGGGVVQPGRSLRLSCAASGF |
| | | | TFSSYGMHWVRQAPGKGLEWVAFIRYeGSNK |
| | | | YYADSVKGRFTISRDNSKNTLYLQMNSLRAED |
| | | | TAVYYCKTHGSHDNWGQGTMVTVSS** |
| 63 | WW0697 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, scFv | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | Imdpkrqifldqnmlavidelmqalnfnsetvpqkssleepdfyktki |
| | | | klcillhafriravtidrvmsylnassggpALFKSSFPpgsggggsg |
| | | | gggsggggggggggggsggggsQSVLTQPPSVSGAPG |
| | | | QRVTISCSGSRSNIGSeTVKWYQQLPGTAPKLLI |
| | | | YYNDQRPSGVPDRFSGSKSGTSASLAITGLQAE |
| | | | DEADYYCQSYDRYTHPALLFGTGTKVTVLgggg |
| | | | sggggsggggsQVQLVESGGGVVQPGRSLRLSCAA |
| | | | SGFTFSSYGMHWVRQAPGKGLEWVAFIRYeGS |
| | | | NKYYADSVKGRFTISRDNSKNTLYLQMNSLRA |
| | | | EDTAVYYCKTHGSHDNWGQGTMVTVSS** |
| 64 | WW0698 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggggggsggggsggggsQSVLTQPPSVSGAPGQR |
| | | | VTISCSGSRSNIGSNTVKWYQQLPGTAPKLLIY |
| | | | YNDQRPSGVPDRFSGSKSGTSASLAITGLQAED |
| | | | EADYYCQSYDRYTHPALLFGTGTKVTVLgqpka |
| | | | apsvtlfppsseelqankatlvclisdfypgavtvawkadsspvkagve |
| | | | tttpskqsnnkyaassylsltpeqwkshrsyscqvthegstvektvapte |
| | | | CS** |
| 65 | WW0699 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggsggggsQSVLTQPPSVSGAP |
| | | | GQRVTISCSGSRSNIGSNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 66 | WW0700 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggggggsQSVLTQPPSVSGAPGQR |
| | | | VTISCSGSRSNIGSeTVKWYQQLPGTAPKLLIYY |
| | | | NDQRPSGVPDRFSGSKSGTSASLAITGLQAEDE |
| | | | ADYYCQSYDRYTHPALLFGTGTKVTVLgqpkaa |
| | | | psvtlfppsseelqankatlvclisdfypgavtvawkadsspvkagvett |
| | | | tpskqsnnkyaassylsltpeqwkshrsyscqvthegstvektvaptec |
| | | | S** |
| 67 | WW0701 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggggggsggggsggggsggggsQSVLTQPPSVSGAP |
| | | | GQRVTISCSGSRSNIGSeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 68 | WW0702 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggggggsggggsggggsQSVLTQPPSVSGAPGQR |
| | | | VTISCSGSRSNIGdNTVKWYQQLPGTAPKLLIY |
| | | | YNDQRPSGVPDRFSGSKSGTSASLAITGLQAED |
| | | | EADYYCQSYDRYTHPALLFGTGTKVTVLgqpka |
| | | | apsvtlfppsseelqankatlvclisdfypgavtvawkadsspvkagve |
| | | | tttpskqsnnkyaassylsltpeqwkshrsyscqvthegstvektvapte |
| | | | CS** |
| 69 | WW0703 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggggggsQSVLTQPPSVSGAP |
| | | | GQRVTISCSGSRSNIGdNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 70 | WW0704 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGdeTVKWYQQLPGTAPKLLIYY |
| | | | NDQRPSGVPDRFSGSKSGTSASLAITGLQAEDE |
| | | | ADYYCQSYDRYTHPALLFGTGTKVTVLgqpkaa |
| | | | psvtlfppsseelqankatlvclisdfypgavtvawkadsspvkagvett |
| | | | tpskqsnnkyaassylsltpeqwkshrsyscqvthegstvektvaptec |
| | | | s** |
| 71 | WW0705 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggggggsQSVLTQPPSVSGAP |
| | | | GQRVTISCSGSRSNIGdeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 72 | WW0706 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSeSNIGSNdVKWYQQLPGTAPKLLIYY |
| | | | NDQRPSGVPDRFSGSKSGTSASLAITGLQAEDE |
| | | | ADYYCQSYDRYTHPALLFGTGTKVTVLgqpkaa |
| | | | psvtlfppsseelqankatlvclisdfypgavtvawkadsspvkagvett |
| | | | tpskqsnnkyaassylsltpeqwkshrsyscqvthegstvektvaptec |
| | | | s** |
| 73 | WW0707 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggggggsQSVLTQPPSVSGAP |
| | | | GQRVTISCSGSeSNIGSNdVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 74 | WW0708 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGeNTVKWYQQLPGTAPKLLIY |
| | | | YNDQRPSGVPDRFSGSKSGTSASLAITGLQAED |
| | | | EADYYCQSYDRYTHPALLFGTGTKVTVLgqpka |
| | | | apsvtlfppsseelqankatlvclisdfypgavtvawkadsspvkagve |
| | | | tttpskqsnnkyaassylsltpeqwkshrsyscqvthegstvektvapte |
| | | | CS** |
| 75 | WW0709 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggggggsggggggggsQSVLTQPPSVSGAP |
| | | | GQRVTISCSGSRSNIGeNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 76 | WW0710 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsi |
| | | Blocker, 2 | welkkdvyvveldwypdapgemvvltcdtpeedgitwtldqssevl |
| | | cleavage sites | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsggggsggg |
| | | | gsggggsggggsggggsggggsQSVLTQPPSVSGAPGQR |
| | | | VTISCSGSRSNIGeeTVKWYQQLPGTAPKLLIYY |
| | | | NDQRPSGVPDRFSGSKSGTSASLAITGLQAEDE |
| | | | ADYYCQSYDRYTHPALLFGTGTKVTVLgqpkaa |
| | | | psvtlfppsseelqankatlvclisdfypgavtvawkadsspvkagvett |
| | | | tpskqsnnkyaassylsltpeqwkshrsyscqvthegstvektvaptec |
| | | | s** |
| 77 | WW0711 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpGPAGLYAQpgsi |
| | | cleavage sites | welkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsgggg |
| | | | sggggsggggsggggsggggggggsQSVLTQPPSVSGAP |
| | | | GQRVTISCSGSRSNIGeeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 78 | WW0712 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggsggggsggggsggggsQSVLTQPPSVSGAPGQRV |
| | | | TISCSGSRSNIGSNTVKWYQQLPGTAPKLLIYY |
| | | | NDQRPSGVPDRFSGSKSGTSASLAITGLQAEDE |
| | | | ADYYCQSYDRYTHPALLFGTGTKVTVLgqpkaa |
| | | | psvtlfppsseelqankatlvclisdfypgavtvawkadsspvkagvett |
| | | | tpskqsnnkyaassylsltpeqwkshrsyscqvthegstvektvaptec |
| | | | s** |
| 79 | WW0713 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgq |
| | | | pkaapsvtlfppsseelqankatlvclisdfypgavtvawkadsspvka |
| | | | gvetttpskqsnnkyaassylsltpeqwkshrsyscqvthegstvektv |
| | | | aptecs** |
| 80 | WW0714 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggggggs |
| | | | ggggsggggsggggsggggsQSVLTQPPSVSGAPGQRV |
| | | | TISCSGSRSNIGSeTVKWYQQLPGTAPKLLIYYN |
| | | | DQRPSGVPDRFSGSKSGTSASLAITGLQAEDEA |
| | | | DYYCQSYDRYTHPALLFGTGTKVTVLgqpkaaps |
| | | | vtlfppsseelqankatlvclisdfypgavtvawkadsspvkagvetttp |
| | | | skqsnnkyaassylsltpeqwkshrsyscqvthegstvektvaptecs** |
| 81 | WW0715 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggsggggsggggggggsQSVLTQPPSVSGAPG |
| | | | QRVTISCSGSRSNIGSeTVKWYQQLPGTAPKLLI |
| | | | YYNDQRPSGVPDRFSGSKSGTSASLAITGLQAE |
| | | | DEADYYCQSYDRYTHPALLFGTGTKVTVLgqpk |
| | | | aapsvtlfppsseelqankatlvclisdfypgavtvawkadsspvkagv |
| | | | etttpskqsnnkyaassylsltpeqwkshrsyscqvthegstvektvapt |
| | | | ecs** |
| 82 | WW0716 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggsggggs |
| | | | ggggsggggsggggsggggsQSVLTQPPSVSGAPGQRV |
| | | | TISCSGSRSNIGdNTVKWYQQLPGTAPKLLIYY |
| | | | NDQRPSGVPDRFSGSKSGTSASLAITGLQAEDE |
| | | | ADYYCQSYDRYTHPALLFGTGTKVTVLgqpkaa |
| | | | psvtlfppsseelqankatlvclisdfypgavtvawkadsspvkagvett |
| | | | tpskqsnnkyaassylsltpeqwkshrsyscqvthegstvektvaptec |
| | | | s** |
| 83 | WW0717 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | ggggggggggggsggggggggsQSVLTQPPSVSGAPG |
| | | | QRVTISCSGSRSNIGdNTVKWYQQLPGTAPKLL |
| | | | IYYNDQRPSGVPDRFSGSKSGTSASLAITGLQA |
| | | | EDEADYYCQSYDRYTHPALLFGTGTKVTVLgq |
| | | | pkaapsvtlfppsseelqankatlvclisdfypgavtvawkadsspvka |
| | | | gvetttpskqsnnkyaassylsltpeqwkshrsyscqvthegstvektv |
| | | | aptecs** |
| 84 | WW0718 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggggggs |
| | | | ggggsggggsggggsggggsQSVLTQPPSVSGAPGQRV |
| | | | TISCSGSRSNIGdeTVKWYQQLPGTAPKLLIYYN |
| | | | DQRPSGVPDRFSGSKSGTSASLAITGLQAEDEA |
| | | | DYYCQSYDRYTHPALLFGTGTKVTVLgqpkaaps |
| | | | vtlfppsseelqankatlvclisdfypgavtvawkadsspvkagvetttp |
| | | | skqsnnkyaassylsltpeqwkshrsyscqvthegstvektvaptecs** |
| 85 | WW0719 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggggggsggggggggsQSVLTQPPSVSGAPG |
| | | | QRVTISCSGSRSNIGdeTVKWYQQLPGTAPKLLI |
| | | | YYNDQRPSGVPDRFSGSKSGTSASLAITGLQAE |
| | | | DEADYYCQSYDRYTHPALLFGTGTKVTVLgqpk |
| | | | aapsvtlfppsseelqankatlvclisdfypgavtvawkadsspvkagv |
| | | | etttpskqsnnkyaassylsltpeqwkshrsyscqvthegstvektvapt |
| | | | ecs** |
| 86 | WW0720 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrokppvgeadpyrvkmklcill |
| | | | hafstrvvtinrvmgylssasggpALFKSSFPpgsggggsggggs |
| | | | ggggsggggggggsggggsQSVLTQPPSVSGAPGQRV |
| | | | TISCSGSeSNIGSNdVKWYQQLPGTAPKLLIYYN |
| | | | DQRPSGVPDRFSGSKSGTSASLAITGLQAEDEA |
| | | | DYYCQSYDRYTHPALLFGTGTKVTVLgqpkaaps |
| | | | vtlfppsseelqankatlvclisdfypgavtvawkadsspvkagvetttp |
| | | | skqsnnkyaassylsltpeqwkshrsyscqvthegstvektvaptecs** |
| 87 | WW0721 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | Imdpkrqifldqnmlavidelmqalnfnsetvpqkssleepdfyktki |
| | | | klcillhafriravtidrvmsylnassggpALFKSSFPpgsggggsg |
| | | | gggsggggsggggsggggsggggsQSVLTQPPSVSGAPG |
| | | | QRVTISCSGSeSNIGSNdVKWYQQLPGTAPKLLI |
| | | | YYNDQRPSGVPDRFSGSKSGTSASLAITGLQAE |
| | | | DEADYYCQSYDRYTHPALLFGTGTKVTVLgqpk |
| | | | aapsvtlfppsseelqankatlvclisdfypgavtvawkadsspvkagv |
| | | | etttpskqsnnkyaassylsltpeqwkshrsyscqvthegstvektvapt |
| | | | ecs** |
| 88 | WW0722 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggggggs |
| | | | ggggsggggsggggsggggsQSVLTQPPSVSGAPGQRV |
| | | | TISCSGSRSNIGeNTVKWYQQLPGTAPKLLIYY |
| | | | NDQRPSGVPDRFSGSKSGTSASLAITGLQAEDE |
| | | | ADYYCQSYDRYTHPALLFGTGTKVTVLgqpkaa |
| | | | psvtlfppsseelqankatlvclisdfypgavtvawkadsspvkagvett |
| | | | tpskqsnnkyaassylsltpeqwkshrsyscqvthegstvektvaptec |
| | | | s** |
| 89 | WW0723 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggggggggggsggggsQSVLTQPPSVSGAPG |
| | | | QRVTISCSGSRSNIGeNTVKWYQQLPGTAPKLL |
| | | | IYYNDQRPSGVPDRFSGSKSGTSASLAITGLQA |
| | | | EDEADYYCQSYDRYTHPALLFGTGTKVTVLgq |
| | | | pkaapsvtlfppsseelqankatlvclisdfypgavtvawkadsspvka |
| | | | gvetttpskqsnnkyaassylsltpeqwkshrsyscqvthegstvektv |
| | | | aptecs** |
| 90 | WW0724 | Monomeric IL-12 | EVQLVESGGGLVQPGNSLRLSCAASGFTFSKFG |
| | | (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsiwe |
| | | Blocker, 2 | lkkdvyvveldwypdapgemvvltcdtpeedgitwtldqssevlgsg |
| | | cleavage sites | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrvipvsgparclsqsrnllkttdd |
| | | | mvktareklkhysctaedidheditrdqtstlktclplelhknesclatret |
| | | | ssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsggggggggs |
| | | | ggggsggggggggsggggsQSVLTQPPSVSGAPGQRV |
| | | | TISCSGSRSNIGeeTVKWYQQLPGTAPKLLIYYN |
| | | | DQRPSGVPDRFSGSKSGTSASLAITGLQAEDEA |
| | | | DYYCQSYDRYTHPALLFGTGTKVTVLgqpkaaps |
| | | | vtlfppsseelqankatlvclisdfypgavtvawkadsspvkagvetttp |
| | | | skqsnnkyaassylsltpeqwkshrsyscqvthegstvektvaptecs** |
| 91 | WW0725 | Monomeric IL-12 | EVQLVESGGGLVQPGNSLRLSCAASGFTFSKFG |
| | | polypeptide, anti- | MSWVRQAPGKGLEWVSSISGSGRDTLYAESV |
| | | HSA sdAb, Fab | KGRFTISRDNAKTTLYLQMNSLRPEDTAVYYC |
| | | Blocker, 2 | TIGGSLSVSSQGTLVTVSSsggpALFKSSFPpgsiwe |
| | | cleavage sites | lkkdvyvveldwypdapgemvvltcdtpeedgitwtldqssevlgsg |
| | | | ktltiqvkefgdagqytchkggevlshsllllhkkedgiwstdilkdqke |
| | | | pknktflrceaknysgrftcwwlttistdltfsvkssrgssdpqgvtcgaa |
| | | | tlsaervrgdnkeyeysvecqedsacpaaeeslpievmvdavhklky |
| | | | enytssffirdiikpdppknlqlkplknsrqvevsweypdtwstphsyf |
| | | | sltfcvqvqgkskrekkdrvftdktsatvicrknasisvraqdryysssw |
| | | | sewasvpcsggggsggggsggggsrnlpvatpdpgmfpclhhsqnl |
| | | | lravsnmlqkarqtlefypctseeidheditkdktstveaclpleltknes |
| | | | clnsretsfitngsclasrktsfmmalclssiyedlkmyqvefktmnakl |
| | | | lmdpkrqifldqnmlavidelmqalnfnsetvpqkssleepdfyktki |
| | | | klcillhafriravtidrvmsylnassggpALFKSSFPpgsggggsg |
| | | | gggsggggsggggsggggggggsQSVLTQPPSVSGAPG |
| | | | QRVTISCSGSRSNIGeeTVKWYQQLPGTAPKLLI |
| | | | YYNDQRPSGVPDRFSGSKSGTSASLAITGLQAE |
| | | | DEADYYCQSYDRYTHPALLFGTGTKVTVLgqpk |
| | | | aapsvtlfppsseelqankatlvclisdfypgavtvawkadsspvkagv |
| | | | etttpskqsnnkyaassylsltpeqwkshrsyscqvthegstvektvapt |
| | | | ecs** |
| 92 | WW0726 | Monomeric IL-12 | QVQLVESGGGVVQPGRSLRLSCAASGFTFSSY |
| | | polypeptide, anti- | GMHWVRQAPGKGLEWVAFIRYDGSNKYYAD |
| | | HSA sdAb, Fab | SVKGRFTISRDNSKNTLYLQMNSLRAEDTAVY |
| | | Blocker, 2 | YCKTHGSHDNWGQGTMVTVSSastkgpsvfplapss |
| | | cleavage sites | kstsggtaalgclvkdyfpepvtvswnsgaltsgvhtfpavlqssglysl |
| | | | ssvvtvpssslgtqtyicnvnhkpsntkvdkrvepksc** |
| 93 | WW0727 | Monomeric IL-12 | QVQLVESGGGVVQPGRSLRLSCAASGFTFSSY |
| | | polypeptide, anti- | GMHWVRQAPGKGLEWVAFIRYeGSNKYYAeS |
| | | HSA sdAb, Fab | VKGRFTISRDNSKNTLYLQMNSLRAEDTAVYY |
| | | Blocker, 2 | CKTHGSHDNWGQGTMVTVSSastkgpsvfplapsskst |
| | | cleavage sites | sggtaalgclvkdyfpepvtvswnsgaltsgvhtfpavlqssglyslssv |
| | | | vtvpssslgtqtyicnvnhkpsntkvdkrvepksc** |
| 94 | WW0728 | Monomeric IL-12 | QVQLVESGGGVVQPGRSLRLSCAASGFTFSSY |
| | | polypeptide, anti- | GMHWVRQAPGKGLEWVAFIRYeGSNKYYADS |
| | | HSA sdAb, Fab | VKGRFTISRDNSKNTLYLQMNSLRAEDTAVYY |
| | | Blocker, 2 | CKTHGSHDNWGQGTMVTVSSastkgpsvfplapsskst |
| | | cleavage sites | sggtaalgclvkdyfpepvtvswnsgaltsgvhtfpavlqssglyslssv |
| | | | vtvpssslgtqtyicnvnhkpsntkvdkrvepksc** |
| 95 | WW0749 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsrv |
| | | Blocker, 2 | ipvsgparclsqsrnllkttddmvktareklkhysctaedidheditrdqt |
| | | cleavage sites | stlktclplelhknesclatretssttrgsclppqktslmmtlclgsiyedlk |
| | | | myqtefqainaalqnhnhqqiildkgmlvaidelmqslnhngetlrqk |
| | | | ppvgeadpyrvkmklcillhafstrvvtinrvmgylssasggpGPA |
| | | | GLYAQpgsggggsggggsggggsggggsggggggggsQSV |
| | | | LTQPPSVSGAPGQRVTISCSGSRSNIGSNTVKW |
| | | | YQQLPGTAPKLLIYYNDQRPSGVPDRFSGSKSG |
| | | | TSASLAITGLQAEDEADYYCQSYDRYTHPALL |
| | | | FGTGTKVTVLggggsggggsggggsQVQLVESGGGV |
| | | | VQPGRSLRLSCAASGFTFSSYGMHWVRQAPGK |
| | | | GLEWVAFIRYeGSNKYYAeSVKGRFTISRDNSK |
| | | | NTLYLQMNSLRAEDTAVYYCKTHGSHDNWG |
| | | | QGTMVTVSS** |
| 96 | WW0750 | Heterodimeric IL- | EVQLVESGGGLVQPGNSLRLSCAASGFTFSKFG |
| | | 12 polypeptide, | MSWVRQAPGKGLEWVSSISGSGRDTLYAESV |
| | | anti-HSA sdAb, | KGRFTISRDNAKTTLYLQMNSLRPEDTAVYYC |
| | | scFv Blocker, 2 | TIGGSLSVSSQGTLVTVSSsggpGPAGLYAQpgsrn |
| | | cleavage sites | lpvatpdpgmfpclhhsqnllravsnmlqkarqtlefypctseeidhed |
| | | | itkdktstveaclpleltknesclnsretsfitngsclasrktsfmmalclss |
| | | | iyedlkmyqvefktmnakllmdpkrqifldqnmlavidelmqalnfn |
| | | | setvpqkssleepdfyktkiklcillhafriravtidrvmsylnassggpG |
| | | | PAGLYAQpgsggggsggggsggggsggggsggggsggggsQ |
| | | | SVLTQPPSVSGAPGQRVTISCSGSRSNIGSNTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLggggsggggsggggsQVQLVESGG |
| | | | GVVQPGRSLRLSCAASGFTFSSYGMHWVRQAP |
| | | | GKGLEWVAFIRYeGSNKYYAeSVKGRFTISRDN |
| | | | SKNTLYLQMNSLRAEDTAVYYCKTHGSHDNW |
| | | | GQGTMVTVSS** |
| 97 | WW0751 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpGPAGLYAQpgsrv |
| | | Blocker, 2 | ipvsgparclsqsrnllkttddmvktareklkhysctaedidheditrdqt |
| | | cleavage sites | stlktclplelhknesclatretssttrgsclppqktslmmtlclgsiyedlk |
| | | | myqtefqainaalqnhnhqqiildkgmlvaidelmqslnhngetlrqk |
| | | | ppvgeadpyrvkmklcillhafstrvvtinrvmgylssasggpGPA |
| | | | GLYAQpgsggggsggggsggggsggggsggggsggggsQSV |
| | | | LTQPPSVSGAPGQRVTISCSGSRSNIGdeTVKWY |
| | | | QQLPGTAPKLLIYYNDQRPSGVPDRFSGSKSGT |
| | | | SASLAITGLQAEDEADYYCQSYDRYTHPALLF |
| | | | GTGTKVTVLggggsggggsggggsQVQLVESGGGV |
| | | | VQPGRSLRLSCAASGFTFSSYGMHWVRQAPGK |
| | | | GLEWVAFIRYeGSNKYYAeSVKGRFTISRDNSK |
| | | | NTLYLQMNSLRAEDTAVYYCKTHGSHDNWG |
| | | | QGTMVTVSS** |
| 98 | WW0752 | Heterodimeric IL- | EVQLVESGGGLVQPGNSLRLSCAASGFTFSKFG |
| | | 12 polypeptide, | MSWVRQAPGKGLEWVSSISGSGRDTLYAESV |
| | | anti-HSA sdAb, | KGRFTISRDNAKTTLYLQMNSLRPEDTAVYYC |
| | | scFv Blocker, 2 | TIGGSLSVSSQGTLVTVSSsggpGPAGLYAQpgsrn |
| | | cleavage sites | lpvatpdpgmfpclhhsqnllravsnmlqkarqtlefypctseeidhed |
| | | | itkdktstveaclpleltknesclnsretsfitngsclasrktsfmmalclss |
| | | | iyedlkmyqvefktmnakllmdpkrqifldqnmlavidelmqalnfn |
| | | | setvpqkssleepdfyktkiklcillhafriravtidrvmsylnassggpG |
| | | | PAGLYAQpgsggggsggggsggggsggggsggggsggggsQ |
| | | | SVLTQPPSVSGAPGQRVTISCSGSRSNIGdeTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLggggsggggsggggsQVQLVESGG |
| | | | GVVQPGRSLRLSCAASGFTFSSYGMHWVRQAP |
| | | | GKGLEWVAFIRYeGSNKYYAeSVKGRFTISRDN |
| | | | SKNTLYLQMNSLRAEDTAVYYCKTHGSHDNW |
| | | | GQGTMVTVSS** |
| 99 | WW0753 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsrv |
| | | Blocker, 2 | ipvsgparclsqsrnllkttddmvktareklkhysctaedidheditrdqt |
| | | cleavage sites | stlktclplelhknesclatretssttrgsclppqktslmmtlclgsiyedlk |
| | | | myqtefqainaalqnhnhqgiildkgmlvaidelmqslnhngetlrgk |
| | | | ppvgeadpyrvkmklcillhafstrvvtinrvmgylssasggpGPA |
| | | | GLYAQpgsggggsggggsggggsggggsggggsggggsQSV |
| | | | LTQPPSVSGAPGQRVTISCSGSRSNIGSNTVKW |
| | | | YQQLPGTAPKLLIYYNDQRPSGVPDRFSGSKSG |
| | | | TSASLAITGLQAEDEADYYCQSYDRYTHPALL |
| | | | FGTGTKVTVLgqpkaapsvtlfppsseelqankatlvclisdfyp |
| | | | gavtvawkadsspvkagvetttpskqsnnkyaassylsltpeqwkshr |
| | | | syscqvthegstvektvaptecs** |
| 100 | WW0754 | Heterodimeric IL- | EVQLVESGGGLVQPGNSLRLSCAASGFTFSKFG |
| | | 12 polypeptide, | MSWVRQAPGKGLEWVSSISGSGRDTLYAESV |
| | | anti-HSA sdAb, | KGRFTISRDNAKTTLYLQMNSLRPEDTAVYYC |
| | | Fab Blocker, 2 | TIGGSLSVSSQGTLVTVSSsggpGPAGLYAQpgsrn |
| | | cleavage sites | lpvatpdpgmfpclhhsqnllravsnmlqkarqtlefypctseeidhed |
| | | | itkdktstveaclpleltknesclnsretsfitngsclasrktsfmmalclss |
| | | | iyedlkmyqvefktmnakllmdpkrqifldqnmlavidelmqalnfn |
| | | | setvpqkssleepdfyktkiklcillhafriravtidrvmsylnassggpG |
| | | | PAGLYAQpgsggggsggggsggggsggggsggggggggsQ |
| | | | SVLTQPPSVSGAPGQRVTISCSGSRSNIGSNTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLgqpkaapsvtlfppsseelqankatlvclisd |
| | | | fypgavtvawkadsspvkagvetttpskqsnnkyaassylsltpeqw |
| | | | kshrsyscqvthegstvektvaptecs** |
| 101 | WW0755 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpGPAGLYAQpgsrv |
| | | Blocker, 2 | ipvsgparclsqsrnllkttddmvktareklkhysctaedidheditrdqt |
| | | cleavage sites | stlktclplelhknesclatretssttrgsclppqktslmmtlclgsiyedlk |
| | | | myqtefqainaalqnhnhqqiildkgmlvaidelmqslnhngetlrqk |
| | | | ppvgeadpyrvkmklcillhafstrvvtinrvmgylssasggpGPA |
| | | | GLYAQpgsggggsggggsggggsggggggggsggggsQSV |
| | | | LTQPPSVSGAPGQRVTISCSGSRSNIGdeTVKWY |
| | | | QQLPGTAPKLLIYYNDQRPSGVPDRFSGSKSGT |
| | | | SASLAITGLQAEDEADYYCQSYDRYTHPALLF |
| | | | GTGTKVTVLgqpkaapsvtlfppsseelqankatlvclisdfypg |
| | | | avtvawkadsspvkagvetttpskqsnnkyaassylsltpeqwkshrs |
| | | | yscqvthegstvektvaptecs** |
| 102 | WW0756 | Heterodimeric IL- | EVQLVESGGGLVQPGNSLRLSCAASGFTFSKFG |
| | | 12 polypeptide, | MSWVRQAPGKGLEWVSSISGSGRDTLYAESV |
| | | anti-HSA sdAb, | KGRFTISRDNAKTTLYLQMNSLRPEDTAVYYC |
| | | Fab Blocker, 2 | TIGGSLSVSSQGTLVTVSSsggpGPAGLYAQpgsrn |
| | | cleavage sites | lpvatpdpgmfpclhhsqnllravsnmlqkarqtlefypctseeidhed |
| | | | itkdktstveaclpleltknesclnsretsfitngsclasrktsfmmalclss |
| | | | iyedlkmyqvefktmnakllmdpkrqifldqnmlavidelmqalnfn |
| | | | setvpqkssleepdfyktkiklcillhafriravtidrvmsylnassggpG |
| | | | PAGLYAQpgsggggsggggsggggsggggsggggggggsQ |
| | | | SVLTQPPSVSGAPGQRVTISCSGSRSNIGdeTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLgqpkaapsvtlfppsseelqankatlvclisd |
| | | | fypgavtvawkadsspvkagvetttpskqsnnkyaassylsltpeqw |
| | | | kshrsyscqvthegstvektvaptecs** |
| 103 | WW0757 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsrvi |
| | | Blocker, 2 | pvsgparclsqsrnllkttddmvktareklkhysctaedidheditrdqts |
| | | cleavage sites | tlktclplelhknesclatretssttrgsclppqktslmmtlclgsiyedlk |
| | | | myqtefqainaalqnhnhqqiildkgmlvaidelmqslnhngetlrqk |
| | | | ppvgeadpyrvkmklcillhafstrvvtinrvmgylssasggpALF |
| | | | KSSFPpgsggggggggsggggsggggsggggggggsQSVL |
| | | | TQPPSVSGAPGQRVTISCSGSRSNIGSNTVKWY |
| | | | QQLPGTAPKLLIYYNDQRPSGVPDRFSGSKSGT |
| | | | SASLAITGLQAEDEADYYCQSYDRYTHPALLF |
| | | | GTGTKVTVLggggsggggsggggsQVQLVESGGGV |
| | | | VQPGRSLRLSCAASGFTFSSYGMHWVRQAPGK |
| | | | GLEWVAFIRYeGSNKYYAeSVKGRFTISRDNSK |
| | | | NTLYLQMNSLRAEDTAVYYCKTHGSHDNWG |
| | | | QGTMVTVSS** |
| 104 | WW0758 | Heterodimeric IL- | EVQLVESGGGLVQPGNSLRLSCAASGFTFSKFG |
| | | 12 polypeptide, | MSWVRQAPGKGLEWVSSISGSGRDTLYAESV |
| | | anti-HSA sdAb, | KGRFTISRDNAKTTLYLQMNSLRPEDTAVYYC |
| | | scFv Blocker, 2 | TIGGSLSVSSQGTLVTVSSsggpALFKSSFPpgsrnl |
| | | cleavage sites | pvatpdpgmfpclhhsqnllravsnmlqkarqtlefypctseeidhedi |
| | | | tkdktstveaclpleltknescInsretsfitngsclasrktsfmmalclssi |
| | | | yedlkmyqvefktmnakllmdpkrqifldqnmlavidelmqalnfn |
| | | | setvpqkssleepdfyktkiklcillhafriravtidrvmsylnassggpA |
| | | | LFKSSFPpgsggggsggggsggggsggggsggggsggggsQS |
| | | | VLTQPPSVSGAPGQRVTISCSGSRSNIGSNTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLggggsggggsggggsQVQLVESGG |
| | | | GVVQPGRSLRLSCAASGFTFSSYGMHWVRQAP |
| | | | GKGLEWVAFIRYeGSNKYYAeSVKGRFTISRDN |
| | | | SKNTLYLQMNSLRAEDTAVYYCKTHGSHDNW |
| | | | GQGTMVTVSS** |
| 105 | WW0759 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, scFv | TIGGSLSVSSQGTLVTVSSsggpALFKSSFPpgsrvi |
| | | Blocker, 2 | pvsgparclsqsrnllkttddmvktareklkhysctaedidheditrdqts |
| | | cleavage sites | tlktclplelhknesclatretssttrgsclppqktslmmtlclgsiyedlk |
| | | | myqtefqainaalqnhnhqqiildkgmlvaidelmqslnhngetlrqk |
| | | | ppvgeadpyrvkmklcillhafstrvvtinrvmgylssasggpALF |
| | | | KSSFPpgsggggggggsggggsggggsggggsggggsQSVL |
| | | | TQPPSVSGAPGQRVTISCSGSRSNIGdeTVKWY |
| | | | QQLPGTAPKLLIYYNDQRPSGVPDRFSGSKSGT |
| | | | SASLAITGLQAEDEADYYCQSYDRYTHPALLF |
| | | | GTGTKVTVLggggggggsggggsQVQLVESGGGV |
| | | | VQPGRSLRLSCAASGFTFSSYGMHWVRQAPGK |
| | | | GLEWVAFIRYeGSNKYYAeSVKGRFTISRDNSK |
| | | | NTLYLQMNSLRAEDTAVYYCKTHGSHDNWG |
| | | | QGTMVTVSS** |
| 106 | WW0760 | Heterodimeric IL- | EVQLVESGGGLVQPGNSLRLSCAASGFTFSKFG |
| | | 12 polypeptide, | MSWVRQAPGKGLEWVSSISGSGRDTLYAESV |
| | | anti-HSA sdAb, | KGRFTISRDNAKTTLYLQMNSLRPEDTAVYYC |
| | | scFv Blocker, 2 | TIGGSLSVSSQGTLVTVSSsggpALFKSSFPpgsrnl |
| | | cleavage sites | pvatpdpgmfpclhhsqnllravsnmlqkarqtlefypctseeidhedi |
| | | | tkdktstveaclpleltknesclnsretsfitngsclasrktsfmmalclssi |
| | | | yedlkmyqvefktmnakllmdpkrqifldqnmlavidelmqalnfn |
| | | | setvpqkssleepdfyktkiklcillhafriravtidrvmsylnassggpA |
| | | | LFKSSFPpgsggggsggggsggggsggggggggsggggsQS |
| | | | VLTQPPSVSGAPGQRVTISCSGSRSNIGdeTVKW |
| | | | YQQLPGTAPKLLIYYNDQRPSGVPDRFSGSKSG |
| | | | TSASLAITGLQAEDEADYYCQSYDRYTHPALL |
| | | | FGTGTKVTVLggggsggggggggsQVQLVESGGGV |
| | | | VQPGRSLRLSCAASGFTFSSYGMHWVRQAPGK |
| | | | GLEWVAFIRYeGSNKYYAeSVKGRFTISRDNSK |
| | | | NTLYLQMNSLRAEDTAVYYCKTHGSHDNWG |
| | | | QGTMVTVSS** |
| 107 | WW0761 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsrvi |
| | | Blocker, 2 | pvsgparclsqsrnllkttddmvktareklkhysctaedidheditrdqts |
| | | cleavage sites | tlktclplelhknesclatretssttrgsclppqktslmmtlclgsiyedlk |
| | | | myqtefqainaalqnhnhqqiildkgmlvaidelmqslnhngetlrqk |
| | | | ppvgeadpyrvkmklcillhafstrvvtinrvmgylssasggpALF |
| | | | KSSFPpgsggggggggsggggsggggsggggsggggsQSVL |
| | | | TQPPSVSGAPGQRVTISCSGSRSNIGSNTVKWY |
| | | | QQLPGTAPKLLIYYNDQRPSGVPDRFSGSKSGT |
| | | | SASLAITGLQAEDEADYYCQSYDRYTHPALLF |
| | | | GTGTKVTVLgqpkaapsvtlfppsseelqankatlvclisdfypg |
| | | | avtvawkadsspvkagvetttpskqsnnkyaassylsltpeqwkshrs |
| | | | yscqvthegstvektvaptecs** |
| 108 | WW0762 | Heterodimeric IL- | EVQLVESGGGLVQPGNSLRLSCAASGFTFSKFG |
| | | 12 polypeptide, | MSWVRQAPGKGLEWVSSISGSGRDTLYAESV |
| | | anti-HSA sdAb, | KGRFTISRDNAKTTLYLQMNSLRPEDTAVYYC |
| | | Fab Blocker, 2 | TIGGSLSVSSQGTLVTVSSsggpALFKSSFPpgsrnl |
| | | cleavage sites | pvatpdpgmfpclhhsqnllravsnmlqkarqtlefypctseeidhedi |
| | | | tkdktstveaclpleltknescInsretsfitngsclasrktsfmmalclssi |
| | | | yedlkmyqvefktmnakllmdpkrqifldqnmlavidelmqalnfn |
| | | | setvpqkssleepdfyktkiklcillhafriravtidrvmsylnassggpA |
| | | | LFKSSFPpgsggggsggggsggggsggggsggggsggggsQS |
| | | | VLTQPPSVSGAPGQRVTISCSGSRSNIGSNTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLgqpkaapsvtlfppsseelqankatlvclisd |
| | | | fypgavtvawkadsspvkagvetttpskqsnnkyaassylsltpeqw |
| | | | kshrsyscqvthegstvektvaptecs** |
| 109 | WW0763 | Heterodimeric IL- | EVQLVESGGGLVQPGNSLRLSCAASGFTFSKFG |
| | | 12 (chimeric) | MSWVRQAPGKGLEWVSSISGSGRDTLYAESV |
| | | polypeptide, anti- | KGRFTISRDNAKTTLYLQMNSLRPEDTAVYYC |
| | | HSA sdAb, Fab | TIGGSLSVSSQGTLVTVSSsggpALFKSSFPpgsrvi |
| | | Blocker, 2 | pvsgparclsqsrnllkttddmvktareklkhysctaedidheditrdqts |
| | | cleavage sites | tlktclplelhknesclatretssttrgsclppqktslmmtlclgsiyedlk |
| | | | myqtefqainaalqnhnhqqiildkgmlvaidelmqslnhngetlrqk |
| | | | ppvgeadpyrvkmklcillhafstrvvtinrvmgylssasggpALF |
| | | | KSSFPpgsggggggggggggsggggggggggggsQSVL |
| | | | TQPPSVSGAPGQRVTISCSGSRSNIGdeTVKWY |
| | | | QQLPGTAPKLLIYYNDQRPSGVPDRFSGSKSGT |
| | | | SASLAITGLQAEDEADYYCQSYDRYTHPALLF |
| | | | GTGTKVTVLgqpkaapsvtlfppsseelqankatlvclisdfypg |
| | | | avtvawkadsspvkagvetttpskqsnnkyaassylsltpeqwkshrs |
| | | | yscqvthegstvektvaptecs** |
| 110 | WW0764 | Heterodimeric IL- | EVQLVESGGGLVQPGNSLRLSCAASGFTFSKFG |
| | | 12 polypeptide, | MSWVRQAPGKGLEWVSSISGSGRDTLYAESV |
| | | anti-HSA sdAb, | KGRFTISRDNAKTTLYLQMNSLRPEDTAVYYC |
| | | Fab Blocker, 2 | TIGGSLSVSSQGTLVTVSSsggpALFKSSFPpgsrnl |
| | | cleavage sites | pvatpdpgmfpclhhsqnllravsnmlqkarqtlefypctseeidhedi |
| | | | tkdktstveaclpleltknescInsretsfitngsclasrktsfmmalclssi |
| | | | yedlkmyqvefktmnakllmdpkrqifldqnmlavidelmqalnfn |
| | | | setvpqkssleepdfyktkiklcillhafriravtidrvmsylnassggpA |
| | | | LFKSSFPpgsggggggggsggggggggggggsggggsQS |
| | | | VLTQPPSVSGAPGQRVTISCSGSRSNIGdeTVKW |
| | | | YQQLPGTAPKLLIYYNDQRPSGVPDRFSGSKSG |
| | | | TSASLAITGLQAEDEADYYCQSYDRYTHPALL |
| | | | FGTGTKVTVLgqpkaapsvtlfppsseelqankatlvclisdfyp |
| | | | gavtvawkadsspvkagvetttpskqsnnkyaassylsltpeqwkshr |
| | | | syscqvthegstvektvaptecs** |
| 111 | WW0765 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, scFv | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsEVQLVE |
| | | | SGGGLVQPGNSLRLSCAASGFTFSKFGMSWVR |
| | | | QAPGKGLEWVSSISGSGRDTLYAESVKGRFTIS |
| | | | RDNAKTTLYLQMNSLRPEDTAVYYCTIGGSLS |
| | | | VSSQGTLVTVSSggggsggggsggggsggggsggggsggg |
| | | | gsQSVLTQPPSVSGAPGQRVTISCSGSRSNIGSN |
| | | | TVKWYQQLPGTAPKLLIYYNDQRPSGVPDRFS |
| | | | GSKSGTSASLAITGLQAEDEADYYCQSYDRYT |
| | | | HPALLFGTGTKVTVLggggsggggsggggsQVQLVE |
| | | | SGGGVVQPGRSLRLSCAASGFTFSSYGMHWVR |
| | | | QAPGKGLEWVAFIRYeGSNKYYAeSVKGRFTIS |
| | | | RDNSKNTLYLQMNSLRAEDTAVYYCKTHGSH |
| | | | DNWGQGTMVTVSS** |
| 112 | WW0766 | Monomeric IL-12 | iwelkkdvyvveldwypdapgemvvltcdtpeedgitwtldqssevl |
| | | polypeptide, anti- | gsgktltiqvkefgdagqytchkggevlshsllllhkkedgiwstdilkd |
| | | HSA sdAb, scFv | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsEVQ |
| | | | LVESGGGLVQPGNSLRLSCAASGFTFSKFGMS |
| | | | WVRQAPGKGLEWVSSISGSGRDTLYAESVKGR |
| | | | FTISRDNAKTTLYLQMNSLRPEDTAVYYCTIGG |
| | | | SLSVSSQGTLVTVSSggggsggggsggggsggggsggggs |
| | | | ggggsQSVLTQPPSVSGAPGQRVTISCSGSRSNIG |
| | | | SNTVKWYQQLPGTAPKLLIYYNDQRPSGVPDR |
| | | | FSGSKSGTSASLAITGLQAEDEADYYCQSYDR |
| | | | YTHPALLFGTGTKVTVLggggsggggsggggsQVQL |
| | | | VESGGGVVQPGRSLRLSCAASGFTFSSYGMHW |
| | | | VRQAPGKGLEWVAFIRYeGSNKYYAeSVKGRF |
| | | | TISRDNSKNTLYLQMNSLRAEDTAVYYCKTHG |
| | | | SHDNWGQGTMVTVSS** |
| 113 | WW0767 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, scFv | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsEVQLVE |
| | | | SGGGLVQPGNSLRLSCAASGFTFSKFGMSWVR |
| | | | QAPGKGLEWVSSISGSGRDTLYAESVKGRFTIS |
| | | | RDNAKTTLYLQMNSLRPEDTAVYYCTIGGSLS |
| | | | VSSQGTLVTVSSggggsggggsggggggggsggggsggg |
| | | | gsQSVLTQPPSVSGAPGQRVTISCSGSRSNIGdeT |
| | | | VKWYQQLPGTAPKLLIYYNDQRPSGVPDRFSG |
| | | | SKSGTSASLAITGLQAEDEADYYCQSYDRYTH |
| | | | PALLFGTGTKVTVLggggsggggsggggsQVQLVES |
| | | | GGGVVQPGRSLRLSCAASGFTFSSYGMHWVR |
| | | | QAPGKGLEWVAFIRYeGSNKYYAeSVKGRFTIS |
| | | | RDNSKNTLYLQMNSLRAEDTAVYYCKTHGSH |
| | | | DNWGQGTMVTVSS** |
| 114 | WW0768 | Monomeric IL-12 | iwelkkdvyvveldwypdapgemvvltcdtpeedgitwtldqssevl |
| | | polypeptide, anti- | gsgktltiqvkefgdagqytchkggevlshsllllhkkedgiwstdilkd |
| | | HSA sdAb, scFv | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsEVQ |
| | | | LVESGGGLVQPGNSLRLSCAASGFTFSKFGMS |
| | | | WVRQAPGKGLEWVSSISGSGRDTLYAESVKGR |
| | | | FTISRDNAKTTLYLQMNSLRPEDTAVYYCTIGG |
| | | | SLSVSSQGTLVTVSSggggsggggsggggsggggsgggg |
| | | | ggggsQSVLTQPPSVSGAPGQRVTISCSGSRSNIG |
| | | | deTVKWYQQLPGTAPKLLIYYNDQRPSGVPDR |
| | | | FSGSKSGTSASLAITGLQAEDEADYYCQSYDR |
| | | | YTHPALLFGTGTKVTVLggggsggggsggggsQVQL |
| | | | VESGGGVVQPGRSLRLSCAASGFTFSSYGMHW |
| | | | VRQAPGKGLEWVAFIRYeGSNKYYAeSVKGRF |
| | | | TISRDNSKNTLYLQMNSLRAEDTAVYYCKTHG |
| | | | SHDNWGQGTMVTVSS** |
| 115 | WW0769 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, Fab | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggsggggggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsEVQLVE |
| | | | SGGGLVQPGNSLRLSCAASGFTFSKFGMSWVR |
| | | | QAPGKGLEWVSSISGSGRDTLYAESVKGRFTIS |
| | | | RDNAKTTLYLQMNSLRPEDTAVYYCTIGGSLS |
| | | | VSSQGTLVTVSSggggsggggsggggggggsggggsggg |
| | | | gsQSVLTQPPSVSGAPGQRVTISCSGSRSNIGSN |
| | | | TVKWYQQLPGTAPKLLIYYNDQRPSGVPDRFS |
| | | | GSKSGTSASLAITGLQAEDEADYYCQSYDRYT |
| | | | HPALLFGTGTKVTVLgqpkaapsvtlfppsseelqankatlv |
| | | | clisdfypgavtvawkadsspvkagvetttpskqsnnkyaassylsltp |
| | | | eqwkshrsyscqvthegstvektvaptecs** |
| 116 | WW0770 | Monomeric IL-12 | iwelkkdvyvveldwypdapgemvvltcdtpeedgitwtldqssevl |
| | | polypeptide, anti- | gsgktltiqvkefgdagqytchkggevlshsllllhkkedgiwstdilkd |
| | | HSA sdAb, Fab | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsEVQ |
| | | | LVESGGGLVQPGNSLRLSCAASGFTFSKFGMS |
| | | | WVRQAPGKGLEWVSSISGSGRDTLYAESVKGR |
| | | | FTISRDNAKTTLYLQMNSLRPEDTAVYYCTIGG |
| | | | SLSVSSQGTLVTVSSggggsggggsggggsggggggggs |
| | | | ggggsQSVLTQPPSVSGAPGQRVTISCSGSRSNIG |
| | | | SNTVKWYQQLPGTAPKLLIYYNDQRPSGVPDR |
| | | | FSGSKSGTSASLAITGLQAEDEADYYCQSYDR |
| | | | YTHPALLFGTGTKVTVLgqpkaapsvtlfppsseelqank |
| | | | atlvclisdfypgavtvawkadsspvkagvetttpskqsnnkyaassyl |
| | | | sltpegwkshrsyscqvthegstvektvaptecs** |
| 117 | WW0771 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, Fab | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggsggggggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpGPAGLYAQpgsEVQLVE |
| | | | SGGGLVQPGNSLRLSCAASGFTFSKFGMSWVR |
| | | | QAPGKGLEWVSSISGSGRDTLYAESVKGRFTIS |
| | | | RDNAKTTLYLQMNSLRPEDTAVYYCTIGGSLS |
| | | | VSSQGTLVTVSSggggsggggsggggggggsggggggg |
| | | | gsQSVLTQPPSVSGAPGQRVTISCSGSRSNIGdeT |
| | | | VKWYQQLPGTAPKLLIYYNDQRPSGVPDRFSG |
| | | | SKSGTSASLAITGLQAEDEADYYCQSYDRYTH |
| | | | PALLFGTGTKVTVLgqpkaapsvtlfppsseelqankatlvcl |
| | | | isdfypgavtvawkadsspvkagvetttpskqsnnkyaassylsltpeq |
| | | | wkshrsyscqvthegstvektvaptecs** |
| 118 | WW0772 | Monomeric IL-12 | iwelkkdvyvveldwypdapgemvvltcdtpeedgitwtldqssevl |
| | | polypeptide, anti- | gsgktltiqvkefgdagqytchkggevlshsllllhkkedgiwstdilkd |
| | | HSA sdAb, Fab | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpGPAGLYAQpgsEVQ |
| | | | LVESGGGLVQPGNSLRLSCAASGFTFSKFGMS |
| | | | WVRQAPGKGLEWVSSISGSGRDTLYAESVKGR |
| | | | FTISRDNAKTTLYLQMNSLRPEDTAVYYCTIGG |
| | | | SLSVSSQGTLVTVSSggggsggggsggggsggggsggggs |
| | | | ggggsQSVLTQPPSVSGAPGQRVTISCSGSRSNIG |
| | | | deTVKWYQQLPGTAPKLLIYYNDQRPSGVPDR |
| | | | FSGSKSGTSASLAITGLQAEDEADYYCQSYDR |
| | | | YTHPALLFGTGTKVTVLgqpkaapsvtlfppsseelqank |
| | | | atlvclisdfypgavtvawkadsspvkagvetttpskqsnnkyaassyl |
| | | | sltpeqwkshrsyscgvthegstvektvaptecs** |
| 119 | WW0773 | Heterodimeric IL- | rvipvsgparclsqsrnllkttddmvktareklkhysctaedidheditrd |
| | | 12 (chimeric) | qtstlktclplelhknesclatretssttrgsclppqktslmmtlclgsiyed |
| | | polypeptide, anti- | lkmyqtefqainaalqnhnhqqiildkgmlvaidelmqslnhngetlr |
| | | HSA sdAb, scFv | qkppvgeadpyrvkmklcillhafstrvvtinrvmgylssasggpGP |
| | | Blocker, 1 | AGLYAQpgsEVQLVESGGGLVQPGNSLRLSCA |
| | | cleavage site | ASGFTFSKFGMSWVRQAPGKGLEWVSSISGSG |
| | | | RDTLYAESVKGRFTISRDNAKTTLYLQMNSLR |
| | | | PEDTAVYYCTIGGSLSVSSQGTLVTVSSggggsgg |
| | | | ggsggggggggsggggggggsQSVLTQPPSVSGAPGQ |
| | | | RVTISCSGSRSNIGSNTVKWYQQLPGTAPKLLI |
| | | | YYNDQRPSGVPDRFSGSKSGTSASLAITGLQAE |
| | | | DEADYYCQSYDRYTHPALLFGTGTKVTVLgggg |
| | | | sggggsggggsQVQLVESGGGVVQPGRSLRLSCAA |
| | | | SGFTFSSYGMHWVRQAPGKGLEWVAFIRYeGS |
| | | | NKYYAeSVKGRFTISRDNSKNTLYLQMNSLRA |
| | | | EDTAVYYCKTHGSHDNWGQGTMVTVSS** |
| 120 | WW0774 | Heterodimeric IL- | rnlpvatpdpgmfpclhhsqnllravsnmlqkarqtlefypctseeidh |
| | | 12 polypeptide, | editkdktstveaclpleltknesclnsretsfitngsclasrktsfmmalcl |
| | | anti-HSA sdAb, | ssiyedlkmyqvefktmnakllmdpkrqifldqnmlavidelmqaln |
| | | scFv Blocker, 1 | fnsetvpqkssleepdfyktkiklcillhafriravtidrvmsylnassgg |
| | | cleavage site | pGPAGLYAQpgsEVQLVESGGGLVQPGNSLRLS |
| | | | CAASGFTFSKFGMSWVRQAPGKGLEWVSSISG |
| | | | SGRDTLYAESVKGRFTISRDNAKTTLYLQMNS |
| | | | LRPEDTAVYYCTIGGSLSVSSQGTLVTVSSgggg |
| | | | sggggsggggsggggsggggsggggsQSVLTQPPSVSGAP |
| | | | GQRVTISCSGSRSNIGSNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 121 | WW0775 | Heterodimeric IL- | rvipvsgparclsqsrnllkttddmvktareklkhysctaedidheditrd |
| | | 12 (chimeric) | qtstlktclplelhknesclatretssttrgsclppqktslmmtlclgsiyed |
| | | polypeptide, anti- | lkmyqtefqainaalqnhnhqqiildkgmlvaidelmqslnhngetlr |
| | | HSA sdAb, scFv | qkppvgeadpyrvkmklcillhafstrvvtinrvmgylssasggpGP |
| | | Blocker, 1 | AGLYAQpgsEVQLVESGGGLVQPGNSLRLSCA |
| | | cleavage site | ASGFTFSKFGMSWVRQAPGKGLEWVSSISGSG |
| | | | RDTLYAESVKGRFTISRDNAKTTLYLQMNSLR |
| | | | PEDTAVYYCTIGGSLSVSSQGTLVTVSSggggsgg |
| | | | ggsggggggggsggggsggggsQSVLTQPPSVSGAPGQ |
| | | | RVTISCSGSRSNIGdeTVKWYQQLPGTAPKLLIY |
| | | | YNDQRPSGVPDRFSGSKSGTSASLAITGLQAED |
| | | | EADYYCQSYDRYTHPALLFGTGTKVTVLggggs |
| | | | ggggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 122 | WW0776 | Heterodimeric IL- | rnlpvatpdpgmfpclhhsqnllravsnmlqkarqtlefypctseeidh |
| | | 12 polypeptide, | editkdktstveaclpleltknesclnsretsfitngsclasrktsfmmalcl |
| | | anti-HSA sdAb, | ssiyedlkmyqvefktmnakllmdpkrqifldqnmlavidelmqaln |
| | | scFv Blocker, 1 | fnsetvpqkssleepdfyktkiklcillhafriravtidrvmsylnassgg |
| | | cleavage site | pGPAGLYAQpgsEVQLVESGGGLVQPGNSLRLS |
| | | | CAASGFTFSKFGMSWVRQAPGKGLEWVSSISG |
| | | | SGRDTLYAESVKGRFTISRDNAKTTLYLQMNS |
| | | | LRPEDTAVYYCTIGGSLSVSSQGTLVTVSSgggg |
| | | | sggggsggggsggggsggggsggggsQSVLTQPPSVSGAP |
| | | | GQRVTISCSGSRSNIGdeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | gggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 123 | WW0777 | Heterodimeric IL- | rvipvsgparclsqsrnllkttddmvktareklkhysctaedidheditrd |
| | | 12 (chimeric) | qtstlktclplelhknesclatretssttrgsclppqktslmmtlclgsiyed |
| | | polypeptide, anti- | lkmyqtefgainaalqnhnhqqiildkgmlvaidelmqslnhngetlr |
| | | HSA sdAb, Fab | qkppvgeadpyrvkmklcillhafstrvvtinrvmgylssasggpGP |
| | | Blocker, 1 | AGLYAQpgsEVQLVESGGGLVQPGNSLRLSCA |
| | | cleavage site | ASGFTFSKFGMSWVRQAPGKGLEWVSSISGSG |
| | | | RDTLYAESVKGRFTISRDNAKTTLYLQMNSLR |
| | | | PEDTAVYYCTIGGSLSVSSQGTLVTVSSggggsgg |
| | | | ggsggggggggggggsggggsQSVLTQPPSVSGAPGQ |
| | | | RVTISCSGSRSNIGSNTVKWYQQLPGTAPKLLI |
| | | | YYNDQRPSGVPDRFSGSKSGTSASLAITGLQAE |
| | | | DEADYYCQSYDRYTHPALLFGTGTKVTVLgqpk |
| | | | aapsvtlfppsseelqankatlvclisdfypgavtvawkadsspvkagv |
| | | | etttpskqsnnkyaassylsltpeqwkshrsyscqvthegstvektvapt |
| | | | ecs** |
| 124 | WW0778 | Heterodimeric IL- | rnlpvatpdpgmfpclhhsqnllravsnmlqkarqtlefypctseeidh |
| | | 12 polypeptide, | editkdktstveaclpleltknesclnsretsfitngsclasrktsfmmalcl |
| | | anti-HSA sdAb, | ssiyedlkmyqvefktmnakllmdpkrqifldqnmlavidelmqaln |
| | | Fab Blocker, 1 | fnsetvpqkssleepdfyktkiklcillhafriravtidrvmsylnassgg |
| | | cleavage site | pGPAGLYAQpgsEVQLVESGGGLVQPGNSLRLS |
| | | | CAASGFTFSKFGMSWVRQAPGKGLEWVSSISG |
| | | | SGRDTLYAESVKGRFTISRDNAKTTLYLQMNS |
| | | | LRPEDTAVYYCTIGGSLSVSSQGTLVTVSSgggg |
| | | | sggggsggggsggggsggggsggggsQSVLTQPPSVSGAP |
| | | | GQRVTISCSGSRSNIGSNTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 125 | WW0779 | Heterodimeric IL- | rvipvsgparclsqsrnllkttddmvktareklkhysctaedidheditrd |
| | | 12 (chimeric) | qtstlktclplelhknesclatretssttrgsclppqktslmmtlclgsiyed |
| | | polypeptide, anti- | lkmyqtefqainaalqnhnhqqiildkgmlvaidelmqslnhngetlr |
| | | HSA sdAb, Fab | qkppvgeadpyrvkmklcillhafstrvvtinrvmgylssasggpGP |
| | | Blocker, 1 | AGLYAQpgsEVQLVESGGGLVQPGNSLRLSCA |
| | | cleavage site | ASGFTFSKFGMSWVRQAPGKGLEWVSSISGSG |
| | | | RDTLYAESVKGRFTISRDNAKTTLYLQMNSLR |
| | | | PEDTAVYYCTIGGSLSVSSQGTLVTVSSggggsgg |
| | | | ggsggggggggsggggggggsQSVLTQPPSVSGAPGQ |
| | | | RVTISCSGSRSNIGdeTVKWYQQLPGTAPKLLIY |
| | | | YNDQRPSGVPDRFSGSKSGTSASLAITGLQAED |
| | | | EADYYCQSYDRYTHPALLFGTGTKVTVLgqpka |
| | | | apsvtlfppsseelqankatlvclisdfypgavtvawkadsspvkagve |
| | | | tttpskqsnnkyaassylsltpeqwkshrsyscqvthegstvektvapte |
| | | | cs** |
| 126 | WW0780 | Heterodimeric IL- | rnlpvatpdpgmfpclhhsqnllravsnmlqkarqtlefypctseeidh |
| | | 12 polypeptide, | editkdktstveaclpleltknescInsretsfitngsclasrktsfmmalcl |
| | | anti-HSA sdAb, | ssiyedlkmyqvefktmnakllmdpkrqifldqnmlavidelmqaln |
| | | Fab Blocker, 1 | fnsetvpqkssleepdfyktkiklcillhafriravtidrvmsylnassgg |
| | | cleavage site | pGPAGLYAQpgsEVQLVESGGGLVQPGNSLRLS |
| | | | CAASGFTFSKFGMSWVRQAPGKGLEWVSSISG |
| | | | SGRDTLYAESVKGRFTISRDNAKTTLYLQMNS |
| | | | LRPEDTAVYYCTIGGSLSVSSQGTLVTVSSgggg |
| | | | sggggsggggsggggsggggsggggsQSVLTQPPSVSGAP |
| | | | GQRVTISCSGSRSNIGdeTVKWYQQLPGTAPKL |
| | | | LIYYNDQRPSGVPDRFSGSKSGTSASLAITGLQ |
| | | | AEDEADYYCQSYDRYTHPALLFGTGTKVTVLg |
| | | | qpkaapsvtlfppsseelqankatlvclisdfypgavtvawkadsspvk |
| | | | agvetttpskqsnnkyaassylsltpeqwkshrsyscqvthegstvekt |
| | | | vaptecs** |
| 127 | WW0796 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, scFv | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsEVQLVES |
| | | | GGGLVQPGNSLRLSCAASGFTFSKFGMSWVRQ |
| | | | APGKGLEWVSSISGSGRDTLYAESVKGRFTISR |
| | | | DNAKTTLYLQMNSLRPEDTAVYYCTIGGSLSV |
| | | | SSQGTLVTVSSggggsggggggggsggggsggggsggggs |
| | | | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYeGSNKYYAeSVKGRFTISR |
| | | | DNSKNTLYLQMNSLRAEDTAVYYCKTHGSHD |
| | | | NWGQGTMVTVSS** |
| 128 | WW0797 | Monomeric IL-12 | iwelkkdvyvveldwypdapgemvvltcdtpeedgitwtldqssevl |
| | | polypeptide, anti- | gsgktltiqvkefgdagqytchkggevlshsllllhkkedgiwstdilkd |
| | | HSA sdAb, scFv | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpALFKSSFPpgsEVQL |
| | | | VESGGGLVQPGNSLRLSCAASGFTFSKFGMSW |
| | | | VRQAPGKGLEWVSSISGSGRDTLYAESVKGRF |
| | | | TISRDNAKTTLYLQMNSLRPEDTAVYYCTIGGS |
| | | | LSVSSQGTLVTVSSggggsggggsggggsggggsggggsg |
| | | | gggsQSVLTQPPSVSGAPGQRVTISCSGSRSNIGS |
| | | | NTVKWYQQLPGTAPKLLIYYNDQRPSGVPDRF |
| | | | SGSKSGTSASLAITGLQAEDEADYYCQSYDRY |
| | | | THPALLFGTGTKVTVLggggsggggggggsQVQLV |
| | | | ESGGGVVQPGRSLRLSCAASGFTFSSYGMHWV |
| | | | RQAPGKGLEWVAFIRYeGSNKYYAeSVKGRFTI |
| | | | SRDNSKNTLYLQMNSLRAEDTAVYYCKTHGS |
| | | | HDNWGQGTMVTVSS** |
| 129 | WW0798 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, scFv | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsEVQLVES |
| | | | GGGLVQPGNSLRLSCAASGFTFSKFGMSWVRQ |
| | | | APGKGLEWVSSISGSGRDTLYAESVKGRFTISR |
| | | | DNAKTTLYLQMNSLRPEDTAVYYCTIGGSLSV |
| | | | SSQGTLVTVSSggggsggggsggggsggggsggggsggggs |
| | | | QSVLTQPPSVSGAPGQRVTISCSGSRSNIGdeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYeGSNKYYAeSVKGRFTISR |
| | | | DNSKNTLYLQMNSLRAEDTAVYYCKTHGSHD |
| | | | NWGQGTMVTVSS** |
| 130 | WW0799 | Monomeric IL-12 | iwelkkdvyvveldwypdapgemvvltcdtpeedgitwtldqssevl |
| | | polypeptide, anti- | gsgktltiqvkefgdagqytchkggevlshsllllhkkedgiwstdilkd |
| | | HSA sdAb, scFv | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpALFKSSFPpgsEVQL |
| | | | VESGGGLVQPGNSLRLSCAASGFTFSKFGMSW |
| | | | VRQAPGKGLEWVSSISGSGRDTLYAESVKGRF |
| | | | TISRDNAKTTLYLQMNSLRPEDTAVYYCTIGGS |
| | | | LSVSSQGTLVTVSSggggsggggsggggsggggsggggsg |
| | | | gggsQSVLTQPPSVSGAPGQRVTISCSGSRSNIGd |
| | | | eTVKWYQQLPGTAPKLLIYYNDQRPSGVPDRF |
| | | | SGSKSGTSASLAITGLQAEDEADYYCQSYDRY |
| | | | THPALLFGTGTKVTVLggggsggggsggggsQVQLV |
| | | | ESGGGVVQPGRSLRLSCAASGFTFSSYGMHWV |
| | | | RQAPGKGLEWVAFIRYeGSNKYYAeSVKGRFTI |
| | | | SRDNSKNTLYLQMNSLRAEDTAVYYCKTHGS |
| | | | HDNWGQGTMVTVSS** |
| 131 | WW0800 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, Fab | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsEVQLVES |
| | | | GGGLVQPGNSLRLSCAASGFTFSKFGMSWVRQ |
| | | | APGKGLEWVSSISGSGRDTLYAESVKGRFTISR |
| | | | DNAKTTLYLQMNSLRPEDTAVYYCTIGGSLSV |
| | | | SSQGTLVTVSSggggggggsggggsggggsggggggggs |
| | | | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLgqpkaapsvtlfppsseelqankatlvcli |
| | | | sdfypgavtvawkadsspvkagvetttpskqsnnkyaassylsltpeq |
| | | | wkshrsyscqvthegstvektvaptecs** |
| 132 | WW0801 | Monomeric IL-12 | iwelkkdvyvveldwypdapgemvvltcdtpeedgitwtldqssevl |
| | | polypeptide, anti- | gsgktltiqvkefgdagqytchkggevlshsllllhkkedgiwstdilkd |
| | | HSA sdAb, Fab | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpALFKSSFPpgsEVQL |
| | | | VESGGGLVQPGNSLRLSCAASGFTFSKFGMSW |
| | | | VRQAPGKGLEWVSSISGSGRDTLYAESVKGRF |
| | | | TISRDNAKTTLYLQMNSLRPEDTAVYYCTIGGS |
| | | | LSVSSQGTLVTVSSggggsggggsggggsggggsggggsg |
| | | | gggsQSVLTQPPSVSGAPGQRVTISCSGSRSNIGS |
| | | | NTVKWYQQLPGTAPKLLIYYNDQRPSGVPDRF |
| | | | SGSKSGTSASLAITGLQAEDEADYYCQSYDRY |
| | | | THPALLFGTGTKVTVLgqpkaapsvtlfppsseelqankat |
| | | | lvclisdfypgavtvawkadsspvkagvetttpskqsnnkyaassylsl |
| | | | tpeqwkshrsyscqvthegstvektvaptecs** |
| 133 | WW0802 | Monomeric IL-12 | iwelkkdvyvveldwypdapgemvvltcdtpeedgitwtldqssevl |
| | | (chimeric) | gsgktltiqvkefgdagqytchkggevlshsllllhkkedgiwstdilkd |
| | | polypeptide, anti- | qkepknktflrceaknysgrftcwwlttistdltfsvkssrgssdpqgvtc |
| | | HSA sdAb, Fab | gaatlsaervrgdnkeyeysvecqedsacpaaeeslpievmvdavhkl |
| | | Blocker, 1 | kyenytssffirdiikpdppknlqlkplknsrqvevsweypdtwstphs |
| | | cleavage site | yfsltfcvqvqgkskrekkdrvftdktsatvicrknasisvraqdryysss |
| | | | wsewasvpcsggggsggggsggggsrvipvsgparclsqsrnllkttd |
| | | | dmvktareklkhysctaedidheditrdqtstlktclplelhknesclatre |
| | | | tssttrgsclppqktslmmtlclgsiyedlkmyqtefqainaalqnhnhq |
| | | | qiildkgmlvaidelmqslnhngetlrqkppvgeadpyrvkmklcill |
| | | | hafstrvvtinrvmgylssasggpALFKSSFPpgsEVQLVES |
| | | | GGGLVQPGNSLRLSCAASGFTFSKFGMSWVRQ |
| | | | APGKGLEWVSSISGSGRDTLYAESVKGRFTISR |
| | | | DNAKTTLYLQMNSLRPEDTAVYYCTIGGSLSV |
| | | | SSQGTLVTVSSggggsggggsggggggggsggggsggggs |
| | | | QSVLTQPPSVSGAPGQRVTISCSGSRSNIGdeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLgqpkaapsvtlfppsseelqankatlvcli |
| | | | sdfypgavtvawkadsspvkagvetttpskqsnnkyaassylsltpeq |
| | | | wkshrsyscqvthegstvektvaptecs** |
| 134 | WW0803 | Monomeric IL-12 | iwelkkdvyvveldwypdapgemvvltcdtpeedgitwtldqssevl |
| | | polypeptide, anti- | gsgktltiqvkefgdagqytchkggevlshsllllhkkedgiwstdilkd |
| | | HSA sdAb, Fab | qkepknktflrceaknysgrftcwwlttistdltfsvkssrgssdpqgvtc |
| | | Blocker, 1 | gaatlsaervrgdnkeyeysvecqedsacpaaeeslpievmvdavhkl |
| | | cleavage site | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcsggggsggggsggggsrnlpvatpdpgmfpclhhsq |
| | | | nllravsnmlqkarqtlefypctseeidheditkdktstveaclpleltkne |
| | | | sclnsretsfitngsclasrktsfmmalclssiyedlkmyqvefktmnak |
| | | | llmdpkrqifldqnmlavidelmqalnfnsetvpqkssleepdfyktki |
| | | | klcillhafriravtidrvmsylnassggpALFKSSFPpgsEVQL |
| | | | VESGGGLVQPGNSLRLSCAASGFTFSKFGMSW |
| | | | VRQAPGKGLEWVSSISGSGRDTLYAESVKGRF |
| | | | TISRDNAKTTLYLQMNSLRPEDTAVYYCTIGGS |
| | | | LSVSSQGTLVTVSSggggsggggsggggsggggsggggsg |
| | | | gggsQSVLTQPPSVSGAPGQRVTISCSGSRSNIGd |
| | | | eTVKWYQQLPGTAPKLLIYYNDQRPSGVPDRF |
| | | | SGSKSGTSASLAITGLQAEDEADYYCQSYDRY |
| | | | THPALLFGTGTKVTVLgqpkaapsvtlfppsseelqankat |
| | | | lvclisdfypgavtvawkadsspvkagvetttpskqsnnkyaassylsl |
| | | | tpeqwkshrsyscqvthegstvektvaptecs** |
| 135 | WW0804 | Heterodimeric IL- | rvipvsgparclsqsrnllkttddmvktareklkhysctaedidheditrd |
| | | 12 (chimeric) | qtstlktclplelhknesclatretssttrgsclppqktslmmtlclgsiyed |
| | | polypeptide, anti- | lkmyqtefqainaalqnhnhqqiildkgmlvaidelmqslnhngetlr |
| | | HSA sdAb, scFv | qkppvgeadpyrvkmklcillhafstrvvtinrvmgylssasggpAL |
| | | Blocker, 1 | FKSSFPpgsEVQLVESGGGLVQPGNSLRLSCAAS |
| | | cleavage site | GFTFSKFGMSWVRQAPGKGLEWVSSISGSGRD |
| | | | TLYAESVKGRFTISRDNAKTTLYLQMNSLRPE |
| | | | DTAVYYCTIGGSLSVSSQGTLVTVSSggggggggs |
| | | | ggggsggggggggsggggsQSVLTQPPSVSGAPGQRV |
| | | | TISCSGSRSNIGSNTVKWYQQLPGTAPKLLIYY |
| | | | NDQRPSGVPDRFSGSKSGTSASLAITGLQAEDE |
| | | | ADYYCQSYDRYTHPALLFGTGTKVTVLggggsg |
| | | | gggsggggsQVQLVESGGGVVQPGRSLRLSCAAS |
| | | | GFTFSSYGMHWVRQAPGKGLEWVAFIRYeGSN |
| | | | KYYAeSVKGRFTISRDNSKNTLYLQMNSLRAE |
| | | | DTAVYYCKTHGSHDNWGQGTMVTVSS** |
| 136 | WW0805 | Heterodimeric IL- | rnlpvatpdpgmfpclhhsqnllravsnmlqkarqtlefypctseeidh |
| | | 12 polypeptide, | editkdktstveaclpleltknesclnsretsfitngsclasrktsfmmalcl |
| | | anti-HSA sdAb, | ssiyedlkmyqvefktmnakllmdpkrqifldqnmlavidelmqaln |
| | | scFv Blocker, 1 | fnsetvpqkssleepdfyktkiklcillhafriravtidrvmsylnassgg |
| | | cleavage site | pALFKSSFPpgsEVQLVESGGGLVQPGNSLRLSC |
| | | | AASGFTFSKFGMSWVRQAPGKGLEWVSSISGS |
| | | | GRDTLYAESVKGRFTISRDNAKTTLYLQMNSL |
| | | | RPEDTAVYYCTIGGSLSVSSQGTLVTVSSggggsg |
| | | | gggsggggsggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 137 | WW0806 | Heterodimeric IL- | rvipvsgparclsqsrnllkttddmvktareklkhysctaedidheditrd |
| | | 12 (chimeric) | qtstlktclplelhknesclatretssttrgsclppqktslmmtlclgsiyed |
| | | polypeptide, anti- | lkmyqtefqainaalqnhnhqqiildkgmlvaidelmqslnhngetlr |
| | | HSA sdAb, scFv | qkppvgeadpyrvkmklcillhafstrvvtinrvmgylssasggpAL |
| | | Blocker, 1 | FKSSFPpgsEVQLVESGGGLVQPGNSLRLSCAAS |
| | | cleavage site | GFTFSKFGMSWVRQAPGKGLEWVSSISGSGRD |
| | | | TLYAESVKGRFTISRDNAKTTLYLQMNSLRPE |
| | | | DTAVYYCTIGGSLSVSSQGTLVTVSSggggsggggs |
| | | | ggggsggggggggggggsQSVLTQPPSVSGAPGQRV |
| | | | TISCSGSRSNIGdeTVKWYQQLPGTAPKLLIYYN |
| | | | DQRPSGVPDRFSGSKSGTSASLAITGLQAEDEA |
| | | | DYYCQSYDRYTHPALLFGTGTKVTVLggggsggg |
| | | | gsggggsQVQLVESGGGVVQPGRSLRLSCAASGF |
| | | | TFSSYGMHWVRQAPGKGLEWVAFIRYeGSNK |
| | | | YYAeSVKGRFTISRDNSKNTLYLQMNSLRAED |
| | | | TAVYYCKTHGSHDNWGQGTMVTVSS** |
| 138 | WW0807 | Heterodimeric IL- | rnlpvatpdpgmfpclhhsqnllravsnmlqkarqtlefypctseeidh |
| | | 12 polypeptide, | editkdktstveaclpleltknesclnsretsfitngsclasrktsfmmalcl |
| | | anti-HSA sdAb, | ssiyedlkmyqvefktmnakllmdpkrqifldqnmlavidelmqaln |
| | | scFv Blocker, 1 | fnsetvpqkssleepdfyktkiklcillhafriravtidrvmsylnassgg |
| | | cleavage site | pALFKSSFPpgsEVQLVESGGGLVQPGNSLRLSC |
| | | | AASGFTFSKFGMSWVRQAPGKGLEWVSSISGS |
| | | | GRDTLYAESVKGRFTISRDNAKTTLYLQMNSL |
| | | | RPEDTAVYYCTIGGSLSVSSQGTLVTVSSggggsg |
| | | | gggsggggsggggsggggggggsQSVLTQPPSVSGAPG |
| | | | QRVTISCSGSRSNIGdeTVKWYQQLPGTAPKLLI |
| | | | YYNDQRPSGVPDRFSGSKSGTSASLAITGLQAE |
| | | | DEADYYCQSYDRYTHPALLFGTGTKVTVLgggg |
| | | | sggggsggggsQVQLVESGGGVVQPGRSLRLSCAA |
| | | | SGFTFSSYGMHWVRQAPGKGLEWVAFIRYeGS |
| | | | NKYYAeSVKGRFTISRDNSKNTLYLQMNSLRA |
| | | | EDTAVYYCKTHGSHDNWGQGTMVTVSS** |
| 139 | WW0808 | Heterodimeric IL- | rvipvsgparclsqsrnllkttddmvktareklkhysctaedidheditrd |
| | | 12 (chimeric) | qtstlktclplelhknesclatretssttrgsclppqktslmmtlclgsiyed |
| | | polypeptide, anti- | lkmyqtefqainaalqnhnhqqiildkgmlvaidelmqslnhngetlr |
| | | HSA sdAb, Fab | qkppvgeadpyrvkmklcillhafstrvvtinrvmgylssasggpAL |
| | | Blocker, 1 | FKSSFPpgsEVQLVESGGGLVQPGNSLRLSCAAS |
| | | cleavage site | GFTFSKFGMSWVRQAPGKGLEWVSSISGSGRD |
| | | | TLYAESVKGRFTISRDNAKTTLYLQMNSLRPE |
| | | | DTAVYYCTIGGSLSVSSQGTLVTVSSggggsggggs |
| | | | ggggggggsggggsggggsQSVLTQPPSVSGAPGQRV |
| | | | TISCSGSRSNIGSNTVKWYQQLPGTAPKLLIYY |
| | | | NDQRPSGVPDRFSGSKSGTSASLAITGLQAEDE |
| | | | ADYYCQSYDRYTHPALLFGTGTKVTVLgqpkaa |
| | | | psvtlfppsseelqankatlvclisdfypgavtvawkadsspvkagvett |
| | | | tpskqsnnkyaassylsltpeqwkshrsyscqvthegstvektvaptec |
| | | | s** |
| 140 | WW0809 | Heterodimeric IL- | rnlpvatpdpgmfpclhhsqnllravsnmlqkarqtlefypctseeidh |
| | | 12 polypeptide, | editkdktstveaclpleltknesclnsretsfitngsclasrktsfmmalcl |
| | | anti-HSA sdAb, | ssiyedlkmyqvefktmnakllmdpkrqifldqnmlavidelmqaln |
| | | Fab Blocker, 1 | fnsetvpqkssleepdfyktkiklcillhafriravtidrvmsylnassgg |
| | | cleavage site | pALFKSSFPpgsEVQLVESGGGLVQPGNSLRLSC |
| | | | AASGFTFSKFGMSWVRQAPGKGLEWVSSISGS |
| | | | GRDTLYAESVKGRFTISRDNAKTTLYLQMNSL |
| | | | RPEDTAVYYCTIGGSLSVSSQGTLVTVSSggggsg |
| | | | gggsggggsggggsggggggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgq |
| | | | pkaapsvtlfppsseelqankatlvclisdfypgavtvawkadsspvka |
| | | | gvetttpskqsnnkyaassylsltpeqwkshrsyscqvthegstvektv |
| | | | aptecs** |
| 141 | WW0810 | Heterodimeric IL- | rvipvsgparclsqsrnllkttddmvktareklkhysctaedidheditrd |
| | | 12 (chimeric) | qtstlktclplelhknesclatretssttrgsclppqktslmmtlclgsiyed |
| | | polypeptide, anti- | lkmyqtefqainaalqnhnhqqiildkgmlvaidelmqslnhngetlr |
| | | HSA sdAb, Fab | qkppvgeadpyrvkmklcillhafstrvvtinrvmgylssasggpAL |
| | | Blocker, 1 | FKSSFPpgsEVQLVESGGGLVQPGNSLRLSCAAS |
| | | cleavage site | GFTFSKFGMSWVRQAPGKGLEWVSSISGSGRD |
| | | | TLYAESVKGRFTISRDNAKTTLYLQMNSLRPE |
| | | | DTAVYYCTIGGSLSVSSQGTLVTVSSggggsggggs |
| | | | ggggsggggsggggsggggsQSVLTQPPSVSGAPGQRV |
| | | | TISCSGSRSNIGdeTVKWYQQLPGTAPKLLIYYN |
| | | | DQRPSGVPDRFSGSKSGTSASLAITGLQAEDEA |
| | | | DYYCQSYDRYTHPALLFGTGTKVTVLgqpkaaps |
| | | | vtlfppsseelqankatlvclisdfypgavtvawkadsspvkagvetttp |
| | | | skqsnnkyaassylsltpeqwkshrsyscqvthegstvektvaptecs** |
| 142 | WW0811 | Heterodimeric IL- | rnlpvatpdpgmfpclhhsqnllravsnmlqkarqtlefypctseeidh |
| | | 12 polypeptide, | editkdktstveaclpleltknesclnsretsfitngsclasrktsfmmalcl |
| | | anti-HSA sdAb, | ssiyedlkmyqvefktmnakllmdpkrqifldqnmlavidelmqaln |
| | | Fab Blocker, 1 | fnsetvpqkssleepdfyktkiklcillhafriravtidrvmsylnassgg |
| | | cleavage site | pALFKSSFPpgsEVQLVESGGGLVQPGNSLRLSC |
| | | | AASGFTFSKFGMSWVRQAPGKGLEWVSSISGS |
| | | | GRDTLYAESVKGRFTISRDNAKTTLYLQMNSL |
| | | | RPEDTAVYYCTIGGSLSVSSQGTLVTVSSggggsg |
| | | | gggsggggggggsggggggggsQSVLTQPPSVSGAPG |
| | | | QRVTISCSGSRSNIGdeTVKWYQQLPGTAPKLLI |
| | | | YYNDQRPSGVPDRFSGSKSGTSASLAITGLQAE |
| | | | DEADYYCQSYDRYTHPALLFGTGTKVTVLgqpk |
| | | | aapsvtlfppsseelqankatlvclisdfypgavtvawkadsspvkagv |
| | | | etttpskqsnnkyaassylsltpeqwkshrsyscqvthegstvektvapt |
| | | | ecs** |
| 143 | WW0814 | Heterodimeric IL- | rnlpvatpdpgmfpclhhsqnllravsnmlqkarqtlefypctseeidh |
| | | 12 | editkdktstveaclpleltknesclnsretsfitngsclasrktsfmmalcl |
| | | | ssiyedlkmyqvefktmnakllmdpkrqifldqnmlavidelmqaln |
| | | | fnsetvpqkssleepdfyktkiklcillhafriravtidrvmsylnasHH |
| | | | HHHH** |
| 144 | | Blocker 1 | QVQLQESGGGLVQAGGSLRLSCAASGRTFSSV |
| | | | YDMGWFRQAPGKDREFVARITESARNTRYAD |
| | | | SVRGRFTISRDNAKNTVYLQMNNLELEDAAVY |
| | | | YCAADPQTVVVGTPDYWGQGTQVTVSSAAAY |
| | | | PYDVPDYGSHHHHHH** |
| 145 | | Blocker 2 | QSVLTQPPSVSGAPGQRVTISCtGSsSNIGSNTV |
| | | | KWYQQLPGTAPKLLIYgNDQRPSGVPDRFSGS |
| | | | KSGTSASLAITGLQAEDEADYYCQSYDRYTHP |
| | | | AyvFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 146 | | Blocker 3 | QSVLTQPPSVSGAPGQRVTISCtGSsSNIGSNTV |
| | | | KWYQQLPGTAPKLLIYYNDQRPSGVPDRFSGS |
| | | | KSGTSASLAITGLQAEDEADYYCQSYDRYTHP |
| | | | AyvFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 147 | | Blocker 4 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYaMHWVRQA |
| | | | PGKGLEWVAvIsYDGSNKYYADSVKGRFTISRD |
| | | | NSKNTLYLQMNSLRAEDTAVYYCarHGSHDN |
| | | | WGQGTMVTVSSHHHHHH** |
| 148 | | Blocker 5 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggggggsQVQLVESG |
| | | | GGVVQPGRSLRLSCAASGFTFSSYGMHWVRQ |
| | | | APGKGLEWVAFIRYeGSNKYYADSVKGRFTIS |
| | | | RDNSKNTLYLQMNSLRAEDTAVYYCKTHGSH |
| | | | DNWGQGTMVTVSSHHHHHH** |
| 149 | | Blocker 6 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYAeSVKGRFTIS |
| | | | RDNSKNTLYLQMNSLRAEDTAVYYCKTHGSH |
| | | | DNWGQGTMVTVSSHHHHHH** |
| 150 | | Blocker 7 | QSVLTQPPSVSGAPGQRVTISCSGSRSNIGSqTV |
| | | | KWYQQLPGTAPKLLIYYNDQRPSGVPDRFSGS |
| | | | KSGTSASLAITGLQAEDEADYYCQSYeRYTHPA |
| | | | LLFGTGTKVTVLggggsggggsggggsQVQLVESGG |
| | | | GVVQPGRSLRLSCAASGFTFSSYGMHWVRQAP |
| | | | GKGLEWVAFIRYDGSNKYYADSVKGRFTISRD |
| | | | NSKNTLYLQMNSLRAEDTAVYYCKTHGSHDN |
| | | | WGQGTMVTVSSHHHHHH** |
| 151 | | Blocker 8 | QSVLTQPPSVSGAPGQRVTISCSGSRSNIGSqTV |
| | | | KWYQQLPGTAPKLLIYYNDQRPSGVPDRFSGS |
| | | | KSGTSASLAITGLQAEDEADYYCQSYSRYTHPA |
| | | | LLFGTGTKVTVLggggggggsggggsQVQLVESGG |
| | | | GVVQPGRSLRLSCAASGFTFSSYGMHWVRQAP |
| | | | GKGLEWVAFIRYDGSNKYYADSVKGRFTISRD |
| | | | NSKNTLYLQMNSLRAEDTAVYYCKTHGSHDN |
| | | | WGQGTMVTVSSHHHHHH** |
| 152 | | Blocker 9 | QSVLTQPPSVSGAPGQRVTISCSGSeSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 153 | | Blocker 10 | QSVLTQPPSVSGAPGQRVTISCSGSsSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 154 | | Blocker 11 | QSVLTQPPSVSGAPGQRVTISCSGSRSNIGdNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 155 | | Blocker 12 | QSVLTQPPSVSGAPGQRVTISCSGSRSNIGeNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 156 | | Blocker 13 | QSVLTQPPSVSGAPGQRVTISCSGSRSNIGSdTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 157 | | Blocker 14 | QSVLTQPPSVSGAPGQRVTISCSGSRSNIGSeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 158 | | Blocker 15 | QSVLTQPPSVSGAPGQRVTISCSGSRSNIGSNdV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 159 | | Blocker 16 | QSVLTQPPSVSGAPGQRVTISCSGSRSNIGSNTV |
| | | | WYQQLPGTAPKLLIYYNDQRPSGVPDRFSGSKS |
| | | | ITSASLAITGLQAEDEADYYCQSYDRYTHPALLF |
| | | | TGTKVTVLggggsggggsggggsQVQLVESGGGVVQ |
| | | | GRSLRLSCAASGFTFSSYGMHWVRQAPGKGLE |
| | | | VVAFIRYDGSNKYYADSVKGRFTISRDNSKNTL |
| | | | LQMNSLRAEDTAVYYCKTHGSHDNWGQGTM |
| | | | TVSSHHHHHH** |
| 160 | | Blocker 17 | QSVLTQPPSVSGAPGQRVTISCSGSRSNIGSNTV |
| | | | eWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 161 | | Blocker 18 | QSVLTQPPSVSGAPGQRVTISCSGSRSNIGSNTV |
| | | | KWYQQLPGTAPKLLIYYNDQdPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 162 | | Blocker 19 | QSVLTQPPSVSGAPGQRVTISCSGSRSNIGSNTV |
| | | | KWYQQLPGTAPKLLIYYNDQePSGVPDRFSGS |
| | | | KSGTSASLAITGLQAEDEADYYCQSYDRYTHP |
| | | | ALLFGTGTKVTVLggggggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 163 | | Blocker 20 | QSVLTQPPSVSGAPGQRVTISCSGSRSNIGSNTV |
| | | | KWYQQLPGTAPKLLIYYNDQRPdGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 164 | | Blocker 21 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDeYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 165 | | Blocker 22 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTdP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 166 | | Blocker 23 | QSVLTQPPSVSGAPGQRVTISCSGSeSNIGSNTV |
| | | | KWYQQLPGTAPKLLIYYNDQePSGVPDRFSGS |
| | | | KSGTSASLAITGLQAEDEADYYCQSYDeYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 167 | | Blocker 24 | QSVLTQPPSVSGAPGQRVTISCSGSeSNIGSNdV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 168 | | Blocker 25 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggggggsggggsQVQLVESG |
| | | | GGVVQPGRSLRLSCAASGFTFeSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 169 | | Blocker 26 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggggggsggggsQVQLVESG |
| | | | GGVVQPGRSLRLSCAASGFTFSeYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 170 | | Blocker 27 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSdYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 171 | | Blocker 28 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggggggsQVQLVESG |
| | | | GGVVQPGRSLRLSCAASGFTFSSYGMHWVRQ |
| | | | APGKGLEWVAFIeYDGSNKYYADSVKGRFTIS |
| | | | RDNSKNTLYLQMNSLRAEDTAVYYCKTHGSH |
| | | | DNWGQGTMVTVSSHHHHHH** |
| 172 | | Blocker 29 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIdYDGSNKYYADSVKGRFTIS |
| | | | RDNSKNTLYLQMNSLRAEDTAVYYCKTHGSH |
| | | | DNWGQGTMVTVSSHHHHHH** |
| 173 | | Blocker 30 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNdYYADSVKGRFTIS |
| | | | RDNSKNTLYLQMNSLRAEDTAVYYCKTHGSH |
| | | | DNWGQGTMVTVSSHHHHHH** |
| 174 | | Blocker 31 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNeYYADSVKGRFTIS |
| | | | RDNSKNTLYLQMNSLRAEDTAVYYCKTHGSH |
| | | | DNWGQGTMVTVSSHHHHHH** |
| 175 | | Blocker 32 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVeGRFTIS |
| | | | RDNSKNTLYLQMNSLRAEDTAVYYCKTHGSH |
| | | | DNWGQGTMVTVSSHHHHHH** |
| 176 | | Blocker 33 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSe |
| | | | DNWGQGTMVTVSSHHHHHH** |
| 177 | | Blocker 34 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIeYDGSNKYYADSVeGRFTISR |
| | | | DNSKNTLYLQMNSLRAEDTAVYYCKTHGSHD |
| | | | NWGQGTMVTVSSHHHHHH** |
| 178 | | Blocker 35 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggggggsQVQLVESG |
| | | | GGVVQPGRSLRLSCAASGFTFSSYGMHWVRQ |
| | | | APGKGLEWVAFIeYDGSNKYYADSVeGRFTISR |
| | | | DNSKNTLYLQMNSLRAEDTAVYYCKTHGSeD |
| | | | NWGQGTMVTVSSHHHHHH** |
| 179 | | Blocker 36 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 180 | | Blocker 37 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYeGSNKYYAeSVKGRFTISR |
| | | | DNSKNTLYLQMNSLRAEDTAVYYCKTHGSHD |
| | | | NWGQGTMVTVSSHHHHHH** |
| 181 | | Blocker 38 | QSVLTQPPSVSGAPGQRVTISCSGSRSNIGSeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYeGSNKYYAeSVKGRFTISR |
| | | | DNSKNTLYLQMNSLRAEDTAVYYCKTHGSHD |
| | | | NWGQGTMVTVSSHHHHHH** |
| 182 | | Blocker 39 | QSVLTQPPSVSGAPGQRVTISCSGSRSNIGdNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggggggsggggsQVQLVESG |
| | | | GGVVQPGRSLRLSCAASGFTFSSYGMHWVRQ |
| | | | APGKGLEWVAFIRYeGSNKYYAeSVKGRFTISR |
| | | | DNSKNTLYLQMNSLRAEDTAVYYCKTHGSHD |
| | | | NWGQGTMVTVSSHHHHHH** |
| 183 | | Blocker 40 | QSVLTQPPSVSGAPGQRVTISCSGSRSNIGdeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 184 | | Blocker 41 | QSVLTQPPSVSGAPGQRVTISCSGSRSNIGdeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggggggsggggsQVQLVESG |
| | | | GGVVQPGRSLRLSCAASGFTFSSYGMHWVRQ |
| | | | APGKGLEWVAFIRYeGSNKYYAeSVKGRFTISR |
| | | | DNSKNTLYLQMNSLRAEDTAVYYCKTHGSHD |
| | | | NWGQGTMVTVSSHHHHHH** |
| 185 | | Blocker 42 | QSVLTQPPSVSGAPGQRVTISCSGSeSNIGSNdV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 186 | | Blocker 43 | QSVLTQPPSVSGAPGQRVTISCSGSRSNIGeNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYeGSNKYYAeSVKGRFTISR |
| | | | DNSKNTLYLQMNSLRAEDTAVYYCKTHGSHD |
| | | | NWGQGTMVTVSSHHHHHH** |
| 187 | | Blocker 44 | QSVLTQPPSVSGAPGQRVTISCSGSRSNIGeeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggggggsQVQLVESG |
| | | | GGVVQPGRSLRLSCAASGFTFSSYGMHWVRQ |
| | | | APGKGLEWVAFIRYDGSNKYYADSVKGRFTIS |
| | | | RDNSKNTLYLQMNSLRAEDTAVYYCKTHGSH |
| | | | DNWGQGTMVTVSSHHHHHH** |
| 188 | | Blocker 45 | QSVLTQPPSVSGAPGQRVTISCSGSRSNIGSeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLggggsggggsggggsQVQLVESG |
| | | | GGVVQPGRSLRLSCAASGFTFSSYGMHWVRQ |
| | | | APGKGLEWVAFIRYeGSNKYYADSVKGRFTIS |
| | | | RDNSKNTLYLQMNSLRAEDTAVYYCKTHGSH |
| | | | DNWGQGTMVTVSSHHHHHH** |
| 189 | | Blocker 46 | QVQLVESGGGVVQPGRSLRLSCAASGFTFSSY |
| | | | GMHWVRQAPGKGLEWVAFIRYDGSNKYYAD |
| | | | SVKGRFTISRDNSKNTLYLQMNSLRAEDTAVY |
| | | | YCKTHGSHDNWGQGTMVTVSSastkgpsvfplapss |
| | | | kstsggtaalgclvkdyfpepvtvswnsgaltsgvhtfpavlqssglysl |
| | | | ssvvtvpssslgtqtyicnvnhkpsntkvdkrvepksc** |
| 190 | | Blocker 47 | QVQLVESGGGVVQPGRSLRLSCAASGFTFSSY |
| | | | GMHWVRQAPGKGLEWVAFIRYeGSNKYYAeS |
| | | | VKGRFTISRDNSKNTLYLQMNSLRAEDTAVYY |
| | | | CKTHGSHDNWGQGTMVTVSSastkgpsvfplapsskst |
| | | | sggtaalgclvkdyfpepvtvswnsgaltsgvhtfpavlqssglyslssv |
| | | | vtvpssslgtqtyicnvnhkpsntkvdkrvepksc** |
| 191 | | Blocker 48 | QVQLVESGGGVVQPGRSLRLSCAASGFTFSSY |
| | | | GMHWVRQAPGKGLEWVAFIRYeGSNKYYADS |
| | | | VKGRFTISRDNSKNTLYLQMNSLRAEDTAVYY |
| | | | CKTHGSHDNWGQGTMVTVSSastkgpsvfplapsskst |
| | | | sggtaalgclvkdyfpepvtvswnsgaltsgvhtfpavlqssglyslssv |
| | | | vtvpssslgtqtyicnvnhkpsntkvdkrvepksc** |
| 192 | | Blocker 49 | QSVLTQPPSVSGAPGQRVTISCSGSRSNIGSNTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLgqpkaapsvtlfppsseelqankatlvcli |
| | | | sdfypgavtvawkadsspvkagvetttpskqsnnkyaassylsltpeq |
| | | | wkshrsyscqvthegstvektvaptecs** |
| 193 | | Blocker 50 | QSVLTQPPSVSGAPGQRVTISCSGSRSNIGdeTV |
| | | | KWYQQLPGTAPKLLIYYNDQRPSGVPDRFSGS |
| | | | KSGTSASLAITGLQAEDEADYYCQSYDRYTHP |
| | | | ALLFGTGTKVTVLgqpkaapsvtlfppsseelqankatlvcli |
| | | | sdfypgavtvawkadsspvkagvetttpskqsnnkyaassylsltpeq |
| | | | wkshrsyscqvthegstvektvaptecs** |
| 194 | | Blocker 51 | QVQLVESGGGVVQPGRSLRLSCAASGFTFSSY |
| | | | GMHWVRQAPGKGLEWVAFIRYeGSNKYYAeS |
| | | | VKGRFTISRDNSKNTLYLQMNSLRAEDTAVYY |
| | | | CKTHGSHDNWGQGTMVTVSSastkgpsvfplapsskst |
| | | | sggtaalgclvkdyfpepvtvswnsgaltsgvhtfpavlqssglyslssv |
| | | | vtvpssslgtqtyicnvnhkpsntkvdkrvepkscHHHHHH** |
| 195 | | MMP14_1 | GPAGLYAQ |
| 196 | | MMP9 | GPAGMKGL |
| 197 | | FAPa_1 | PGGPAGIG |
| 198 | | CTSL1_1 | ALFKSSFP |
| 199 | | CTSL1_2 | ALFFSSPP |
| 200 | | ADAM17_1 | LAQRLRSS |
| 201 | | ADAM17_2 | LAQKLKSS |
| 202 | | ALU30-1 | GALFKSSFPSGGGPAGLYAQGGSGKGGSGK |
| 203 | | ALU30-2 | RGSGGGPAGLYAQGSGGGPAGLYAQGGSGK |
| 204 | | ALU30-3 | KGGGPAGLYAQGPAGLYAQGPAGLYAQGSR |
| 205 | | ALU30-4 | RGGPAGLYAQGGPAGLYAQGGGPAGLYAQK |
| 206 | | ALU30-5 | KGGALFKSSFPGGPAGIGPLAQKLKSSGGS |
| 207 | | ALU30-6 | SGGPGGPAGIGALFKSSFPLAQKLKSSGGG |
| 208 | | ALU30-7 | RGPLAQKLKSSALFKSSFPGGPAGIGGGGK |
| 209 | | ALU30-8 | GGGALFKSSFPLAQKLKSSPGGPAGIGGGR |
| 210 | | ALU30-9 | RGPGGPAGIGPLAQKLKSSALFKSSFPGGG |
| 211 | | ALU30-10 | RGGPLAQKLKSSPGGPAGIGALFKSSFPGK |
| 212 | | ALU30-11 | RSGGPAGLYAQALFKSSFPLAQKLKSSGGG |
| 213 | | ALU30-12 | GGPLAQKLKSSALFKSSFPGPAGLYAQGGR |
| 214 | | ALU30-13 | GGALFKSSFPGPAGLYAQPLAQKLKSSGGK |
| 215 | | ALU30-14 | RGGALFKSSFPLAQKLKSSGPAGLYAQGGK |
| 216 | | ALU30-15 | RGGGPAGLYAQPLAQKLKSSALFKSSFPGG |
| 217 | | ALU30-16 | SGPLAQKLKSSGPAGLYAQALFKSSFPGSK |
| 218 | | ALU30-17 | KGGPGGPAGIGPLAQRLRSSALFKSSFPGR |
| 219 | | ALU30-18 | KSGPGGPAGIGALFFSSPPLAQKLKSSGGR |
| 220 | | ALU30-19 | SGGFPRSGGSFNPRTFGSKRKRRGSRGGGG |
| 221 | | MMP14 substrate | GPLGLKAQ |
| | | motif sequence | |
| 222 | | MMP14 substrate | LPLGLKAQ |
| | | motif sequence | |
| 223 | | MMP14 substrate | SPLGLKAQ |
| | | motif sequence | |
| 224 | | MMP14 substrate | QPLGLKAQ |
| | | motif sequence | |
| 225 | | MMP14 substrate | KPLGLKAQ |
| | | motif sequence | |
| 226 | | MMP14 substrate | FPLGLKAQ |
| | | motif sequence | |
| 227 | | MMP14 substrate | HPLGLKAQ |
| | | motif sequence | |
| 228 | | MMP14 substrate | PPLGLKAQ |
| | | motif sequence | |
| 229 | | MMP14 substrate | APLGLKAQ |
| | | motif sequence | |
| 230 | | MMP14 substrate | DPLGLKAQ |
| | | motif sequence | |
| 231 | | MMP14 substrate | GPHGLKAQ |
| | | motif sequence | |
| 232 | | MMP14 substrate | GPSGLKAQ |
| | | motif sequence | |
| 233 | | MMP14 substrate | GPQGLKAQ |
| | | motif sequence | |
| 234 | | MMP14 substrate | GPPGLKAQ |
| | | motif sequence | |
| 235 | | MMP14 substrate | GPEGLKAQ |
| | | motif sequence | |
| 236 | | MMP14 substrate | GPFGLKAQ |
| | | motif sequence | |
| 237 | | MMP14 substrate | GPRGLKAQ |
| | | motif sequence | |
| 238 | | MMP14 substrate | GPGGLKAQ |
| | | motif sequence | |
| 239 | | MMP14 substrate | GPAGLKAQ |
| | | motif sequence | |
| 240 | | MMP14 substrate | LPAGLKGA |
| | | motif sequence | |
| 241 | | MMP14 substrate | GPAGLYAQ |
| | | motif sequence | |
| 242 | | MMP14 substrate | GPANLVAQ |
| | | motif sequence | |
| 243 | | MMP14 substrate | GPAALVGA |
| | | motif sequence | |
| 244 | | MMP14 substrate | GPANLRAQ |
| | | motif sequence | |
| 245 | | MMP14 substrate | GPAGLRAQ |
| | | motif sequence | |
| 246 | | MMP14 substrate | GPAGLVAQ |
| | | motif sequence | |
| 247 | | MMP14 substrate | GPAGLRGA |
| | | motif sequence | |
| 248 | | MMP14 substrate | LPAGLVGA |
| | | motif sequence | |
| 249 | | MMP14 substrate | GPAGLKGA |
| | | motif sequence | |
| 250 | | MMP14 substrate | GPLALKAQ |
| | | motif sequence | |
| 251 | | MMP14 substrate | GPLNLKAQ |
| | | motif sequence | |
| 252 | | MMP14 substrate | GPLHLKAQ |
| | | motif sequence | |
| 253 | | MMP14 substrate | GPLYLKAQ |
| | | motif sequence | |
| 254 | | MMP14 substrate | GPLPLKAQ |
| | | motif sequence | |
| 255 | | MMP14 substrate | GPLELKAQ |
| | | motif sequence | |
| 256 | | MMP14 substrate | GPLRLKAQ |
| | | motif sequence | |
| 257 | | MMP14 substrate | GPLLLKAQ |
| | | motif sequence | |
| 258 | | MMP14 substrate | GPLSLKAQ |
| | | motif sequence | |
| 259 | | MMP14 substrate | GPLGLYAQ |
| | | motif sequence | |
| 260 | | MMP14 substrate | GPLGLFAQ |
| | | motif sequence | |
| 261 | | MMP14 substrate | GPLGLLAQ |
| | | motif sequence | |
| 262 | | MMP14 substrate | GPLGLHAQ |
| | | motif sequence | |
| 263 | | MMP14 substrate | GPLGLRAQ |
| | | motif sequence | |
| 264 | | MMP14 substrate | GPLGLAAQ |
| | | motif sequence | |
| 265 | | MMP14 substrate | GPLGLEAQ |
| | | motif sequence | |
| 266 | | MMP14 substrate | GPLGLGAQ |
| | | motif sequence | |
| 267 | | MMP14 substrate | GPLGLPAQ |
| | | motif sequence | |
| 268 | | MMP14 substrate | GPLGLQAQ |
| | | motif sequence | |
| 269 | | MMP14 substrate | GPLGLSAQ |
| | | motif sequence | |
| 270 | | MMP14 substrate | GPLGLVAQ |
| | | motif sequence | |
| 271 | | MMP14 substrate | GPLGLKLQ |
| | | motif sequence | |
| 272 | | MMP14 substrate | GPLGLKFQ |
| | | motif sequence | |
| 273 | | MMP14 substrate | GPLGLKEQ |
| | | motif sequence | |
| 274 | | MMP14 substrate | GPLGLKKQ |
| | | motif sequence | |
| 275 | | MMP14 substrate | GPLGLKQQ |
| | | motif sequence | |
| 276 | | MMP14 substrate | GPLGLKSQ |
| | | motif sequence | |
| 277 | | MMP14 substrate | GPLGLKGQ |
| | | motif sequence | |
| 278 | | MMP14 substrate | GPLGLKHQ |
| | | motif sequence | |
| 279 | | MMP14 substrate | GPLGLKPQ |
| | | motif sequence | |
| 280 | | MMP14 substrate | GPLGLKAG |
| | | motif sequence | |
| 281 | | MMP14 substrate | GPLGLKAF |
| | | motif sequence | |
| 282 | | MMP14 substrate | GPLGLKAP |
| | | motif sequence | |
| 283 | | MMP14 substrate | GPLGLKAL |
| | | motif sequence | |
| 284 | | MMP14 substrate | GPLGLKAE |
| | | motif sequence | |
| 285 | | MMP14 substrate | GPLGLKAA |
| | | motif sequence | |
| 286 | | MMP14 substrate | GPLGLKAH |
| | | motif sequence | |
| 287 | | MMP14 substrate | GPLGLKAK |
| | | motif sequence | |
| 288 | | MMP14 substrate | GPLGLKAS |
| | | motif sequence | |
| 289 | | MMP14 substrate | GPLGLFGA |
| | | motif sequence | |
| 290 | | MMP14 substrate | GPLGLQGA |
| | | motif sequence | |
| 291 | | MMP14 substrate | GPLGLVGA |
| | | motif sequence | |
| 292 | | MMP14 substrate | GPLGLAGA |
| | | motif sequence | |
| 293 | | MMP14 substrate | GPLGLLGA |
| | | motif sequence | |
| 294 | | MMP14 substrate | GPLGLRGA |
| | | motif sequence | |
| 295 | | MMP14 substrate | GPLGLYGA |
| | | motif sequence | |
| 296 | | CTSL1 substrate | ALFKSSPP |
| | | motif sequence | |
| 297 | | CTSL1 substrate | SPFRSSRQ |
| | | motif sequence | |
| 298 | | CTSL1 substrate | KLFKSSPP |
| | | motif sequence | |
| 299 | | CTSL1 substrate | HLFKSSPP |
| | | motif sequence | |
| 300 | | CTSL1 substrate | SLFKSSPP |
| | | motif sequence | |
| 301 | | CTSL1 substrate | QLFKSSPP |
| | | motif sequence | |
| 302 | | CTSL1 substrate | LLFKSSPP |
| | | motif sequence | |
| 303 | | CTSL1 substrate | PLFKSSPP |
| | | motif sequence | |
| 304 | | CTSL1 substrate | FLFKSSPP |
| | | motif sequence | |
| 305 | | CTSL1 substrate | GLFKSSPP |
| | | motif sequence | |
| 306 | | CTSL1 substrate | VLFKSSPP |
| | | motif sequence | |
| 307 | | CTSL1 substrate | ELFKSSPP |
| | | motif sequence | |
| 308 | | CTSL1 substrate | AKFKSSPP |
| | | motif sequence | |
| 309 | | CTSL1 substrate | AHFKSSPP |
| | | motif sequence | |
| 310 | | CTSLI substrate | AGFKSSPP |
| | | motif sequence | |
| 311 | | CTSL1 substrate | APFKSSPP |
| | | motif sequence | |
| 312 | | CTSL1 substrate | ANFKSSPP |
| | | motif sequence | |
| 313 | | CTSL1 substrate | AFFKSSPP |
| | | motif sequence | |
| 314 | | CTSL1 substrate | AAFKSSPP |
| | | motif sequence | |
| 315 | | CTSL1 substrate | ASFKSSPP |
| | | motif sequence | |
| 316 | | CTSL1 substrate | AEFKSSPP |
| | | motif sequence | |
| 317 | | CTSL1 substrate | ALRKSSPP |
| | | motif sequence | |
| 318 | | CTSL1 substrate | ALLKSSPP |
| | | motif sequence | |
| 319 | | CTSL1 substrate | ALAKSSPP |
| | | motif sequence | |
| 320 | | CTSL1 substrate | ALQKSSPP |
| | | motif sequence | |
| 321 | | CTSL1 substrate | ALHKSSPP |
| | | motif sequence | |
| 322 | | CTSLI substrate | ALPKSSPP |
| | | motif sequence | |
| 323 | | CTSL1 substrate | ALTKSSPP |
| | | motif sequence | |
| 324 | | CTSL1 substrate | ALGKSSPP |
| | | motif sequence | |
| 325 | | CTSL1 substrate | ALDKSSPP |
| | | motif sequence | |
| 326 | | CTSL1 substrate | ALFFSSPP |
| | | motif sequence | |
| 327 | | CTSL1 substrate | ALFHSSPP |
| | | motif sequence | |
| 328 | | CTSL1 substrate | ALFTSSPP |
| | | motif sequence | |
| 329 | | CTSL1 substrate | ALFASSPP |
| | | motif sequence | |
| 330 | | CTSL1 substrate | ALFQSSPP |
| | | motif sequence | |
| 331 | | CTSL1 substrate | ALFLSSPP |
| | | motif sequence | |
| 332 | | CTSL1 substrate | ALFGSSPP |
| | | motif sequence | |
| 333 | | CTSL1 substrate | ALFESSPP |
| | | motif sequence | |
| 334 | | CTSL1 substrate | ALFPSSPP |
| | | motif sequence | |
| 335 | | CTSL1 substrate | ALFKHSPP |
| | | motif sequence | |
| 336 | | CTSL1 substrate | ALFKLSPP |
| | | motif sequence | |
| 337 | | CTSL1 substrate | ALFKKSPP |
| | | motif sequence | |
| 338 | | CTSL1 substrate | ALFKASPP |
| | | motif sequence | |
| 339 | | CTSL1 substrate | ALFKISPP |
| | | motif sequence | |
| 340 | | CTSL1 substrate | ALFKGSPP |
| | | motif sequence | |
| 341 | | CTSL1 substrate | ALFKNSPP |
| | | motif sequence | |
| 342 | | CTSLI substrate | ALFKRSPP |
| | | motif sequence | |
| 343 | | CTSL1 substrate | ALFKESPP |
| | | motif sequence | |
| 344 | | CTSL1 substrate | ALFKFSPP |
| | | motif sequence | |
| 345 | | CTSL1 substrate | ALFKPSPP |
| | | motif sequence | |
| 346 | | CTSL1 substrate | ALFKSFPP |
| | | motif sequence | |
| 347 | | CTSL1 substrate | ALFKSLPP |
| | | motif sequence | |
| 348 | | CTSL1 substrate | ALFKSIPP |
| | | motif sequence | |
| 349 | | CTSL1 substrate | ALFKSKPP |
| | | motif sequence | |
| 350 | | CTSL1 substrate | ALFKSAPP |
| | | motif sequence | |
| 351 | | CTSL1 substrate | ALFKSQPP |
| | | motif sequence | |
| 352 | | CTSL1 substrate | ALFKSPPP |
| | | motif sequence | |
| 353 | | CTSL1 substrate | ALFKSEPP |
| | | motif sequence | |
| 354 | | CTSL1 substrate | ALFKSGPP |
| | | motif sequence | |
| 355 | | CTSL1 substrate | ALFKSSFP |
| | | motif sequence | |
| 356 | | CTSL1 substrate | ALFKSSLP |
| | | motif sequence | |
| 357 | | CTSL1 substrate | ALFKSSGP |
| | | motif sequence | |
| 358 | | CTSL1 substrate | ALFKSSSP |
| | | motif sequence | |
| 359 | | CTSL1 substrate | ALFKSSVP |
| | | motif sequence | |
| 360 | | CTSL1 substrate | ALFKSSHP |
| | | motif sequence | |
| 361 | | CTSL1 substrate | ALFKSSAP |
| | | motif sequence | |
| 362 | | CTSL1 substrate | ALFKSSNP |
| | | motif sequence | |
| 363 | | CTSLI substrate | ALFKSSKP |
| | | motif sequence | |
| 364 | | CTSL1 substrate | ALFKSSEP |
| | | motif sequence | |
| 365 | | CTSL1 substrate | ALFKSSPF |
| | | motif sequence | |
| 366 | | CTSLI substrate | ALFKSSPH |
| | | motif sequence | |
| 367 | | CTSL1 substrate | ALFKSSPG |
| | | motif sequence | |
| 368 | | CTSLI substrate | ALFKSSPA |
| | | motif sequence | |
| 369 | | CTSL1 substrate | ALFKSSPS |
| | | motif sequence | |
| 370 | | CTSL1 substrate | ALFKSSPV |
| | | motif sequence | |
| 371 | | CTSL1 substrate | ALFKSSPQ |
| | | motif sequence | |
| 372 | | CTSL1 substrate | ALFKSSPK |
| | | motif sequence | |
| 373 | | CTSL1 substrate | ALFKSSPL |
| | | motif sequence | |
| 374 | | CTSL1 substrate | ALFKSSPD |
| | | motif sequence | |
| 375 | | MMP7 | KRALGLPG |
| 376 | | MMP7 | (DE)₈RPLALWRS(DR)₈ |
| 377 | | MMP9 | PR(S/T)(L/I)(S/T) |
| 378 | | MMP9 | LEATA |
| 379 | | MMP11 | GGAANLVRGG |
| 380 | | MMP14 | SGRIGFLRTA |
| 381 | | MMP | PLGLAG |
| 382 | | MMP | PLGLAX |
| 383 | | MMP | PLGC(me)AG |
| 384 | | MMP | ESPAYYTA |
| 385 | | MMP | RLQLKL |
| 386 | | MMP | RLQLKAC |
| 387 | | MMP2, MMP9, | EP(Cit)G(Hof)YL |
| | | MMP14 | |
| 388 | | Urokinase | SGRSA |
| | | plasminogen | |
| | | activator (uPA) | |
| 389 | | Urokinase | DAFK |
| | | plasminogen | |
| | | activator (uPA) | |
| 390 | | Urokinase | GGGRR |
| | | plasminogen | |
| | | activator (uPA) | |
| 391 | | Lysosomal | GFLG |
| | | Enzyme | |
| 392 | | Lysosomal | ALAL |
| | | Enzyme | |
| 393 | | Lysosomal | FK |
| | | Enzyme | |
| 394 | | Cathepsin B | NLL |
| 395 | | Cathepsin D | PIC(Et)FF |
| 396 | | Cathepsin K | GGPRGLPG |
| 397 | | Prostate Specific | HSSKLQ |
| | | Antigen | |
| 398 | | Prostate Specific | HSSKLQL |
| | | Antigen | |
| 399 | | Prostate Specific | HSSKLQEDA |
| | | Antigen | |
| 400 | | Herpes Simplex | LVLASSSFGY |
| | | Virus Protease | |
| 401 | | HIV Protease | GVSQNYPIVG |
| 402 | | CMV Protease | GVVQASCRLA |
| 403 | | Thrombin | F(Pip)RS |
| 404 | | Thrombin | DPRSFL |
| 405 | | Thrombin | PPRSFL |
| 406 | | Caspase-3 | DEVD |
| 407 | | Caspase-3 | DEVDP |
| 408 | | Caspase-3 | KGSGDVEG |
| 409 | | Interleukin 1ß | GWEHDG |
| | | converting | |
| | | enzyme | |
| 410 | | Enterokinase | EDDDDKA |
| 411 | | FAP | KQEQNPGST |
| 412 | | Kallikrein 2 | GKAFRR |
| 413 | | Plasmin | DAFK |
| 114 | | Plasmin | DVLK |
| 415 | | Plasmin | DAFK |
| 416 | | TOP | ALLLALL |
| 417 | | | GPLGVRG |
| 418 | | | IPVSLRSG |
| 419 | | | VPLSLYSG |
| 420 | | | SGESPAYYTA |
| 421 | | IL-12 subunit beta | MCHQQLVISWFSLVFLASPLVAIwelkkdvyvveld |
| | | precursor | wypdapgemvvltcdtpeedgitwtldqssevlgsgktltiqvkefgd |
| | | | agqytchkggevlshsllllhkkedgiwstdilkdqkepknktflrceak |
| | | | nysgrftcwwlttistdltfsvkssrgssdpqgvtcgaatlsaervrgdnk |
| | | | eyeysvecqedsacpaaeeslpievmvdavhklkyenytssffirdiik |
| | | | pdppknlqlkplknsrqvevsweypdtwstphsyfsltfcvqvqgks |
| | | | krekkdrvftdktsatvicrknasisvraqdryyssswsewasvpcs |
| 422 | WW50009 | Monomeric mouse | EVQLVESGGGLVQPGNSLRLSCAASGFTFSKFG |
| | | IL-23 polypeptide | MSWVRQAPGKGLEWVSSISGSGRDTLYAESV |
| | | and anti-HSA | KGRFTISRDNAKTTLYLQMNSLRPEDTAVYYC |
| | | (HSA-L- | TIGGSLSVSSQGTLVTVSSggggsggggsggggsMWE |
| | | Mouse_IL23) | LEKDVYVVEVDWTPDAPGETVNLTCDTPEED |
| | | | DITWTSDQRHGVIGSGKTLTITVKEFLDAGQYT |
| | | | CHKGGETLSHSHLLLHKKENGIWSTEILKNFKN |
| | | | KTFLKCEAPNYSGRFTCSWLVQRNMDLKFNIK |
| | | | SSSSSPDSRAVTCGMASLSAEKVTLDQRDYEK |
| | | | YSVSCQEDVTCPTAEETLPIELALEARQQNKYE |
| | | | NYSTSFFIRDIIKPDPPKNLQMKPLKNSQVEVS |
| | | | WEYPDSWSTPHSYFSLKFFVRIQRKKEKMKET |
| | | | EEGCNQKGAFLVEKTSTEVQCKGGNVCVQAQ |
| | | | DRYYNSSCSKWACVPCRVRSggggsggggsggggsg |
| | | | gggsVPRSSSPDWAQCQQLSRNLCMLAWNAHA |
| | | | PAGHMNLLREEEDEETKNNVPRIQCEDGCDPQ |
| | | | GLKDNSQFCLQRIRQGLAFYKHLLDSDIFKGEP |
| | | | ALLPDSPMEQLHTSLLGLSQLLQPEDHPRETQQ |
| | | | MPSLSSSQQWQRPLLRSKILRSLQAFLAIAARV |
| | | | FAHGAATLTEPLVPTA** |
| 423 | WW50055 | Heterodimeric | VPRSSSPDWAQCQQLSRNLCMLAWNAHAPAG |
| | | mouse IL-23 | HMNLLREEEDEETKNNVPRIQCEDGCDPQGLK |
| | | polypeptide | DNSQFCLQRIRQGLAFYKHLLDSDIFKGEPALL |
| | | | PDSPMEQLHTSLLGLSQLLQPEDHPRETQQMPS |
| | | | LSSSQQWQRPLLRSKILRSLQAFLAIAARVFAH |
| | | | GAATLTEPLVPTAHHHHHH** |
| 424 | WW50056 | Heterodimeric | RAVPGGSSPAWTQCQQLSQKLCTLAWSAHPL |
| | | human IL-23 | VGHMDLREEGDEETTNDVPHIQCGDGCDPQG |
| | | polypeptide | LRDNSQFCLQRIHQGLIFYEKLLGSDIFTGEPSL |
| | | | LPDSPVGQLHASLLGLSQLLQPEGHHWETQQIP |
| | | | SLSPSQPWQRLLLRFKILRSLQAFVAVAARVFA |
| | | | HGAATLSPHHHHHH** |
| 425 | WW50057 | Heterodimeric IL- | EVQLVESGGGLVQPGNSLRLSCAASGFTFSKFG |
| | | 23 polypeptide | MSWVRQAPGKGLEWVSSISGSGRDTLYAESV |
| | | and anti-HSA | KGRFTISRDNAKTTLYLQMNSLRPEDTAVYYC |
| | | | TIGGSLSVSSQGTLVTVSSsggpGggGsgggpgsVPR |
| | | | SSSPDWAQCQQLSRNLCMLAWNAHAPAGHM |
| | | | NLLREEEDEETKNNVPRIQCEDGCDPQGLKDN |
| | | | SQFCLQRIRQGLAFYKHLLDSDIFKGEPALLPD |
| | | | SPMEQLHTSLLGLSQLLQPEDHPRETQQMPSLS |
| | | | SSQQWQRPLLRSKILRSLQAFLAIAARVFAHGA |
| | | | ATLTEPLVPTA** |
| 426 | WW50058 | Heterodimeric | EVQLVESGGGLVQPGNSLRLSCAASGFTFSKFG |
| | | human IL-23 | MSWVRQAPGKGLEWVSSISGSGRDTLYAESV |
| | | polypeptide and | KGRFTISRDNAKTTLYLQMNSLRPEDTAVYYC |
| | | anti-HSA | TIGGSLSVSSQGTLVTVSSsggpGggGsgggpgsRA |
| | | (Anti-HSA-L- | VPGGSSPAWTQCQQLSQKLCTLAWSAHPLVG |
| | | Human_IL23A/ | HMDLREEGDEETTNDVPHIQCGDGCDPQGLRD |
| | | Human_IL12B) | NSQFCLQRIHQGLIFYEKLLGSDIFTGEPSLLPD |
| | | | SPVGQLHASLLGLSQLLQPEGHHWETQQIPSLS |
| | | | PSQPWQRLLLRFKILRSLQAFVAVAARVFAHG |
| | | | AATLSP** |
| 427 | WW50059 | Heterodimeric | EVQLVESGGGLVQPGNSLRLSCAASGFTFSKFG |
| | | human IL-23 | MSWVRQAPGKGLEWVSSISGSGRDTLYAESV |
| | | polypeptide, anti- | KGRFTISRDNAKTTLYLQMNSLRPEDTAVYYC |
| | | HSA, blocker, | TIGGSLSVSSQGTLVTVSSsggpALFKSSFPpgsVP |
| | | scFv, linker | RSSSPDWAQCQQLSRNLCMLAWNAHAPAGH |
| | | | MNLLREEEDEETKNNVPRIQCEDGCDPQGLKD |
| | | | NSQFCLQRIRQGLAFYKHLLDSDIFKGEPALLP |
| | | | DSPMEQLHTSLLGLSQLLQPEDHPRETQQMPSL |
| | | | SSSQQWQRPLLRSKILRSLQAFLAIAARVFAHG |
| | | | AATLTEPLVPTAsggpALFKSSFPpgsggggsggggsg |
| | | | gggsggggsggggsggggsQSVLTQPPSVSGAPGQRVT |
| | | | ISCSGSRSNIGSNTVKWYQQLPGTAPKLLIYYN |
| | | | DQRPSGVPDRFSGSKSGTSASLAITGLQAEDEA |
| | | | DYYCQSYDRYTHPALLFGTGTKVTVLggggsggg |
| | | | gsggggsQVQLVESGGGVVQPGRSLRLSCAASGF |
| | | | TFSSYGMHWVRQAPGKGLEWVAFIRYeGSNK |
| | | | YYAeSVKGRFTISRDNSKNTLYLQMNSLRAED |
| | | | TAVYYCKTHGSHDNWGQGTMVTVSS** |
| 428 | WW50060 | Heterodimeric | EVQLVESGGGLVQPGNSLRLSCAASGFTFSKFG |
| | | human IL-23 | MSWVRQAPGKGLEWVSSISGSGRDTLYAESV |
| | | polypeptide, anti- | KGRFTISRDNAKTTLYLQMNSLRPEDTAVYYC |
| | | HSA, blocker, | TIGGSLSVSSQGTLVTVSSsggpALFKSSFPpgsRA |
| | | scFv, linker | VPGGSSPAWTQCQQLSQKLCTLAWSAHPLVG |
| | | | HMDLREEGDEETTNDVPHIQCGDGCDPQGLRD |
| | | | NSQFCLQRIHQGLIFYEKLLGSDIFTGEPSLLPD |
| | | | SPVGQLHASLLGLSQLLQPEGHHWETQQIPSLS |
| | | | PSQPWQRLLLRFKILRSLQAFVAVAARVFAHG |
| | | | AATLSPsggpALFKSSFPpgsggggggggsggggsggggs |
| | | | ggggsggggsQSVLTQPPSVSGAPGQRVTISCSGSR |
| | | | SNIGSNTVKWYQQLPGTAPKLLIYYNDQRPSG |
| | | | VPDRFSGSKSGTSASLAITGLQAEDEADYYCQS |
| | | | YDRYTHPALLFGTGTKVTVLggggsggggsggggsQ |
| | | | VQLVESGGGVVQPGRSLRLSCAASGFTFSSYG |
| | | | MHWVRQAPGKGLEWVAFIRYeGSNKYYAeSV |
| | | | KGRFTISRDNSKNTLYLQMNSLRAEDTAVYYC |
| | | | KTHGSHDNWGQGTMVTVSS** |
| 429 | WW50087 | Chimeric | mdmrvpaqllgllllwlrgarcEVQLVESGGGLVQPGNS |
| | | monomeric mouse | LRLSCAASGFTFSKFGMSWVRQAPGKGLEWV |
| | | IL-23 polypeptide | SSISGSGRDTLYAESVKGRFTISRDNAKTTLYL |
| | | | QMNSLRPEDTAVYYCTIGGSLSVSSQGTLVTVS |
| | | | Sggggsggggsggggsiwelkkdvyvveldwypdapgemvvltc |
| | | | dtpeedgitwtldqssevlgsgktltiqvkefgdagqytchkggevlshs |
| | | | llllhkkedgiwstdilkdqkepknktflrceaknysgrftcwwlttistd |
| | | | ltfsvkssrgssdpqgvtcgaatlsaervrgdnkeyeysvecqedsacp |
| | | | aaeeslpievmvdavhklkyenytssffirdiikpdppknlqlkplkns |
| | | | rqvevsweypdtwstphsyfsltfcvqvqgkskrekkdrvftdktsatv |
| | | | icrknasisvraqdryyssswsewasvpcsggggsggggsggggsgg |
| | | | ggsVPRSSSPDWAQCQQLSRNLCMLAWNAHAP |
| | | | AGHMNLLREEEDEETKNNVPRIQCEDGCDPQG |
| | | | LKDNSQFCLQRIRQGLAFYKHLLDSDIFKGEPA |
| | | | LLPDSPMEQLHTSLLGLSQLLQPEDHPRETQQ |
| | | | MPSLSSSQQWQRPLLRSKILRSLQAFLAIAARV |
| | | | FAHGAATLTEPLVPTA** |
| 430 | WW50088 | Chimeric | mdmrvpaqllgllllwlrgarcEVQLVESGGGLVQPGNS |
| | | monomeric human | LRLSCAASGFTFSKFGMSWVRQAPGKGLEWV |
| | | IL-23 polypeptide | SSISGSGRDTLYAESVKGRFTISRDNAKTTLYL |
| | | | QMNSLRPEDTAVYYCTIGGSLSVSSQGTLVTVS |
| | | | Sggggsggggsggggsiwelkkdvyvveldwypdapgemvvltc |
| | | | dtpeedgitwtldqssevlgsgktltiqvkefgdagqytchkggevlshs |
| | | | llllhkkedgiwstdilkdqkepknktflrceaknysgrftcwwlttistd |
| | | | ltfsvkssrgssdpqgvtcgaatlsaervrgdnkeyeysvecqedsacp |
| | | | aaeeslpievmvdavhklkyenytssffirdiikpdppknlqlkplkns |
| | | | rqvevsweypdtwstphsyfsltfcvqvqgkskrekkdrvftdktsatv |
| | | | icrknasisvraqdryyssswsewasvpcsggggsggggsggggsgg |
| | | | ggsRAVPGGSSPAWTQCQQLSQKLCTLAWSAH |
| | | | PLVGHMDLREEGDEETTNDVPHIQCGDGCDPQ |
| | | | GLRDNSQFCLQRIHQGLIFYEKLLGSDIFTGEPS |
| | | | LLPDSPVGQLHASLLGLSQLLQPEGHHWETQQI |
| | | | PSLSPSQPWQRLLLRFKILRSLQAFVAVAARVF |
| | | | AHGAATLSP** |
| 431 | WW50089 | Chimeric | mdmrvpaqllgllllwlrgarcEVQLVESGGGLVQPGNS |
| | | monomeric IL-23 | LRLSCAASGFTFSKFGMSWVRQAPGKGLEWV |
| | | polypeptide, | SSISGSGRDTLYAESVKGRFTISRDNAKTTLYL |
| | | blocker, scFv | QMNSLRPEDTAVYYCTIGGSLSVSSQGTLVTVS |
| | | | SsggpALFKSSFPpgsiwelkkdvyvveldwypdapgemvvl |
| | | | tcdtpeedgitwtldqssevlgsgktltiqvkefgdagqytchkggevls |
| | | | hsllllhkkedgiwstdilkdqkepknktflrceaknysgrftcwwlttis |
| | | | tdltfsvkssrgssdpqgvtcgaatlsaervrgdnkeyeysvecqedsac |
| | | | paaeeslpievmvdavhklkyenytssffirdiikpdppknlqlkplkn |
| | | | srqvevsweypdtwstphsyfsltfcvqvqgkskrekkdrvftdktsat |
| | | | vicrknasisvraqdryyssswsewasvpcsggggggggsggggsg |
| | | | gggsVPRSSSPDWAQCQQLSRNLCMLAWNAHA |
| | | | PAGHMNLLREEEDEETKNNVPRIQCEDGCDPQ |
| | | | GLKDNSQFCLQRIRQGLAFYKHLLDSDIFKGEP |
| | | | ALLPDSPMEQLHTSLLGLSQLLQPEDHPRETQQ |
| | | | MPSLSSSQQWQRPLLRSKILRSLQAFLAIAARV |
| | | | FAHGAATLTEPLVPTAsggpALFKSSFPpgsggggsg |
| | | | gggsggggggggggggsggggsQSVLTQPPSVSGAPG |
| | | | QRVTISCSGSRSNIGSNTVKWYQQLPGTAPKLL |
| | | | IYYNDQRPSGVPDRFSGSKSGTSASLAITGLQA |
| | | | EDEADYYCQSYDRYTHPALLFGTGTKVTVLgg |
| | | | ggsggggsggggsQVQLVESGGGVVQPGRSLRLSC |
| | | | AASGFTFSSYGMHWVRQAPGKGLEWVAFIRYe |
| | | | GSNKYYAeSVKGRFTISRDNSKNTLYLQMNSL |
| | | | RAEDTAVYYCKTHGSHDNWGQGTMVTVSS** |
| 432 | WW50090 | Chimeric | mdmrvpaqllgllllwlrgarcEVQLVESGGGLVQPGNS |
| | | monomeric human | LRLSCAASGFTFSKFGMSWVRQAPGKGLEWV |
| | | IL-23 polypeptide, | SSISGSGRDTLYAESVKGRFTISRDNAKTTLYL |
| | | blocker, scFv | QMNSLRPEDTAVYYCTIGGSLSVSSQGTLVTVS |
| | | | SsggpALFKSSFPpgsiwelkkdvyvveldwypdapgemvvl |
| | | | tcdtpeedgitwtldqssevlgsgktltiqvkefgdagqytchkggevls |
| | | | hsllllhkkedgiwstdilkdqkepknktflrceaknysgrftcwwlttis |
| | | | tdltfsvkssrgssdpqgvtcgaatlsaervrgdnkeyeysvecqedsac |
| | | | paaeeslpievmvdavhklkyenytssffirdiikpdppknlqlkplkn |
| | | | srqvevsweypdtwstphsyfsltfcvqvqgkskrekkdrvftdktsat |
| | | | vicrknasisvraqdryyssswsewasvpcsggggsggggsggggsg |
| | | | gggsRAVPGGSSPAWTQCQQLSQKLCTLAWSA |
| | | | HPLVGHMDLREEGDEETTNDVPHIQCGDGCDP |
| | | | QGLRDNSQFCLQRIHQGLIFYEKLLGSDIFTGEP |
| | | | SLLPDSPVGQLHASLLGLSQLLQPEGHHWETQ |
| | | | QIPSLSPSQPWQRLLLRFKILRSLQAFVAVAAR |
| | | | VFAHGAATLSPsggpALFKSSFPpgsggggsggggsgg |
| | | | ggsggggsggggsggggsQSVLTQPPSVSGAPGQRVTI |
| | | | SCSGSRSNIGSNTVKWYQQLPGTAPKLLIYYND |
| | | | QRPSGVPDRFSGSKSGTSASLAITGLQAEDEAD |
| | | | YYCQSYDRYTHPALLFGTGTKVTVLggggsggggs |
| | | | ggggsQVQLVESGGGVVQPGRSLRLSCAASGFTF |
| | | | SSYGMHWVRQAPGKGLEWVAFIRYeGSNKYY |
| | | | AeSVKGRFTISRDNSKNTLYLQMNSLRAEDTA |
| | | | VYYCKTHGSHDNWGQGTMVTVSS** |
| 433 | WW00141 | Mouse_IL12B | mwelekdvyvvevdwtpdapgetvnltcdtpeedditwtsdqrhgv |
| | | | igsgktltitvkefldagqytchkggetlshshlllhkkengiwsteilknf |
| | | | knktflkceapnysgrftcswlvqrnmdlkfnikssssspdsravtcg |
| | | | maslsaekvtldqrdyekysvscqedvtcptaeetlpielalearqqnk |
| | | | yenystsffirdiikpdppknlqmkplknsqvevsweypdswstphs |
| | | | yfslkffvriqrkkekmketeegcnqkgaflvektstevqckggnvcv |
| | | | qaqdryynsscskwacvpcrvrs** |
| 434 | WW00636 | Human_IL12B | iwelkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcs** |
| 435 | WW00636 | Human_IL12B | iwelkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcs** |
| 436 | WW00141 | Mouse_IL12B | mwelekdvyvvevdwtpdapgetvnltcdtpeedditwtsdqrhgv |
| | | | igsgktltitvkefldagqytchkggetlshshlllhkkengiwsteilknf |
| | | | knktflkceapnysgrftcswlvqrnmdlkfnikssssspdsravtcg |
| | | | maslsaekvtldqrdyekysvscqedvtcptaeetlpielalearqqnk |
| | | | yenystsffirdiikpdppknlqmkplknsqvevsweypdswstphs |
| | | | yfslkffvriqrkkekmketeegcnqkgaflvektstevqckggnvcv |
| | | | qaqdryynsscskwacvpcrvrs** |
| 437 | WW00636 | Human_IL12B | iwelkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcs** |
| 438 | WW00636 | Human_IL12B | iwelkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcs** |
| 439 | WW00636 | Human_IL12B | iwelkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcs** |
| 440 | WW00636 | Human_IL12B | iwelkkdvyvveldwypdapgemvvltcdtpeedgitwtldqssevl |
| | | | gsgktltiqvkefgdagqytchkggevlshsllllhkkedgiwstdilkd |
| | | | qkepknktflrceaknysgrftcwwlttistdltfsvkssrgssdpqgvtc |
| | | | gaatlsaervrgdnkeyeysvecqedsacpaaeeslpievmvdavhkl |
| | | | kyenytssffirdiikpdppknlqlkplknsrqvevsweypdtwstphs |
| | | | yfsltfcvqvqgkskrekkdrvftdktsatvicrknasisvraqdryysss |
| | | | wsewasvpcs** |
| 441 | WW00758 | HSA-X- | EVQLVESGGGLVQPGNSLRLSCAASGFTFSKFG |
| | | Human_p35-XL- | MSWVRQAPGKGLEWVSSISGSGRDTLYAESV |
| | | Blocker_(Blocker = | KGRFTISRDNAKTTLYLQMNSLRPEDTAVYYC |
| | | Opt1_Hv_D53E_ | TIGGSLSVSSQGTLVTVSSsggpALFKSSFPpgsrnl |
| | | D61E_Vl- | pvatpdpgmfpclhhsqnllravsnmlqkarqtlefypctseeidhedi |
| | | Vh X = Linker3) | tkdktstveaclpleltknescInsretsfitngsclasrktsfmmalclssi |
| | | | yedlkmyqvefktmnakllmdpkrqifldqnmlavidelmqalnfn |
| | | | setvpqkssleepdfyktkiklcillhafriravtidrvmsylnassggpA |
| | | | LFKSSFPpgsggggsggggggggsggggsggggsggggsQS |
| | | | VLTQPPSVSGAPGQRVTISCSGSRSNIGSNTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLggggggggsggggsQVQLVESGG |
| | | | GVVQPGRSLRLSCAASGFTFSSYGMHWVRQAP |
| | | | GKGLEWVAFIRYeGSNKYYAeSVKGRFTISRDN |
| | | | SKNTLYLQMNSLRAEDTAVYYCKTHGSHDNW |
| | | | GQGTMVTVSS** |
| 442 | WW00924 | HSA-X- | EVQLVESGGGLVQPGNSLRLSCAASGFTFSKFG |
| | | Human_p35-XL- | MSWVRQAPGKGLEWVSSISGSGRDTLYAESV |
| | | Blocker_(Blocker = | KGRFTISRDNAKTTLYLQMNSLRPEDTAVYYC |
| | | Opt1_Hv_D53E_ | TIGGSLSVSSQGTLVTVSSsggpALFKSSFPpgsrnl |
| | | D61E_Vl- | pvatpdpgmfpclhhsqnllravsnmlqkarqtlefypctseeidhedi |
| | | Vh X = | tkdktstveaclpleltkQesclnsretsfitQgsclasrktsfmmalclss |
| | | Linker3)_ | iyedlkmyqvefktmnakllmdpkrqifldqnmlavidelmqalnfn |
| | | Deglycosylated | setvpqkssleepdfyktkiklcillhafriravtidrvmsylQassggp |
| | | | ALFKSSFPpgsggggsggggsggggggggsggggsggggsQ |
| | | | SVLTQPPSVSGAPGQRVTISCSGSRSNIGSNTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLggggsggggsggggsQVQLVESGG |
| | | | GVVQPGRSLRLSCAASGFTFSSYGMHWVRQAP |
| | | | GKGLEWVAFIRYeGSNKYYAeSVKGRFTISRDN |
| | | | SKNTLYLQMNSLRAEDTAVYYCKTHGSHDNW |
| | | | GQGTMVTVSS** |
| 443 | WW00925 | Human_IL12B_ | iwelkkdvyvveldwypdapgemvvltcdtpeedgitwtldqssevl |
| | | Deglycosylated | gsgktltiqvkefgdagqytchkggevlshsllllhkkedgiwstdilkd |
| | | | qkepkQktflrceakQysgrftcwwlttistdltfsvkssrgssdpqgvt |
| | | | cgaatlsaervrgdnkeyeysvecqedsacpaaeeslpievmvdavh |
| | | | klkyeQytssffirdiikpdppknlqlkplknsrqvevsweypdtwst |
| | | | phsyfsltfcvqvqgkskrekkdrvftdktsatvicrkQasisvraqdry |
| | | | yssswsewasvpcs** |
| 444 | WW00935 | Human_IL12B_ | iwelkkdvyvveldwypdapgemvvltcdtpeedgitwtldqssevl |
| | | (WW0636)_partially_ | gsgktltiqvkefgdagqytchkggevlshsllllhkkedgiwstdilkd |
| | | Deglycosylated | qkepkNktflrceakQysgrftcwwlttistdltfsvkssrgssdpqgvt |
| | | | cgaatlsaervrgdnkeyeysvecqedsacpaaeeslpievmvdavh |
| | | | klkyeQytssffirdiikpdppknlqlkplknsrqvevsweypdtwst |
| | | | phsyfsltfcvqvqgkskrekkdrvftdktsatvicrkNasisvraqdry |
| | | | yssswsewasvpcs** |
| 445 | WW00936 | HSA-X- | EVQLVESGGGLVQPGNSLRLSCAASGFTFSKFG |
| | | Human_p35-XL- | MSWVRQAPGKGLEWVSSISGSGRDTLYAESV |
| | | Blocker_(Blocker = | KGRFTISRDNAKTTLYLQMNSLRPEDTAVYYC |
| | | Opt1_Hv_D53E_ | TIGGSLSVSSQGTLVTVSSsggpALFKSSFPpgsrnl |
| | | D61E_Vl- | pvatpdpgmfpclhhsqnllravsnmlqkarqtlefypctseeidhedi |
| | | Vh_X = Linker3) | tkdktstveaclpleltkQesclnsretsfitQgsclasrktsfmmalclss |
| | | Partially_ | iyedlkmyqvefktmnakllmdpkrqifldqnmlavidelmqalnfn |
| | | deglycosylated | setvpqkssleepdfyktkiklcillhafriravtidrvmsylNassggp |
| | | | ALFKSSFPpgsggggggggsggggsggggsggggsggggsQ |
| | | | SVLTQPPSVSGAPGQRVTISCSGSRSNIGSNTVK |
| | | | WYQQLPGTAPKLLIYYNDQRPSGVPDRFSGSK |
| | | | SGTSASLAITGLQAEDEADYYCQSYDRYTHPA |
| | | | LLFGTGTKVTVLggggggggsggggsQVQLVESGG |
| | | | GVVQPGRSLRLSCAASGFTFSSYGMHWVRQAP |
| | | | GKGLEWVAFIRYeGSNKYYAeSVKGRFTISRDN |
| | | | SKNTLYLQMNSLRAEDTAVYYCKTHGSHDNW |
| | | | GQGTMVTVSS |

1. A polypeptide complex comprising IL-12, a half-life extension element, an IL-12 blocking element and a protease cleavable linker, wherein the IL-12 blocking element is a single chain antibody the binds IL-12 or an antigen binding fragment thereof, and the complex comprises:
i. a first polypeptide comprising an IL-12 subunit, and optionally the IL-12 blocking element, wherein the IL-12 blocking element when present is operably linked to the IL-12 subunit through a first protease cleavable linker;
ii. a second polypeptide chain comprising an IL-12 subunit operably linked to a half-life extension element through a second protease cleavable linker, and optionally the IL-12 blocking element, wherein the IL-12 blocking element when present is operably linked to the IL-12 subunit through a first protease cleavable linker or is operably linked to the half-life extension element through a linker that is optionally protease cleavable;
wherein only one of the first and second polypeptide contains the IL-12 blocking element; and
wherein when the IL-12 subunit in the first polypeptide is p35 the IL-12 subunit in the second polypeptide is p40, and when the IL-12 subunit in the first polypeptide is p40 the IL-12 subunit in the second polypeptide is p35.

2. The polypeptide of claim 1, wherein the first protease cleavable linker and the second protease cleavable linker are the same.

3-7. (canceled)

8. The polypeptide complex of claim 1, wherein the first polypeptide does not comprise a blocking element and the second polypeptide has the formula:
[A]-[L1]-[B]-[L3]-[D] or [D]-[L3]-[B]-[L1]-[A] or [B]-[L1]-[A]-[L2]-[D] or [D]-[L1]-[A]-[L2]-[B], wherein,
A is the IL-12 subunit;
L1 is the first protease-cleavable linker;
L2 is the second protease cleavable linker;
L3 is the optionally cleavable linker;
B is the half-life extension element; and
D is the blocking element.

9. The polypeptide complex of claim 1, wherein the first polypeptide comprises the formula:
[A]-[L1]-[D] or [D]-[L1]-[A]; and the second polypeptide has the formula:
[A′]-[L2]-[B] or [B]-[L2]-[A′], wherein A is either p35 or p40, wherein when A is p35, A′ is p40 and when A is p40, A′ is p35;
A′ is either p35 or p40;
L1 is the first protease cleavable linker;
L2 is the second protease cleavable linker;
B is the half-life extension element; and
D is the blocking element.

10. (canceled)

11. The polypeptide complex of claim 1, wherein the half-life extension element is a human serum albumin, an antigen binding polypeptide that binds human serum albumin, or an immunoglobulin Fc or fragment thereof.

12. The polypeptide complex of claim 1, wherein the protease cleavable linker comprises a sequence that is capable of being cleaved by a protease selected from kallikrein, thrombin, chymase, carboxypeptidase A, cathepsin, elastase, PR-3, granzyme M, a calpain, a matrix metalloproteinase (MMP), an ADAM, a FAP, a plasminogen activator, a caspase, a tryptase, or a tumor protease.

13. The polypeptide complex of claim 1, wherein the protease is selected from cathepsin B, cathepsin C, cathepsin D, cathepsin E, cathepsin K, cathepsin L, or cathepsin G.

14. The polypeptide complex of claim 1, wherein protease is selected from matrix metalloprotease (MMP) is MMP1, MMP2, MMP3, MMP8, MMP9, MMP10, MMP11, MMP12, MMP13, or MMP14.

15. The polypeptide complex of claim 1, wherein the protease cleavable linker comprises at least two sequences that are independently capable of being cleaved by a protease.

16-17. (canceled)

18. The polypeptide complex of claim 1, wherein the single chain antibody is a single chain variable fragment (scFv).

19. (canceled)

20. The polypeptide complex of claim 1, wherein the blocking element binds the IL-12.

21. The polypeptide complex of claim 18, wherein the blocking element binds p35, p40, or to the p35p40 complex.

22. A nucleic acid encoding a polypeptides as defined in claim 1.

23. The nucleic acid of claim 22, wherein the nucleic acid does not encode only p35 or p40.

24-34. (canceled)

35. A pharmaceutical composition comprising a protein complex of claim 1.

36. A method for treating a tumor, comprising administering to a subject in need thereof an effective amount of the polypeptide complex of claim 1.

37. An IL-12 polypeptide complex comprising a first polypeptide selected from the group consisting of SEQ ID NOs: 95-110, SEQ ID NOs: 119-126, and SEQ ID NOs: 135-143, or an amino acid sequence that has at least 80% identity to SEQ ID NOs: 95-110, SEQ ID NOs: 119-126, and SEQ ID NOs: 135-143.

38. (canceled)

39. The IL-12 polypeptide complex of claim 37, wherein the first polypeptide chain comprises the amino acid sequence of SEQ ID NO: 104 and a second polypeptide chain comprises the amino acid sequence of SEQ ID NO: 18.

40. The polypeptide complex of claim 37, wherein the first polypeptide chain comprises the amino acid sequence of SEQ ID NO: 136 and a second polypeptide chain comprises the amino acid sequence of SEQ ID NO: 18.

41-42. (canceled)

43. A nucleic acid encoding a polypeptide as defined in claim 37.

44. The nucleic acid composition of claim 37, comprising a circular vector, DNA, or RNA.

45-46. (canceled)

47. An expression vector comprising the nucleic acid claim 37.

48. An isolated host cell comprising the vector of claim 47.

49-51. (canceled)

52. A method for treating a tumor, comprising administering to a subject in need thereof an effective amount of the polypeptide complex of claim 37.

53-56. (canceled)

57. A polypeptide complex comprising IL-23, a half-life extension element, an IL-23 blocking element and a protease cleavable linker, wherein the IL-23 blocking element is a single chain antibody the binds IL-23 or an antigen binding fragment thereof, and the complex comprises:
iii. a first polypeptide comprising an IL-23 subunit, and optionally the IL-23 blocking element, wherein the IL-23 blocking element when present is operably linked to the IL-23 subunit through a first protease cleavable linker;
iv. a second polypeptide chain comprising an IL-23 subunit operably linked to a half-life extension element through a second protease cleavable linker, and optionally the IL-23 blocking element, wherein the IL-23 blocking element when present is operably linked to the IL-23 subunit through a first protease cleavable linker or is operably linked to the half-life extension element through a linker that is optionally protease cleavable;
wherein only one of the first and second polypeptide contains the IL-23 blocking element; and
wherein when the IL-23 subunit in the first polypeptide is p19 the IL-23 subunit in the second polypeptide is p40, and when the IL-23 subunit in the first polypeptide is p40 the IL-23 subunit in the second polypeptide is p19.

58-110. (canceled)


SEQUENCE LISTING (448 sequences)

SEQ ID NO: 1 - protein, 520 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 2 - protein, 524 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASHHHHHH

SEQ ID NO: 3 - protein, 940 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSGGGGSGGGGSGGGGSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 4 - protein, 940 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSGGGGSGGGGSGGGGSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHHEPEA

SEQ ID NO: 5 - protein, 940 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHHEPEA

SEQ ID NO: 6 - protein, 940 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 7 - protein, 940 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 8 - protein, 810 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHHEPEA

SEQ ID NO: 9 - protein, 520 aa, synthetic construct; identical to SEQ ID NO: 1

SEQ ID NO: 10 - protein, 936 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 11 - protein, 936 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 12 - protein, 1405 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 13 - protein, 1405 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 14 - protein, 1048 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 15 - protein, 1048 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSSGGPGPAGMKGLPGSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKHHHHHH

SEQ ID NO: 16 - protein, 936 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLSGGPGPAGMKGLPGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 17 - protein, 817 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQVQLQESGGGLVQAGGSLRLSCAASGRTFSSVYDMGWFRQAPGKDREFVARITESARNTRYADSVRGRFTISRDNAKNTVYLQMNNLELEDAAVYYCAADPQTVVVGTPDYWGQGTQVTVSSHHHHHH

SEQ ID NO: 18 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 19 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 20 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESSLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 21 - protein, 935 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 22 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGCGTKVTVLGGGGSGGGGSGGGGSQVQLVESGGGVVQPGRSLRLSCAASGFTFSSYGMHWVRQAPGKCLEWVAFIRYDGSNKYYADSVKGRFTISRDNSKNTLYLQMNSLRAEDTAVYYCKTHGSHDNWGQGTMVTVSS

SEQ ID NO: 23 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTCPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGCGTMVTVSS

SEQ ID NO: 24 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 25 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 26 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 27 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 28 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 29 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 30 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 31 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 32 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 33 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 34 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 35 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 36 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 37 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 38 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 39 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 40 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 41 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 42 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 43 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 44 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSS

SEQ ID NO: 45 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSS

SEQ ID NO: 46 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 47 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 48 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 49 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 50 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 51 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 52 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 53 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 54 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 55 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 56 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 57 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 58 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 59 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 60 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 61 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 62 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSS

SEQ ID NO: 63 - protein, 934 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSS

SEQ ID NO: 64 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 66 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 68 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 69 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 70 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 71 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 72 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 73 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 76 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 80 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 81 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 82 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 84 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 85 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 86 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 87 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 88 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 89 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 90 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 91 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 92 - protein, 218 aa, synthetic construct
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 93 - protein, 218 aa, synthetic construct
QVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 94 - protein, 218 aa, synthetic construct
QVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 95 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 96 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 97 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 98 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 99 - protein, 585 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 100 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 101 - protein, 585 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 102 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 104 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 105 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 106 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 107 - protein, 585 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 108 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 109 - protein, 585 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 110 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - protein, 915 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 112 - protein, 919 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 113 - protein, 915 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 114 - protein, 919 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 115 - protein, 891 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 116 - protein, 895 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 117 - protein, 891 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 118 - protein, 895 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 119 - protein, 594 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 120 - protein, 598 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 121 - protein, 594 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 122 - protein, 598 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 123 - protein, 570 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 124 - protein, 574 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 125 - protein, 570 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 126 - protein, 574 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 127 - protein, 915 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 128 - protein, 919 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 129 - protein, 915 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 130 - protein, 919 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 131 - protein, 891 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 132 - protein, 895 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 133 - protein, 891 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 134 - protein, 895 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 135 - protein, 594 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 136 - protein, 598 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 137 - protein, 594 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 138 - protein, 598 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 139 - protein, 570 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 140 - protein, 574 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 141 - protein, 570 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 142 - protein, 574 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 143 - protein, 203 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASHHHHHH

SEQ ID NO: 144 - protein, 141 aa, synthetic construct
QVQLQESGGGLVQAGGSLRLSCAASGRTFSSVYDMGWFRQAPGKDREFVARITESARNTRYADSVRGRFTISRDNAKNTVYLQMNNLELEDAAVYYCAADPQTVVVGTPDYWGQGTQVTVSSAAAYPYDVPDYGSHHHHHH

SEQ ID NO: 145 - protein, 247 aa, synthetic construct
QSVLTQPPSVSGAPGQRVTISCTGSSSNIGSNTVKWYQQLPGTAPKLLIYGNDQRPSGVPDRFSGSKSGTSASLAITGLQAEDEADYYCQSYDRYTHPAYVFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 146 - protein, 247 aa, synthetic construct
QSVLTQPPSVSGAPGQRVTISCTGSSSNIGSNTVKWYQQLPGTAPKLLIYYNDQRPSGVPDRFSGSKSGTSASLAITGLQAEDEADYYCQSYDRYTHPAYVFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 147 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARHGSHDNWGQGTMVTVSSHHHHHH

SEQ ID NO: 148 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSSHHHHHH

SEQ ID NO: 149 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYAESVKGRFTISRDNSKNTLYLQMNSLRAEDTAVYYCKTHGSHDNWGQGTMVTVSSHHHHHH

SEQ ID NO: 150 - protein, 247 aa, synthetic construct
QSVLTQPPSVSGAPGQRVTISCSGSRSNIGSQTVKWYQQLPGTAPKLLIYYNDQRPSGVPDRFSGSKSGTSASLAITGLQAEDEADYYCQSYERYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 151 - protein, 247 aa, synthetic construct
QSVLTQPPSVSGAPGQRVTISCSGSRSNIGSQTVKWYQQLPGTAPKLLIYYNDQRPSGVPDRFSGSKSGTSASLAITGLQAEDEADYYCQSYSRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 152 - protein, 247 aa, synthetic construct
QSVLTQPPSVSGAPGQRVTISCSGSESNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 153 - protein, 247 aa, synthetic construct
QSVLTQPPSVSGAPGQRVTISCSGSSSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 154 - protein, 247 aa, synthetic construct
QSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 155 - protein, 247 aa, synthetic construct
QSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 156 - protein, 247 aa, synthetic construct
QSVLTQPPSVSGAPGQRVTISCSGSRSNIGSDTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 157 - protein, 247 aa, synthetic construct
QSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 158 - protein, 247 aa, synthetic construct
QSVLTQPPSVSGAPGQRVTISCSGSRSNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 159 - protein, 247 aa, synthetic construct
QSVLTQPPSVSGAPGQRVTISCSGSRSNIGSNTVDWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 160 - protein, 247 aa, synthetic construct
QSVLTQPPSVSGAPGQRVTISCSGSRSNIGSNTVEWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 161 - protein, 247 aa, synthetic construct
QSVLTQPPSVSGAPGQRVTISCSGSRSNIGSNTVKWYQQLPGTAPKLLIYYNDQDPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 162 - protein, 247 aa, synthetic construct
QSVLTQPPSVSGAPGQRVTISCSGSRSNIGSNTVKWYQQLPGTAPKLLIYYNDQEPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 163 - protein, 247 aa, synthetic construct
QSVLTQPPSVSGAPGQRVTISCSGSRSNIGSNTVKWYQQLPGTAPKLLIYYNDQRPDGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 164 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDEYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 165 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTDPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 166 - protein, 247 aa, synthetic construct
QSVLTQPPSVSGAPGQRVTISCSGSESNIGSNTVKWYQQLPGTAPKLLIYYNDQEPSGVPDRFSGSKSGTSASLAITGLQAEDEADYYCQSYDEYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 167 - protein, 247 aa, synthetic construct
QSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 168 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFESYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 169 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSEYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 170 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSDYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 171 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVKGRFTISRDNSKNTLYLQMNSLRAEDTAVYYCKTHGSHDNWGQGTMVTVSSHHHHHH

SEQ ID NO: 172 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIDYDGSNKYYADSVKGRFTISRDNSKNTLYLQMNSLRAEDTAVYYCKTHGSHDNWGQGTMVTVSSHHHHHH

SEQ ID NO: 173 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNDYYADSVKGRFTISRDNSKNTLYLQMNSLRAEDTAVYYCKTHGSHDNWGQGTMVTVSSHHHHHH

SEQ ID NO: 174 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNEYYADSVKGRFTISRDNSKNTLYLQMNSLRAEDTAVYYCKTHGSHDNWGQGTMVTVSSHHHHHH

SEQ ID NO: 175 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVEGRFTISRDNSKNTLYLQMNSLRAEDTAVYYCKTHGSHDNWGQGTMVTVSSHHHHHH

SEQ ID NO: 176 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSEDNWGQGTMVTVSSHHHHHH

SEQ ID NO: 177 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVEGRFTISRDNSKNTLYLQMNSLRAEDTAVYYCKTHGSHDNWGQGTMVTVSSHHHHHH

SEQ ID NO: 178 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVEGRFTISRDNSKNTLYLQMNSLRAEDTAVYYCKTHGSEDNWGQGTMVTVSSHHHHHH

SEQ ID NO: 179 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 180 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 181 - protein, 247 aa, synthetic construct
QSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 182 - protein, 247 aa, synthetic construct
QSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 183 - protein, 247 aa, synthetic construct
QSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 184 - protein, 247 aa, synthetic construct
QSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 185 - protein, 247 aa, synthetic construct; identical to SEQ ID NO: 167

SEQ ID NO: 186 - protein, 247 aa, synthetic construct
QSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 187 - protein, 247 aa, synthetic construct
QSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 188 - protein, 247 aa, synthetic construct
QSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSSHHHHHH

SEQ ID NO: 189 - protein, 218 aa, synthetic construct; identical to SEQ ID NO: 92

SEQ ID NO: 190 - protein, 218 aa, synthetic construct; identical to SEQ ID NO: 93

SEQ ID NO: 191 - protein, 218 aa, synthetic construct; identical to SEQ ID NO: 94

SEQ ID NO: 192 - protein, 217 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 193 - protein, 217 aa, synthetic construct
QSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 194 - protein, 224 aa, synthetic construct
QVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCHHHHHH

SEQ ID NO: 195 - protein, 8 aa, synthetic construct
GPAGLYAQ

SEQ ID NO: 196 - protein, 8 aa, synthetic construct
GPAGMKGL

SEQ ID NO: 197 - protein, 8 aa, synthetic construct
PGGPAGIG

SEQ ID NO: 198 - protein, 8 aa, synthetic construct
ALFKSSFP

SEQ ID NO: 199 - protein, 8 aa, synthetic construct
ALFFSSPP

SEQ ID NO: 200 - protein, 8 aa, synthetic construct
LAQRLRSS

SEQ ID NO: 201 - protein, 8 aa, synthetic construct
LAQKLKSS

SEQ ID NO: 202 - protein, 30 aa, synthetic construct
GALFKSSFPSGGGPAGLYAQGGSGKGGSGK

SEQ ID NO: 203 - protein, 30 aa, synthetic construct
RGSGGGPAGLYAQGSGGGPAGLYAQGGSGK

SEQ ID NO: 204 - protein, 30 aa, synthetic construct
KGGGPAGLYAQGPAGLYAQGPAGLYAQGSR

SEQ ID NO: 205 - protein, 30 aa, synthetic construct
RGGPAGLYAQGGPAGLYAQGGGPAGLYAQK

SEQ ID NO: 206 - protein, 30 aa, synthetic construct
KGGALFKSSFPGGPAGIGPLAQKLKSSGGS

SEQ ID NO: 207 - protein, 30 aa, synthetic construct
SGGPGGPAGIGALFKSSFPLAQKLKSSGGG

SEQ ID NO: 208 - protein, 30 aa, synthetic construct
RGPLAQKLKSSALFKSSFPGGPAGIGGGGK

SEQ ID NO: 209 - protein, 30 aa, synthetic construct
GGGALFKSSFPLAQKLKSSPGGPAGIGGGR

SEQ ID NO: 210 - protein, 30 aa, synthetic construct
RGPGGPAGIGPLAQKLKSSALFKSSFPGGG

SEQ ID NO: 211 - protein, 30 aa, synthetic construct
RGGPLAQKLKSSPGGPAGIGALFKSSFPGK

SEQ ID NO: 212 - protein, 30 aa, synthetic construct
RSGGPAGLYAQALFKSSFPLAQKLKSSGGG

SEQ ID NO: 213 - protein, 30 aa, synthetic construct
GGPLAQKLKSSALFKSSFPGPAGLYAQGGR

SEQ ID NO: 214 - protein, 30 aa, synthetic construct
GGALFKSSFPGPAGLYAQPLAQKLKSSGGK

SEQ ID NO: 215 - protein, 30 aa, synthetic construct
RGGALFKSSFPLAQKLKSSGPAGLYAQGGK

SEQ ID NO: 216 - protein, 30 aa, synthetic construct
RGGGPAGLYAQPLAQKLKSSALFKSSFPGG

SEQ ID NO: 217 - protein, 30 aa, synthetic construct
SGPLAQKLKSSGPAGLYAQALFKSSFPGSK

SEQ ID NO: 218 - protein, 30 aa, synthetic construct
KGGPGGPAGIGPLAQRLRSSALFKSSFPGR

SEQ ID NO: 219 - protein, 30 aa, synthetic construct
KSGPGGPAGIGALFFSSPPLAQKLKSSGGR

SEQ ID NO: 220 - protein, 30 aa, synthetic construct
SGGFPRSGGSFNPRTFGSKRKRRGSRGGGG

SEQ ID NO: 221 - protein, 8 aa, synthetic construct
GPLGLKAQ

SEQ ID NO: 222 - protein, 8 aa, synthetic construct
LPLGLKAQ

SEQ ID NO: 223 - protein, 8 aa, synthetic construct
SPLGLKAQ

SEQ ID NO: 224 - protein, 8 aa, synthetic construct
QPLGLKAQ

SEQ ID NO: 225 - protein, 8 aa, synthetic construct
KPLGLKAQ

SEQ ID NO: 226 - protein, 8 aa, synthetic construct
FPLGLKAQ

SEQ ID NO: 227 - protein, 8 aa, synthetic construct
HPLGLKAQ

SEQ ID NO: 228 - protein, 8 aa, synthetic construct
PPLGLKAQ

SEQ ID NO: 229 - protein, 8 aa, synthetic construct
APLGLKAQ

SEQ ID NO: 230 - protein, 8 aa, synthetic construct
DPLGLKAQ

SEQ ID NO: 231 - protein, 8 aa, synthetic construct
GPHGLKAQ

SEQ ID NO: 232 - protein, 8 aa, synthetic construct
GPSGLKAQ

SEQ ID NO: 233 - protein, 8 aa, synthetic construct
GPQGLKAQ

SEQ ID NO: 234 - protein, 8 aa, synthetic construct
GPPGLKAQ

SEQ ID NO: 235 - protein, 8 aa, synthetic construct
GPEGLKAQ

SEQ ID NO: 236 - protein, 8 aa, synthetic construct
GPFGLKAQ

SEQ ID NO: 237 - protein, 8 aa, synthetic construct
GPRGLKAQ

SEQ ID NO: 238 - protein, 8 aa, synthetic construct
GPGGLKAQ

SEQ ID NO: 239 - protein, 8 aa, synthetic construct
GPAGLKAQ

SEQ ID NO: 240 - protein, 8 aa, synthetic construct
LPAGLKGA

SEQ ID NO: 241 - protein, 8 aa, synthetic construct
GPAGLYAQ

SEQ ID NO: 242 - protein, 8 aa, synthetic construct
GPANLVAQ

SEQ ID NO: 243 - protein, 8 aa, synthetic construct
GPAALVGA

SEQ ID NO: 244 - protein, 8 aa, synthetic construct
GPANLRAQ

SEQ ID NO: 245 - protein, 8 aa, synthetic construct
GPAGLRAQ

SEQ ID NO: 246 - protein, 8 aa, synthetic construct
GPAGLVAQ

SEQ ID NO: 247 - protein, 8 aa, synthetic construct
GPAGLRGA

SEQ ID NO: 248 - protein, 8 aa, synthetic construct
LPAGLVGA

SEQ ID NO: 249 - protein, 8 aa, synthetic construct
GPAGLKGA

SEQ ID NO: 250 - protein, 8 aa, synthetic construct
GPLALKAQ

SEQ ID NO: 251 - protein, 8 aa, synthetic construct
GPLNLKAQ

SEQ ID NO: 252 - protein, 8 aa, synthetic construct
GPLHLKAQ

SEQ ID NO: 253 - protein, 8 aa, synthetic construct
GPLYLKAQ

SEQ ID NO: 254 - protein, 8 aa, synthetic construct
GPLPLKAQ

SEQ ID NO: 255 - protein, 8 aa, synthetic construct
GPLELKAQ

SEQ ID NO: 256 - protein, 8 aa, synthetic construct
GPLRLKAQ

SEQ ID NO: 257 - protein, 8 aa, synthetic construct
GPLLLKAQ

SEQ ID NO: 258 - protein, 8 aa, synthetic construct
GPLSLKAQ

SEQ ID NO: 259 - protein, 8 aa, synthetic construct
GPLGLYAQ

SEQ ID NO: 260 - protein, 8 aa, synthetic construct
GPLGLFAQ

SEQ ID NO: 261 - protein, 8 aa, synthetic construct
GPLGLLAQ

SEQ ID NO: 262 - protein, 8 aa, synthetic construct
GPLGLHAQ

SEQ ID NO: 263 - protein, 8 aa, synthetic construct
GPLGLRAQ

SEQ ID NO: 264 - protein, 8 aa, synthetic construct
GPLGLAAQ

SEQ ID NO: 265 - protein, 8 aa, synthetic construct
GPLGLEAQ

SEQ ID NO: 266 - protein, 8 aa, synthetic construct
GPLGLGAQ

SEQ ID NO: 267 - protein, 8 aa, synthetic construct
GPLGLPAQ

SEQ ID NO: 268 - protein, 8 aa, synthetic construct
GPLGLQAQ

SEQ ID NO: 269 - protein, 8 aa, synthetic construct
GPLGLSAQ

SEQ ID NO: 270 - protein, 8 aa, synthetic construct
GPLGLVAQ

SEQ ID NO: 271 - protein, 8 aa, synthetic construct
GPLGLKLQ

SEQ ID NO: 272 - protein, 8 aa, synthetic construct
GPLGLKFQ

SEQ ID NO: 273 - protein, 8 aa, synthetic construct
GPLGLKEQ

SEQ ID NO: 274 - protein, 8 aa, synthetic construct
GPLGLKKQ

SEQ ID NO: 275 - protein, 8 aa, synthetic construct
GPLGLKQQ

SEQ ID NO: 276 - protein, 8 aa, synthetic construct
GPLGLKSQ

SEQ ID NO: 277 - protein, 8 aa, synthetic construct
GPLGLKGQ

SEQ ID NO: 278 - protein, 8 aa, synthetic construct
GPLGLKHQ

SEQ ID NO: 279 - protein, 8 aa, synthetic construct
GPLGLKPQ

SEQ ID NO: 280 - protein, 8 aa, synthetic construct
GPLGLKAG

SEQ ID NO: 281 - protein, 8 aa, synthetic construct
GPLGLKAF

SEQ ID NO: 282 - protein, 8 aa, synthetic construct
GPLGLKAP

SEQ ID NO: 283 - protein, 8 aa, synthetic construct
GPLGLKAL

SEQ ID NO: 284 - protein, 8 aa, synthetic construct
GPLGLKAE

SEQ ID NO: 285 - protein, 8 aa, synthetic construct
GPLGLKAA

SEQ ID NO: 286 - protein, 8 aa, synthetic construct
GPLGLKAH

SEQ ID NO: 287 - protein, 8 aa, synthetic construct
GPLGLKAK

SEQ ID NO: 288 - protein, 8 aa, synthetic construct
GPLGLKAS

SEQ ID NO: 289 - protein, 8 aa, synthetic construct
GPLGLFGA

SEQ ID NO: 290 - protein, 8 aa, synthetic construct
GPLGLQGA

SEQ ID NO: 291 - protein, 8 aa, synthetic construct
GPLGLVGA

SEQ ID NO: 292 - protein, 8 aa, synthetic construct
GPLGLAGA

SEQ ID NO: 293 - protein, 8 aa, synthetic construct
GPLGLLGA

SEQ ID NO: 294 - protein, 8 aa, synthetic construct
GPLGLRGA

SEQ ID NO: 295 - protein, 8 aa, synthetic construct
GPLGLYGA

SEQ ID NO: 296 - protein, 8 aa, synthetic construct
ALFKSSPP

SEQ ID NO: 297 - protein, 8 aa, synthetic construct
SPFRSSRQ

SEQ ID NO: 298 - protein, 8 aa, synthetic construct
KLFKSSPP

SEQ ID NO: 299 - protein, 8 aa, synthetic construct
HLFKSSPP

SEQ ID NO: 300 - protein, 8 aa, synthetic construct
SLFKSSPP

SEQ ID NO: 301 - protein, 8 aa, synthetic construct
QLFKSSPP

SEQ ID NO: 302 - protein, 8 aa, synthetic construct
LLFKSSPP

SEQ ID NO: 303 - protein, 8 aa, synthetic construct
PLFKSSPP

SEQ ID NO: 304 - protein, 8 aa, synthetic construct
FLFKSSPP

SEQ ID NO: 305 - protein, 8 aa, synthetic construct
GLFKSSPP

SEQ ID NO: 306 - protein, 8 aa, synthetic construct
VLFKSSPP

SEQ ID NO: 307 - protein, 8 aa, synthetic construct
ELFKSSPP

SEQ ID NO: 308 - protein, 8 aa, synthetic construct
AKFKSSPP

SEQ ID NO: 309 - protein, 8 aa, synthetic construct
AHFKSSPP

SEQ ID NO: 310 - protein, 8 aa, synthetic construct
AGFKSSPP

SEQ ID NO: 311 - protein, 8 aa, synthetic construct
APFKSSPP

SEQ ID NO: 312 - protein, 8 aa, synthetic construct
ANFKSSPP

SEQ ID NO: 313 - protein, 8 aa, synthetic construct
AFFKSSPP

SEQ ID NO: 314 - protein, 8 aa, synthetic construct
AAFKSSPP

SEQ ID NO: 315 - protein, 8 aa, synthetic construct
ASFKSSPP

SEQ ID NO: 316 - protein, 8 aa, synthetic construct
AEFKSSPP

SEQ ID NO: 317 - protein, 8 aa, synthetic construct
ALRKSSPP

SEQ ID NO: 318 - protein, 8 aa, synthetic construct
ALLKSSPP

SEQ ID NO: 319 - protein, 8 aa, synthetic construct
ALAKSSPP

SEQ ID NO: 320 - protein, 8 aa, synthetic construct
ALQKSSPP

SEQ ID NO: 321 - protein, 8 aa, synthetic construct
ALHKSSPP

SEQ ID NO: 322 - protein, 8 aa, synthetic construct
ALPKSSPP

SEQ ID NO: 323 - protein, 8 aa, synthetic construct
ALTKSSPP

SEQ ID NO: 324 - protein, 8 aa, synthetic construct
ALGKSSPP

SEQ ID NO: 325 - protein, 8 aa, synthetic construct
ALDKSSPP

SEQ ID NO: 326 - protein, 8 aa, synthetic construct
ALFFSSPP

SEQ ID NO: 327 - protein, 8 aa, synthetic construct
ALFHSSPP

SEQ ID NO: 328 - protein, 8 aa, synthetic construct
ALFTSSPP

SEQ ID NO: 329 - protein, 8 aa, synthetic construct
ALFASSPP

SEQ ID NO: 330 - protein, 8 aa, synthetic construct
ALFQSSPP

SEQ ID NO: 331 - protein, 8 aa, synthetic construct
ALFLSSPP

SEQ ID NO: 332 - protein, 8 aa, synthetic construct
ALFGSSPP

SEQ ID NO: 333 - protein, 8 aa, synthetic construct
ALFESSPP

SEQ ID NO: 334 - protein, 8 aa, synthetic construct
ALFPSSPP

SEQ ID NO: 335 - protein, 8 aa, synthetic construct
ALFKHSPP

SEQ ID NO: 336 - protein, 8 aa, synthetic construct
ALFKLSPP

SEQ ID NO: 337 - protein, 8 aa, synthetic construct
ALFKKSPP

SEQ ID NO: 338 - protein, 8 aa, synthetic construct
ALFKASPP

SEQ ID NO: 339 - protein, 8 aa, synthetic construct
ALFKISPP

SEQ ID NO: 340 - protein, 8 aa, synthetic construct
ALFKGSPP

SEQ ID NO: 341 - protein, 8 aa, synthetic construct
ALFKNSPP

SEQ ID NO: 342 - protein, 8 aa, synthetic construct
ALFKRSPP

SEQ ID NO: 343 - protein, 8 aa, synthetic construct
ALFKESPP

SEQ ID NO: 344 - protein, 8 aa, synthetic construct
ALFKFSPP

SEQ ID NO: 345 - protein, 8 aa, synthetic construct
ALFKPSPP

SEQ ID NO: 346 - protein, 8 aa, synthetic construct
ALFKSFPP

SEQ ID NO: 347 - protein, 8 aa, synthetic construct
ALFKSLPP

SEQ ID NO: 348 - protein, 8 aa, synthetic construct
ALFKSIPP

SEQ ID NO: 349 - protein, 8 aa, synthetic construct
ALFKSKPP

SEQ ID NO: 350 - protein, 8 aa, synthetic construct
ALFKSAPP

SEQ ID NO: 351 - protein, 8 aa, synthetic construct
ALFKSQPP

SEQ ID NO: 352 - protein, 8 aa, synthetic construct
ALFKSPPP

SEQ ID NO: 353 - protein, 8 aa, synthetic construct
ALFKSEPP

SEQ ID NO: 354 - protein, 8 aa, synthetic construct
ALFKSGPP

SEQ ID NO: 355 - protein, 8 aa, synthetic construct
ALFKSSFP

SEQ ID NO: 356 - protein, 8 aa, synthetic construct
ALFKSSLP

SEQ ID NO: 357 - protein, 8 aa, synthetic construct
ALFKSSGP

SEQ ID NO: 358 - protein, 8 aa, synthetic construct
ALFKSSSP

SEQ ID NO: 359 - protein, 8 aa, synthetic construct
ALFKSSVP

SEQ ID NO: 360 - protein, 8 aa, synthetic construct
ALFKSSHP

SEQ ID NO: 361 - protein, 8 aa, synthetic construct
ALFKSSAP

SEQ ID NO: 362 - protein, 8 aa, synthetic construct
ALFKSSNP

SEQ ID NO: 363 - protein, 8 aa, synthetic construct
ALFKSSKP

SEQ ID NO: 364 - protein, 8 aa, synthetic construct
ALFKSSEP

SEQ ID NO: 365 - protein, 8 aa, synthetic construct
ALFKSSPF

SEQ ID NO: 366 - protein, 8 aa, synthetic construct
ALFKSSPH

SEQ ID NO: 367 - protein, 8 aa, synthetic construct
ALFKSSPG

SEQ ID NO: 368 - protein, 8 aa, synthetic construct
ALFKSSPA

SEQ ID NO: 369 - protein, 8 aa, synthetic construct
ALFKSSPS

SEQ ID NO: 370 - protein, 8 aa, synthetic construct
ALFKSSPV

SEQ ID NO: 371 - protein, 8 aa, synthetic construct
ALFKSSPQ

SEQ ID NO: 372 - protein, 8 aa, synthetic construct
ALFKSSPK

SEQ ID NO: 373 - protein, 8 aa, synthetic construct
ALFKSSPL

SEQ ID NO: 374 - protein, 8 aa, synthetic construct
ALFKSSPD

SEQ ID NO: 375 - protein, 8 aa, synthetic construct
KRALGLPG

SEQ ID NO: 376 - protein, 40 aa, synthetic construct
DEDEDEDEDEDEDEDERPLALWRSDRDRDRDRDRDRDRDR

SEQ ID NO: 377 - skipped

SEQ ID NO: 378 - protein, 5 aa, synthetic construct
LEATA

SEQ ID NO: 379 - protein, 10 aa, synthetic construct
GGAANLVRGG

SEQ ID NO: 380 - protein, 10 aa, synthetic construct
SGRIGFLRTA

SEQ ID NO: 381 - protein, 6 aa, synthetic construct
PLGLAG

SEQ ID NO: 382 - protein, 6 aa, synthetic construct
PLGLAX

SEQ ID NO: 383 - protein, 6 aa, synthetic construct
  MOD_RES 4: note = Cysteine(Me)
PLGCAG

SEQ ID NO: 384 - protein, 8 aa, synthetic construct
ESPAYYTA

SEQ ID NO: 385 - protein, 6 aa, synthetic construct
RLQLKL

SEQ ID NO: 386 - protein, 7 aa, synthetic construct
RLQLKAC

SEQ ID NO: 387 - protein, 7 aa, synthetic construct
  MOD_RES 3: note = Citrulline
  MOD_RES 5: note = Homo-Phenylalanine
EPXGXYL

SEQ ID NO: 388 - protein, 5 aa, synthetic construct
SGRSA

SEQ ID NO: 389 - protein, 4 aa, synthetic construct
DAFK

SEQ ID NO: 390 - protein, 5 aa, synthetic construct
GGGRR

SEQ ID NO: 391 - protein, 4 aa, synthetic construct
GFLG

SEQ ID NO: 392 - protein, 4 aa, synthetic construct
ALAL

SEQ ID NO: 393-394 - skipped (2 entries)

SEQ ID NO: 395 - protein, 5 aa, synthetic construct
  MOD_RES 3: note = Cysteine(Et)
PICFF

SEQ ID NO: 396 - protein, 8 aa, synthetic construct
GGPRGLPG

SEQ ID NO: 397 - protein, 6 aa, synthetic construct
HSSKLQ

SEQ ID NO: 398 - protein, 7 aa, synthetic construct
HSSKLQL

SEQ ID NO: 399 - protein, 9 aa, synthetic construct
HSSKLQEDA

SEQ ID NO: 400 - protein, 10 aa, synthetic construct
LVLASSSFGY

SEQ ID NO: 401 - protein, 10 aa, synthetic construct
GVSQNYPIVG

SEQ ID NO: 402 - protein, 10 aa, synthetic construct
GVVQASCRLA

SEQ ID NO: 403 - skipped

SEQ ID NO: 404 - protein, 6 aa, synthetic construct
DPRSFL

SEQ ID NO: 405 - protein, 6 aa, synthetic construct
PPRSFL

SEQ ID NO: 406 - protein, 4 aa, synthetic construct
DEVD

SEQ ID NO: 407 - protein, 5 aa, synthetic construct
DEVDP

SEQ ID NO: 408 - protein, 8 aa, synthetic construct
KGSGDVEG

SEQ ID NO: 409 - protein, 6 aa, synthetic construct
GWEHDG

SEQ ID NO: 410 - protein, 7 aa, synthetic construct
EDDDDKA

SEQ ID NO: 411 - protein, 9 aa, synthetic construct
KQEQNPGST

SEQ ID NO: 412 - protein, 6 aa, synthetic construct
GKAFRR

SEQ ID NO: 413 - protein, 4 aa, synthetic construct
DAFK

SEQ ID NO: 414 - protein, 4 aa, synthetic construct
DVLK

SEQ ID NO: 415 - protein, 4 aa, synthetic construct
DAFK

SEQ ID NO: 416 - protein, 7 aa, synthetic construct
ALLLALL

SEQ ID NO: 417 - protein, 7 aa, synthetic construct
GPLGVRG

SEQ ID NO: 418 - protein, 8 aa, synthetic construct
IPVSLRSG

SEQ ID NO: 419 - protein, 8 aa, synthetic construct
VPLSLYSG

SEQ ID NO: 420 - protein, 10 aa, synthetic construct
SGESPAYYTA

SEQ ID NO: 421 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 422 - protein, 638 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSGGGGSVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 423 - protein, 181 aa, synthetic construct
VPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTAHHHHHH

SEQ ID NO: 424 - protein, 176 aa, synthetic construct
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPHHHHHH

SEQ ID NO: 425 - protein, 305 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGGGGSGGGPGSVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 426 - protein, 300 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGGGGSGGGPGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 427 - protein, 591 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 428 - protein, 586 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 429 - protein, 653 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 430 - protein, 648 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 431 - protein, 939 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 432 - protein, 934 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 433 - protein, 313 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 434 - protein, 306 aa, synthetic construct; identical to SEQ ID NO: 18

SEQ ID NO: 435 - protein, 306 aa, synthetic construct; identical to SEQ ID NO: 18

SEQ ID NO: 436 - protein, 313 aa, synthetic construct; identical to SEQ ID NO: 433

SEQ ID NO: 437 - protein, 306 aa, synthetic construct; identical to SEQ ID NO: 18

SEQ ID NO: 438 - protein, 306 aa, synthetic construct; identical to SEQ ID NO: 18

SEQ ID NO: 439 - protein, 306 aa, synthetic construct; identical to SEQ ID NO: 18

SEQ ID NO: 440 - protein, 306 aa, synthetic construct; identical to SEQ ID NO: 18

SEQ ID NO: 441 - protein, 613 aa, synthetic construct; identical to SEQ ID NO: 104

SEQ ID NO: 442 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 443 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCS

SEQ ID NO: 444 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 445 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 446 - protein, 6 aa, synthetic construct
HHHHHH

SEQ ID NO: 447 - protein, 7 aa, synthetic construct
PAGLYAQ

SEQ ID NO: 448 - protein, 7 aa, synthetic construct
LFKSSFP